(19) United States
(12) Patent Application Publication
(10) Pub. No.: US 2025/0345458 A1
(43) Pub. Date: Nov. 13, 2025

(54) COMPLEXING AGENT SALT FORMULATIONS OF PHARMACEUTICAL COMPOUNDS AT LOW STOICHIOMETRIC RATIOS

(71) Applicant: Bexson Biomedical, Inc., Santa Barbara, CA (US)

(72) Inventors: Jeffrey BECKER, Santa Barbara, CA (US); Jason WALLACH, Lansdowne, PA (US); Gregg PETERSON, Santa Barbara, CA (US)

(21) Appl. No.: 18/866,494

(22) PCT Filed: May 17, 2023
(86) PCT No.: PCT/US2023/022578

§ 371 Date: Nov. 15, 2024

Related U.S. Application Data
(60) Provisional application No. 63/343,416, filed on May 18, 2022.

(51) Int. Cl. A61K 47/69; A61K 47/14

(52) U.S. Cl. CPC A61K 47/6951; A61K 47/14

(57) ABSTRACT

Provided herein are pharmaceutical formulations and pharmaceutical compound salts which utilize complexing agents as counterions. Such formulations and salts are useful for treating a variety of disease and disorders.

### CROSS-REFERENCE

This application claims the benefit of U.S. Provisional Patent Application No. 63/343,416 filed on May 18, 2022, which is incorporated herein by reference in its entirety.

### BACKGROUND

Pharmaceutical compounds and their derivatives, such as rotigotine, eletriptan, copanlisib, remdesivir, nafamostat (nafamostat mesylate), melevodopa, tigecycline, naloxone, amikacin, 1-amantadine, 2-amantadine, rimantadine, amifampridine, rapamycin, clonidine, or caspofungin, are useful for a variety of medicinal purposes. These compounds can be used to treat, for example, Parkinson's disease, migraine, cancer, viral infection, bacterial infection, autoimmune disease, inflammatory disease, opioid dependence, pain, or other disorders. However, the compounds may possess many physico-chemical properties that make suitable formulations for widespread use as pharmaceutical agents difficult, including the presence of basic amines, limited solubility, hydrophobicity, and inherently ionic functional groups.

### BRIEF SUMMARY OF THE INVENTION

In an aspect, provided herein is a pharmaceutical composition, comprising (i) a pharmaceutical compound, or an enantiomer, a mixture of enantiomers, or an isotopic variant thereof, wherein the pharmaceutical compound comprises a protonated nitrogen atom; and (ii) a complexing agent, wherein the complexing agent is an acid-substituted cyclodextrin comprising a plurality of acidic functional groups, wherein the plurality of acidic functional groups comprise an acidic group which acts as a counterion for the protonated nitrogen atom of the pharmaceutical compound, wherein the pharmaceutical composition has a molar ratio of the complexing agent to the pharmaceutical compound that is from about 1:1 to about 1:4. In some embodiments, the complexing agent comprises a substituted cyclodextrin. In some embodiments, the complexing agent comprises a cyclodextrin substituted with at least one acidic functional group. In some embodiments, the cyclodextrin is substituted with 3 to 8 acidic functional groups. In some embodiments, the cyclodextrin is sulfobutylether-β-cyclodextrin. In some embodiments, the pharmaceutical composition has lower osmolality than a composition comprising a salt of the pharmaceutical compound and a salt of the complexing agent. In some embodiments, the pharmaceutical is formulated for subcutaneous, intramuscular, sublingual, oral, rectal, transvaginal, or intranasal administration. In some embodiments, the pharmaceutical composition has an osmolality of no more than about 850 mOsm/kg. In some embodiments, the pharmaceutical composition has a pH of about 4 to about 7. In some embodiments, the complexing agent is present in an amount of about 10 mg/mL to about 600 mg/mL. In some embodiments, the complexing agent acts as the counterion to between 1 to 4 molecules of the pharmaceutical compound. In some embodiments, the complexing agent further comprises a non-polar pore. In some embodiments, the pharmaceutical composition further comprises an additional molar equivalent of the pharmaceutical compound, wherein the additional molar equivalent of the pharmaceutical compound is unionized and complexed to the non-polar pore. In some embodiments, the ratio of complexing agent to the pharmaceutical compound is about 1:1. In some embodiments, the ratio of complexing agent to the pharmaceutical compound is about 1:2. In some embodiments, the ratio of complexing agent to the pharmaceutical compound is about 1:3. In some embodiments, the ratio of complexing agent to the pharmaceutical compound is about 1:4. In some embodiments, the pharmaceutical compound has a solubility of less than about 50 mg/ml as salt in an aqueous medium. In some embodiments, the pharmaceutical compound has a solubility of less than about 10 mg/ml as salt in an aqueous medium. In some embodiments, the pharmaceutical compound has a solubility of less than about 5 mg/ml as salt in an aqueous medium. In some embodiments, the pharmaceutical compound has a solubility of less than about 0.5 mg/ml as salt in an aqueous medium. In some embodiments, the pharmaceutical compound has a solubility of less than about 0.1 mg/ml as salt in an aqueous medium. In some embodiments, a precipitate forms when an amount of the pharmaceutical compound is mixed with the pharmaceutical composition in an aqueous medium and becomes ionized after being mixed with the pharmaceutical composition. In some embodiments, the amount of the pharmaceutical compound being mixed with the pharmaceutical composition is about 1 molar equivalent compared to the complexing agent of the pharmaceutical composition. In some embodiments, the amount of the pharmaceutical compound being mixed with the pharmaceutical composition is about 2 molar equivalents compared to the complexing agent of the pharmaceutical composition. In some embodiments, the amount of the pharmaceutical compound being mixed with the pharmaceutical composition is about 3 molar equivalents compared to the complexing agent of the pharmaceutical composition. In some embodiments, the pharmaceutical compound comprises rotigotine, eletriptan, copanlisib, nafamostat (nafamostat mesylate), melevodopa, tigecycline, naloxone, amikacin, 1-amantadine, 2-amantadine, rimantadine, amifampridine, rapamycin, clonidine, or caspofungin.

In another aspect, provided herein is a pharmaceutically acceptable salt of a compound pharmaceutical comprising: (i) a pharmaceutical compound, or an enantiomer, a mixture of enantiomers, or an isotopic variant thereof; or a pharmaceutically acceptable salt, solvate or hydrate thereof, wherein the pharmaceutical compound comprises a protonated nitrogen atom; and (ii) a conjugate base of a complexing agent comprising a plurality of acidic functional groups, wherein at least one acidic functional group of the plurality of acidic functional groups acts as a counterion of the pharmaceutical compound, wherein the molar ratio of the conjugate base of the complexing agent to the pharmaceutical compound is from about 1:1 to about 1:4. In some embodiments, the salt is in a crystalline form, an amorphous form, a lyophilized powder, dissolved or suspended in an aqueous medium, or dissolved or suspended in an organic solvent. In some embodiments, the complexing agent comprises a substituted cyclodextrin. In some embodiments, the pharmaceutical compound comprises rotigotine, eletriptan, copanlisib, nafamostat (nafamostat mesylate), melevodopa, tigecycline, naloxone, amikacin, 1-amantadine, 2-amantadine, rimantadine, amifampridine, rapamycin, clonidine, or caspofungin. In some embodiments, the conjugate base of the complexing agent further comprises a non-polar pore. In some embodiments, the pharmaceutically acceptable salt further comprises an additional molar equivalent of the pharmaceutical compound compared to the conjugate base of the complexing agent, wherein the additional molar equivalent of the pharmaceutical compound is unionized and complexed to the non-polar pore.

In another aspect, provided herein is a pharmaceutically acceptable salt of a pharmaceutical compound having the formula:

[A]ₐ[B]

wherein:
A is a pharmaceutical compound comprising at least one basic nitrogen atom;
B is a complexing agent comprising a plurality of acidic functional groups; and
a is a number from 1-4, wherein the number is selected such that a portion, but not all, of the acidic functional groups of B act as a counterion to the basic nitrogen atom of A. In some embodiments, the at least one basic nitrogen atom is comprised in a heterocycle. In some embodiments, the pharmaceutical compound comprises only a single basic nitrogen atom. In some embodiments, a is equal to 1, 2, 3, or 4. In some embodiments, the pharmaceutical compound comprises two or more basic nitrogen atoms. In some embodiments, the complexing agent is a cyclodextrin. In some embodiments, the complexing agent is a compound of Formula (I):

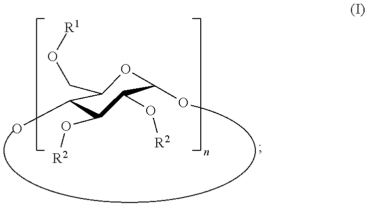

wherein:
each R1 is independently H or optionally substituted alkyl; wherein at least one R1 is substituted with an acidic functional group;
each R2 is independently H or optionally substituted alkyl; and
n is 6, 7, or 8;
   or a stereoisomer, a mixture of stereoisomers, or an isotopic variant thereof; or a pharmaceutically acceptable salt, solvate or hydrate thereof. In some embodiments, the complexing agent is SBEBCD. In some embodiments, the pharmaceutical compound is rotigotine, eletriptan, copanlisib, nafamostat (nafamostat mesylate), melevodopa, tigecycline, naloxone, amikacin, 1-amantadine, 2-amantadine, rimantadine, amifampridine, rapamycin, clonidine, or caspofungin. In some embodiments, the complexing agent further comprises a non-polar pore. In some embodiments, the pharmaceutically acceptable salt further comprises an additional molar equivalent of the pharmaceutical compound compared to the complexing agent, wherein the additional molar equivalent of the pharmaceutical compound is unionized and complexed to the non-polar pore.

In another aspect, provided herein is a pharmaceutical composition, comprising: (i) a pharmaceutical compound, or an enantiomer, a mixture of enantiomers, or an isotopic variant thereof, wherein the pharmaceutical compound comprises a protonated nitrogen atom; (ii) a complexing agent, wherein the complexing agent comprises a plurality of acidic functional groups, wherein the plurality of acidic functional groups comprise a conjugate base of an acid which acts as a counterion for the protonated nitrogen atom of the pharmaceutical compound, wherein the pharmaceutical composition has a molar ratio of the complexing agent to the pharmaceutical compound that is from about 1:1 to about 1:4; and (iii) an additional molar equivalent of the pharmaceutical compound, wherein the additional molar equivalent of the pharmaceutical compound is unionized. In some embodiments, the pharmaceutical compound comprises rotigotine, eletriptan, copanlisib, nafamostat (nafamostat mesylate), melevodopa, tigecycline, naloxone, amikacin, 1-amantadine, 2-amantadine, rimantadine, amifampridine, rapamycin, clonidine, or caspofungin. In some embodiments, the complexing agent comprises a substituted cyclodextrin. In some embodiments, the complexing agent comprises a cyclodextrin substituted with at least one acidic functional group. In some embodiments, the cyclodextrin is substituted with 3 to 8 acidic functional groups. In some embodiments, the cyclodextrin is sulfobutylether-β-cyclodextrin. In some embodiments, the pharmaceutical composition has lower osmolality than a composition comprising a salt of the pharmaceutical compound and a salt of the complexing agent. In some embodiments, the pharmaceutical is formulated for subcutaneous, intramuscular, sublingual, oral, rectal, transvaginal, or intranasal administration. In some embodiments, the pharmaceutical composition has an osmolality of no more than about 850 mOsm/kg. In some embodiments, the pharmaceutical composition has a pH of about 4 to about 7. In some embodiments, the complexing agent is present in an amount of about 10 mg/mL to about 600 mg/mL. In some embodiments, the complexing agent acts as the counterion to between 1 to 4 molecules of the pharmaceutical compound. In some embodiments, the complexing agent further comprises a non-polar pore. In some embodiments, the additional molar equivalent of the unionized pharmaceutical compound is complexed to the non-polar pore. In some embodiments, the molar ratio of complexing agent to the pharmaceutical compound is about 1:1. In some embodiments, the molar ratio of complexing agent to the pharmaceutical compound is about 1:2. In some embodiments, the molar ratio of complexing agent to the pharmaceutical compound is about 1:3. In some embodiments, the molar ratio of complexing agent to the pharmaceutical compound is about 1:4. In some embodiments, the pharmaceutical compound has a solubility of less than about 50 mg/ml as salt in an aqueous medium. In some embodiments, the pharmaceutical compound has a solubility of less than about 10 mg/ml as salt in an aqueous medium. In some embodiments, the pharmaceutical compound has a solubility of less than about 5 mg/ml as salt in an aqueous medium. In some embodiments, the pharmaceutical compound has a solubility of less than about 0.5 mg/ml as salt in an aqueous medium. In some embodiments, the pharmaceutical compound has a solubility of less than about 0.1 mg/ml as salt in an aqueous medium. In some embodiments, a precipitate forms when an amount of the pharmaceutical compound is mixed with the pharmaceutical composition in an aqueous medium and becomes ionized after being mixed with the pharmaceutical composition. In some embodiments, the amount of the pharmaceutical compound being mixed with the pharmaceutical composition is about 1 molar equivalent compared to the complexing agent of the pharmaceutical composition. In some embodiments, the amount of the pharmaceutical compound being mixed with the pharmaceutical composition is about 2 molar equivalents compared to the complexing agent of the pharmaceutical composition. In some embodiments, the amount of the pharmaceutical compound being mixed with the pharmaceutical composition is about 3 molar equivalents compared to the complexing agent of the pharmaceutical composition.

In another aspect, provided herein is a method of preparing a pharmaceutical composition, comprising combining in a suitable liquid medium: a) a free base form of a pharmaceutical compound, or an enantiomer, a mixture of enantiomers, or an isotopic variant thereof, wherein the pharmaceutical compound comprises at least one basic nitrogen atom; and b) a free acid form of a complexing agent comprising at least one acidic functional group, wherein the molar ratio of the complexing agent to the pharmaceutical compound is from about 1:1 to about 1:4. In some embodiments, the method further comprises the step of adding an additional molar equivalent of the free base form of the pharmaceutical compound to the suitable liquid medium. In some embodiments, the adding the additional molar equivalent of the free base form of the pharmaceutical compound occurs after removing the liquid medium from the pharmaceutical composition. In some embodiments, the additional molar equivalent of the free base form of the pharmaceutical compound is unionized after being added. In some embodiments, a precipitate forms after the additional molar equivalent of the free base form of the pharmaceutical compound is added and becomes ionized. In some embodiments, the additional molar equivalent of the free base form of the pharmaceutical compound is about 1 molar equivalent compared to the complexing agent. In some embodiments, the additional molar equivalent of the free base form of the pharmaceutical compound is about 2 molar equivalents compared to the complexing agent. In some embodiments, the additional molar equivalent of the free base form of the pharmaceutical compound is about 3 molar equivalents compared to the complexing agent. In some embodiments, the pharmaceutical compound has a solubility of less than about 50 mg/ml as salt in an aqueous medium. In some embodiments, the pharmaceutical compound has a solubility of less than about 10 mg/ml as salt in an aqueous medium. In some embodiments, the pharmaceutical compound has a solubility of less than about 5 mg/ml as salt in an aqueous medium. In some embodiments, the pharmaceutical compound has a solubility of less than about 0.5 mg/ml as salt in an aqueous medium. In some embodiments, the pharmaceutical compound has a solubility of less than about 0.1 mg/ml as salt in an aqueous medium. In some embodiments, the complexing agent is sulfobutylether-β-cyclodextrin. In some embodiments, the method further comprises subjecting the pharmaceutical composition to an ion exchange process to generate a conjugate acid form of the complexing agent. In some embodiments, the ion exchange process comprises a resin ion exchange process.

In another aspect, provided herein is a pharmaceutical composition, comprising: (i) a pharmaceutical compound, or an enantiomer, a mixture of enantiomers, or an isotopic variant thereof, wherein the pharmaceutical compound comprises a prodrug comprising an unionized substance conjugated to a chemical entity, wherein the chemical entity comprises a protonated nitrogen atom; and (ii) a complexing agent, wherein the complexing agent is an acid-substituted cyclodextrin comprising a plurality of acidic functional groups, wherein the plurality of acidic functional groups comprise an acidic group which acts as a counterion for the protonated nitrogen atom of the pharmaceutical compound. In some embodiments, the complexing agent comprises a substituted cyclodextrin. In some embodiments, the complexing agent comprises a cyclodextrin substituted with at least one acidic functional group. In some embodiments, the cyclodextrin is substituted with 3 to 8 acidic functional groups. In some embodiments, the cyclodextrin is sulfobutylether-β-cyclodextrin. In some embodiments, the pharmaceutical composition has lower osmolality than (i) a composition comprising a salt of the pharmaceutical compound; or (ii) a composition comprising the pharmaceutical compound in freebase form that is complexing to a non-polar pore of the complexing agent, wherein the pharmaceutical compound has the same concentration in the pharmaceutical composition, the composition comprising the salt of the pharmaceutical compound, and the composition comprising the pharmaceutical compound in freebase form. In some embodiments, the pharmaceutical composition is formulated for subcutaneous, intramuscular, sublingual, oral, rectal, transvaginal, or intranasal administration. In some embodiments, the pharmaceutical composition has a molar ratio of complexing agent to the pharmaceutical compound that is from about 1:4 to about 1:10. In some embodiments, the pharmaceutical composition has an osmolality of no more than about 850 mOsm/kg. In some embodiments, the pharmaceutical composition has a pH of about 4 to about 7. In some embodiments, the complexing agent is present in an amount of about 10 mg/mL to about 600 mg/mL. In some embodiments, the pharmaceutical composition further comprises about 0.1 to about 20 molar equivalents of the unionized substance compared to the complexing agent. In some embodiments, the unionized substance comprises brexanolone. In some embodiments, the chemical entity comprises γ-aminobutyric acid (GABA). In some embodiments, the complexing agent further comprises a non-polar pore. In some embodiments, the about 0.1 to about 20 molar equivalents of the unionized substance is complexed to the non-polar pore. In some embodiments, the unionized substance is cleaved off from the GABA and released from the pharmaceutical composition after the pharmaceutical composition is administered to an individual.

In another aspect, provided herein is a pharmaceutical composition, comprising: (i) a first pharmaceutical compound, or an enantiomer, a mixture of enantiomers, or an isotopic variant thereof, wherein the pharmaceutical compound comprises a protonated nitrogen atom; (ii) a complexing agent, wherein the complexing agent comprises a plurality of acidic functional groups, wherein the plurality of acidic functional groups comprise a conjugate base of an acid which acts as a counterion for the protonated nitrogen atom of the first pharmaceutical compound; and (iii) a second pharmaceutical compound, wherein the second pharmaceutical compound is unionized. In some embodiments, the complexing agent is sulfobutylether-β-cyclodextrin. In some embodiments, the first pharmaceutical compound comprises ketamine. In some embodiments, the second pharmaceutical compound comprises rapamycin. In some embodiments, the first pharmaceutical compound comprises ketamine and the second pharmaceutical compound comprises rapamycin. In some embodiments, the first pharmaceutical compound comprises no ketamine and the second pharmaceutical compound comprises rapamycin. In some embodiments, the complexing agent comprises a substituted cyclodextrin. In some embodiments, the complexing agent comprises a cyclodextrin substituted with at least one acidic functional group. In some embodiments, the cyclodextrin is substituted with 3 to 8 acidic functional groups. In some embodiments, the cyclodextrin is sulfobutylether-β-cyclodextrin. In some embodiments, the pharmaceutical composition has lower osmolality than a composition comprising a salt of the first pharmaceutical compound, a salt of the complexing agent and a salt of the second pharmaceutical compound. In some embodiments, the pharmaceutical composition is formulated for subcutaneous, intramuscular, sublingual, oral, rectal, transvaginal, or intranasal administration. In some embodiments, the pharmaceutical composition has a molar ratio of the complexing agent to the first pharmaceutical compound that is from about 1:4 to about 1:10. In some embodiments, the pharmaceutical composition has a molar ratio of the complexing agent to the second pharmaceutical compound that is about 1:1. In some embodiments, the pharmaceutical composition has an osmolality of no more than about 850 mOsm/kg. In some embodiments, the pharmaceutical composition has a pH of about 4 to about 7. In some embodiments, the complexing agent is present in an amount of about 10 mg/mL to about 600 mg/mL. In some embodiments, the pharmaceutical composition further comprises an amount of the first pharmaceutical compound in an unionized form. In some embodiments, the complexing agent comprises a non-polar pore. In some embodiments, the second pharmaceutical compound is complexed to the non-polar pore. In some embodiments, the first pharmaceutical compound in the unionized form is complexed to the non-polar pore. In some embodiments, the second pharmaceutical compound comprises clonidine. In some embodiments, the first pharmaceutical compound comprises ketamine and the second pharmaceutical compound comprises clonidine. In some embodiments, the first pharmaceutical compound comprises no ketamine and the second pharmaceutical compound comprises clonidine.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE FIGURES

FIG. 1A shows an example of a formulation of Tigecycline FB with 1.1 molar equivalents of CapAcid.

FIG. 1B shows an example of the structure of Tigecycline-Captisol® salt.

FIG. 2 shows an example of the structure of Naloxone-Captisol® salt.

FIG. 3 shows an example of the structure of Amikacin-Captisol® salt.

FIG. 4 shows an example of the structure of 1-Amantadine-Captisol® salt.

FIG. 5 shows an example of the structure of 2-Amantadine-Captisol® salt.

FIG. 6A shows an example of a formulation of Rimantadine HCl being fully dissolved.

FIG. 6B shows an example of a formulation of Rimantadine HCl being incompletely dissolved.

FIG. 7 shows an example of the structure of Rimantadine-Captisol® salt.

FIG. 8 shows an example of the structure of Amifampridine-Captisol® salt.

FIG. 9 shows an example of the structure of Caspofungin-Captisol® salt.

FIG. 10A shows an example of a formulation of Melevodopa FB.

FIG. 10B shows an example of a formulation of Melevodopa FB with 1.1 molar equivalents of CapAcid being fully dissolved.

FIG. 10C shows an example of the structure of Melevodopa-Captisol® salt.

FIG. 11A shows an example of the structure of Eletriptan-Captisol® salt at 1:1 Ratio of API to CapAcid.

FIG. 11B shows an example of a formulation of Eletriptan FB-CapAcid at 2:1 ratio of API to CapAcid being fully dissolved.

FIG. 11C shows an example of the structure of Eletriptan-Captisol® salt at 2:1 ratio of API to CapAcid.

FIG. 11D shows an example of a formulation of Eletriptan FB-CapAcid at 4:1 ratio of API to CapAcid being fully dissolved.

FIG. 11E shows an example of the structure of Eletriptan-Captisol® salt at 4:1 ratio of API to CapAcid.

FIG. 12A shows an example of a formulation of Rotigotine FB CapAcid at 1:1 ratio of API to CapAcid being fully dissolved.

FIG. 12B shows an example of the structure of Rotigotine-Captisol® salt at 1:1 Ratio of API to CapAcid.

FIG. 12C shows an example of a formulation of Rotigotine-FB CapAcid at 2:1 Ratio of API to CapAcid being fully dissolved.

FIG. 12D shows an example of the structure of Rotigotine-Captisol® salt at 2:1 Ratio of API to CapAcid.

FIG. 13A shows an example of a formulation of Copanlisib FB-CapAcid at 1:1 Ratio of API to CapAcid being fully dissolved.

FIG. 13B shows an example of the structure of Copanlisib-Captisol® salt at 1:1 Ratio of API to CapAcid.

FIG. 13C shows an example of a mixture of Copanlisib FB-CapAcid at 2:1 Ratio of API to CapAcid being incompletely dissolved after stirring for 1 hour.

FIG. 13D shows an example of a mixture of Copanlisib FB-CapAcid at 2:1 Ratio of API to CapAcid being incompletely dissolved after stirring overnight.

FIG. 13E shows an example of a mixture of Copanlisib FB-CapAcid at 2:1 Ratio of API to Cap Acid in which the freebase precipitates out of the solution at the pH of 2.2.

FIG. 14A shows an example of a formulation of Nafamostat FB-Cap Acid at 1:1 Ratio of API to CapAcid being fully dissolved.

FIG. 14B shows an example of the structure of Nafamostat-Captisol® salt at 1:1 Ratio of API to CapAcid.

FIG. 14C shows an example of a formulation of Nafamostat FB-CapAcid at 2:1 Ratio of API to CapAcid being fully dissolved.

FIG. 14D shows an example of the structure of Nafamostat-Captisol® salt at 2:1 Ratio of API to CapAcid.

FIG. 14E shows an example of a formulation of Nafamostat FB Captisol® Na⁺ at 1:1 Ratio of API to CapAcid being completely dissolved.

FIG. 15A shows an example of a formulation of Remdesivir FB Cap Acid at 1:1 Ratio of API to CapAcid being completely dissolved.

FIG. 15B shows an example of a mixture of Remdesivir FB CapAcid at 1:1 Ratio of API to Cap Acid in which the freebase precipitates out of the solution at the pH of 1.83.

FIG. 16A shows an example of undissolved Midazolam CapAcid 1:1 ratio relative to H⁺, as seen by the cloudy solution.

FIG. 16B shows an example of completely dissolved Midazolam Cap Acid 3:1 ratio relative to cyclodextrin, as seen by the clear solution.

FIG. 16C shows an example of Midazolam CapAcid 3:1 salt after lyophilization, crushed into an off-white powder.

FIG. 17A shows an example of a mixture of Eletriptan and Cap Acid being incompletely dissolved in ethanol after stirring for 1 hour.

FIG. 17B shows an example of a mixture of Eletriptan and Cap Acid being incompletely dissolved in water.

FIG. 17C shows an example of a mixture of Eletriptan and Cap Acid after lyophilization.

FIG. 17D shows an example of a mixture of Eletriptan and CapAcid being ground into powder after lyophilization.

FIG. 17E shows an example of a powder of Eletriptan and CapAcid being incompletely dissolved in water.

FIG. 18A shows an example of a mixture of Carvedilol and CapAcid being incompletely dissolved in ethanol after stirring for 1 hour.

FIG. 18B shows an example of a mixture of Carvedilol and CapAcid being incompletely dissolved in ethanol after stirring for 2 hours.

FIG. 18C shows an example of a mixture of Carvedilol and CapAcid being incompletely dissolved in water.

FIG. 18D shows an example of a mixture of Carvedilol and CapAcid after lyophilization.

FIG. 18E shows an example of a mixture of Carvedilol and CapAcid being ground into powder after lyophilization.

FIG. 18F shows an example of a powder of Carvedilol and CapAcid being incompletely dissolved in water.

FIG. 19A shows an example of a mixture of Carvedilol and CapAcid being incompletely dissolved in ethanol after stirring for 1 hour.

FIG. 19B shows an example of a mixture of Carvedilol and CapAcid after lyophilization.

FIG. 19C shows an example of a mixture of Carvedilol and CapAcid being ground into powder after lyophilization.

FIG. 19D shows an example of a powder of Carvedilol and Cap Acid being incompletely dissolved in water.

FIG. 20A shows an example of a mixture of Copanlisib and Cap Acid being incompletely dissolved in ethanol after stirring for 1 hour.

FIG. 20B shows an example of a mixture of Copanlisib and Cap Acid being incompletely dissolved in water after evaporation.

FIG. 20C shows an example of a mixture of Copanlisib and CapAcid after lyophilization.

FIG. 20D shows an example of a mixture of Copanlisib and Cap Acid being ground into powder after lyophilization.

FIG. 20E shows an example of a powder of Copanlisib and CapAcid being incompletely dissolved in water.

FIG. 21A shows an example of a mixture of Rotigotine and CapAcid being incompletely dissolved in ethanol after stirring for 1 hour.

FIG. 21B shows an example of a mixture of Rotigotine and CapAcid being incompletely dissolved in ethanol after stirring for 2 hours.

FIG. 21C shows an example of a mixture of Rotigotine and CapAcid being incompletely dissolved in water after evaporation.

FIG. 21D shows an example of a mixture of Rotigotine and CapAcid after lyophilization.

FIG. 21E shows an example of a mixture of Rotigotine and CapAcid being ground into powder after lyophilization.

FIG. 21F shows an example of a powder of Rotigotine and Cap Acid being incompletely dissolved in water.

FIG. 22A shows an example of a mixture of Remdesivir and CapAcid being incompletely dissolved in ethanol after stirring for 1 hour.

FIG. 22B shows an example of a mixture of Remdesivir and CapAcid being incompletely dissolved in water after evaporation.

FIG. 22C shows an example of a mixture of Remdesivir and CapAcid after lyophilization.

FIG. 22D shows an example of a mixture of Remdesivir and CapAcid being ground into powder after lyophilization.

FIG. 22E shows an example of a powder of Remdesivir and Cap Acid being incompletely dissolved in water.

FIG. 23A shows an example of HPLC Trace of Caspofungin Acetate.

FIG. 23B shows an example of HPLC Trace of Caspofungin FB.

FIG. 23C shows an example of HPLC Trace of Eletriptan HBr.

FIG. 23D shows an example of HPLC Trace of Eletriptan FB.

FIG. 23E shows an example of HPLC Trace of Naloxone HCl Dihydrate.

FIG. 23F shows an example of HPLC Trace of Naloxone FB.

FIG. 23G shows an example of HPLC Trace of Rotigotine HCl.

FIG. 23H shows an example of HPLC Trace of Rotigotine FB.

FIG. 24 shows an example of Brexanolone Prodrug Captisol Salt at 6-7:1 ratio of API to CapAcid without freebase complexing in the cyclodextrin pore.

FIG. 25A shows an example of Brexanolone Prodrug Captisol Salt at 4:1 molar ratio of API to CapAcid without an unionized Brexanolone in the non-polar pore.

FIG. 25B shows an example of Brexanolone Prodrug Captisol Salt at 4:1 molar ratio of API to CapAcid with an unionized Brexanolone in the non-polar pore.

FIG. 25C shows an example of a non-ionized prodrug of Brexanolone conjugated to the γ-aminobutyric acid (GABA).

FIG. 25D shows an example of an ionized prodrug of Brexanolone conjugated to GABA.

FIG. 26 shows an example of Esketamine-Captisol salt at 4:1 ratio of API to CapAcid having non-ionized rapamycin in complexing pore.

FIG. 27 shows an example of a generalized ionized API-Captisol salt at 6-7:1 ratio of API to CapAcid having non-ionized rapamycin in complexing pore.

FIG. 28 shows an example of a generalized ionized API-1-Captisol salt at 6-7:1 ratio of API to CapAcid having non-ionized API-2 in complexing pore.

FIG. 29 shows an example of Ketamine-Captisol Salt at 6-7:1 ratio of API to CapAcid having non-ionized ketamine in complexing pore.

FIG. 30 shows an example of Ketamine-Captisol Salt at 6-7:1 ratio of API to CapAcid having non-ionized rapamycin in complexing pore.

FIG. 31 shows an example of Esketamine-Captisol Salt at 4:1 ratio of API to CapAcid with non-ionized clonidine in complexing pore and cationic balance provided by sodium.

FIG. 32 shows an example of a resin ion exchange process to replace the sodium ions occupying the acidic sites of complexing agent with protons.

### DETAILED DESCRIPTION

Provided herein are, for example, compositions comprising pharmaceutical compound salts with complexing agents as counterions. Such salts are useful in a variety of pharmaceutical compositions, including reduced irritant effect to tissues and/or dermal tissues, subcutaneous, intramuscular, intranasal, and sublingual formulations. In some aspects, use of the salts provided herein in subcutaneous, intranasal, or sublingual formulation is associated with reduced irritant effect to tissues at the administration site, as well as increased solubility and bioavailability. In certain aspects, the compositions comprising low molar ratios of pharmaceutical compounds with basic nitrogen atoms to complexing agents are formulated for subcutaneous, sublingual, or intranasal administration. In certain aspects, the compositions comprising prodrug pharmaceutical compounds with basic nitrogen atoms are formulated for subcutaneous, sublingual, or intranasal administration. In certain aspects, the compositions comprising both ionized and unionized pharmaceutical compounds are formulated for subcutaneous, sublingual, or intranasal administration. Also provided herein are, for example, methods of treating, preventing or managing, viral infections, bacterial infections, fungal infections, autoimmune disorders, inflammatory disorders, depression or opioid overdose, psychiatric disorders, cognitive disorders, neurological disorders, and other various disorders.

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “about” as used herein, when referring to a numerical value or range, allows for a degree of variability in the value or range, for example, within 10%, or within 5% of a stated value or of a stated limit of a range. Unless otherwise stated, “about” refers to a degree of variability within 10% of the stated value or of a stated limit of a range.

The term “pharmaceutical compound” and similar such terms used herein refer to any compound which has the potential to be administered to a subject and may imbue any type of therapeutic benefit to a subject (such as treatment or prevention of a disease, mitigation of symptoms of a disease or condition, or any purpose for which a pharmaceutical or drug can be used). Generally, these compounds will be organic small molecules, though other compounds such as peptides are also considered to be pharmaceutical compounds as used herein. In preferred embodiments, the pharmaceutical compounds will comprise basic nitrogen atoms (e.g. amine groups) which can be protonated upon interaction with an acidic functional group, such as a carboxylic acid or a sulfonic acid. When referring to pharmaceutical compositions, these compounds may be referred to generally as “active pharmaceutical ingredient” or “API.” In some cases, the pharmaceutical compounds herein may simply be referred to as “compounds.”

The terms “opioid pharmaceutical compound,” “opioid pharmaceutical,” or “opioid,” and similar such terms are all used interchangeably, and the same meaning is meant by each term unless otherwise specified. The term may refer to any naturally occurring opioid or any synthetic homolog or analog. Additionally, any synthetic compound which has similar bioactivity on the opioid receptors of a subject is also intended to be encompassed, as well as any compound which has an opioid antagonist activity (e.g. naltrexone or naloxone). In some cases, the pharmaceutical composition or method for manufacture or use thereof does not include naloxone. When referring to pharmaceutical compositions, these compounds may be referred to generally as “active pharmaceutical ingredient” or “API.”

As used herein, the terms “comprising,” “comprises,” or the like are used in their typical sense of leaving any claim or embodiment where such language is used able to accommodate additional elements, components, or features. However, it is also contemplated that in each formulation, salt, method, or other disclosure provided herein that uses the term “comprising,” the formulation, salt, method or other disclosure may also be closed to other elements, components, or features as if the term “consisting of” were used in its place. Additionally, it is also contemplated the term “comprising” or similar can also be replaced in the same manner as if the term “consisting essentially of.”

A “molar equivalent” as used herein refers to a comparison on the number of moles of a substance compared to the number of moles of another substance and reflects that comparison should be a moles or molarity basis (e.g. the ratio of the moles of one compound to the moles of another). The molar equivalent need not be an integer value. For example, embodiments stating that a pharmaceutical composition comprises a “molar equivalent” of a substance indicates that that the amount of the substance which is present will be measured in some kind of molarity descriptor, such as an additional equivalent of the substance from 0.001 to 100 molar equivalents, or any other range specified herein.

All percent compositions are given as weight-percentages, unless otherwise stated.

All average molecular weights of polymers are weight-average molecular weights, unless otherwise specified.

As used herein, “individual” (as in the subject of the treatment) means both mammals and non-mammals. Mammals include, for example, humans; non-human primates, e.g. apes and monkeys; and non-primates, e.g. dogs, cats, cattle, horses, sheep, and goats. Non-mammals include, for example, fish and birds.

The terms “disease,” “disorder,” or “condition” refer to a state of being or health status of a patient or subject capable of being treated with the compounds or methods provided herein. The disease may be physical disorder. The disease may be a mental or psychiatric disorder. The disease may be an infection, such as a viral infection, a bacterial infection, or a fungal infection. The disease may be an autoimmune disease. The disease may be a mood disorder. The disease may be an inflammatory disease. The disease may be a brain tumor. The disease may be a neurological condition or disorder. The disease may be migraine headache. The disease may be pancreatitis. The disease may be lymphoma. The disease may be opioid overdose. The disease may be flu infection. In some further instances, “mental or psychiatric disorder” refers to human mental or psychiatric disorders including major depressive disorder, treatment resistant major depressive disorder, Suicidality, Suicidal Ideation, dysthymia, bipolar I disorder, bipolar II disorder, post-traumatic stress disorder (PTSD), complex trauma, anorexia nervosa, bulimia nervosa, eating disorder NOS, obsessive compulsive disorder, a substance-related disorder (e.g., cannabis dependence or withdrawal, barbiturate dependence or withdrawal, benzodiazepine dependence or withdrawal, amphetamine dependence or withdrawal, opioid dependence or withdrawal, opioid dependence and detoxification, alcohol dependence or withdrawal, cocaine dependence or withdrawal), a pain disorder and an inflammatory disorder, management of pain including but not limited to neuropathic pain, complex regional pain syndrome and post herpetic neuralgia. In some further instances, “neurological disease or disorder” refers to human neurological diseases or disorders including chronic fatigue syndrome, chronic fatigue and immunodeficiency syndrome, neuropathy, fibromyalgia, fibromyalgia syndrome, myalgic encephalomyelitis, migraine, traumatic brain injury (TBI), stroke, dementia, amyotrophic lateral sclerosis, spinal cord injury, shingles, herpes zoster, radiculopathy, polyneuropathy, dyskinesia, dystonia, tinnitus, postherpetic neuralgia, complex regional pain syndrome, central pain syndrome, chronic pain, acute pain, phantom limb syndrome with pain, phantom limb syndrome without pain, myelitis, dysthymia, complex trauma, anorexia nervosa, bulimia nervosa, eating disorder NOS, obsessive compulsive disorder, intermittent explosive disorder, a sleep disorder, a pain disorder or an inflammatory disorder. In some further instances, a brain tumor may be acoustic neuroma, astrocytoma, brain metastases, choroid plexus carcinoma, craniopharyngioma, embryonal tumors, ependymoma, glioblastoma, glioma, medulloblastoma, meningioma, oligodendroglioma, pediatric brain tumors, pineoblastoma, or pituitary tumors. In some instances, the disease, disorder, or condition is one that is associated with substantial or significant pain. In some aspects, the subject is administered the salts of formulations provided herein in order to manage pain. The pain can be associated with a suitable conditions for which an opioid pain management regiment is acceptable. In some aspects, the subject is administered the salts of formulations provided herein in order to treat a brain tumor. In some aspects, the subject is administered the following salts of formulations in order to treat a brain tumor: a pharmaceutical compound with basic nitrogen atoms including a dissociative medication compound, a dissociative hallucinogen compound, a dissociative anesthetic compound, an arylcyclo-hexylamine, a 1,2-diarylethylamine, a β-keto-arylcyclohexylamine, or a compound that modulates the NMDA receptor, ketamine, a derivative or analog of ketamine, methoxetamine, deschloroketamine, N-ethyl deschloroketamine (eticyclidone), 3-methoxyphencyclidine, methoxieticyclidine, ephenidine, lanicemine, dextromethorphan, dextrorphan, or methoxyketamine.

The expression “effective amount,” when used to describe therapy to an individual suffering from a disorder, refers to the amount of a compound described herein that is effective to inhibit or otherwise act on relevant receptors in the individual's tissues, wherein such inhibition or other action occurs to an extent sufficient to produce a beneficial therapeutic effect. The effective amount will vary based on the pharmaceutical compound, including but not limited to opioid, or other API used in the formulation and the indication intended to be treated by said compound, including but not limited to opioid, or other API.

“Substantially” as the term is used herein means completely or almost completely. For example, a composition that is “substantially free” of a component either has none of the component or contains such a trace amount that any relevant functional property of the composition is unaffected by the presence of the trace amount. For example, a compound that is “substantially pure” has only negligible traces of impurities present.

All chiral, diastereomeric, and/or racemic forms of a structure are intended, unless a particular stereochemistry or isomeric form is specifically indicated. Compounds described herein can include enriched or resolved optical isomers at any or all asymmetric atoms as are apparent from the depictions, at any degree of enrichment. Both racemic and diastereomeric mixtures, as well as the individual optical isomers can be isolated or synthesized so as to be substantially free of their enantiomeric or diastereomeric partners, and these are all within the scope of the present disclosure.

The inclusion of an isotopic form of one or more atoms in a molecule that is different from the naturally occurring isotopic distribution of the atom in nature is referred to as an “isotopically labeled form” of the molecule. All isotopic forms of atoms are included as options in the composition of any molecule, unless a specific isotopic form of an atom is indicated. For example, any hydrogen atom or set thereof in a molecule can be any of the isotopic forms of hydrogen, e.g., protium (¹H), deuterium (²H), or tritium (³H) in any combination. Similarly, any carbon atom or set thereof in a molecule can be any of the isotopic form of carbons, such as ¹¹C, ¹²C, ¹³C, or ¹⁴C, or any nitrogen atom or set thereof in a molecule can be any of the isotopic forms of nitrogen, such as ¹³N, ¹⁴N, or ¹⁵N. A molecule can include any combination of isotopic forms in the component atoms making up the molecule, the isotopic form of every atom forming the molecule being independently selected. In a multi-molecular sample of a compound, not every individual molecule necessarily has the same isotopic composition. For example, a sample of a compound can include molecules containing various different isotopic compositions, such as in a tritium or ¹⁴C radiolabeled sample where only some fraction of the set of molecules making up the macroscopic sample contains a radioactive atom. It is also understood that many elements that are not artificially isotopically enriched themselves are mixtures of naturally occurring isotopic forms, such as ¹⁴N and ¹⁵N, ³²S and ³⁴S, and so forth. A molecule as recited herein is defined as including isotopic forms of all its constituent elements at each position in the molecule. As is well known in the art, isotopically labeled compounds can be prepared by the usual methods of chemical synthesis, except substituting an isotopically labeled precursor molecule. The isotopes, radiolabeled or stable, can be obtained by any method known in the art, such as generation by neutron absorption of a precursor nuclide in a nuclear reactor, by cyclotron reactions, or by isotopic separation such as by mass spectrometry. The isotopic forms are incorporated into precursors as required for use in any particular synthetic route. For example, ¹⁴C and ³H can be prepared using neutrons generated in a nuclear reactor. Following nuclear transformation, ¹⁴C and ³H are incorporated into precursor molecules, followed by further elaboration as needed.

A “hydrate” is a compound that exists in a composition with water molecules. The composition can include water in stoichiometric quantities, such as a monohydrate or a dihydrate, or can include water in random amounts. As the term is used herein a “hydrate” refers to a solid form, e.g., a compound in water solution, while it may be hydrated, is not a hydrate as the term is used herein.

A “solvate” is a similar composition except that a solvent other that water replaces the water. For example, methanol or ethanol can form an “alcoholate”, which can again be stoichiometric or non-stoichiometric. As the term is used herein a “solvate” refers to a solid form, e.g., a compound in solution in a solvent, while it may be solvated, is not a solvate as the term is used herein.

A “prodrug” as is well known in the artis a substance that can be administered to a patient where the substance is converted in vivo by the action of biochemicals within the patient's body, such as enzymes, to the active pharmaceutical ingredient. Examples of prodrugs include esters of carboxylic acid groups, which can be hydrolyzed by endogenous esterases as are found in the bloodstream of humans and other mammals. Further examples of prodrugs include boronate esters which can be hydrolyzed under physiological conditions to afford the corresponding boronic acid. Conventional procedures for the selection and preparation of suitable prodrug derivatives are described, for example, in “Design of Prodrugs”, ed. H. Bundgaard, Elsevier, 1985.

In various embodiments, a compound as shown in any of the Examples, or among the exemplary compounds, is provided.

Provisos may apply to any of the disclosed categories or embodiments wherein any one or more of the other above disclosed embodiments or species may be excluded from such categories or embodiments.

### Isomerism in Compounds Described Herein

### Optical Isomerism

It will be understood that when compounds of the present disclosure contain one or more chiral centers, the compounds may exist in, and may be isolated as pure enantiomeric or diastereomeric forms or as racemic mixtures. The present disclosure therefore includes any possible enantiomers, diastereomers, racemates or mixtures thereof of the compounds described herein.

The isomers resulting from the presence of a chiral center comprise a pair of non-superimposable isomers that are called “enantiomers.” Single enantiomers of a pure compound are optically active, e.g., they are capable of rotating the plane of plane polarized light. Single enantiomers are designated according to the Cahn-Ingold-Prelog system. The priority of substituents is ranked based on atomic weights, a higher atomic weight, as determined by the systematic procedure, having a higher priority ranking. Once the priority ranking of the four groups is determined, the molecule is oriented so that the lowest ranking group is pointed away from the viewer. Then, if the descending rank order of the other groups proceeds clockwise, the molecule is designated (R) and if the descending rank of the other groups proceeds counterclockwise, the molecule is designated(S). In the example below, the Cahn-Ingold-Prelog rankingis A>B>C>D. The lowest ranking atom, D is oriented away from the viewer.

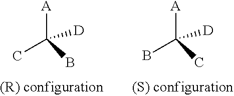

The present disclosure is meant to encompass diastereomers as well as their racemic and resolved, diastereomerically and enantiomerically pure forms and salts thereof. Diastereomeric pairs may be resolved by known separation techniques including normal and reverse phase chromatography, and crystallization.

“Isolated optical isomer” means a compound which has been substantially purified from the corresponding optical isomer(s) of the same formula. Preferably, the isolated isomer is at least about 80%, more preferably at least 90% pure, even more preferably at least 98% pure, most preferably at least about 99% pure, by weight.

Isolated optical isomers may be purified from racemic mixtures by well-known chiral separation techniques. According to one such method, a racemic mixture of a compound described herein, or a chiral intermediate thereof, is separated into 99% wt. % pure optical isomers by HPLC using a suitable chiral column, such as a member of the series of DAICEL® CHIRALPAK® family of columns (Daicel Chemical Industries, Ltd., Tokyo, Japan). The column is operated according to the manufacturer's instructions.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)-or (S)-or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; e.g., the R and S configurations for each asymmetric center. Therefore, single stereochemical isomers as well as enantiomeric and diastereomeric mixtures of the present compounds are within the scope of the disclosure.

“Alkyl” refers to a straight or branched hydrocarbon chain radical consisting solely of carbon and hydrogen atoms, which may optionally be unsaturated with one or more double or triple bonds, and preferably having from one to fifteen carbon atoms (i.e., C₁-C₁₅ alkyl). In certain embodiments, an alkyl comprises one to six carbon atoms (i.e., C₁-C₆ alkyl). In certain embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless otherwise specified, the term “alkyl” and its equivalents encompass linear, branched, and/or cyclic alkyl groups. In some instances, an “alkyl” comprises both cyclic and acyclic (linear and/or branched) alkyl components.

The term “substituted” refers to moieties having substituents replacing a hydrogen on one or more carbons or heteroatoms of the structure. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents may be one or more and the same or different for appropriate organic compounds.

Substituents may include any substituent, for example, a halogen, a hydroxyl, a carbonyl (such as an oxo (═O), a carboxyl, an alkoxycarbonyl, a formyl, or an acyl), a thiocarbonyl (such as a thioxo (═S), a thioester, a thioacetate, or a thioformate), an alkoxyl, a phosphoryl, a phosphate, a phosphonate, a phosphinate, an amino, an amido, an amidine, an imine, an oximo, a hydrazino, a cyano, a nitro, an azido, a sulfhydryl, an alkyl, an alkylthio, a sulfate, a sulfonate, a sulfamoyl, a sulfonamido, a sulfonyl, an aralkyl, a carbocycle, a heterocycle, a cycloalkyl, a heterocycloalkyl, an aromatic and heteroaromatic moiety.

As used herein, an “acidic functional group” or similar term (e.g. “acidic functionality”) refers to a chemical moiety which contains at least one dissociable proton (or isotopic variant thereof), or the conjugate base (e.g. the deprotonated anion) of the acidic functional group. In certain embodiments, the dissociable proton dissociates from the chemical moiety at a pH common in aqueous systems (e.g. pHs from about 1 to about 14). In certain preferred embodiments, the dissociable proton dissociates from the chemical moiety in an aqueous system at a pH of less than 7 (e.g. having a pKa value of less than 7, such as a pKa of less than 6, less than 5, less than 4, less than 3, less than 2, or less than 1). As is understood by those in the art, whether an acidic functional group contains the dissociable proton will depend on the conditions of the system in which the chemical moiety is present (e.g., the pH of an aqueous system containing molecule with the acidic functional group or the presence of any base molecule). As such, the term “acidic functional group” (or reference to a specific acidic functional group such as a carboxylic acid or a sulfonic acid) as used herein is intended to cover the protonated version of the moiety, the deprotonated version of the moiety, and any salt of the moiety, unless otherwise specified.

Unless otherwise stated, structures depicted herein are also meant to include compounds which differ only in the presence of one or more isotopically enriched atoms (e.g., isotopic variant(s)). For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

A “salt,” as is well known in the art, includes an organic compound such as a carboxylic acid, a sulfonic acid, or an amine, in ionic form, in combination with a counterion. For example, acids in their anionic form can form salts with cations such as metal cations, for example sodium, potassium, and the like; with ammonium salts such as NH₄⁺ or the cations of various amines, including tetraalkyl ammonium salts such as tetramethylammonium, or other cations such as trimethylsulfonium, and the like. The terms “pharmaceutically acceptable salts” and/or or “pharmacologically acceptable salts” are meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogen sulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents. In certain embodiments, compounds of the present disclosure contain both basic and acidic functionalities that allow the compounds to be converted into either base or acid addition salts. The neutral forms of the compounds may be regenerated by contacting the salt with a base or acid and isolating the parent compound in a conventional manner. The parent form of the compounds differs from the various salt forms in certain physical properties, such as solubility in polar solvents, but, unless specifically indicated, the salts disclosed herein are equivalent to the parent form of the compound for the purposes of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of a compound to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, complexing agents (e.g. cyclodextrins), binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The terms “treating” or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In certain embodiments, treating is preventing. In certain embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (e.g., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things. The relevant symptoms will vary depending upon the intended indication of a particular API.

“Treating” and “treatment” as used herein include prophylactic treatment. Treatment methods include administering to a subject a therapeutically effective amount of a compound described herein. The administering step may consist of a single administration or may include a series of administrations. The length of the treatment period depends on a variety of factors, such as the severity of the condition, the age of the patient, the concentration of the compound, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient.

The term “prevent” refers to a decrease in the occurrence of disease symptoms in a patient. As indicated above, the prevention may be complete (no detectable symptoms) or partial, such that fewer symptoms are observed than would likely occur absent treatment. In certain embodiments, prevent refers to slowing the progression of the disease, disorder or condition or inhibiting progression thereof to a harmful or otherwise undesired state.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). The therapeutically effective amount can be ascertained by measuring relevant physiological effects, and it can be adjusted in connection with the dosing regimen and diagnostic analysis of the subject's condition, and the like. By way of example, measurement of the serum level of an inhibitor (or, e.g., a metabolite thereof) at a particular time post-administration may be indicative of whether a therapeutically effective amount has been administered.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan. Adjusting the dose to achieve maximal therapeutic window efficacy or toxicity in humans based on the methods described above and other methods is well within the capabilities of the ordinarily skilled artisan.

The term “therapeutically effective amount,” as used herein, refers to that amount of the therapeutic agent sufficient to ameliorate the disorder, as described herein. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means subcutaneous (i.e., “SC,” “subQ,” or “SQ”) administration, oral administration, administration as a suppository, topical contact or administration, intravenous, parenteral, intraperitoneal, intramuscular, intraosseous, intralesional, intrathecal, intracranial, intranasal, epidural, or the implantation of a slow-release device, e.g., a mini-osmotic pump, to a subject. Administration is by any route, including parenteral and transmucosal (e.g., buccal, sublingual, palatal, gingival, nasal, vaginal, rectal, transvaginal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g. anti-cancer agent, chemotherapeutic, or treatment for a neurodegenerative disease). The compound of the disclosure can be administered alone or can be coadministered to the patient. Coadministration is meant to include simultaneous or sequential administration of the compound individually or in combination (more than one compound or agent). Thus, the preparations can also be combined, when desired, with other active substances (e.g., to reduce metabolic degradation). The compositions of the present disclosure can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present disclosure may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present disclosure can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present disclosure can be delivered by the use of liposomes which fuse with the cellular membrane or are endocytosed, e.g., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present disclosure into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present disclosure can also be delivered as nanoparticles.

By “co-administer” it is meant that a composition described herein is administered at the same time, just prior to, or just after the administration of one or more additional therapies. The compounds of the disclosure can be administered alone or can be coadministered to the patient. Coadministration is meant to include simultaneous or sequential administration of the compounds individually or in combination (more than one compound). The compositions of the present disclosure can be delivered transdermally, by a topical route, or formulated as applicator sticks, solutions, suspensions, emulsions, gels, creams, ointments, pastes, jellies, paints, powders, and aerosols.

Utilizing the teachings provided herein an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The compounds described herein can be used in combination with one another, with other active agents known to be useful in treating a mental or psychiatric disorder, a mood disorder, a neurological condition or disorder, a metabolic disorder (e.g., type 2 diabetes mellitus and/or complications thereof), endometriosis, glaucoma, pain, Parkinson's disease, migraine headache, viral infection, bacterial infection, fungal infection, autoimmune disease, lymphoma, pancreatitis, opioid overdose, flu infection, or an inflammatory disorder.

In some embodiments, co-administration includes administering one active agent within 0.5, 1, 2, 4, 6, 8, 10, 12, 16, 20, 24 hours, 2 days, 4 days, 1 week or 1 month of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, e.g., preparing a single pharmaceutical composition including both active agents. In other embodiments, the active agents can be formulated separately. In another embodiment, the active and/or adjunctive agents may be linked or conjugated to one another. In some embodiments, the compounds described herein may be combined with treatments for infections (e.g. bacterial infections), inflammation, and/or vasodilation.

The compounds described herein can be administered to treat a metabolic disease or disorder (e.g., type 2 diabetes mellitus and/or complications thereof), a mental or psychiatric disorder, a mood disorder, a neurological condition or disorder, endometriosis, glaucoma, pain, Parkinson's disease, migraine headache, viral infection, bacterial infection, fungal infection, autoimmune disease, lymphoma, pancreatitis, opioid overdose, flu infection, or an inflammatory disorder. In this regard, the compounds disclosed herein may be administered either alone to treat such diseases or disorders or may be co-administered with another therapeutic agent to treat such diseases or disorders.

The compounds disclosed herein may be co-administered with other active agents including but not limited to antidepressants, antipsychotics, anti-inflammatoires, anxiolytics, and/or analgesics.

The APIs (e.g., rotigotine, eletriptan, copanlisib, remdesivir, nafamostat (nafamostat mesylate), melevodopa, tigecycline, naloxone, amikacin, 1-amantadine, 2-amantadine, rimantadine, midazolam, amifampridine, caspofungin, rapamycin, clonidine, ketamine, methoxetamine, deschloroketamine, tryptamines, phenethylamines, lysergamide compounds, opioids, cathinone compounds, 3,4-methylenedioxyamphetamine compound derivatives, aminoalkyl-substituted benzofurans, substituted amphetamines, aminoindanes, stimulants, diphenhydramine, hydroxazine, phenylephrine, dopamine, adrenaline, lidocaine, oxymetazoline, clemastine, chlorpheniramine, or 6-chloro-2-aminotetralin, etc.) disclosed herein may be administered once daily until study reached endpoint. The inhibitors disclosed herein may be administered at least three times but in some studies four or more times depending on the length of the study and/or the design of the study.

The term “bioavailability (F),” as used herein, refers to the fraction of a dose of drug (e.g., epinephrine) that is absorbed from its site of administration and reaches, in an unchanged form, the systemic circulation. The term “absolute bioavailability” is used when the fraction of absorbed drug is related to its I.V. bioavailability. It may be calculated using the following formula:

$$F = \frac{{AUC}_{extravascular}}{{AUC}_{intravenous}} \times \frac{{Dose}_{intravenous}}{{Dose}_{extravascular}}$$

The term relative bioavailability (Fᵣₑₗ) is used to compare two different extravascular routes of drug administration and it may be calculated using the following formula:

$${F}_{rel} = \frac{{AUC}_{extravascular 1}}{{AUC}_{extravascular 2}} \times \frac{{Dose}_{extravascular 2}}{{Dose}_{extravascular 1}}$$

The term “clearance (CL),” as used herein, refers to the rate at which a drug is eliminated divided by its plasma concentration, giving a volume of plasma from which drug is completely removed per unit of time. CL is equal to the elimination rate constant (λ) multiplied by the volume of distribution (V_{d}), wherein “V_{d}” is the fluid volume that would be required to contain the amount of drug present in the body at the same concentration as in the plasma. The term “apparent clearance (CL/F),” as used herein, refers to clearance that does not take into account the bioavailability of the drug. It is the ratio of the dose over the AUC.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

Generally, dosage levels of pharmaceutical compounds (API) in the compositions can range from about 5 μg/kg to about 10 mg/kg, from about 0.5 mg/kg to about 5 mg/kg, from about 1 mg/kg to about 3 mg/kg, or a fixed dose from about 10-100 mg, or 20-75 mg, or 3-60 mg, or 10-250 mg, or 10-400 mg, or an amount greater than 400 mg.

“Substantially pure” indicates that a component makes up greater than about 50% of the total content of the composition, and typically greater than about 60% of the total content. More typically, “substantially pure” refers to compositions in which at least 75%, at least 85%, at least 90% or more of the total composition is the component of interest. In some cases, the polypeptide will make up greater than about 90%, or greater than about 95% of the total content of the composition (percentage in a weight per weight basis).

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Contacting” is used in accordance with its plain ordinary meaning and refers to the process of allowing at least two distinct species (e.g., chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway (e.g., MAP kinase pathway).

As defined herein, the terms “activation,” “activate,” “activating,” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

The terms “agonist,” “activator,” “upregulator,” etc., refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist. In embodiments, an agonist is a molecule that interacts with a target to cause or promote an increase in the activation of the target. In embodiments, activators are molecules that increase, activate, facilitate, enhance activation, sensitize, or up-regulate, e.g., a gene, protein, ligand, receptor, or cell.

The “activity” of a molecule may describe or refer to the binding of the molecule to a ligand or to a receptor; to catalytic activity; to the ability to stimulate gene expression or cell signaling, differentiation, or maturation; to antigenic activity; to the modulation of activities of other molecules; and the like.

The term “osmolality” as described herein is defined as the number of osmoles (Osm) of solute per kilogram of solvent (osmol/kg or Osm/kg).

The term “osmolarity” as described herein is defined is defined as the number of osmoles of solute per liter (L) of solution (osmol/L or Osm/L).

Osmolarity may be calculated from osmolality as follows: osmolarity=osmolality×(ρₛₒₗ-cₐ); where ρₛₒₗ is the density of the solution in g/mL and cₐ is the (anhydrous) solute concentration in g/mL. Unless expressly stated otherwise, osmolarity is calculated using osmolality according to the preceding formula. Alternatively, osmolarity may be calculated experimentally.

### II. Compositions

### Complexing Agent Salts of Pharmaceutical Compounds

Provided herein are salts of conjugate acid forms of pharmaceutical compounds comprising at least one basic nitrogen and conjugate base forms of complexing agents. Such salts have advantages over other salts of compounds because they are more soluble than many other salt forms owing to the nature of the complexing agent and its ability to solubilize compounds. Additionally, in some embodiments, the preparation of such complexing agent/pharmaceutical compound salts results in a composition that will have a lower osmolality upon dissolution or otherwise in solution than a combination of individual salts of each component, or of each component individually.

In an aspect, provided herein is a pharmaceutically acceptable salt of a compound pharmaceutical comprising: (i) a pharmaceutical compound, or an enantiomer, a mixture of enantiomers, or an isotopic variant thereof; or a pharmaceutically acceptable salt, solvate or hydrate thereof, wherein the pharmaceutical compound comprises a protonated nitrogen atom; and (ii) a conjugate base of a complexing agent comprising a plurality of acidic functional groups, wherein at least one acidic functional group of the plurality of acidic functional groups acts as a counterion of the pharmaceutical compound, wherein the pharmaceutically acceptable salt has a ratio of the conjugate base of the complexing agent to the pharmaceutical compound that is from about 1:1 to about 1:4. In some embodiments, the pharmaceutical compound is a an antiviral compound, an antibacterial compound, an anti-fungal compound, a compound for treatment of a neurological disorder, a compound for treatment of Parkinson's disease, a treatment for migraine headache, a treatment for autoimmune disease, a treatment for cancer, a treatment for lymphoma, a treatment for pancreatitis, a treatment for opioid overdose, a treatment for flu infection, or a treatment for an inflammatory disorder. In some embodiments, the pharmaceutical compound comprises rotigotine, eletriptan, copanlisib, remdesivir, nafamostat (nafamostat mesylate), melevodopa, tigecycline, naloxone, amikacin, 1-amantadine, 2-amantadine, rimantadine, amifampridine, rapamycin, clonidine, or caspofungin. In some embodiments, the pharmaceutical compound has a solubility below a threshold value. In some embodiments, the pharmaceutical compound has a solubility above a threshold value. In some embodiments, the pharmaceutical compound comprises a pKa above a threshold value. In some embodiments, the pharmaceutical compound comprises a pKa below a threshold value.

In some embodiments, the pharmaceutical compound has a solubility of more than 100 mg/ml as salt. In some embodiments, the pharmaceutical compound has a solubility of more than 90 mg/ml as salt. In some embodiments, the pharmaceutical compound has a solubility of more than 80 mg/ml as salt. In some embodiments, the pharmaceutical compound has a solubility of more than 70 mg/ml as salt. In some embodiments, the pharmaceutical compound has a solubility of more than 60 mg/ml as salt. In some embodiments, the pharmaceutical compound has a solubility of more than 50 mg/ml as salt. In some embodiments, the pharmaceutical compound has a solubility of more than 45 mg/ml as salt. In some embodiments, the pharmaceutical compound has a solubility of more than 40 mg/ml as salt. In some embodiments, the pharmaceutical compound has a solubility of more than 35 mg/ml as salt. In some embodiments, the pharmaceutical compound has a solubility of more than 30 mg/ml as salt. In some embodiments, the pharmaceutical compound has a solubility of more than 25 mg/ml as salt. In some embodiments, the pharmaceutical compound has a solubility of more than 20 mg/ml as salt. In some embodiments, the pharmaceutical compound has a solubility of more than 10 mg/ml as salt. In some embodiments, the pharmaceutical compound has a solubility of more than 9 mg/ml as salt. In some embodiments, the pharmaceutical compound has a solubility of more than 8 mg/ml as salt. In some embodiments, the pharmaceutical compound has a solubility of more than 7 mg/ml as salt. In some embodiments, the pharmaceutical compound has a solubility of more than 6 mg/ml as salt. In some embodiments, the pharmaceutical compound has a solubility of more than 5 mg/ml as salt. In some embodiments, the pharmaceutical compound has a solubility of more than 4 mg/ml as salt. In some embodiments, the pharmaceutical compound has a solubility of more than 3 mg/ml as salt. In some embodiments, the pharmaceutical compound has a solubility of more 2 mg/ml as salt. In some embodiments, the pharmaceutical compound has a solubility of more than 1 mg/ml as salt. In some embodiments, the pharmaceutical compound has a solubility of more than 0.9 mg/ml as salt. In some embodiments, the pharmaceutical compound has a solubility of more than 0.8 mg/ml as salt. In some embodiments, the pharmaceutical compound has a solubility of more than 0.7 mg/ml as salt. In some embodiments, the pharmaceutical compound has a solubility of more than 0.6 mg/ml as salt. In some embodiments, the pharmaceutical compound has a solubility of more than 0.5 mg/ml as salt. In some embodiments, the pharmaceutical compound has a solubility of more than 0.4 mg/ml as salt. In some embodiments, the pharmaceutical compound has a solubility of more than 0.3 mg/ml as salt. In some embodiments, the pharmaceutical compound has a solubility of more than 0.2 mg/ml as salt. In some embodiments, the pharmaceutical compound has a solubility of more than 0.1 mg/ml as salt. In some embodiments, the pharmaceutical compound has a solubility of more than 0.09 mg/ml as salt. In some embodiments, the pharmaceutical compound has a solubility of more than 0.08 mg/ml as salt. In some embodiments, the pharmaceutical compound has a solubility of more than 0.07 mg/ml as salt. In some embodiments, the pharmaceutical compound has a solubility of more than 0.06 mg/ml as salt. In some embodiments, the pharmaceutical compound has a solubility of more than 0.05 mg/ml as salt. In some embodiments, the pharmaceutical compound has a solubility of more than 0.04 mg/ml as salt. In some embodiments, the pharmaceutical compound has a solubility of more than 0.03 mg/ml as salt. In some embodiments, the pharmaceutical compound has a solubility of more than 0.02 mg/ml as salt. In some embodiments, the pharmaceutical compound has a solubility of more than 0.01 mg/ml as salt. In some embodiments, the pharmaceutical compound has a solubility of more than 0.009 mg/ml as salt. In some embodiments, the pharmaceutical compound has a solubility of more than 0.008 mg/ml as salt. In some embodiments, the pharmaceutical compound has a solubility of more than 0.007 mg/ml as salt. In some embodiments, the pharmaceutical compound has a solubility of more than 0.006 mg/ml as salt. In some embodiments, the pharmaceutical compound has a solubility of more than 0.005 mg/ml as salt. In some embodiments, the pharmaceutical compound has a solubility of more than 0.004 mg/ml as salt. In some embodiments, the pharmaceutical compound has a solubility of more than 0.003 mg/ml as salt. In some embodiments, the pharmaceutical compound has a solubility of more than 0.002 mg/ml as salt. In some embodiments, the pharmaceutical compound has a solubility of more than 0.001 mg/ml as salt. In some embodiments, the pharmaceutical compound has a solubility of more than 0.9 μg/ml as salt. In some embodiments, the pharmaceutical compound has a solubility of more than 0.8 μg/ml as salt. In some embodiments, the pharmaceutical compound has a solubility of more than 0.7 μg/ml as salt. In some embodiments, the pharmaceutical compound has a solubility of more than 0.6 μg/ml as salt. In some embodiments, the pharmaceutical compound has a solubility of more than 0.5 μg/ml as salt. In some embodiments, the pharmaceutical compound has a solubility of more than 0.4 μg/ml as salt. In some embodiments, the pharmaceutical compound has a solubility of more than 0.3 μg/ml as salt. In some embodiments, the pharmaceutical compound has a solubility of more than 0.2 μg/ml as salt. In some embodiments, the pharmaceutical compound has a solubility of more than 0.1 μg/ml as salt. In some embodiments, the pharmaceutical compound has a solubility of more than 0.09 μg/ml as salt. In some embodiments, the pharmaceutical compound has a solubility of more than 0.08 μg/ml as salt. In some embodiments, the pharmaceutical compound has a solubility of more than 0.07 μg/ml as salt. In some embodiments, the pharmaceutical compound has a solubility of more than 0.06 μg/ml as salt. In some embodiments, the pharmaceutical compound has a solubility of more than 0.05 μg/ml as salt. In some embodiments, the pharmaceutical compound has a solubility of more than 0.04 μg/ml as salt. In some embodiments, the pharmaceutical compound has a solubility of more than 0.03 μg/ml as salt. In some embodiments, the pharmaceutical compound has a solubility of more than 0.02 μg/ml as salt. In some embodiments, the pharmaceutical compound has a solubility of more than 0.01 μg/ml as salt. In some embodiments, the pharmaceutical compound has a solubility of more than 0.009 μg/ml as salt. In some embodiments, the pharmaceutical compound has a solubility of more than 0.008 μg/ml as salt. In some embodiments, the pharmaceutical compound has a solubility of more than 0.007 μg/ml as salt. In some embodiments, the pharmaceutical compound has a solubility of more than 0.006 μg/ml as salt. In some embodiments, the pharmaceutical compound has a solubility of more than 0.005 μg/ml as salt. In some embodiments, the pharmaceutical compound has a solubility of more than 0.004 μg/ml as salt. In some embodiments, the pharmaceutical compound has a solubility of more than 0.003 μg/ml as salt. In some embodiments, the pharmaceutical compound has a solubility of more than 0.002 μg/ml as salt. In some embodiments, the pharmaceutical compound has a solubility of more than 0.001 μg/ml as salt. In some embodiments, the solubility of the pharmaceutical compound as salt is measured in an aqueous medium. In some embodiments, the solubility of the pharmaceutical compound as salt is measured in an organic solvent. In some embodiments, the aqueous medium comprises water. In some embodiments, the salt of the pharmaceutical compound comprises a HCl salt.

In some embodiments, the pharmaceutical compound has a solubility of between about 0.001 mg/ml and 100 mg/ml as salt. In some embodiments, the pharmaceutical compound has a solubility of between about 0.01 mg/ml and 50 mg/ml as salt. In some embodiments, the pharmaceutical compound has a solubility of between about 0.02 mg/ml and 40 mg/ml as salt. In some embodiments, the pharmaceutical compound has a solubility of between about 0.03 mg/ml and 30 mg/ml as salt. In some embodiments, the pharmaceutical compound has a solubility of between about 0.04 mg/ml and 20 mg/ml as salt. In some embodiments, the pharmaceutical compound has a solubility of between about 0.05 mg/ml and 10 mg/ml as salt. In some embodiments, the pharmaceutical compound has a solubility of between about 0.06 mg/ml and 5 mg/ml as salt. In some embodiments, the pharmaceutical compound has a solubility of between about 0.07 mg/ml and 4 mg/ml as salt. In some embodiments, the pharmaceutical compound has a solubility of between about 0.09 mg/ml and 3 mg/ml as salt. In some embodiments, the pharmaceutical compound has a solubility of between about 0.1 mg/ml and 2 mg/ml as salt. In some embodiments, the solubility of the pharmaceutical compound as salt is measured in an aqueous medium. In some embodiments, the solubility of the pharmaceutical compound as salt is measured in an organic solvent. In some embodiments, the aqueous medium comprises water. In some embodiments, the salt of the pharmaceutical compound comprises a HCl salt.

In some embodiments, the pharmaceutical compound has a solubility of less than 100 mg/ml as salt. In some embodiments, the pharmaceutical compound has a solubility of less than 90 mg/ml as salt. In some embodiments, the pharmaceutical compound has a solubility of less than 80 mg/ml as salt. In some embodiments, the pharmaceutical compound has a solubility of less than 70 mg/ml as salt. In some embodiments, the pharmaceutical compound has a solubility of less than 60 mg/ml as salt. In some embodiments, the pharmaceutical compound has a solubility of less than 50 mg/ml as salt. In some embodiments, the pharmaceutical compound has a solubility of less than 45 mg/ml as salt. In some embodiments, the pharmaceutical compound has a solubility of less than 40 mg/ml as salt. In some embodiments, the pharmaceutical compound has a solubility of less than 35 mg/ml as salt. In some embodiments, the pharmaceutical compound has a solubility of less than 30 mg/ml as salt. In some embodiments, the pharmaceutical compound has a solubility of less than 25 mg/ml as salt. In some embodiments, the pharmaceutical compound has a solubility of less than 20 mg/ml as salt. In some embodiments, the pharmaceutical compound has a solubility of less than 10 mg/ml as salt. In some embodiments, the pharmaceutical compound has a solubility of less than 9 mg/ml as salt. In some embodiments, the pharmaceutical compound has a solubility of less than 8 mg/ml as salt. In some embodiments, the pharmaceutical compound has a solubility of less than 7 mg/ml as salt. In some embodiments, the pharmaceutical compound has a solubility of less than 6 mg/ml as salt. In some embodiments, the pharmaceutical compound has a solubility of less than 5 mg/ml as salt. In some embodiments, the pharmaceutical compound has a solubility of less than 4 mg/ml as salt. In some embodiments, the pharmaceutical compound has a solubility of less than 3 mg/ml as salt. In some embodiments, the pharmaceutical compound has a solubility of more 2 mg/ml as salt. In some embodiments, the pharmaceutical compound has a solubility of less than 1 mg/ml as salt. In some embodiments, the pharmaceutical compound has a solubility of less than 0.9 mg/ml as salt. In some embodiments, the pharmaceutical compound has a solubility of less than 0.8 mg/ml as salt. In some embodiments, the pharmaceutical compound has a solubility of less than 0.7 mg/ml as salt. In some embodiments, the pharmaceutical compound has a solubility of less than 0.6 mg/ml as salt. In some embodiments, the pharmaceutical compound has a solubility of less than 0.5 mg/ml as salt. In some embodiments, the pharmaceutical compound has a solubility of less than 0.4 mg/ml as salt. In some embodiments, the pharmaceutical compound has a solubility of less than 0.3 mg/ml as salt. In some embodiments, the pharmaceutical compound has a solubility of less than 0.2 mg/ml as salt. In some embodiments, the pharmaceutical compound has a solubility of less than 0.1 mg/ml as salt. In some embodiments, the pharmaceutical compound has a solubility of less than 0.09 mg/ml as salt. In some embodiments, the pharmaceutical compound has a solubility of less than 0.08 mg/ml as salt. In some embodiments, the pharmaceutical compound has a solubility of less than 0.07 mg/ml as salt. In some embodiments, the pharmaceutical compound has a solubility of less than 0.06 mg/ml as salt. In some embodiments, the pharmaceutical compound has a solubility of less than 0.05 mg/ml as salt. In some embodiments, the pharmaceutical compound has a solubility of less than 0.04 mg/ml as salt. In some embodiments, the pharmaceutical compound has a solubility of less than 0.03 mg/ml as salt. In some embodiments, the pharmaceutical compound has a solubility of less than 0.02 mg/ml as salt. In some embodiments, the pharmaceutical compound has a solubility of less than 0.01 mg/ml as salt. In some embodiments, the pharmaceutical compound has a solubility of less than 0.009 mg/ml as salt. In some embodiments, the pharmaceutical compound has a solubility of less than 0.008 mg/ml as salt. In some embodiments, the pharmaceutical compound has a solubility of less than 0.007 mg/ml as salt. In some embodiments, the pharmaceutical compound has a solubility of less than 0.006 mg/ml as salt. In some embodiments, the pharmaceutical compound has a solubility of less than 0.005 mg/ml as salt. In some embodiments, the pharmaceutical compound has a solubility of less than 0.004 mg/ml as salt. In some embodiments, the pharmaceutical compound has a solubility of less than 0.003 mg/ml as salt. In some embodiments, the pharmaceutical compound has a solubility of less than 0.002 mg/ml as salt. In some embodiments, the pharmaceutical compound has a solubility of less than 0.001 mg/ml as salt. In some embodiments, the pharmaceutical compound has a solubility of less than 0.9 μg/ml as salt. In some embodiments, the pharmaceutical compound has a solubility of less than 0.8 μg/ml as salt. In some embodiments, the pharmaceutical compound has a solubility of less than 0.7 μg/ml as salt. In some embodiments, the pharmaceutical compound has a solubility of less than 0.6 μg/ml as salt. In some embodiments, the pharmaceutical compound has a solubility of less than 0.5 μg/ml as salt. In some embodiments, the pharmaceutical compound has a solubility of less than 0.4 μg/ml as salt. In some embodiments, the pharmaceutical compound has a solubility of less than 0.3 μg/ml as salt. In some embodiments, the pharmaceutical compound has a solubility of less than 0.2 μg/ml as salt. In some embodiments, the pharmaceutical compound has a solubility of less than 0.1 μg/ml as salt. In some embodiments, the pharmaceutical compound has a solubility of less than 0.09 μg/ml as salt. In some embodiments, the pharmaceutical compound has a solubility of less than 0.08 μg/ml as salt. In some embodiments, the pharmaceutical compound has a solubility of less than 0.07 μg/ml as salt. In some embodiments, the pharmaceutical compound has a solubility of less than 0.06 μg/ml as salt. In some embodiments, the pharmaceutical compound has a solubility of less than 0.05 μg/ml as salt. In some embodiments, the pharmaceutical compound has a solubility of less than 0.04 μg/ml as salt. In some embodiments, the pharmaceutical compound has a solubility of less than 0.03 μg/ml as salt. In some embodiments, the pharmaceutical compound has a solubility of less than 0.02 μg/ml as salt. In some embodiments, the pharmaceutical compound has a solubility of less than 0.01 μg/ml as salt. In some embodiments, the pharmaceutical compound has a solubility of less than 0.009 μg/ml as salt. In some embodiments, the pharmaceutical compound has a solubility of less than 0.008 μg/ml as salt. In some embodiments, the pharmaceutical compound has a solubility of less than 0.007 μg/ml as salt. In some embodiments, the pharmaceutical compound has a solubility of less than 0.006 μg/ml as salt. In some embodiments, the pharmaceutical compound has a solubility of less than 0.005 μg/ml as salt. In some embodiments, the pharmaceutical compound has a solubility of less than 0.004 μg/ml as salt. In some embodiments, the pharmaceutical compound has a solubility of less than 0.003 μg/ml as salt. In some embodiments, the pharmaceutical compound has a solubility of less than 0.002 μg/ml as salt. In some embodiments, the pharmaceutical compound has a solubility of less than 0.001 μg/ml as salt. In some embodiments, the solubility of the pharmaceutical compound as salt is measured in an aqueous medium. In some embodiments, the solubility of the pharmaceutical compound as salt is measured in an organic solvent. In some embodiments, the aqueous medium comprises water. In some embodiments, the salt of the pharmaceutical compound comprises a HCl salt.

In some embodiments, the pharmaceutical compound has a solubility of more than 100 mg/ml as freebase. In some embodiments, the pharmaceutical compound has a solubility of more than 90 mg/ml as freebase. In some embodiments, the pharmaceutical compound has a solubility of more than 80 mg/ml as freebase. In some embodiments, the pharmaceutical compound has a solubility of more than 70 mg/ml as freebase. In some embodiments, the pharmaceutical compound has a solubility of more than 60 mg/ml as freebase. In some embodiments, the pharmaceutical compound has a solubility of more than 50 mg/ml as freebase. In some embodiments, the pharmaceutical compound has a solubility of more than 45 mg/ml as freebase. In some embodiments, the pharmaceutical compound has a solubility of more than 40 mg/ml as freebase. In some embodiments, the pharmaceutical compound has a solubility of more than 35 mg/ml as freebase. In some embodiments, the pharmaceutical compound has a solubility of more than 30 mg/ml as freebase. In some embodiments, the pharmaceutical compound has a solubility of more than 25 mg/ml as freebase. In some embodiments, the pharmaceutical compound has a solubility of more than 20 mg/ml as freebase. In some embodiments, the pharmaceutical compound has a solubility of more than 10 mg/ml as freebase. In some embodiments, the pharmaceutical compound has a solubility of more than 9 mg/ml as freebase. In some embodiments, the pharmaceutical compound has a solubility of more than 8 mg/ml as freebase. In some embodiments, the pharmaceutical compound has a solubility of more than 7 mg/ml as freebase. In some embodiments, the pharmaceutical compound has a solubility of more than 6 mg/ml as freebase. In some embodiments, the pharmaceutical compound has a solubility of more than 5 mg/ml as freebase. In some embodiments, the pharmaceutical compound has a solubility of more than 4 mg/ml as freebase. In some embodiments, the pharmaceutical compound has a solubility of more than 3 mg/ml as freebase. In some embodiments, the pharmaceutical compound has a solubility of more 2 mg/ml as freebase. In some embodiments, the pharmaceutical compound has a solubility of more than 1 mg/ml as freebase. In some embodiments, the pharmaceutical compound has a solubility of more than 0.9 mg/ml as freebase. In some embodiments, the pharmaceutical compound has a solubility of more than 0.8 mg/ml as freebase. In some embodiments, the pharmaceutical compound has a solubility of more than 0.7 mg/ml as freebase. In some embodiments, the pharmaceutical compound has a solubility of more than 0.6 mg/ml as freebase. In some embodiments, the pharmaceutical compound has a solubility of more than 0.5 mg/ml as freebase. In some embodiments, the pharmaceutical compound has a solubility of more than 0.4 mg/ml as freebase. In some embodiments, the pharmaceutical compound has a solubility of more than 0.3 mg/ml as freebase. In some embodiments, the pharmaceutical compound has a solubility of more than 0.2 mg/ml as freebase. In some embodiments, the pharmaceutical compound has a solubility of more than 0.1 mg/ml as freebase. In some embodiments, the pharmaceutical compound has a solubility of more than 0.09 mg/ml as freebase. In some embodiments, the pharmaceutical compound has a solubility of more than 0.08 mg/ml as freebase. In some embodiments, the pharmaceutical compound has a solubility of more than 0.07 mg/ml as freebase. In some embodiments, the pharmaceutical compound has a solubility of more than 0.06 mg/ml as freebase. In some embodiments, the pharmaceutical compound has a solubility of more than 0.05 mg/ml as freebase. In some embodiments, the pharmaceutical compound has a solubility of more than 0.04 mg/ml as freebase. In some embodiments, the pharmaceutical compound has a solubility of more than 0.03 mg/ml as freebase. In some embodiments, the pharmaceutical compound has a solubility of more than 0.02 mg/ml as freebase. In some embodiments, the pharmaceutical compound has a solubility of more than 0.01 mg/ml as freebase. In some embodiments, the pharmaceutical compound has a solubility of more than 0.009 mg/ml as freebase. In some embodiments, the pharmaceutical compound has a solubility of more than 0.008 mg/ml as freebase. In some embodiments, the pharmaceutical compound has a solubility of more than 0.007 mg/ml as freebase. In some embodiments, the pharmaceutical compound has a solubility of more than 0.006 mg/ml as freebase. In some embodiments, the pharmaceutical compound has a solubility of more than 0.005 mg/ml as freebase. In some embodiments, the pharmaceutical compound has a solubility of more than 0.004 mg/ml as freebase. In some embodiments, the pharmaceutical compound has a solubility of more than 0.003 mg/ml as freebase. In some embodiments, the pharmaceutical compound has a solubility of more than 0.002 mg/ml as freebase. In some embodiments, the pharmaceutical compound has a solubility of more than 0.001 mg/ml as freebase. In some embodiments, the pharmaceutical compound has a solubility of more than 0.9 μg/ml as freebase. In some embodiments, the pharmaceutical compound has a solubility of more than 0.8 μg/ml as freebase. In some embodiments, the pharmaceutical compound has a solubility of more than 0.7 μg/ml as freebase. In some embodiments, the pharmaceutical compound has a solubility of more than 0.6 μg/ml as freebase. In some embodiments, the pharmaceutical compound has a solubility of more than 0.5 μg/ml as freebase. In some embodiments, the pharmaceutical compound has a solubility of more than 0.4 μg/ml as freebase. In some embodiments, the pharmaceutical compound has a solubility of more than 0.3 μg/ml as freebase. In some embodiments, the pharmaceutical compound has a solubility of more than 0.2 μg/ml as freebase. In some embodiments, the pharmaceutical compound has a solubility of more than 0.1 μg/ml as freebase. In some embodiments, the pharmaceutical compound has a solubility of more than 0.09 μg/ml as freebase. In some embodiments, the pharmaceutical compound has a solubility of more than 0.08 μg/ml as freebase. In some embodiments, the pharmaceutical compound has a solubility of more than 0.07 μg/ml as freebase. In some embodiments, the pharmaceutical compound has a solubility of more than 0.06 μg/ml as freebase. In some embodiments, the pharmaceutical compound has a solubility of more than 0.05 μg/ml as freebase. In some embodiments, the pharmaceutical compound has a solubility of more than 0.04 μg/ml as freebase. In some embodiments, the pharmaceutical compound has a solubility of more than 0.03 μg/ml as freebase. In some embodiments, the pharmaceutical compound has a solubility of more than 0.02 μg/ml as freebase. In some embodiments, the pharmaceutical compound has a solubility of more than 0.01 μg/ml as freebase. In some embodiments, the pharmaceutical compound has a solubility of more than 0.009 μg/ml as freebase. In some embodiments, the pharmaceutical compound has a solubility of more than 0.008 μg/ml as freebase. In some embodiments, the pharmaceutical compound has a solubility of more than 0.007 μg/ml as freebase. In some embodiments, the pharmaceutical compound has a solubility of more than 0.006 μg/ml as freebase. In some embodiments, the pharmaceutical compound has a solubility of more than 0.005 μg/ml as freebase. In some embodiments, the pharmaceutical compound has a solubility of more than 0.004 μg/ml as freebase. In some embodiments, the pharmaceutical compound has a solubility of more than 0.003 μg/ml as freebase. In some embodiments, the pharmaceutical compound has a solubility of more than 0.002 μg/ml as freebase. In some embodiments, the pharmaceutical compound has a solubility of more than 0.001 μg/ml as freebase. In some embodiments, the solubility of the pharmaceutical compound as freebase is measured in an aqueous medium. In some embodiments, the solubility of the pharmaceutical compound as freebase is measured in an organic solvent. In some embodiments, the aqueous medium comprises water.

In some embodiments, the pharmaceutical compound has a solubility of between about 0.001 mg/ml and 100 mg/ml as freebase. In some embodiments, the pharmaceutical compound has a solubility of between about 0.01 mg/ml and 50 mg/ml as freebase. In some embodiments, the pharmaceutical compound has a solubility of between about 0.02 mg/ml and 40 mg/ml as freebase. In some embodiments, the pharmaceutical compound has a solubility of between about 0.03 mg/ml and 30 mg/ml as freebase. In some embodiments, the pharmaceutical compound has a solubility of between about 0.04 mg/ml and 20 mg/ml as freebase. In some embodiments, the pharmaceutical compound has a solubility of between about 0.05 mg/ml and 10 mg/ml as freebase. In some embodiments, the pharmaceutical compound has a solubility of between about 0.06 mg/ml and 5 mg/ml as freebase. In some embodiments, the pharmaceutical compound has a solubility of between about 0.07 mg/ml and 4 mg/ml as freebase. In some embodiments, the pharmaceutical compound has a solubility of between about 0.09 mg/ml and 3 mg/ml as freebase. In some embodiments, the pharmaceutical compound has a solubility of between about 0.1 mg/ml and 2 mg/ml as freebase. In some embodiments, the solubility of the pharmaceutical compound as freebase is measured in an aqueous medium. In some embodiments, the solubility of the pharmaceutical compound as freebase is measured in an organic solvent. In some embodiments, the aqueous medium comprises water.

In some embodiments, the pharmaceutical compound has a solubility of less than 100 mg/ml as freebase. In some embodiments, the pharmaceutical compound has a solubility of less than 90 mg/ml as freebase. In some embodiments, the pharmaceutical compound has a solubility of less than 80 mg/ml as freebase. In some embodiments, the pharmaceutical compound has a solubility of less than 70 mg/ml as freebase. In some embodiments, the pharmaceutical compound has a solubility of less than 60 mg/ml as freebase. In some embodiments, the pharmaceutical compound has a solubility of less than 50 mg/ml as freebase. In some embodiments, the pharmaceutical compound has a solubility of less than 45 mg/ml as freebase. In some embodiments, the pharmaceutical compound has a solubility of less than 40 mg/ml as freebase. In some embodiments, the pharmaceutical compound has a solubility of less than 35 mg/ml as freebase. In some embodiments, the pharmaceutical compound has a solubility of less than 30 mg/ml as freebase. In some embodiments, the pharmaceutical compound has a solubility of less than 25 mg/ml as freebase. In some embodiments, the pharmaceutical compound has a solubility of less than 20 mg/ml as freebase. In some embodiments, the pharmaceutical compound has a solubility of less than 10 mg/ml as freebase. In some embodiments, the pharmaceutical compound has a solubility of less than 9 mg/ml as freebase. In some embodiments, the pharmaceutical compound has a solubility of less than 8 mg/ml as freebase. In some embodiments, the pharmaceutical compound has a solubility of less than 7 mg/ml as freebase. In some embodiments, the pharmaceutical compound has a solubility of less than 6 mg/ml as freebase. In some embodiments, the pharmaceutical compound has a solubility of less than 5 mg/ml as freebase. In some embodiments, the pharmaceutical compound has a solubility of less than 4 mg/ml as freebase. In some embodiments, the pharmaceutical compound has a solubility of less than 3 mg/ml as freebase. In some embodiments, the pharmaceutical compound has a solubility of more 2 mg/ml as freebase. In some embodiments, the pharmaceutical compound has a solubility of less than 1 mg/ml as freebase. In some embodiments, the pharmaceutical compound has a solubility of less than 0.9 mg/ml as freebase. In some embodiments, the pharmaceutical compound has a solubility of less than 0.8 mg/ml as freebase. In some embodiments, the pharmaceutical compound has a solubility of less than 0.7 mg/ml as freebase. In some embodiments, the pharmaceutical compound has a solubility of less than 0.6 mg/ml as freebase. In some embodiments, the pharmaceutical compound has a solubility of less than 0.5 mg/ml as freebase. In some embodiments, the pharmaceutical compound has a solubility of less than 0.4 mg/ml as freebase. In some embodiments, the pharmaceutical compound has a solubility of less than 0.3 mg/ml as freebase. In some embodiments, the pharmaceutical compound has a solubility of less than 0.2 mg/ml as freebase. In some embodiments, the pharmaceutical compound has a solubility of less than 0.1 mg/ml as freebase. In some embodiments, the pharmaceutical compound has a solubility of less than 0.09 mg/ml as freebase. In some embodiments, the pharmaceutical compound has a solubility of less than 0.08 mg/ml as freebase. In some embodiments, the pharmaceutical compound has a solubility of less than 0.07 mg/ml as freebase. In some embodiments, the pharmaceutical compound has a solubility of less than 0.06 mg/ml as freebase. In some embodiments, the pharmaceutical compound has a solubility of less than 0.05 mg/ml as freebase. In some embodiments, the pharmaceutical compound has a solubility of less than 0.04 mg/ml as freebase. In some embodiments, the pharmaceutical compound has a solubility of less than 0.03 mg/ml as freebase. In some embodiments, the pharmaceutical compound has a solubility of less than 0.02 mg/ml as freebase. In some embodiments, the pharmaceutical compound has a solubility of less than 0.01 mg/ml as freebase. In some embodiments, the pharmaceutical compound has a solubility of less than 0.009 mg/ml as freebase. In some embodiments, the pharmaceutical compound has a solubility of less than 0.008 mg/ml as freebase. In some embodiments, the pharmaceutical compound has a solubility of less than 0.007 mg/ml as freebase. In some embodiments, the pharmaceutical compound has a solubility of less than 0.006 mg/ml as freebase. In some embodiments, the pharmaceutical compound has a solubility of less than 0.005 mg/ml as freebase. In some embodiments, the pharmaceutical compound has a solubility of less than 0.004 mg/ml as freebase. In some embodiments, the pharmaceutical compound has a solubility of less than 0.003 mg/ml as freebase. In some embodiments, the pharmaceutical compound has a solubility of less than 0.002 mg/ml as freebase. In some embodiments, the pharmaceutical compound has a solubility of less than 0.001 mg/ml as freebase. In some embodiments, the pharmaceutical compound has a solubility of less than 0.9 μg/ml as freebase. In some embodiments, the pharmaceutical compound has a solubility of less than 0.8 μg/ml as freebase. In some embodiments, the pharmaceutical compound has a solubility of less than 0.7 μg/ml as freebase. In some embodiments, the pharmaceutical compound has a solubility of less than 0.6 μg/ml as freebase. In some embodiments, the pharmaceutical compound has a solubility of less than 0.5 μg/ml as freebase. In some embodiments, the pharmaceutical compound has a solubility of less than 0.4 μg/ml as freebase. In some embodiments, the pharmaceutical compound has a solubility of less than 0.3 μg/ml as freebase. In some embodiments, the pharmaceutical compound has a solubility of less than 0.2 μg/ml as freebase. In some embodiments, the pharmaceutical compound has a solubility of less than 0.1 μg/ml as freebase. In some embodiments, the pharmaceutical compound has a solubility of less than 0.09 μg/ml as freebase. In some embodiments, the pharmaceutical compound has a solubility of less than 0.08 μg/ml as freebase. In some embodiments, the pharmaceutical compound has a solubility of less than 0.07 μg/ml as freebase. In some embodiments, the pharmaceutical compound has a solubility of less than 0.06 μg/ml as freebase. In some embodiments, the pharmaceutical compound has a solubility of less than 0.05 μg/ml as freebase. In some embodiments, the pharmaceutical compound has a solubility of less than 0.04 μg/ml as freebase. In some embodiments, the pharmaceutical compound has a solubility of less than 0.03 μg/ml as freebase. In some embodiments, the pharmaceutical compound has a solubility of less than 0.02 μg/ml as freebase. In some embodiments, the pharmaceutical compound has a solubility of less than 0.01 μg/ml as freebase. In some embodiments, the pharmaceutical compound has a solubility of less than 0.009 μg/ml as freebase. In some embodiments, the pharmaceutical compound has a solubility of less than 0.008 μg/ml as freebase. In some embodiments, the pharmaceutical compound has a solubility of less than 0.007 μg/ml as freebase. In some embodiments, the pharmaceutical compound has a solubility of less than 0.006 μg/ml as freebase. In some embodiments, the pharmaceutical compound has a solubility of less than 0.005 μg/ml as freebase. In some embodiments, the pharmaceutical compound has a solubility of less than 0.004 μg/ml as freebase. In some embodiments, the pharmaceutical compound has a solubility of less than 0.003 μg/ml as freebase. In some embodiments, the pharmaceutical compound has a solubility of less than 0.002 μg/ml as freebase. In some embodiments, the pharmaceutical compound has a solubility of less than 0.001 μg/ml as freebase. In some embodiments, the solubility of the pharmaceutical compound as freebase is measured in an aqueous medium. In some embodiments, the solubility of the pharmaceutical compound as freebase is measured in an organic solvent. In some embodiments, the aqueous medium comprises water.

In some embodiments, the ratio of the conjugate base of the complexing agent to the pharmaceutical compound in the pharmaceutically acceptable salt is from about 1:1 to about 1:4. In some embodiments, the ratio of the conjugate base of the complexing agent to the pharmaceutical compound in the pharmaceutically acceptable salt is from about 1:1.1 to about 1:3.9. In some embodiments, the ratio of the conjugate base of the complexing agent to the pharmaceutical compound in the pharmaceutically acceptable salt is from about 1:1.2 to about 1:3.8. In some embodiments, the ratio of the conjugate base of the complexing agent to the pharmaceutical compound in the pharmaceutically acceptable salt is from about 1:1.3 to about 1:3.7. In some embodiments, the ratio of the conjugate base of the complexing agent to the pharmaceutical compound in the pharmaceutically acceptable salt is from about 1:1.4 to about 1:3.6. In some embodiments, the ratio of the conjugate base of the complexing agent to the pharmaceutical compound in the pharmaceutically acceptable salt is from about 1:1.5 to about 1:3.5. In some embodiments, the ratio of the conjugate base of the complexing agent to the pharmaceutical compound in the pharmaceutically acceptable salt is from about 1:1.6 to about 1:3.4. In some embodiments, the ratio of the conjugate base of the complexing agent to the pharmaceutical compound in the pharmaceutically acceptable salt is from about 1:1.7 to about 1:3.3. In some embodiments, the ratio of the conjugate base of the complexing agent to the pharmaceutical compound in the pharmaceutically acceptable salt is from about 1:1.8 to about 1:3.2. In some embodiments, the ratio of the conjugate base of the complexing agent to the pharmaceutical compound in the pharmaceutically acceptable salt is from about 1:1.9 to about 1:3.1. In some embodiments, the ratio of the conjugate base of the complexing agent to the pharmaceutical compound in the pharmaceutically acceptable salt is from about 1:2 to about 1:3. In some embodiments, the ratio of the conjugate base of the complexing agent to the pharmaceutical compound in the pharmaceutically acceptable salt is from about 1:2.1 to about 1:2.9. In some embodiments, the ratio of the conjugate base of the complexing agent to the pharmaceutical compound in the pharmaceutically acceptable salt is from about 1:2.2 to about 1:2.8. In some embodiments, the ratio of the conjugate base of the complexing agent to the pharmaceutical compound in the pharmaceutically acceptable salt is from about 1:2.3 to about 1:2.7. In some embodiments, the ratio of the conjugate base of the complexing agent to the pharmaceutical compound in the pharmaceutically acceptable salt is from about 1:2.4 to about 1:2.6. In some embodiments, the ratio of the conjugate base of the complexing agent to the pharmaceutical compound in the pharmaceutically acceptable salt is from about 1:1 to about 1:4, about 1:1.1, 1:1.2, 1:1.3, 1:1.4, 1:1.5, 1:1.6, 1:1.7, 1:1.8, 1:1.9, 1:2.0, 1:2. 1, 1:2.2, 1:2.3, 1:2.4, 1:2.5, 1:2.6, 1:2.7, 1:2.8, 1:2.9, 1:3.0, 1:3.1, 1:3.2, 1:3.3, 1:3.4, 1:3.5, 1:3.6, 1:3.7, 1:3.8, 1:3.9, or about 1:4.0, or any ratio therebetween.

In some embodiments, the complexing agent acts as the counterion to between 1 to 4 molecules of the pharmaceutical compound in the pharmaceutically acceptable salt. In some embodiments, the complexing agent further comprises a non-polar region. In some embodiments, the pharmaceutically acceptable salt comprises an additional molar equivalent of the pharmaceutical compound in an unionized form compared to the amount of complexing agent. In some embodiments, the additional molar equivalent of the pharmaceutical compound in the unionized form is complexed to the complexing agent through the non-polar region.

In some embodiments, the solubility of the pharmaceutical compound in the pharmaceutically acceptable salt is higher than (i) the solubility of the pharmaceutical compound as a salt; or (ii) the solubility of the pharmaceutical compound in a salt comprising the pharmaceutical compound in freebase form that is complexing to a non-polar pore of the complexing agent, wherein the pharmaceutical compound has the same concentration in the pharmaceutically acceptable salt, the pharmaceutical compound as a salt, and the salt comprising the pharmaceutical compound in freebase form. In some embodiments, the solubility of the pharmaceutical compound in the pharmaceutically acceptable salt is higher than the solubility of the pharmaceutical compound in a salt comprising the pharmaceutical compound and complexing agent with a higher molar ratio of the pharmaceutical compound to the complexing agent, wherein the pharmaceutical compound has the same concentration in the pharmaceutically acceptable salt and the salt with a higher molar ratio of the pharmaceutical compound to the complexing agent. In some embodiments, the solubility of the pharmaceutical compound in the pharmaceutically acceptable salt has an increase of at least 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, or 100-fold compared with (i) the solubility of the pharmaceutical compound as a salt; or (ii) the solubility of the pharmaceutical compound in a salt comprising the pharmaceutical compound in freebase form that is complexing to a non-polar pore of the complexing agent, wherein the pharmaceutical compound has the same concentration in the pharmaceutically acceptable salt, the pharmaceutical compound as a salt, and the salt comprising the pharmaceutical compound in freebase form. In some embodiments, the solubility of the pharmaceutical compound in the pharmaceutically acceptable salt has an increase of at least 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, or 100-fold compared with the solubility of the pharmaceutical compound in a salt comprising the pharmaceutical compound and complexing agent with a higher molar ratio of the pharmaceutical compound to the complexing agent, wherein the pharmaceutical compound has the same concentration in the pharmaceutically acceptable salt and the salt with a higher molar ratio of the pharmaceutical compound to the complexing agent.

In an aspect, provided herein is a pharmaceutically acceptable salt of a compound pharmaceutical comprising: (i) a pharmaceutical compound, or an enantiomer, a mixture of enantiomers, or an isotopic variant thereof; or a pharmaceutically acceptable salt, solvate or hydrate thereof, wherein the pharmaceutical compound comprises a protonated nitrogen atom; (ii) a conjugate base of a complexing agent comprising a plurality of acidic functional groups, wherein the plurality of acidic functional groups comprise a conjugate base of an acid which acts as a counterion for the protonated nitrogen atom of the pharmaceutical compound, wherein the pharmaceutically acceptable salt has a ratio of the conjugate base of the complexing agent to the pharmaceutical compound that is from about 1:1 to about 1:4; and an additional molar equivalent of the pharmaceutical compound, wherein the additional molar equivalent of the pharmaceutical compound is unionized. In some embodiments, the complexing agent acts as the counterion to between 1 to 4 molecules of the pharmaceutical compound in the pharmaceutically acceptable salt. In some embodiments, the pharmaceutical compound comprises a solubility below a threshold value. In some embodiments, the pharmaceutical compound comprises a solubility above a threshold value. In some embodiments, the pharmaceutical compound comprises a pKa above a threshold value. In some embodiments, the pharmaceutical compound comprises a pKa below a threshold value.

In some embodiments, the complexing agent further comprises a non-polar region. In some embodiments, the additional molar equivalent of the pharmaceutical compound in the unionized form is complexed to the complexing agent through the non-polar region. In some embodiments, the pharmaceutical compound is an antiviral compound, an antibacterial compound, an anti-fungal compound, a compound for treatment of a neurological disorder, a compound for treatment of Parkinson's disease, a treatment for migraine headache, a treatment for autoimmune disease, a treatment for cancer, a treatment for lymphoma, a treatment for pancreatitis, a treatment for opioid overdose, a treatment for flu infection, or a treatment for an inflammatory disorder. In some embodiments, the pharmaceutical compound comprises rotigotine, eletriptan, copanlisib, remdesivir, nafamostat (nafamostat mesylate), melevodopa, tigecycline, naloxone, amikacin, 1-amantadine, 2-amantadine, rimantadine, amifampridine, rapamycin, clonidine, or caspofungin. In some embodiments, the complexing agent is sulfobutylether-β-cyclodextrin.

In some embodiments, the pharmaceutical compound has a solubility of more than 100 mg/ml as salt. In some embodiments, the pharmaceutical compound has a solubility of more than 90 mg/ml as salt. In some embodiments, the pharmaceutical compound has a solubility of more than 80 mg/ml as salt. In some embodiments, the pharmaceutical compound has a solubility of more than 70 mg/ml as salt. In some embodiments, the pharmaceutical compound has a solubility of more than 60 mg/ml as salt. In some embodiments, the pharmaceutical compound has a solubility of more than 50 mg/ml as salt. In some embodiments, the pharmaceutical compound has a solubility of more than 45 mg/ml as salt. In some embodiments, the pharmaceutical compound has a solubility of more than 40 mg/ml as salt. In some embodiments, the pharmaceutical compound has a solubility of more than 35 mg/ml as salt. In some embodiments, the pharmaceutical compound has a solubility of more than 30 mg/ml as salt. In some embodiments, the pharmaceutical compound has a solubility of more than 25 mg/ml as salt. In some embodiments, the pharmaceutical compound has a solubility of more than 20 mg/ml as salt. In some embodiments, the pharmaceutical compound has a solubility of more than 10 mg/ml as salt. In some embodiments, the pharmaceutical compound has a solubility of more than 9 mg/ml as salt. In some embodiments, the pharmaceutical compound has a solubility of more than 8 mg/ml as salt. In some embodiments, the pharmaceutical compound has a solubility of more than 7 mg/ml as salt. In some embodiments, the pharmaceutical compound has a solubility of more than 6 mg/ml as salt. In some embodiments, the pharmaceutical compound has a solubility of more than 5 mg/ml as salt. In some embodiments, the pharmaceutical compound has a solubility of more than 4 mg/ml as salt. In some embodiments, the pharmaceutical compound has a solubility of more than 3 mg/ml as salt. In some embodiments, the pharmaceutical compound has a solubility of more 2 mg/ml as salt. In some embodiments, the pharmaceutical compound has a solubility of more than 1 mg/ml as salt. In some embodiments, the pharmaceutical compound has a solubility of more than 0.9 mg/ml as salt. In some embodiments, the pharmaceutical compound has a solubility of more than 0.8 mg/ml as salt. In some embodiments, the pharmaceutical compound has a solubility of more than 0.7 mg/ml as salt. In some embodiments, the pharmaceutical compound has a solubility of more than 0.6 mg/ml as salt. In some embodiments, the pharmaceutical compound has a solubility of more than 0.5 mg/ml as salt. In some embodiments, the pharmaceutical compound has a solubility of more than 0.4 mg/ml as salt. In some embodiments, the pharmaceutical compound has a solubility of more than 0.3 mg/ml as salt. In some embodiments, the pharmaceutical compound has a solubility of more than 0.2 mg/ml as salt. In some embodiments, the pharmaceutical compound has a solubility of more than 0.1 mg/ml as salt. In some embodiments, the pharmaceutical compound has a solubility of more than 0.09 mg/ml as salt. In some embodiments, the pharmaceutical compound has a solubility of more than 0.08 mg/ml as salt. In some embodiments, the pharmaceutical compound has a solubility of more than 0.07 mg/ml as salt. In some embodiments, the pharmaceutical compound has a solubility of more than 0.06 mg/ml as salt. In some embodiments, the pharmaceutical compound has a solubility of more than 0.05 mg/ml as salt. In some embodiments, the pharmaceutical compound has a solubility of more than 0.04 mg/ml as salt. In some embodiments, the pharmaceutical compound has a solubility of more than 0.03 mg/ml as salt. In some embodiments, the pharmaceutical compound has a solubility of more than 0.02 mg/ml as salt. In some embodiments, the pharmaceutical compound has a solubility of more than 0.01 mg/ml as salt. In some embodiments, the pharmaceutical compound has a solubility of more than 0.009 mg/ml as salt. In some embodiments, the pharmaceutical compound has a solubility of more than 0.008 mg/ml as salt. In some embodiments, the pharmaceutical compound has a solubility of more than 0.007 mg/ml as salt. In some embodiments, the pharmaceutical compound has a solubility of more than 0.006 mg/ml as salt. In some embodiments, the pharmaceutical compound has a solubility of more than 0.005 mg/ml as salt. In some embodiments, the pharmaceutical compound has a solubility of more than 0.004 mg/ml as salt. In some embodiments, the pharmaceutical compound has a solubility of more than 0.003 mg/ml as salt. In some embodiments, the pharmaceutical compound has a solubility of more than 0.002 mg/ml as salt. In some embodiments, the pharmaceutical compound has a solubility of more than 0.001 mg/ml as salt. In some embodiments, the pharmaceutical compound has a solubility of more than 0.9 μg/ml as salt. In some embodiments, the pharmaceutical compound has a solubility of more than 0.8 μg/ml as salt. In some embodiments, the pharmaceutical compound has a solubility of more than 0.7 μg/ml as salt. In some embodiments, the pharmaceutical compound has a solubility of more than 0.6 μg/ml as salt. In some embodiments, the pharmaceutical compound has a solubility of more than 0.5 μg/ml as salt. In some embodiments, the pharmaceutical compound has a solubility of more than 0.4 μg/ml as salt. In some embodiments, the pharmaceutical compound has a solubility of more than 0.3 μg/ml as salt. In some embodiments, the pharmaceutical compound has a solubility of more than 0.2 μg/ml as salt. In some embodiments, the pharmaceutical compound has a solubility of more than 0.1 μg/ml as salt. In some embodiments, the pharmaceutical compound has a solubility of more than 0.09 μg/ml as salt. In some embodiments, the pharmaceutical compound has a solubility of more than 0.08 μg/ml as salt. In some embodiments, the pharmaceutical compound has a solubility of more than 0.07 μg/ml as salt. In some embodiments, the pharmaceutical compound has a solubility of more than 0.06 μg/ml as salt. In some embodiments, the pharmaceutical compound has a solubility of more than 0.05 μg/ml as salt. In some embodiments, the pharmaceutical compound has a solubility of more than 0.04 μg/ml as salt. In some embodiments, the pharmaceutical compound has a solubility of more than 0.03 μg/ml as salt. In some embodiments, the pharmaceutical compound has a solubility of more than 0.02 μg/ml as salt. In some embodiments, the pharmaceutical compound has a solubility of more than 0.01 μg/ml as salt. In some embodiments, the pharmaceutical compound has a solubility of more than 0.009 μg/ml as salt. In some embodiments, the pharmaceutical compound has a solubility of more than 0.008 μg/ml as salt. In some embodiments, the pharmaceutical compound has a solubility of more than 0.007 μg/ml as salt. In some embodiments, the pharmaceutical compound has a solubility of more than 0.006 μg/ml as salt. In some embodiments, the pharmaceutical compound has a solubility of more than 0.005 μg/ml as salt. In some embodiments, the pharmaceutical compound has a solubility of more than 0.004 μg/ml as salt. In some embodiments, the pharmaceutical compound has a solubility of more than 0.003 μg/ml as salt. In some embodiments, the pharmaceutical compound has a solubility of more than 0.002 μg/ml as salt. In some embodiments, the pharmaceutical compound has a solubility of more than 0.001 μg/ml as salt. In some embodiments, the solubility of the pharmaceutical compound as salt is measured in an aqueous medium. In some embodiments, the solubility of the pharmaceutical compound as salt is measured in an organic solvent. In some embodiments, the aqueous medium comprises water. In some embodiments, the salt of the pharmaceutical compound comprises a HCl salt.

In some embodiments, the pharmaceutical compound has a solubility of between about 0.001 mg/ml and 100 mg/ml as salt. In some embodiments, the pharmaceutical compound has a solubility of between about 0.01 mg/ml and 50 mg/ml as salt. In some embodiments, the pharmaceutical compound has a solubility of between about 0.02 mg/ml and 40 mg/ml as salt. In some embodiments, the pharmaceutical compound has a solubility of between about 0.03 mg/ml and 30 mg/ml as salt. In some embodiments, the pharmaceutical compound has a solubility of between about 0.04 mg/ml and 20 mg/ml as salt. In some embodiments, the pharmaceutical compound has a solubility of between about 0.05 mg/ml and 10 mg/ml as salt. In some embodiments, the pharmaceutical compound has a solubility of between about 0.06 mg/ml and 5 mg/ml as salt. In some embodiments, the pharmaceutical compound has a solubility of between about 0.07 mg/ml and 4 mg/ml as salt. In some embodiments, the pharmaceutical compound has a solubility of between about 0.09 mg/ml and 3 mg/ml as salt. In some embodiments, the pharmaceutical compound has a solubility of between about 0.1 mg/ml and 2 mg/ml as salt. In some embodiments, the solubility of the pharmaceutical compound as salt is measured in an aqueous medium. In some embodiments, the solubility of the pharmaceutical compound as salt is measured in an organic solvent. In some embodiments, the aqueous medium comprises water. In some embodiments, the salt of the pharmaceutical compound comprises a HCl salt.

In some embodiments, the pharmaceutical compound has a solubility of less than 100 mg/ml as salt. In some embodiments, the pharmaceutical compound has a solubility of less than 90 mg/ml as salt. In some embodiments, the pharmaceutical compound has a solubility of less than 80 mg/ml as salt. In some embodiments, the pharmaceutical compound has a solubility of less than 70 mg/ml as salt. In some embodiments, the pharmaceutical compound has a solubility of less than 60 mg/ml as salt. In some embodiments, the pharmaceutical compound has a solubility of less than 50 mg/ml as salt. In some embodiments, the pharmaceutical compound has a solubility of less than 45 mg/ml as salt. In some embodiments, the pharmaceutical compound has a solubility of less than 40 mg/ml as salt. In some embodiments, the pharmaceutical compound has a solubility of less than 35 mg/ml as salt. In some embodiments, the pharmaceutical compound has a solubility of less than 30 mg/ml as salt. In some embodiments, the pharmaceutical compound has a solubility of less than 25 mg/ml as salt. In some embodiments, the pharmaceutical compound has a solubility of less than 20 mg/ml as salt. In some embodiments, the pharmaceutical compound has a solubility of less than 10 mg/ml as salt. In some embodiments, the pharmaceutical compound has a solubility of less than 9 mg/ml as salt. In some embodiments, the pharmaceutical compound has a solubility of less than 8 mg/ml as salt. In some embodiments, the pharmaceutical compound has a solubility of less than 7 mg/ml as salt. In some embodiments, the pharmaceutical compound has a solubility of less than 6 mg/ml as salt. In some embodiments, the pharmaceutical compound has a solubility of less than 5 mg/ml as salt. In some embodiments, the pharmaceutical compound has a solubility of less than 4 mg/ml as salt. In some embodiments, the pharmaceutical compound has a solubility of less than 3 mg/ml as salt. In some embodiments, the pharmaceutical compound has a solubility of more 2 mg/ml as salt. In some embodiments, the pharmaceutical compound has a solubility of less than 1 mg/ml as salt. In some embodiments, the pharmaceutical compound has a solubility of less than 0.9 mg/ml as salt. In some embodiments, the pharmaceutical compound has a solubility of less than 0.8 mg/ml as salt. In some embodiments, the pharmaceutical compound has a solubility of less than 0.7 mg/ml as salt. In some embodiments, the pharmaceutical compound has a solubility of less than 0.6 mg/ml as salt. In some embodiments, the pharmaceutical compound has a solubility of less than 0.5 mg/ml as salt. In some embodiments, the pharmaceutical compound has a solubility of less than 0.4 mg/ml as salt. In some embodiments, the pharmaceutical compound has a solubility of less than 0.3 mg/ml as salt. In some embodiments, the pharmaceutical compound has a solubility of less than 0.2 mg/ml as salt. In some embodiments, the pharmaceutical compound has a solubility of less than 0.1 mg/ml as salt. In some embodiments, the pharmaceutical compound has a solubility of less than 0.09 mg/ml as salt. In some embodiments, the pharmaceutical compound has a solubility of less than 0.08 mg/ml as salt. In some embodiments, the pharmaceutical compound has a solubility of less than 0.07 mg/ml as salt. In some embodiments, the pharmaceutical compound has a solubility of less than 0.06 mg/ml as salt. In some embodiments, the pharmaceutical compound has a solubility of less than 0.05 mg/ml as salt. In some embodiments, the pharmaceutical compound has a solubility of less than 0.04 mg/ml as salt. In some embodiments, the pharmaceutical compound has a solubility of less than 0.03 mg/ml as salt. In some embodiments, the pharmaceutical compound has a solubility of less than 0.02 mg/ml as salt. In some embodiments, the pharmaceutical compound has a solubility of less than 0.01 mg/ml as salt. In some embodiments, the pharmaceutical compound has a solubility of less than 0.009 mg/ml as salt. In some embodiments, the pharmaceutical compound has a solubility of less than 0.008 mg/ml as salt. In some embodiments, the pharmaceutical compound has a solubility of less than 0.007 mg/ml as salt. In some embodiments, the pharmaceutical compound has a solubility of less than 0.006 mg/ml as salt. In some embodiments, the pharmaceutical compound has a solubility of less than 0.005 mg/ml as salt. In some embodiments, the pharmaceutical compound has a solubility of less than 0.004 mg/ml as salt. In some embodiments, the pharmaceutical compound has a solubility of less than 0.003 mg/ml as salt. In some embodiments, the pharmaceutical compound has a solubility of less than 0.002 mg/ml as salt. In some embodiments, the pharmaceutical compound has a solubility of less than 0.001 mg/ml as salt. In some embodiments, the pharmaceutical compound has a solubility of less than 0.9 μg/ml as salt. In some embodiments, the pharmaceutical compound has a solubility of less than 0.8 μg/ml as salt. In some embodiments, the pharmaceutical compound has a solubility of less than 0.7 μg/ml as salt. In some embodiments, the pharmaceutical compound has a solubility of less than 0.6 μg/ml as salt. In some embodiments, the pharmaceutical compound has a solubility of less than 0.5 μg/ml as salt. In some embodiments, the pharmaceutical compound has a solubility of less than 0.4 μg/ml as salt. In some embodiments, the pharmaceutical compound has a solubility of less than 0.3 μg/ml as salt. In some embodiments, the pharmaceutical compound has a solubility of less than 0.2 μg/ml as salt. In some embodiments, the pharmaceutical compound has a solubility of less than 0.1 μg/ml as salt. In some embodiments, the pharmaceutical compound has a solubility of less than 0.09 μg/ml as salt. In some embodiments, the pharmaceutical compound has a solubility of less than 0.08 μg/ml as salt. In some embodiments, the pharmaceutical compound has a solubility of less than 0.07 μg/ml as salt. In some embodiments, the pharmaceutical compound has a solubility of less than 0.06 μg/ml as salt. In some embodiments, the pharmaceutical compound has a solubility of less than 0.05 μg/ml as salt. In some embodiments, the pharmaceutical compound has a solubility of less than 0.04 μg/ml as salt. In some embodiments, the pharmaceutical compound has a solubility of less than 0.03 μg/ml as salt. In some embodiments, the pharmaceutical compound has a solubility of less than 0.02 μg/ml as salt. In some embodiments, the pharmaceutical compound has a solubility of less than 0.01 μg/ml as salt. In some embodiments, the pharmaceutical compound has a solubility of less than 0.009 μg/ml as salt. In some embodiments, the pharmaceutical compound has a solubility of less than 0.008 μg/ml as salt. In some embodiments, the pharmaceutical compound has a solubility of less than 0.007 μg/ml as salt. In some embodiments, the pharmaceutical compound has a solubility of less than 0.006 μg/ml as salt. In some embodiments, the pharmaceutical compound has a solubility of less than 0.005 μg/ml as salt. In some embodiments, the pharmaceutical compound has a solubility of less than 0.004 μg/ml as salt. In some embodiments, the pharmaceutical compound has a solubility of less than 0.003 μg/ml as salt. In some embodiments, the pharmaceutical compound has a solubility of less than 0.002 μg/ml as salt. In some embodiments, the pharmaceutical compound has a solubility of less than 0.001 μg/ml as salt. In some embodiments, the solubility of the pharmaceutical compound as salt is measured in an aqueous medium. In some embodiments, the solubility of the pharmaceutical compound as salt is measured in an organic solvent. In some embodiments, the aqueous medium comprises water. In some embodiments, the salt of the pharmaceutical compound comprises a HCl salt.

In some embodiments, the pharmaceutical compound has a solubility of more than 100 mg/ml as freebase. In some embodiments, the pharmaceutical compound has a solubility of more than 90 mg/ml as freebase. In some embodiments, the pharmaceutical compound has a solubility of more than 80 mg/ml as freebase. In some embodiments, the pharmaceutical compound has a solubility of more than 70 mg/ml as freebase. In some embodiments, the pharmaceutical compound has a solubility of more than 60 mg/ml as freebase. In some embodiments, the pharmaceutical compound has a solubility of more than 50 mg/ml as freebase. In some embodiments, the pharmaceutical compound has a solubility of more than 45 mg/ml as freebase. In some embodiments, the pharmaceutical compound has a solubility of more than 40 mg/ml as freebase. In some embodiments, the pharmaceutical compound has a solubility of more than 35 mg/ml as freebase. In some embodiments, the pharmaceutical compound has a solubility of more than 30 mg/ml as freebase. In some embodiments, the pharmaceutical compound has a solubility of more than 25 mg/ml as freebase. In some embodiments, the pharmaceutical compound has a solubility of more than 20 mg/ml as freebase. In some embodiments, the pharmaceutical compound has a solubility of more than 10 mg/ml as freebase. In some embodiments, the pharmaceutical compound has a solubility of more than 9 mg/ml as freebase. In some embodiments, the pharmaceutical compound has a solubility of more than 8 mg/ml as freebase. In some embodiments, the pharmaceutical compound has a solubility of more than 7 mg/ml as freebase. In some embodiments, the pharmaceutical compound has a solubility of more than 6 mg/ml as freebase. In some embodiments, the pharmaceutical compound has a solubility of more than 5 mg/ml as freebase. In some embodiments, the pharmaceutical compound has a solubility of more than 4 mg/ml as freebase. In some embodiments, the pharmaceutical compound has a solubility of more than 3 mg/ml as freebase. In some embodiments, the pharmaceutical compound has a solubility of more 2 mg/ml as freebase. In some embodiments, the pharmaceutical compound has a solubility of more than 1 mg/ml as freebase. In some embodiments, the pharmaceutical compound has a solubility of more than 0.9 mg/ml as freebase. In some embodiments, the pharmaceutical compound has a solubility of more than 0.8 mg/ml as freebase. In some embodiments, the pharmaceutical compound has a solubility of more than 0.7 mg/ml as freebase. In some embodiments, the pharmaceutical compound has a solubility of more than 0.6 mg/ml as freebase. In some embodiments, the pharmaceutical compound has a solubility of more than 0.5 mg/ml as freebase. In some embodiments, the pharmaceutical compound has a solubility of more than 0.4 mg/ml as freebase. In some embodiments, the pharmaceutical compound has a solubility of more than 0.3 mg/ml as freebase. In some embodiments, the pharmaceutical compound has a solubility of more than 0.2 mg/ml as freebase. In some embodiments, the pharmaceutical compound has a solubility of more than 0.1 mg/ml as freebase. In some embodiments, the pharmaceutical compound has a solubility of more than 0.09 mg/ml as freebase. In some embodiments, the pharmaceutical compound has a solubility of more than 0.08 mg/ml as freebase. In some embodiments, the pharmaceutical compound has a solubility of more than 0.07 mg/ml as freebase. In some embodiments, the pharmaceutical compound has a solubility of more than 0.06 mg/ml as freebase. In some embodiments, the pharmaceutical compound has a solubility of more than 0.05 mg/ml as freebase. In some embodiments, the pharmaceutical compound has a solubility of more than 0.04 mg/ml as freebase. In some embodiments, the pharmaceutical compound has a solubility of more than 0.03 mg/ml as freebase. In some embodiments, the pharmaceutical compound has a solubility of more than 0.02 mg/ml as freebase. In some embodiments, the pharmaceutical compound has a solubility of more than 0.01 mg/ml as freebase. In some embodiments, the pharmaceutical compound has a solubility of more than 0.009 mg/ml as freebase. In some embodiments, the pharmaceutical compound has a solubility of more than 0.008 mg/ml as freebase. In some embodiments, the pharmaceutical compound has a solubility of more than 0.007 mg/ml as freebase. In some embodiments, the pharmaceutical compound has a solubility of more than 0.006 mg/ml as freebase. In some embodiments, the pharmaceutical compound has a solubility of more than 0.005 mg/ml as freebase. In some embodiments, the pharmaceutical compound has a solubility of more than 0.004 mg/ml as freebase. In some embodiments, the pharmaceutical compound has a solubility of more than 0.003 mg/ml as freebase. In some embodiments, the pharmaceutical compound has a solubility of more than 0.002 mg/ml as freebase. In some embodiments, the pharmaceutical compound has a solubility of more than 0.001 mg/ml as freebase. In some embodiments, the pharmaceutical compound has a solubility of more than 0.9 μg/ml as freebase. In some embodiments, the pharmaceutical compound has a solubility of more than 0.8 μg/ml as freebase. In some embodiments, the pharmaceutical compound has a solubility of more than 0.7 μg/ml as freebase. In some embodiments, the pharmaceutical compound has a solubility of more than 0.6 μg/ml as freebase. In some embodiments, the pharmaceutical compound has a solubility of more than 0.5 μg/ml as freebase. In some embodiments, the pharmaceutical compound has a solubility of more than 0.4 μg/ml as freebase. In some embodiments, the pharmaceutical compound has a solubility of more than 0.3 μg/ml as freebase. In some embodiments, the pharmaceutical compound has a solubility of more than 0.2 μg/ml as freebase. In some embodiments, the pharmaceutical compound has a solubility of more than 0.1 μg/ml as freebase. In some embodiments, the pharmaceutical compound has a solubility of more than 0.09 μg/ml as freebase. In some embodiments, the pharmaceutical compound has a solubility of more than 0.08 μg/ml as freebase. In some embodiments, the pharmaceutical compound has a solubility of more than 0.07 μg/ml as freebase. In some embodiments, the pharmaceutical compound has a solubility of more than 0.06 μg/ml as freebase. In some embodiments, the pharmaceutical compound has a solubility of more than 0.05 μg/ml as freebase. In some embodiments, the pharmaceutical compound has a solubility of more than 0.04 μg/ml as freebase. In some embodiments, the pharmaceutical compound has a solubility of more than 0.03 μg/ml as freebase. In some embodiments, the pharmaceutical compound has a solubility of more than 0.02 μg/ml as freebase. In some embodiments, the pharmaceutical compound has a solubility of more than 0.01 μg/ml as freebase. In some embodiments, the pharmaceutical compound has a solubility of more than 0.009 μg/ml as freebase. In some embodiments, the pharmaceutical compound has a solubility of more than 0.008 μg/ml as freebase. In some embodiments, the pharmaceutical compound has a solubility of more than 0.007 μg/ml as freebase. In some embodiments, the pharmaceutical compound has a solubility of more than 0.006 μg/ml as freebase. In some embodiments, the pharmaceutical compound has a solubility of more than 0.005 μg/ml as freebase. In some embodiments, the pharmaceutical compound has a solubility of more than 0.004 μg/ml as freebase. In some embodiments, the pharmaceutical compound has a solubility of more than 0.003 μg/ml as freebase. In some embodiments, the pharmaceutical compound has a solubility of more than 0.002 μg/ml as freebase. In some embodiments, the pharmaceutical compound has a solubility of more than 0.001 μg/ml as freebase. In some embodiments, the solubility of the pharmaceutical compound as freebase is measured in an aqueous medium. In some embodiments, the solubility of the pharmaceutical compound as freebase is measured in an organic solvent. In some embodiments, the aqueous medium comprises water.

In some embodiments, the pharmaceutical compound has a solubility of between about 0.001 mg/ml and 100 mg/ml as freebase. In some embodiments, the pharmaceutical compound has a solubility of between about 0.01 mg/ml and 50 mg/ml as freebase. In some embodiments, the pharmaceutical compound has a solubility of between about 0.02 mg/ml and 40 mg/ml as freebase. In some embodiments, the pharmaceutical compound has a solubility of between about 0.03 mg/ml and 30 mg/ml as freebase. In some embodiments, the pharmaceutical compound has a solubility of between about 0.04 mg/ml and 20 mg/ml as freebase. In some embodiments, the pharmaceutical compound has a solubility of between about 0.05 mg/ml and 10 mg/ml as freebase. In some embodiments, the pharmaceutical compound has a solubility of between about 0.06 mg/ml and 5 mg/ml as freebase. In some embodiments, the pharmaceutical compound has a solubility of between about 0.07 mg/ml and 4 mg/ml as freebase. In some embodiments, the pharmaceutical compound has a solubility of between about 0.09 mg/ml and 3 mg/ml as freebase. In some embodiments, the pharmaceutical compound has a solubility of between about 0.1 mg/ml and 2 mg/ml as freebase. In some embodiments, the solubility of the pharmaceutical compound as freebase is measured in an aqueous medium. In some embodiments, the solubility of the pharmaceutical compound as freebase is measured in an organic solvent. In some embodiments, the aqueous medium comprises water.

In some embodiments, the pharmaceutical compound has a solubility of less than 100 mg/ml as freebase. In some embodiments, the pharmaceutical compound has a solubility of less than 90 mg/ml as freebase. In some embodiments, the pharmaceutical compound has a solubility of less than 80 mg/ml as freebase. In some embodiments, the pharmaceutical compound has a solubility of less than 70 mg/ml as freebase. In some embodiments, the pharmaceutical compound has a solubility of less than 60 mg/ml as freebase. In some embodiments, the pharmaceutical compound has a solubility of less than 50 mg/ml as freebase. In some embodiments, the pharmaceutical compound has a solubility of less than 45 mg/ml as freebase. In some embodiments, the pharmaceutical compound has a solubility of less than 40 mg/ml as freebase. In some embodiments, the pharmaceutical compound has a solubility of less than 35 mg/ml as freebase. In some embodiments, the pharmaceutical compound has a solubility of less than 30 mg/ml as freebase. In some embodiments, the pharmaceutical compound has a solubility of less than 25 mg/ml as freebase. In some embodiments, the pharmaceutical compound has a solubility of less than 20 mg/ml as freebase. In some embodiments, the pharmaceutical compound has a solubility of less than 10 mg/ml as freebase. In some embodiments, the pharmaceutical compound has a solubility of less than 9 mg/ml as freebase. In some embodiments, the pharmaceutical compound has a solubility of less than 8 mg/ml as freebase. In some embodiments, the pharmaceutical compound has a solubility of less than 7 mg/ml as freebase. In some embodiments, the pharmaceutical compound has a solubility of less than 6 mg/ml as freebase. In some embodiments, the pharmaceutical compound has a solubility of less than 5 mg/ml as freebase. In some embodiments, the pharmaceutical compound has a solubility of less than 4 mg/ml as freebase. In some embodiments, the pharmaceutical compound has a solubility of less than 3 mg/ml as freebase. In some embodiments, the pharmaceutical compound has a solubility of more 2 mg/ml as freebase. In some embodiments, the pharmaceutical compound has a solubility of less than 1 mg/ml as freebase. In some embodiments, the pharmaceutical compound has a solubility of less than 0.9 mg/ml as freebase. In some embodiments, the pharmaceutical compound has a solubility of less than 0.8 mg/ml as freebase. In some embodiments, the pharmaceutical compound has a solubility of less than 0.7 mg/ml as freebase. In some embodiments, the pharmaceutical compound has a solubility of less than 0.6 mg/ml as freebase. In some embodiments, the pharmaceutical compound has a solubility of less than 0.5 mg/ml as freebase. In some embodiments, the pharmaceutical compound has a solubility of less than 0.4 mg/ml as freebase. In some embodiments, the pharmaceutical compound has a solubility of less than 0.3 mg/ml as freebase. In some embodiments, the pharmaceutical compound has a solubility of less than 0.2 mg/ml as freebase. In some embodiments, the pharmaceutical compound has a solubility of less than 0.1 mg/ml as freebase. In some embodiments, the pharmaceutical compound has a solubility of less than 0.09 mg/ml as freebase. In some embodiments, the pharmaceutical compound has a solubility of less than 0.08 mg/ml as freebase. In some embodiments, the pharmaceutical compound has a solubility of less than 0.07 mg/ml as freebase. In some embodiments, the pharmaceutical compound has a solubility of less than 0.06 mg/ml as freebase. In some embodiments, the pharmaceutical compound has a solubility of less than 0.05 mg/ml as freebase. In some embodiments, the pharmaceutical compound has a solubility of less than 0.04 mg/ml as freebase. In some embodiments, the pharmaceutical compound has a solubility of less than 0.03 mg/ml as freebase. In some embodiments, the pharmaceutical compound has a solubility of less than 0.02 mg/ml as freebase. In some embodiments, the pharmaceutical compound has a solubility of less than 0.01 mg/ml as freebase. In some embodiments, the pharmaceutical compound has a solubility of less than 0.009 mg/ml as freebase. In some embodiments, the pharmaceutical compound has a solubility of less than 0.008 mg/ml as freebase. In some embodiments, the pharmaceutical compound has a solubility of less than 0.007 mg/ml as freebase. In some embodiments, the pharmaceutical compound has a solubility of less than 0.006 mg/ml as freebase. In some embodiments, the pharmaceutical compound has a solubility of less than 0.005 mg/ml as freebase. In some embodiments, the pharmaceutical compound has a solubility of less than 0.004 mg/ml as freebase. In some embodiments, the pharmaceutical compound has a solubility of less than 0.003 mg/ml as freebase. In some embodiments, the pharmaceutical compound has a solubility of less than 0.002 mg/ml as freebase. In some embodiments, the pharmaceutical compound has a solubility of less than 0.001 mg/ml as freebase. In some embodiments, the pharmaceutical compound has a solubility of less than 0.9 μg/ml as freebase. In some embodiments, the pharmaceutical compound has a solubility of less than 0.8 μg/ml as freebase. In some embodiments, the pharmaceutical compound has a solubility of less than 0.7 μg/ml as freebase. In some embodiments, the pharmaceutical compound has a solubility of less than 0.6 μg/ml as freebase. In some embodiments, the pharmaceutical compound has a solubility of less than 0.5 μg/ml as freebase. In some embodiments, the pharmaceutical compound has a solubility of less than 0.4 μg/ml as freebase. In some embodiments, the pharmaceutical compound has a solubility of less than 0.3 μg/ml as freebase. In some embodiments, the pharmaceutical compound has a solubility of less than 0.2 μg/ml as freebase. In some embodiments, the pharmaceutical compound has a solubility of less than 0.1 μg/ml as freebase. In some embodiments, the pharmaceutical compound has a solubility of less than 0.09 μg/ml as freebase. In some embodiments, the pharmaceutical compound has a solubility of less than 0.08 μg/ml as freebase. In some embodiments, the pharmaceutical compound has a solubility of less than 0.07 μg/ml as freebase. In some embodiments, the pharmaceutical compound has a solubility of less than 0.06 μg/ml as freebase. In some embodiments, the pharmaceutical compound has a solubility of less than 0.05 μg/ml as freebase. In some embodiments, the pharmaceutical compound has a solubility of less than 0.04 μg/ml as freebase. In some embodiments, the pharmaceutical compound has a solubility of less than 0.03 μg/ml as freebase. In some embodiments, the pharmaceutical compound has a solubility of less than 0.02 μg/ml as freebase. In some embodiments, the pharmaceutical compound has a solubility of less than 0.01 μg/ml as freebase. In some embodiments, the pharmaceutical compound has a solubility of less than 0.009 μg/ml as freebase. In some embodiments, the pharmaceutical compound has a solubility of less than 0.008 μg/ml as freebase. In some embodiments, the pharmaceutical compound has a solubility of less than 0.007 μg/ml as freebase. In some embodiments, the pharmaceutical compound has a solubility of less than 0.006 μg/ml as freebase. In some embodiments, the pharmaceutical compound has a solubility of less than 0.005 μg/ml as freebase. In some embodiments, the pharmaceutical compound has a solubility of less than 0.004 μg/ml as freebase. In some embodiments, the pharmaceutical compound has a solubility of less than 0.003 μg/ml as freebase. In some embodiments, the pharmaceutical compound has a solubility of less than 0.002 μg/ml as freebase. In some embodiments, the pharmaceutical compound has a solubility of less than 0.001 μg/ml as freebase. In some embodiments, the solubility of the pharmaceutical compound as freebase is measured in an aqueous medium. In some embodiments, the solubility of the pharmaceutical compound as freebase is measured in an organic solvent. In some embodiments, the aqueous medium comprises water.

In some embodiments, the ratio of the conjugate base of the complexing agent to the pharmaceutical compound in the pharmaceutically acceptable salt is from about 1:1 to about 1:4. In some embodiments, the ratio of the conjugate base of the complexing agent to the pharmaceutical compound in the pharmaceutically acceptable salt is from about 1:1.1 to about 1:3.9. In some embodiments, the ratio of the conjugate base of the complexing agent to the pharmaceutical compound in the pharmaceutically acceptable salt is from about 1:1.2 to about 1:3.8. In some embodiments, the ratio of the conjugate base of the complexing agent to the pharmaceutical compound in the pharmaceutically acceptable salt is from about 1:1.3 to about 1:3.7. In some embodiments, the ratio of the conjugate base of the complexing agent to the pharmaceutical compound in the pharmaceutically acceptable salt is from about 1:1.4 to about 1:3.6. In some embodiments, the ratio of the conjugate base of the complexing agent to the pharmaceutical compound in the pharmaceutically acceptable salt is from about 1:1.5 to about 1:3.5. In some embodiments, the ratio of the conjugate base of the complexing agent to the pharmaceutical compound in the pharmaceutically acceptable salt is from about 1:1.6 to about 1:3.4. In some embodiments, the ratio of the conjugate base of the complexing agent to the pharmaceutical compound in the pharmaceutically acceptable salt is from about 1:1.7 to about 1:3.3. In some embodiments, the ratio of the conjugate base of the complexing agent to the pharmaceutical compound in the pharmaceutically acceptable salt is from about 1:1.8 to about 1:3.2. In some embodiments, the ratio of the conjugate base of the complexing agent to the pharmaceutical compound in the pharmaceutically acceptable salt is from about 1:1.9 to about 1:3.1. In some embodiments, the ratio of the conjugate base of the complexing agent to the pharmaceutical compound in the pharmaceutically acceptable salt is from about 1:2 to about 1:3. In some embodiments, the ratio of the conjugate base of the complexing agent to the pharmaceutical compound in the pharmaceutically acceptable salt is from about 1:2.1 to about 1:2.9. In some embodiments, the ratio of the conjugate base of the complexing agent to the pharmaceutical compound in the pharmaceutically acceptable salt is from about 1:2.2 to about 1:2.8. In some embodiments, the ratio of the conjugate base of the complexing agent to the pharmaceutical compound in the pharmaceutically acceptable salt is from about 1:2.3 to about 1:2.7. In some embodiments, the ratio of the conjugate base of the complexing agent to the pharmaceutical compound in the pharmaceutically acceptable salt is from about 1:2.4 to about 1:2.6. In some embodiments, the ratio of the conjugate base of the complexing agent to the pharmaceutical compound in the pharmaceutically acceptable salt is from about 1:1 to about 1:4, about 1:1.1, 1:1.2, 1:1.3, 1:1.4, 1:1.5, 1:1.6, 1:1.7, 1:1.8, 1:1.9, 1:2.0, 1:2.1, 1:2.2, 1:2.3, 1:2.4, 1:2.5, 1:2.6, 1:2.7, 1:2.8, 1:2.9, 1:3.0, 1:3.1, 1:3.2, 1:3.3, 1:3.4, 1:3.5, 1:3.6, 1:3.7, 1:3.8, 1:3.9, or about 1:4.0, or any ratio therebetween.

In some embodiments, the solubility of the pharmaceutical compound in the pharmaceutically acceptable salt is higher than (i) the solubility of the pharmaceutical compound as a salt; or (ii) the solubility of the pharmaceutical compound in a salt comprising the pharmaceutical compound in freebase form that is complexing to a non-polar pore of the complexing agent, wherein the pharmaceutical compound has the same concentration in the pharmaceutically acceptable salt, the pharmaceutical compound as a salt, and the salt comprising the pharmaceutical compound in freebase form. In some embodiments, the solubility of the pharmaceutical compound in the pharmaceutically acceptable salt is higher than the solubility of the pharmaceutical compound in a salt comprising the pharmaceutical compound and complexing agent with a higher molar ratio of the pharmaceutical compound to the complexing agent, wherein the pharmaceutical compound has the same concentration in the pharmaceutically acceptable salt and the salt with a higher molar ratio of the pharmaceutical compound to the complexing agent. In some embodiments, the solubility of the pharmaceutical compound in the pharmaceutically acceptable salt has an increase of at least 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, or 100-fold compared with (i) the solubility of the pharmaceutical compound as a salt; or (ii) the solubility of the pharmaceutical compound in a salt comprising the pharmaceutical compound in freebase form that is complexing to a non-polar pore of the complexing agent, wherein the pharmaceutical compound has the same concentration in the pharmaceutically acceptable salt, the pharmaceutical compound as a salt, and the salt comprising the pharmaceutical compound in freebase form. In some embodiments, the solubility of the pharmaceutical compound in the pharmaceutically acceptable salt has an increase of at least 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, or 100-fold compared with the solubility of the pharmaceutical compound in a salt comprising the pharmaceutical compound and complexing agent with a higher molar ratio of the pharmaceutical compound to the complexing agent, wherein the pharmaceutical compound has the same concentration in the pharmaceutically acceptable salt and the salt with a higher molar ratio of the pharmaceutical compound to the complexing agent.

In an aspect, provided herein is a pharmaceutically acceptable salt of a compound pharmaceutical comprising: (i) a pharmaceutical compound, or an enantiomer, a mixture of enantiomers, or an isotopic variant thereof; or a pharmaceutically acceptable salt, solvate or hydrate thereof, wherein the pharmaceutical compound comprises a prodrug comprising an unionized substance conjugated to a chemical entity, wherein the chemical entity comprises a protonated nitrogen atom; and (ii) a conjugate base of a complexing agent comprising a plurality of acidic functional groups, wherein the plurality of acidic functional groups comprise a conjugate base of an acid which acts as a counterion for the protonated nitrogen atom of the pharmaceutical compound. In some embodiments, the unionized substance comprises brexanolone. In some embodiments, the chemical entity comprises γ-aminobutyric acid (GABA). In some embodiments, the pharmaceutically acceptable salt further comprises an additional molar equivalent of the unionized substance compared to the amount of complexing agent. In some embodiments, the complexing agent acts as the counterion to between 1 to 8 molecules of the pharmaceutical compound in the pharmaceutically acceptable salt. In some embodiments, the complexing agent further comprises a non-polar region. In some embodiments, the about 1 molar equivalent of the unionized substance is complexed to the complexing agent through the non-polar region. In some embodiments, the complexing agent comprises a substituted cyclodextrin. In some embodiments, the complexing agent comprises a cyclodextrin substituted with at least one acidic functional group. In some embodiments, the cyclodextrin is substituted with 3 to 8 acidic functional groups. In some embodiments, the cyclodextrin is sulfobutylether-β-cyclodextrin. In some embodiments, the pharmaceutically acceptable salt has a ratio of the conjugate base of the complexing agent to the pharmaceutical compound that is from about 1:4 to about 1:10. In some embodiments, the pharmaceutically acceptable salt has a ratio of the conjugate base of the complexing agent to the pharmaceutical compound that is about 1:4. 1:5, 1:6, 1:7, 1:8, 1:9, or about 1:10, or any ratio therebetween.

In some embodiments, the complexing agent further comprises a non-polar region. In some embodiments, the non-polar region is a non-polar pore. In some embodiments, the additional molar equivalent of the unionized substance is complexed to the non-polar region of the complexing agent. In some embodiments, the additional molar equivalent of the unionized substance is complexed to the non-polar pore of the complexing agent.

In some embodiments, the pharmaceutically acceptable salt comprises 0.1-20 molar equivalents of the unionized substance compared to the complexing agent. In some embodiments, the pharmaceutically acceptable salt comprises 0.2-15 molar equivalents of unionized substance compared to the complexing agent. In some embodiments, the pharmaceutically acceptable salt comprises 0.5-10 molar equivalents of the unionized substance compared to the complexing agent. In some embodiments, the pharmaceutically acceptable salt comprises 1-5 molar equivalents of the unionized substance compared to the complexing agent. In some embodiments, the pharmaceutically acceptable salt comprises 0.1-20 molar equivalents of the unionized substance compared to the complexing agent, e.g., about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, or 20.0 molar equivalents of the unionized substance, or any amount therebetween. In some embodiments, the pharmaceutically acceptable salt comprises 1-2 molar equivalents of the unionized substance compared to the complexing agent. In some embodiments, the pharmaceutically acceptable salt comprises about 1.1, 1.2, 1.3, 1.4, 1.5, 1.6, 1.7, 1.8, 1.9, or about 2 molar equivalents of the unionized substance compared to the complexing agent.

In some embodiments, the molar ratio of the cyclodextrin to the pharmaceutical compound comprising a protonated nitrogen atom is from about 1:4 to about 1:8. In some embodiments, the molar ratio of the cyclodextrin to the pharmaceutical compound comprising a protonated nitrogen atom is from about 1:4 to about 1:10. In some embodiments, the molar ratio of the cyclodextrin to the pharmaceutical compound comprising a protonated nitrogen atom is from about 1:5 to about 1:7. In some embodiments, the molar ratio of the cyclodextrin to the pharmaceutical compound comprising a protonated nitrogen atom is about 1:4. In some embodiments, the molar ratio of the cyclodextrin to the pharmaceutical compound comprising a protonated nitrogen atom is about 1:5. In some embodiments, the molar ratio of the cyclodextrin to the pharmaceutical compound comprising a protonated nitrogen atom is about 1:6. In some embodiments, the molar ratio of the cyclodextrin to the pharmaceutical compound comprising a protonated nitrogen atom is about 1:7. In some embodiments, the molar ratio of the cyclodextrin to the pharmaceutical compound comprising a protonated nitrogen atom is about 1:8. In some embodiments, the molar ratio of the cyclodextrin to the pharmaceutical compound comprising a protonated nitrogen atom is about 1:9. In some embodiments, the molar ratio of the cyclodextrin to the pharmaceutical compound comprising a protonated nitrogen atom is about 1:10.

In some embodiments, molar ratio of acidic functional groups of the complexing agent to the pharmaceutical compound comprising a protonated nitrogen atom is from about 2:1 to about 1:2. In some embodiments, molar ratio of acidic functional groups of the complexing agent to the pharmaceutical compound comprising a protonated nitrogen atom is from about 1.75:1 to about 1:1.75. In some embodiments, molar ratio of acidic functional groups of the complexing agent to the pharmaceutical compound comprising a protonated nitrogen atom is from about 1.5:1 to about 1:1.5. In some embodiments, molar ratio of acidic functional groups of the complexing agent to the pharmaceutical compound comprising a protonated nitrogen atom is from about 1.4:1 to about 1:1.4. In some embodiments, molar ratio of acidic functional groups of the complexing agent to the pharmaceutical compound comprising a protonated nitrogen atom is from 1.3:1 to about 1:1.3. In some embodiments, molar ratio of acidic functional groups of the complexing agent to the pharmaceutical compound comprising a protonated nitrogen atom is from about 1.25:1 to about 1:1.25. In some embodiments, molar ratio of acidic functional groups of the complexing agent to the pharmaceutical compound comprising a protonated nitrogen atom is from about 1.2:1 to about 1:1.2. In some embodiments, molar ratio of acidic functional groups of the complexing agent to the pharmaceutical compound comprising a protonated nitrogen atom is from about 1.15:1 to about 1:1.15. In some embodiments, molar ratio of acidic functional groups of the complexing agent to the pharmaceutical compound comprising a protonated nitrogen atom is from about 1.1:1 to about 1:1.1. In some embodiments, molar ratio of acidic functional groups of the complexing agent to the pharmaceutical compound comprising a protonated nitrogen atom is from about 1.05:1 to about 1:1.05. In some embodiments, molar ratio of acidic functional groups of the complexing agent to the pharmaceutical compound comprising a protonated nitrogen atom is about 1:1.

In some embodiments, the solubility of the ununionized substance in the pharmaceutically acceptable salt is higher than (i) the solubility of the unionized substance as a salt; or (ii) the solubility of the unionized substance in a salt comprising the unionized substance in freebase form that is complexing to a non-polar pore of the complexing agent, wherein the unionized substance has the same concentration in the pharmaceutically acceptable salt, the unionized substance as a salt, and the salt comprising the unionized substance in freebase form. In some embodiments, the solubility of the unionized substance in the pharmaceutically acceptable salt is higher than the solubility of the unionized substance in a salt comprising the unionized substance and complexing agent with a higher molar ratio of the unionized substance to the complexing agent, wherein the unionized substance has the same concentration in the pharmaceutically acceptable salt and the salt with a higher molar ratio of the unionized substance to the complexing agent. In some embodiments, the solubility of the unionized substance in the pharmaceutically acceptable salt has an increase of at least 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, or 100-fold compared with (i) the solubility of the unionized substance as a salt; or (ii) the solubility of the unionized substance in a salt comprising the unionized substance in freebase form that is complexing to a non-polar pore of the complexing agent, wherein the unionized substance has the same concentration in the pharmaceutically acceptable salt, the unionized substance as a salt, and the salt comprising the unionized substance in freebase form. In some embodiments, the solubility of the unionized substance in the pharmaceutically acceptable salt has an increase of at least 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, or 100-fold compared with the solubility of the unionized substance in a salt comprising the unionized substance and complexing agent with a higher molar ratio of the unionized substance to the complexing agent, wherein the unionized substance has the same concentration in the pharmaceutically acceptable salt and the salt with a higher molar ratio of the unionized substance to the complexing agent.

In an aspect, provided herein is a pharmaceutically acceptable salt of a compound pharmaceutical comprising: (i) a first pharmaceutical compound, or an enantiomer, a mixture of enantiomers, or an isotopic variant thereof; or a pharmaceutically acceptable salt, solvate or hydrate thereof, wherein the pharmaceutical compound comprises a protonated nitrogen atom; (ii) a conjugate base of a complexing agent comprising a plurality of acidic functional groups, wherein at least one acidic functional group of the plurality of acidic functional groups acts as a counterion of the first pharmaceutical compound; and (iii) a second pharmaceutical compound, or an enantiomer, a mixture of enantiomers, or an isotopic variant thereof, or a pharmaceutically acceptable salt, solvate or hydrate thereof, wherein the second pharmaceutical compound is unionized. In some embodiments, the pharmaceutically acceptable salt has a ratio of the conjugate base of the complexing agent to the first pharmaceutical compound that is from about 1:4 to about 1:10. In some embodiments, the pharmaceutically acceptable salt has a ratio of the conjugate base of the complexing agent to the first pharmaceutical compound that is about 1:4. 1:5, 1:6, 1:7, 1:8, 1:9, or about 1:10, or any ratio therebetween. In some embodiments, the pharmaceutically acceptable salt has a ratio of the conjugate base of the complexing agent to the second pharmaceutical compound that is about 1:1. In some embodiments, the first pharmaceutical compound comprises an ionized form. In some embodiments, the first pharmaceutical compound comprises an unionized form. In some embodiments, the second pharmaceutical compound does not comprise an ionizable nitrogen atom. In some embodiments, the second pharmaceutical compound has a pKa value above a threshold value.

In some embodiments, the complexing agent is sulfobutylether-β-cyclodextrin. In some embodiments, the first pharmaceutical compound comprises a dissociative medication compound, a dissociative hallucinogen compound, a dissociative anesthetic compound, an arylcyclo-hexylamine, a 1,2-diarylethylamine, a β-keto-arylcyclohexylamine, or a compound that modulates the NMDA receptor. In some embodiments, the first pharmaceutical compound is ketamine, arylcyclo-hexylamine, 1,2-diarylethylamine, β-keto-arylcyclohexylamine, methoxetamine, deschloroketamine, N-ethyl deschloroketamine (eticyclidone), 3-methoxyphencyclidine, methoxieticyclidine, ephenidine, lanicemine, dextromethorphan, dextrorphan, methoxyketamine, a N,N-dimethyltryptamine, a N,N-diethyltryptamine, a N,N-dipropyltryptamine, a N-Methyl-N-propyltryptamine, a N-methyl-N-isopropyltryptamine, a N,N-diallyltryptamine, a N-methyl-N-allyltryptamine, N-methyl-N-ethyltryptamine, a N,N-Diisopropyltryptamine, 4-hydroxy-N-methyl-N-ethyltryptamine, 5-methoxy-N,N-diisopropyltryptamine, O-acetylpsilocin, methylisopropyllysergamide, ethylisopropyllysergamide, 6-allyl-6-nor-LSD, 6-ethyl-6-nor-lysergic acid diethylamide, 1-acetyl-LSD, 1-propionyl-6-ethyl-6-nor-lysergic acid diethylamide, 1-propionyl-lysergic acid diethylamide, 1-Cyclopropionyl-d-lysergic acid diethylamide, N1-butyryl-lysergic acid diethylamide, 6-propyl-6-nor-Lysergic acid diethylamide, mescaline, 2,5-dimethoxy-4-bromophenethylamine (2C—B), 2-(4-Iodo-2,5-dimethoxyphenyl) ethan-1-amine (2C—I), 2-(4-Chloro-2,5-dimethoxyphenyl) ethan-1-amine (2C—C), 2,5-Dimethoxy-4-iodoamphetamine, 2-[2,5-Dimethoxy-4-(propylsulfanyl)phenyl]ethan-1-amine, 2-(4-iodo-2,5-dimethoxyphenyl)-N-[(2-methoxyphenyl)methyl]ethanamine, racemorphan, levorphanol, racemethorphan, buprenorphine, morphine, loperamide, morphine, codeine, hydrocodone, oxymorphone, buprenorphine, fentanyl, methadone, tramadol, alpha-methyl acetyl fentanyl, alfentanil, butyryl fentanyl, butyrfentanyl, carfentanil, 3-methylcarfentanil, 4-fluorofentanyl, beta-hydroxyfentanyl, alpha-methylfentanyl, cis-3-methylfentanyl, beta-hydroxy-3-methylfentanyl, remifentanil, sufentanil, 3-methylthiofentanyl, naloxone, naltrexone, a cathinone, a 3,4-methylenedioxyamphetamine derivative, an aminoalkyl-substituted benzofuran, a substituted amphetamine, an aminoindane, diphenhydramine, hydroxazine, phenylephrine, dopamine, adrenaline, lidocaine, oxymetazoline, clemastine, chlorpheniramine, and 6-chloro-2-aminotetralin. In some embodiments, the first pharmaceutical compound comprises ketamine.

In some embodiments, the second pharmaceutical compound comprises rapamycin. In some embodiments, the first pharmaceutical compound comprises ketamine and the second pharmaceutical compound comprises rapamycin. In some embodiments, the first pharmaceutical compound comprises no ketamine and the second pharmaceutical compound comprises rapamycin. In some embodiments, the second pharmaceutical compound comprises clonidine. In some embodiments, the first pharmaceutical compound comprises ketamine and the second pharmaceutical compound comprises clonidine. In some embodiments, the first pharmaceutical compound comprises no ketamine and the second pharmaceutical compound comprises clonidine. In some embodiments, the complexing agent comprises a substituted cyclodextrin. In some embodiments, the complexing agent comprises a cyclodextrin substituted with at least one acidic functional group. In some embodiments, the cyclodextrin is substituted with 3 to 8 acidic functional groups. In some embodiments, the cyclodextrin is sulfobutylether-β-cyclodextrin. In some embodiments, the pharmaceutically acceptable salt is formulated for subcutaneous, intramuscular, sublingual, oral, rectal, transvaginal, or intranasal administration. In some embodiments, the complexing agent comprises a non-polar region. In some embodiments, the second pharmaceutical compound is complexed to the complexing agent through the non-polar region.

In some embodiments, the solubility of the second pharmaceutical compound in the pharmaceutically acceptable salt is higher than the solubility of the second pharmaceutical compound as a salt, wherein the second pharmaceutical compound has the same concentration in the pharmaceutically acceptable salt, and the second pharmaceutical compound as a salt. In some embodiments, the solubility of the second pharmaceutical compound in the pharmaceutically acceptable salt has an increase of at least 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, or 100-fold compared with the solubility of the second pharmaceutical compound as a salt, wherein the second pharmaceutical compound has the same concentration in the pharmaceutically acceptable salt, and the second pharmaceutical compound as a salt.

In some embodiments, the plurality of acidic functional groups comprise an acidic group which acts as a counterion for the protonated nitrogen atom of the pharmaceutical compound. In some embodiments, the acidic group is the conjugate base of the acidic group. In some embodiments, the acidic group is a carboxylic acid or carboxylate. In some embodiments, the acidic group is a carboxylate. In some embodiments, the acidic group is a sulfonic acid or sulfonate. In some embodiments, the acidic group is a sulfonate. In some embodiments, the conjugate base of the complexing agent acts as the counterion for a plurality of the pharmaceutical compound. In some embodiments, each acidic group of the plurality of acidic functional groups acts as a counterion for a plurality of the pharmaceutical compound. In some embodiments, each acidic group of the plurality of acidic functional groups acts as a counterion for a protonated amine of a plurality of the pharmaceutical compound. In some embodiments, each of the plurality of acidic functional groups acts as a counterion for a pronated amine.

In some embodiments, the complexing agent is a cyclodextrin substituted with the plurality of acidic functional group. In some embodiments, the plurality of acidic functional groups is a carboxylic acid, sulfonic acid, sulfonic acid, phosphonic acid, or phosphonic acid, or any combination thereof. In some embodiments, the cyclodextrin is substituted with at least 1, at least 2, at least 3, at least 4, at least 5, or at least 6 acidic functional groups. In some embodiments, the cyclodextrin is substituted with 3 to 8 acidic functional groups, 3 to 7 acidic functional groups, 4 to 8 acidic functional groups, 4 to 7 acidic functional groups, 5 to 8 acidic functional groups, 6 to 8 acidic functional groups, or 7 to 8 acidic functional groups.

In some embodiments, the complexing agent is a substituted cyclodextrin. In some cases, substituted cyclodextrins provided herein are complex mixtures wherein individual cyclodextrin molecules may comprise different numbers of substituents from other individual cyclodextrin molecules. In such cases, the number of substituents (e.g. the number of acidic functional groups) described as being present on the cyclodextrins provided herein may refer to an average degree of substitution of the mixture. For example, when a cyclodextrin is described as substituted with 3 to 8 acidic functional groups, it is intended that a complex mixture of cyclodextrins having an average degree of substitution from 3 to 8 acidic functional groups is covered. The average degree of substitution need not be an integer value and will often be a decimal value. For example, commercially available SBEBCD has an average degree of substitution of about 6.5.

In some embodiments, the complexing agent is a substituted cyclodextrin. In some embodiments, the substituted cyclodextrin is substituted with one or more acidic functional groups, or a pharmaceutically acceptable salt thereof. In some embodiments, the substituted cyclodextrin is substituted with a plurality of carboxylic acid, sulfonic acid, sulfonic acid, phosphonic acid, or phosphonic acid functional groups. In some embodiments, the cyclodextrin is substituted with at least 1, at least 2, at least 3, at least 4, at least 5, or at least 6 acidic functional groups. In some embodiments, the cyclodextrin is substituted with 3 to 8 acidic functional groups, 3 to 7 acidic functional groups, 4 to 8 acidic functional groups, 4 to 7 acidic functional groups, 5 to 8 acidic functional groups, 6 to 8 acidic functional groups, or 7 to 8 acidic functional groups.

In some embodiments, the molar ratio of the cyclodextrin to the pharmaceutical compound comprising a protonated nitrogen atom is from about 1:4 to about 1:8. In some embodiments, the molar ratio of the cyclodextrin to the pharmaceutical compound comprising a protonated nitrogen atom is from about 1:4 to about 1:10. In some embodiments, the molar ratio of the cyclodextrin to the pharmaceutical compound comprising a protonated nitrogen atom is from about 1:5 to about 1:7. In some embodiments, the molar ratio of the cyclodextrin to the pharmaceutical compound comprising a protonated nitrogen atom is about 1:4. In some embodiments, the molar ratio of the cyclodextrin to the pharmaceutical compound comprising a protonated nitrogen atom is about 1:5. In some embodiments, the molar ratio of the cyclodextrin to the pharmaceutical compound comprising a protonated nitrogen atom is about 1:6. In some embodiments, the molar ratio of the cyclodextrin to the pharmaceutical compound comprising a protonated nitrogen atom is about 1:7. In some embodiments, the molar ratio of the cyclodextrin to the pharmaceutical compound comprising a protonated nitrogen atom is about 1:8. In some embodiments, the molar ratio of the cyclodextrin to the pharmaceutical compound comprising a protonated nitrogen atom is about 1:9. In some embodiments, the molar ratio of the cyclodextrin to the pharmaceutical compound comprising a protonated nitrogen atom is about 1:10.

In some embodiments, molar ratio of acidic functional groups of the complexing agent to the pharmaceutical compound comprising a protonated nitrogen atom is from about 2:1 to about 1:2. In some embodiments, molar ratio of acidic functional groups of the complexing agent to the pharmaceutical compound comprising a protonated nitrogen atom is from about 1.75:1 to about 1:1.75. In some embodiments, molar ratio of acidic functional groups of the complexing agent to the pharmaceutical compound comprising a protonated nitrogen atom is from about 1.5:1 to about 1:1.5. In some embodiments, molar ratio of acidic functional groups of the complexing agent to the pharmaceutical compound comprising a protonated nitrogen atom is from about 1.4:1 to about 1:1.4. In some embodiments, molar ratio of acidic functional groups of the complexing agent to the pharmaceutical compound comprising a protonated nitrogen atom is from 1.3:1 to about 1:1.3. In some embodiments, molar ratio of acidic functional groups of the complexing agent to the pharmaceutical compound comprising a protonated nitrogen atom is from about 1.25:1 to about 1:1.25. In some embodiments, molar ratio of acidic functional groups of the complexing agent to the pharmaceutical compound comprising a protonated nitrogen atom is from about 1.2:1 to about 1:1.2. In some embodiments, molar ratio of acidic functional groups of the complexing agent to the pharmaceutical compound comprising a protonated nitrogen atom is from about 1.15:1 to about 1:1.15. In some embodiments, molar ratio of acidic functional groups of the complexing agent to the pharmaceutical compound comprising a protonated nitrogen atom is from about 1.1:1 to about 1:1.1. In some embodiments, molar ratio of acidic functional groups of the complexing agent to the pharmaceutical compound comprising a protonated nitrogen atom is from about 1.05:1 to about 1:1.05. In some embodiments, molar ratio of acidic functional groups of the complexing agent to the pharmaceutical compound comprising a protonated nitrogen atom is about 1:1.

In some embodiments, the cyclodextrin is a compound of Formula (I):

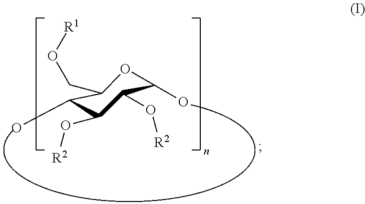

wherein:
each R¹ is independently H or optionally substituted alkyl;
each R² is independently H or optionally substituted alkyl; and
n is 6, 7, or 8;
   or a stereoisomer, a mixture of stereoisomers, or an isotopic variant thereof, or a pharmaceutically acceptable salt, solvate or hydrate thereof.

In some embodiments, each R¹ is independently H or alkyl optionally substituted with a polar functional group. In some embodiments, the polar functional group is an amido functional group, an acidic functional group, an ester functional group, a hydroxyl functional group, an alkoxy functional group, or a poly(alkylene oxide) functional group. In some embodiments, each R¹ is independently H or alkyl optionally substituted with an acidic functional group or a hydroxyl functional group.

In some embodiments, each R¹ is independently H or alkyl optionally substituted with an acidic functional group. In some embodiments, each R¹ is independently H or alkyl substituted with an acidic functional group. In some embodiments, each R¹ is independently H or C₁-C₆ alkyl substituted with an acidic functional group. In some embodiments, each R¹ is independently H or C₁-C₆ alkyl substituted with an acidic functional group selected from a carboxylic acid, a sulfonic acid, a sulfonic acid, a phosphonic acid, or a phosphonic acid. In some embodiments, each R¹ is independently H,

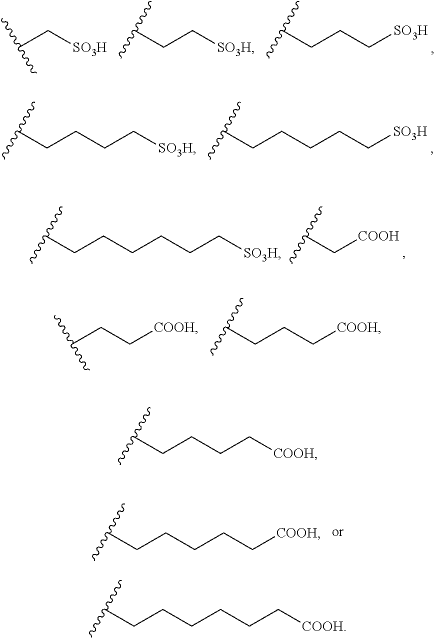

In some embodiments, each R¹ is independently H,

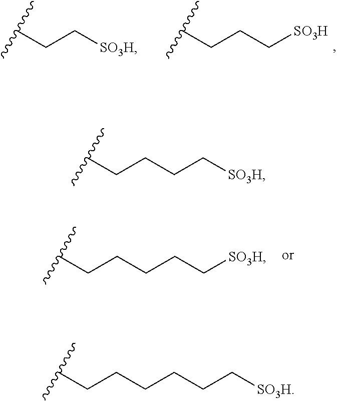 [SMILES: *CCS(=O)(=O)O ; *CCCS(=O)(=O)O ; *CCCCS(=O)(=O)O ; *CCCCCS(=O)(=O)O ; "or" ; *CCCCCCS(=O)(=O)O]

In some embodiments wherein R¹ comprises an acidic functional group, each R² is H or acetyl. In some embodiments wherein R¹ comprises an acidic functional group, each R² is H.

In some embodiments, each R¹ is independently H or alkyl optionally substituted with a hydroxyl functional group. In some embodiments, each R¹ is independently H or alkyl substituted with a hydroxyl functional group. In some embodiments, each R¹ is independently H or C₁-C₆ alkyl substituted with a hydroxyl functional group. In some embodiments, each R¹ is independently H or hydroxypropyl, hydroxybutyl, hydroxypentyl, or hydroxyhexyl. In some embodiments, each R¹ and R² is independently H or hydroxypropyl, hydroxybutyl, hydroxypentyl, or hydroxyhexyl.

In some embodiments, each R² is independently H or alkyl optionally substituted with a polar functional group. In some embodiments, each R² is independently H or alkyl optionally substituted with a hydroxyl functional group. In some embodiments, each R² is independently H or alkyl substituted with a hydroxyl functional group. In some embodiments, each R² is independently H or C₁-C₆ alkyl substituted with a hydroxyl functional group. In some embodiments, each R² is independently H or hydroxypropyl, hydroxybutyl, hydroxypentyl, or hydroxyhexyl. In some embodiments, each R² is H. In some embodiments, each R² is H or acetyl.

In some embodiments, each R² is independently H or alkyl optionally substituted with an acidic functional group. In some embodiments, each R² is independently H or C₁-C₆ alkyl optionally substituted with an acidic functional group. In some embodiments, each R² is independently H or C₁-C₆ alkyl optionally substituted with a sulfonic acid or carboxylic acid functional group.

In some embodiments, n is 6 or 7. In some embodiments, n is 7 or 8. In some embodiments, n is 6. In some embodiments, n is 7. In some embodiments, n is 8.

In some embodiments, the cyclodextrin is a SBEBCD.

In an aspect, provided herein is a pharmaceutically acceptable salt of an pharmaceutical compound having the formula

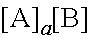

wherein:
A is an pharmaceutical compound comprising at least one basic nitrogen atom, wherein the pharmaceutical compound is an antiviral compound, an antibacterial compound, an anti-fungal compound, a compound for treatment of a neurological disorder, a compound for treatment of Parkinson's disease, a treatment for migraine headache, a treatment for autoimmune disease, a treatment for cancer, a treatment for lymphoma, a treatment for pancreatitis, a treatment for opioid overdose, a treatment for flu infection, or a treatment for an inflammatory disorder. In some embodiments, the pharmaceutical compound comprises rotigotine, eletriptan, copanlisib, remdesivir, nafamostat (nafamostat mesylate), midazolam, melevodopa, tigecycline, naloxone, amikacin, 1-amantadine, 2-amantadine, rimantadine, amifampridine, rapamycin, clonidine, or caspofungin;
B is a complexing agent comprising a plurality of acidic functional groups; and a is a number from 1-5, wherein the number is selected such that a portion, but not all, of the acidic functional groups of B act as a counterion to the total number of basic nitrogen atoms of A and total number of basic nitrogen atoms of A is less than the number of acidic functional groups of B.

In some embodiments, B further comprises a non-polar region. In some embodiments,

B acts as the counterion to between 1 to 4 molecules of the pharmaceutical compound.

In some embodiments, the pharmaceutically acceptable salt comprises about 1 molar equivalent of the pharmaceutical compound compared to the amount of the complexing agent and the about 1 molar equivalent of the pharmaceutical compound is unionized and complexed to the complexing agent through the non-polar region.

B can be any of the complexing agents provided herein, including without limitations any of the cyclodextrins or compounds of Formula (I) provided herein, the number of acidic groups of such compounds influencing the value of a.

In some embodiments, the solubility of the pharmaceutical compound in the pharmaceutically acceptable salt is higher than (i) the solubility of the pharmaceutical compound as a salt; or (ii) the solubility of the pharmaceutical compound in a salt comprising the pharmaceutical compound in freebase form that is complexing to a non-polar pore of the complexing agent, wherein the pharmaceutical compound has the same concentration in the pharmaceutically acceptable salt, the pharmaceutical compound as a salt, and the salt comprising the pharmaceutical compound in freebase form. In some embodiments, the solubility of the pharmaceutical compound in the pharmaceutically acceptable salt is higher than the solubility of the pharmaceutical compound in a salt comprising the pharmaceutical compound and complexing agent with a higher molar ratio of the pharmaceutical compound to the complexing agent, wherein the pharmaceutical compound has the same concentration in the pharmaceutically acceptable salt and the salt with a higher molar ratio of the pharmaceutical compound to the complexing agent. In some embodiments, the solubility of the pharmaceutical compound in the pharmaceutically acceptable salt has an increase of at least 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, or 100-fold compared with (i) the solubility of the pharmaceutical compound as a salt; or (ii) the solubility of the pharmaceutical compound in a salt comprising the pharmaceutical compound in freebase form that is complexing to a non-polar pore of the complexing agent, wherein the pharmaceutical compound has the same concentration in the pharmaceutically acceptable salt, the pharmaceutical compound as a salt, and the salt comprising the pharmaceutical compound in freebase form. In some embodiments, the solubility of the pharmaceutical compound in the pharmaceutically acceptable salt has an increase of at least 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, or 100-fold compared with the solubility of the pharmaceutical compound in a salt comprising the pharmaceutical compound and complexing agent with a higher molar ratio of the pharmaceutical compound to the complexing agent, wherein the pharmaceutical compound has the same concentration in the pharmaceutically acceptable salt and the salt with a higher molar ratio of the pharmaceutical compound to the complexing agent.

A can be any of the pharmaceutical compounds provided herein, the properties of which (e.g. the number of basic nitrogen atoms) will affect the value of a. In some cases, the pharmaceutical compound may comprise multiple basic nitrogen atoms, one or more of which (though not necessarily all) may be considered basic. However, depending on the differences in pKa value between the multiple basic nitrogen atoms, not every nitrogen atom need be protonated. In some embodiments, it is contemplated that only basic nitrogen atoms having a pKa value above a threshold pKa (e.g. a pKa of 3, 4, 5, 6, 7, 8, 9, 10, or 11) will actually be protonated in a salt provided herein, and any nitrogen atoms having a pKa below the threshold value will not. Thus, in some embodiments, the at least one basic nitrogen atom comprises any nitrogen of the pharmaceutical compound having a pKa of at least 3, at least 4, at least 5, at least 6, at least 7, at least 8, at least 9, at least 10 or at least 11.

The value of a need not be an integer value. For example, if the complexing agent of B acts as a counterion of 2.5 molecules of the pharmaceutical compound of A each of which comprising two protonated nitrogen atoms, the structure of the overall salt complex will result in a charged molecule of A being effectively “shared” between molecules of B.

Additionally, any complexing agent B need not be a uniform species of identical substitution of acidic functional groups, and it is explicitly contemplated by the instant disclosure that this will frequently not be the case. For example, commercially available SBEBCD has an average degree of substitution of about 6.5 acidic functional groups. In such a case, the compound having the formula [A]ₐ[B] is intended to cover such a heterogenous mixture of complexing agents.

In some embodiments, the pharmaceutical compound of A comprises one, two, or three basic nitrogen atoms. In some embodiments, the pharmaceutical compound of A comprises one basic nitrogen atom. In some embodiments, the pharmaceutical compound of A comprises two basic nitrogen atoms. In some embodiments, the pharmaceutical compound of A comprises three basic nitrogen atoms.

In some embodiments, B comprises about 1 to about 8 acidic functional groups. In some embodiments, B comprises about 1 to about 2, about 1 to about 3, about 1 to about 4, about 1 to about 5, about 1 to about 6, about 1 to about 7, about 1 to about 8, about 2 to about 3, about 2 to about 4, about 2 to about 5, about 2 to about 6, about 2 to about 7, about 2 to about 8, about 3 to about 4, about 3 to about 5, about 3 to about 6, about 3 to about 7, about 3 to about 8, about 4 to about 5, about 4 to about 6, about 4 to about 7, about 4 to about 8, about 5 to about 6, about 5 to about 7, about 5 to about 8, about 6 to about 7, about 6 to about 8, or about 7 to about 8 acidic functional groups. In some embodiments, B comprises about 1, about 2, about 3, about 4, about 5, about 6, about 7, or about 8 acidic functional groups. In some embodiments, B comprises at least about 1, about 2, about 3, about 4, about 5, about 6, or about 7 acidic functional groups. In some embodiments, B comprises at most about 2, about 3, about 4, about 5, about 6, about 7, or about 8 acidic functional groups. In some embodiments, the acidic functional groups are strongly acidic (e.g. a pKa of <2). In some embodiments, the acidic functional groups are sulfonic acid functional groups, phosphoric acid functional groups, or carboxylic acid functional groups. In some embodiments, the acidic functional groups are sulfonic acid functional groups.

In some embodiments, the pKa of the basic nitrogen atom is about 4 to about 12. In some embodiments, the pKa of the basic nitrogen atom is about 4 to about 5, about 4 to about 6, about 4 to about 7, about 4 to about 8, about 4 to about 9, about 4 to about 10, about 4 to about 11, about 4 to about 12, about 5 to about 6, about 5 to about 7, about 5 to about 8, about 5 to about 9, about 5 to about 10, about 5 to about 11, about 5 to about 12, about 6 to about 7, about 6 to about 8, about 6 to about 9, about 6 to about 10, about 6 to about 11, about 6 to about 12, about 7 to about 8, about 7 to about 9, about 7 to about 10, about 7 to about 11, about 7 to about 12, about 8 to about 9, about 8 to about 10, about 8 to about 11, about 8 to about 12, about 9 to about 10, about 9 to about 11, about 9 to about 12, about 10 to about 11, about 10 to about 12, or about 11 to about 12. In some embodiments, the pKa of the basic nitrogen atom is about 4, about 5, about 6, about 7, about 8, about 9, about 10, about 11, or about 12. In some embodiments, the pKa of the basic nitrogen atom is at least about 4, about 5, about 6, about 7, about 8, about 9, about 10, or about 11. In some embodiments, the pKa of the basic nitrogen atom is at most about 5, about 6, about 7, about 8, about 9, about 10, about 11, or about 12.

In some embodiments, the pKa of the basic nitrogen atom is about 4 to about 7. In some embodiments, the pKa of the basic nitrogen atom is about 4 to about 4.5, about 4 to about 5, about 4 to about 5.5, about 4 to about 6, about 4 to about 6.5, about 4 to about 7, about 4.5 to about 5, about 4.5 to about 5.5, about 4.5 to about 6, about 4.5 to about 6.5, about 4.5 to about 7, about 5 to about 5.5, about 5 to about 6, about 5 to about 6.5, about 5 to about 7, about 5.5 to about 6, about 5.5 to about 6.5, about 5.5 to about 7, about 6 to about 6.5, about 6 to about 7, or about 6.5 to about 7. In some embodiments, the pKa of the basic nitrogen atom is about 4, about 4.5, about 5, about 5.5, about 6, about 6.5, or about 7. In some embodiments, the pKa of the basic nitrogen atom is at least about 4, about 4.5, about 5, about 5.5, about 6, or about 6.5. In some embodiments, the pKa of the basic nitrogen atom is at most about 4.5, about 5, about 5.5, about 6, about 6.5, or about 7.

In some embodiments, the pKa of the basic nitrogen atom is about 7 to about 11. In some embodiments, the pKa of the basic nitrogen atom is about 7 to about 7.5, about 7 to about 8, about 7 to about 8.5, about 7 to about 9, about 7 to about 9.5, about 7 to about 10, about 7 to about 10.5, about 7 to about 11, about 7.5 to about 8, about 7.5 to about 8.5, about 7.5 to about 9, about 7.5 to about 9.5, about 7.5 to about 10, about 7.5 to about 10.5, about 7.5 to about 11, about 8 to about 8.5, about 8 to about 9, about 8 to about 9.5, about 8 to about 10, about 8 to about 10.5, about 8 to about 11, about 8.5 to about 9, about 8.5 to about 9.5, about 8.5 to about 10, about 8.5 to about 10.5, about 8.5 to about 11, about 9 to about 9.5, about 9 to about 10, about 9 to about 10.5, about 9 to about 11, about 9.5 to about 10, about 9.5 to about 10.5, about 9.5 to about 11, about 10 to about 10.5, about 10 to about 11, or about 10.5 to about 11. In some embodiments, the pKa of the basic nitrogen atom is about 7, about 7.5, about 8, about 8.5, about 9, about 9.5, about 10, about 10.5, or about 11. In some embodiments, the pKa of the basic nitrogen atom is at least about 7, about 7.5, about 8, about 8.5, about 9, about 9.5, about 10, or about 10.5. In some embodiments, the pKa of the basic nitrogen atom is at most about 7.5, about 8, about 8.5, about 9, about 9.5, about 10, about 10.5, or about 11.

In some embodiments, pKa of the basic nitrogen atom is such that compound will be partially protonated at a physiologically tolerable pH. In some embodiments, the pKa is from about 4 to about 11. In some embodiments, the pKa is from about 4 to about 10. In some embodiments, the pKa is form about 4 to about 9. In some embodiments, the pKa is from about 5 to about 11. In some embodiments, the pKa is from about 5 to about 10. In some embodiments, the pKa is from about 5 to about 9. In some embodiments, the pKa is form about 6 to about 11. In some embodiments, the pKa is from about 6 to about 10. In some embodiments, the pKa is from about 6 to about 9. In some embodiments, the pKa is from about 7 to about 11. In some embodiments, the pKa is from about 7 to about 10. In some embodiments, the pKa is from about 7 to about 9. In some embodiments, the pKa is from about 8 to about 11. In some embodiments, the pKa is from about 8 to about 10. In some embodiments, the pKa is from about 8 to about 10.

In some embodiments, the basic nitrogen atom is an amine. In some embodiments, the amine is a primary amine, a secondary amine, or a tertiary amine. In some embodiments, the amine is an alkyl amine. In some embodiments, the amine is an aryl amine (e.g. an aniline).

In some embodiments, the basic nitrogen is comprised in a heterocycle. In some embodiments, the amine is comprised in an aromatic heterocycle. Non-limiting examples of such aromatic heterocycles include pyrroles, pyrazoles, imidazoles, azaindoles, indazoles, benzoxazoles, benzimidazoles, quinolines, isoquinolines, quinazolines, pyridines, pyrimidines, pyrazines, napthyridines, quinoxalines, phenazines, and the like, each of which may be substituted.

In an aspect, provided herein is a pharmaceutically acceptable salt of an pharmaceutical compound comprising: (i) an pharmaceutical compound, or an enantiomer, a mixture of enantiomers, or an isotopic variant thereof; or a pharmaceutically acceptable salt, solvate or hydrate thereof, wherein the pharmaceutical compound comprises a protonated nitrogen atom; and (ii) a conjugate base of complexing agent, wherein the conjugate base of complexing agent acts as the counterion of the pharmaceutical compound, wherein the pharmaceutically acceptable salt has a ratio of the conjugate base of complexing agent to the pharmaceutical compound that is from about 1:1 to about 1:4; wherein the pharmaceutical compound an antiviral compound, an antibacterial compound, an anti-fungal compound, a compound for treatment of a neurological disorder, a compound for treatment of Parkinson's disease, a treatment for migraine headache, a treatment for autoimmune disease, a treatment for cancer, a treatment for lymphoma, a treatment for pancreatitis, a treatment for opioid overdose, a treatment for flu infection, or a treatment for an inflammatory disorder. In some embodiments, the pharmaceutical compound is rotigotine, eletriptan, copanlisib, remdesivir, nafamostat (nafamostat mesylate), melevodopa, tigecycline, naloxone, amikacin, 1-amantadine, 2-amantadine, rimantadine, amifampridine, rapamycin, clonidine, or caspofungin. In some embodiments, the complexing agent comprises SBEBCD.

In some embodiments, the pharmaceutically acceptable salt provided herein consists essentially of the complexing agent and the pharmaceutical compound. In some embodiments, the pharmaceutically acceptable salt provided herein consists of the complexing agent and the pharmaceutical compound. In some embodiments, the pharmaceutically acceptable salt provided herein consists of the protonated pharmaceutical compound and the deprotonated complexing agent.

In some embodiments, the pharmaceutically acceptable salt is in a solid form. In some embodiments, the solid form is a crystalline form or an amorphous form. In some embodiments, the solid form is an amorphous powder. In some embodiments, the solid form is a lyophilized powder. In some embodiments, the solid form is a crystalline form.

In some embodiments, the pharmaceutically acceptable salt is dissolved or suspended in a liquid medium. In some embodiments, the liquid medium is an aqueous medium, an organic solvent, or a combination thereof. In some embodiments, the liquid medium is an aqueous medium. In some embodiments, the liquid medium is an organic solvent. In some embodiments, the organic solvent comprises acetic acid, acetone, acetonitrile, benzene, tert-butyl alcohol, tert-butyl methyl ether, carbon tetrachloride, chloroform, cyclohexane, 1,2-dichloroethane, dichloromethane, diethyl ether, diglyme, 1,2,-dimethoxyethane, dimethyl acetamide, dimethylformamide, dimethyl sulfoxide, dioxane, ethanol, ethyl acetate, ethyl methyl ketone, ethylene glycol, hexanes, hexamethylphosphoramide, methanol, nitromethane, pentanes, 2-propanol, pyridine, tetrahydrofuran, toluene, xylenes, or any combination thereof. In some embodiments, the pharmaceutically acceptable salt is dissolved or suspended in the liquid medium as an intermediate step in its preparation or in the preparation of a pharmaceutical composition comprising the salt.

In some embodiments, the pharmaceutically acceptable salt is substantially free of excess ions. Examples of such ions include other salts that may be left over from the preparation of the salts or byproducts of the production of the salts (e.g. sodium chloride, lithium chloride, potassium chloride, sodium bromide, and the like). In some embodiments, the excess ions are counterions to excess complexing agent or pharmaceutical compound in the salt preparation, such as excess sodium ions occupying the deprotonated acidic sites or chloride ions associated with excess protonated pharmaceutical compound.

In some embodiments, the conjugate base of complexing agent acts a counterion to 1 to 4 molecules of the pharmaceutical compound. In some embodiments, the total number of functional acidic groups of complexing agent is more than the total number of protonated nitrogen atoms of the pharmaceutical compound. In some embodiments, a portion, but not all, of the functional acidic groups of the complexing agent act as a counterion of the pharmaceutical compound. In some embodiments, the pharmaceutically acceptable salt may comprise an excess of the pharmaceutical compound, wherein the excess pharmaceutical compound is unionized. In some embodiments, the amount of excess pharmaceutical compound is 1 molar equivalent compared to the conjugate base of the complexing agent. In some embodiments, the complexing agent comprises a non-polar region. In some embodiments, the complexing agent comprises a non-polar pore. In some embodiments, the unionized pharmaceutical compound forms a complex with the conjugate base of the complexing agent through the no-polar region. In some embodiments, the unionized pharmaceutical compound forms a complex with the conjugate base of the complexing agent through the no-polar pore. The presence of excess pharmaceutical compound can have numerous benefits in certain contexts, including increasing the dose per unit weight or volume of the salt when the salt is used in a pharmaceutical composition. additionally, when used in a pharmaceutical composition, the presence of free base or unionized pharmaceutical compound can be used to raise the pH of the composition as it is administered, thus potentially facilitating both bioavailability and tolerability in certain contexts (e.g. when the pKa of the pharmaceutical compound is lower than the pH at which the compound can be comfortably administered to the target tissue). Additionally, many of the complexing agents contemplated herein have an additional coordination site for unionized APIs (e.g. the middle complexing site of a cyclodextrin). Thus, in some embodiments, the complexing agents used herein can offer additional solubilization of pharmaceutical compounds beyond that accomplished merely by acid/base chemistry and ion exchange/ion pairing.

In some embodiments, the pharmaceutically acceptable salt comprises additional equivalents of the pharmaceutical compound. In some embodiments, the additional equivalents are measured as compared to the moles of complexing agent. In some embodiments, the pharmaceutically acceptable salt comprises additional molar equivalents of the pharmaceutical compound. In some embodiments, the additional molar equivalents are measured as compared to the moles of complexing agent. In some embodiments, the additional equivalents are measured as compared to the moles of the pharmaceutical compound which forms a salt with the complexing agent (e.g. the protonated pharmaceutical compound).

In some embodiments, the pharmaceutically acceptable salt comprises additional molar equivalents of unionized pharmaceutical compound compared to the protonated pharmaceutical compound of the complexing agent/protonated pharmaceutical compound salt. In some embodiments, the pharmaceutically acceptable salt comprises about 0.01 molar equivalents to about 10 molar equivalents of the unionized pharmaceutical compound. In some embodiments, the pharmaceutically acceptable salt comprises about 0.1 molar equivalents to about 1 molar equivalents of the unionized pharmaceutical compound. In some embodiments, the pharmaceutically acceptable salt comprises about 0.1 molar equivalents to about 10 molar equivalents of unionized opioid. In some embodiments, the pharmaceutically acceptable salt comprises about 0.1 molar equivalents to about 0.2 molar equivalents, about 0.1 molar equivalents to about 0.5 molar equivalents, about 0.1 molar equivalents to about 0.75 molar equivalents, about 0.1 molar equivalents to about 1 molar equivalents, about 0.2 molar equivalents to about 0.5 molar equivalents, about 0.2 molar equivalents to about 0.75 molar equivalents, about 0.2 molar equivalents to about 1 molar equivalents, about 0.5 molar equivalents to about 0.75 molar equivalents, about 0.5 molar equivalents to about 1 molar equivalents, or about 0.75 molar equivalents to about 1 molar equivalents. In some embodiments, the pharmaceutically acceptable salt comprises about 0.1 molar equivalents, about 0.2 molar equivalents, about 0.5 molar equivalents, about 0.75 molar equivalents, or about 1 molar equivalents of the unionized pharmaceutical compound. In some embodiments, the pharmaceutically acceptable salt comprises at least about 0.1 molar equivalents, about 0.2 molar equivalents, about 0.5 molar equivalents, or about 0.75 molar equivalents of the unionized pharmaceutical compound. In some embodiments, the pharmaceutically acceptable salt comprises at most about 0.2 molar equivalents, about 0.5 molar equivalents, about 0.75 molar equivalents, or about 1 molar equivalents of the unionized pharmaceutical compound. In some embodiments, the pharmaceutically acceptable salt comprises about 0.5 equivalents to about 5 equivalents of the unionized pharmaceutical compound. In some embodiments, the pharmaceutically acceptable salt comprises about 0.5 equivalents to about 1 equivalents, about 0.5 equivalents to about 2 equivalents, about 0.5 equivalents to about 3 equivalents, about 0.5 equivalents to about 4 equivalents, about 0.5 equivalents to about 5 equivalents, about 1 equivalents to about 2 equivalents, about 1 equivalents to about 3 equivalents, about 1 equivalents to about 4 equivalents, about 1 equivalents to about 5 equivalents, about 2 equivalents to about 3 equivalents, about 2 equivalents to about 4 equivalents, about 2 equivalents to about 5 equivalents, about 3 equivalents to about 4 equivalents, about 3 equivalents to about 5 equivalents, or about 4 equivalents to about 5 equivalents of the unionized pharmaceutical compound. In some embodiments, the pharmaceutically acceptable salt comprises about 0.5 equivalents, about 1 equivalents, about 2 equivalents, about 3 equivalents, about 4 equivalents, or about 5 equivalents of the unionized pharmaceutical compound. In some embodiments, the pharmaceutically acceptable salt comprises at least about 0.5 equivalents, about 1 equivalents, about 2 equivalents, about 3 equivalents, or about 4 equivalents of the unionized pharmaceutical compound. In some embodiments, the pharmaceutically acceptable salt comprises at most about 1 equivalents, about 2 equivalents, about 3 equivalents, about 4 equivalents, or about 5 equivalents of the unionized pharmaceutical compound.

In some embodiments, the pharmaceutically acceptable salt comprises additional molar equivalents of unionized pharmaceutical compound compared to complexing agent of the complexing agent/protonated pharmaceutical compound salt. In some embodiments, the pharmaceutically acceptable salt comprises about 0.01 molar equivalents to about 10 molar equivalents of unionized pharmaceutical compound. In some embodiments, the pharmaceutically acceptable salt comprises about 0.1 molar equivalents to about 10 molar equivalents of unionized opioid. In some embodiments, the pharmaceutically acceptable salt comprises about 0.1 molar equivalents to about 0.5 molar equivalents, about 0.1 molar equivalents to about 1 molar equivalents, about 0.1 molar equivalents to about 2 molar equivalents, about 0.1 molar equivalents to about 3 molar equivalents, about 0.1 molar equivalents to about 5 molar equivalents, about 0.1 molar equivalents to about 7 molar equivalents, about 0.1 molar equivalents to about 10 molar equivalents, about 0.5 molar equivalents to about 1 molar equivalents, about 0.5 molar equivalents to about 2 molar equivalents, about 0.5 molar equivalents to about 3 molar equivalents, about 0.5 molar equivalents to about 5 molar equivalents, about 0.5 molar equivalents to about 7 molar equivalents, about 0.5 molar equivalents to about 10 molar equivalents, about 1 molar equivalents to about 2 molar equivalents, about 1 molar equivalents to about 3 molar equivalents, about 1 molar equivalents to about 5 molar equivalents, about 1 molar equivalents to about 7 molar equivalents, about 1 molar equivalents to about 10 molar equivalents, about 2 molar equivalents to about 3 molar equivalents, about 2 molar equivalents to about 5 molar equivalents, about 2 molar equivalents to about 7 molar equivalents, about 2 molar equivalents to about 10 molar equivalents, about 3 molar equivalents to about 5 molar equivalents, about 3 molar equivalents to about 7 molar equivalents, about 3 molar equivalents to about 10 molar equivalents, about 5 molar equivalents to about 7 molar equivalents, about 5 molar equivalents to about 10 molar equivalents, or about 7 molar equivalents to about 10 molar equivalents of unionized pharmaceutical compound of unionized pharmaceutical compound. In some embodiments, the pharmaceutically acceptable salt comprises about 0.1 molar equivalents, about 0.5 molar equivalents, about 1 molar equivalents, about 2 molar equivalents, about 3 molar equivalents, about 5 molar equivalents, about 7 molar equivalents, or about 10 molar equivalents of unionized pharmaceutical compound. In some embodiments, the pharmaceutically acceptable salt comprises at least about 0.1 molar equivalents, about 0.5 molar equivalents, about 1 molar equivalents, about 2 molar equivalents, about 3 molar equivalents, about 5 molar equivalents, or about 7 molar equivalents of unionized pharmaceutical compound. In some embodiments, the pharmaceutically acceptable salt comprises at most about 0.5 molar equivalents, about 1 molar equivalents, about 2 molar equivalents, about 3 molar equivalents, about 5 molar equivalents, about 7 molar equivalents, or about 10 molar equivalents of unionized pharmaceutical compound. In some embodiments, the pharmaceutically acceptable salt comprises about 0.01 molar equivalents to about 20 molar equivalents of unionized pharmaceutical compound compared to the complexing agent. In some embodiments, the pharmaceutically acceptable salt comprises about 0.1 molar equivalents to about 20 molar equivalents of unionized pharmaceutical compound compared to the complexing agent. In some embodiments, the pharmaceutical composition comprises about 1 molar equivalents to about 20 molar equivalents of unionized pharmaceutical compound. compared to the complexing agent. In some embodiments, at least a portion of these additional equivalents of the unionized pharmaceutical compound relative to the complexing agent are complexed to the complexing agent. (e.g. up to about 1 molar equivalent of the unionized pharmaceutical compound relative to the moles of complexing agent).

In some embodiments, the solubility of the pharmaceutical compound in the pharmaceutically acceptable salt is higher than (i) the solubility of the pharmaceutical compound as a salt; or (ii) the solubility of the pharmaceutical compound in a salt comprising the pharmaceutical compound in freebase form that is complexing to a non-polar pore of the complexing agent, wherein the pharmaceutical compound has the same concentration in the pharmaceutically acceptable salt, the pharmaceutical compound as a salt, and the salt comprising the pharmaceutical compound in freebase form. In some embodiments, the solubility of the pharmaceutical compound in the pharmaceutically acceptable salt is higher than the solubility of the pharmaceutical compound in a salt comprising the pharmaceutical compound and complexing agent with a higher molar ratio of the pharmaceutical compound to the complexing agent, wherein the pharmaceutical compound has the same concentration in the pharmaceutically acceptable salt and the salt with a higher molar ratio of the pharmaceutical compound to the complexing agent. In some embodiments, the solubility of the pharmaceutical compound in the pharmaceutically acceptable salt has an increase of at least 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, or 100-fold compared with (i) the solubility of the pharmaceutical compound as a salt; or (ii) the solubility of the pharmaceutical compound in a salt comprising the pharmaceutical compound in freebase form that is complexing to a non-polar pore of the complexing agent, wherein the pharmaceutical compound has the same concentration in the pharmaceutically acceptable salt, the pharmaceutical compound as a salt, and the salt comprising the pharmaceutical compound in freebase form. In some embodiments, the solubility of the pharmaceutical compound in the pharmaceutically acceptable salt has an increase of at least 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, or 100-fold compared with the solubility of the pharmaceutical compound in a salt comprising the pharmaceutical compound and complexing agent with a higher molar ratio of the pharmaceutical compound to the complexing agent, wherein the pharmaceutical compound has the same concentration in the pharmaceutically acceptable salt and the salt with a higher molar ratio of the pharmaceutical compound to the complexing agent.

In some embodiments, the pharmaceutical compound is an antiviral compound, an antibacterial compound, an anti-fungal compound, a compound for treatment of a neurological disorder, a compound for treatment of Parkinson's disease, a treatment for migraine headache, a treatment for autoimmune disease, a treatment for cancer, a treatment for lymphoma, a treatment for pancreatitis, a treatment for opioid overdose, a treatment for flu infection, or a treatment for an inflammatory disorder. In some embodiments, the pharmaceutical compound comprises rotigotine, eletriptan, copanlisib, remdesivir, nafamostat (nafamostat mesylate), midazolam, melevodopa, tigecycline, naloxone, amikacin, 1-amantadine, 2-amantadine, rimantadine, amifampridine, rapamycin, clonidine, or caspofungin.

In an aspect, provided herein is a pharmaceutically acceptable salt of an pharmaceutical compound comprising: (i) an pharmaceutical compound, or an enantiomer, a mixture of enantiomers, or an isotopic variant thereof; or a pharmaceutically acceptable salt, solvate or hydrate thereof, wherein the pharmaceutical compound comprises a protonated nitrogen atom; (ii) a conjugate base of a complexing agent, wherein the conjugate base of the complexing agent acts as a counterion of the pharmaceutical compound, wherein the pharmaceutically acceptable salt has a ratio of the conjugate base of the complexing agent to the pharmaceutical compound that is from about 1:1 to about 1:4; and an additional molar equivalent of the pharmaceutical compound in an unionized form. In some embodiments, the pharmaceutical compound comprises an antiviral compound, an antibacterial compound, an anti-fungal compound, a compound for treatment of a neurological disorder, a compound for treatment of Parkinson's disease, a treatment for migraine headache, a treatment for autoimmune disease, a treatment for cancer, a treatment for lymphoma, a treatment for pancreatitis, a treatment for opioid overdose, a treatment for flu infection, or a treatment for an inflammatory disorder. In some embodiments, the pharmaceutical compound is rotigotine, eletriptan, copanlisib, remdesivir, nafamostat (nafamostat mesylate), midazolam, melevodopa, tigecycline, naloxone, amikacin, 1-amantadine, 2-amantadine, rimantadine, amifampridine, rapamycin, clonidine, or caspofungin. In some embodiments, the complexing agent comprises SBEBCD.

In some embodiments, the pharmaceutically acceptable salt provided herein consists essentially of the complexing agent and the pharmaceutical compound. In some embodiments, the pharmaceutically acceptable salt provided herein consists of the complexing agent and the pharmaceutical compound. In some embodiments, the pharmaceutically acceptable salt provided herein consists of the protonated pharmaceutical compound, deprotonated pharmaceutical compound and the deprotonated complexing agent.

In some embodiments, the pharmaceutically acceptable salt is in a solid form. In some embodiments, the solid form is a crystalline form or an amorphous form. In some embodiments, the solid form is an amorphous powder. In some embodiments, the solid form is a lyophilized powder. In some embodiments, the solid form is a crystalline form.

In some embodiments, the pharmaceutically acceptable salt is dissolved or suspended in a liquid medium. In some embodiments, the liquid medium is an aqueous medium, an organic solvent, or a combination thereof. In some embodiments, the liquid medium is an aqueous medium. In some embodiments, the liquid medium is an organic solvent. In some embodiments, the organic solvent comprises acetic acid, acetone, acetonitrile, benzene, tert-butyl alcohol, tert-butyl methyl ether, carbon tetrachloride, chloroform, cyclohexane, 1,2-dichloroethane, dichloromethane, diethyl ether, diglyme, 1,2-dimethoxyethane, dimethyl acetamide, dimethylformamide, dimethyl sulfoxide, dioxane, ethanol, ethyl acetate, ethyl methyl ketone, ethylene glycol, hexanes, hexamethylphosphoramide, methanol, nitromethane, pentanes, 2-proponal, pyridine, tetrahydrofuran, toluene, xylenes, or any combination thereof. In some embodiments, the pharmaceutically acceptable salt is dissolved or suspended in the liquid medium as an intermediate step in its preparation or in the preparation of a pharmaceutical composition comprising the salt.

In some embodiments, the solubility of the pharmaceutical compound in the pharmaceutically acceptable salt is higher than (i) the solubility of the pharmaceutical compound as a salt; or (ii) the solubility of the pharmaceutical compound in a salt comprising the pharmaceutical compound in freebase form that is complexing to a non-polar pore of the complexing agent, wherein the pharmaceutical compound has the same concentration in the pharmaceutically acceptable salt, the pharmaceutical compound as a salt, and the salt comprising the pharmaceutical compound in freebase form. In some embodiments, the solubility of the pharmaceutical compound in the pharmaceutically acceptable salt is higher than the solubility of the pharmaceutical compound in a salt comprising the pharmaceutical compound and complexing agent with a higher molar ratio of the pharmaceutical compound to the complexing agent, wherein the pharmaceutical compound has the same concentration in the pharmaceutically acceptable salt and the salt with a higher molar ratio of the pharmaceutical compound to the complexing agent. In some embodiments, the solubility of the pharmaceutical compound in the pharmaceutically acceptable salt has an increase of at least 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, or 100-fold compared with (i) the solubility of the pharmaceutical compound as a salt; or (ii) the solubility of the pharmaceutical compound in a salt comprising the pharmaceutical compound in freebase form that is complexing to a non-polar pore of the complexing agent, wherein the pharmaceutical compound has the same concentration in the pharmaceutically acceptable salt, the pharmaceutical compound as a salt, and the salt comprising the pharmaceutical compound in freebase form. In some embodiments, the solubility of the pharmaceutical compound in the pharmaceutically acceptable salt has an increase of at least 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, or 100-fold compared with the solubility of the pharmaceutical compound in a salt comprising the pharmaceutical compound and complexing agent with a higher molar ratio of the pharmaceutical compound to the complexing agent, wherein the pharmaceutical compound has the same concentration in the pharmaceutically acceptable salt and the salt with a higher molar ratio of the pharmaceutical compound to the complexing agent.

In some embodiments, the pharmaceutically acceptable salt is substantially free of excess ions. Examples of such ions include other salts that may be left over from the preparation of the salts or byproducts of the production of the salts (e.g. sodium chloride, lithium chloride, potassium chloride, sodium bromide, and the like). In some embodiments, the excess ions are counterions to excess complexing agent or pharmaceutical compound in the salt preparation, such as excess sodium ions occupying the deprotonated acidic sites or chloride ions associated with excess protonated pharmaceutical compound.

In some embodiments, the conjugate base of the complexing agent acts a counterion to 1 to 4 molecules of the pharmaceutical compound. In some embodiments, the total number of functional acidic groups of the complexing agent is more than the total number of protonated nitrogen atoms of the pharmaceutical compound. In some embodiments, a portion, but not all, of the functional acidic groups of the complexing agent act as a counterion of the pharmaceutical compound. In some embodiments, the amount of unionized pharmaceutical compound is about 0.1 to about 1 molar equivalent compared to the conjugate base of the complexing agent. In some embodiments, the complexing agent comprises a non-polar region. In some embodiments, the complexing agent comprises a non-polar pore. In some embodiments, the unionized pharmaceutical compound forms a complex with the conjugate base of the complexing agent through the no-polar region. In some embodiments, the unionized pharmaceutical compound forms a complex with the conjugate base of the complexing agent through the no-polar pore. The presence of unionized pharmaceutical compound can have numerous benefits in certain contexts, including increasing the dose per unit weight or volume of the salt when the salt is used in a pharmaceutical composition. Additionally, when used in a pharmaceutical composition, the presence of free base or unionized pharmaceutical compound can be used to raise the pH of the composition as it is administered, thus potentially facilitating both bioavailability and tolerability in certain contexts (e.g. when the pKa of the pharmaceutical compound is lower than the pH at which the compound can be comfortably administered to the target tissue). Additionally, many of the complexing agents contemplated herein have an additional coordination site for unionized APIs (e.g. the middle complexing site of a cyclodextrin). Thus, in some embodiments, the complexing agents used herein can offer additional solubilization of pharmaceutical compounds beyond that accomplished merely by acid/base chemistry and ion exchange/ion pairing.

In some embodiments, the pharmaceutically acceptable salt comprises about 0.01 molar equivalents to about 10 molar equivalents of the unionized pharmaceutical compound. In some embodiments, the pharmaceutically acceptable salt comprises about 0.1 molar equivalents to about 1 molar equivalents of the unionized pharmaceutical compound. In some embodiments, the pharmaceutically acceptable salt comprises about 0.1 molar equivalents to about 10 molar equivalents of unionized opioid. In some embodiments, the pharmaceutically acceptable salt comprises about 0.1 molar equivalents to about 0.2 molar equivalents, about 0.1 molar equivalents to about 0.5 molar equivalents, about 0.1 molar equivalents to about 0.75 molar equivalents, about 0.1 molar equivalents to about 1 molar equivalents, about 0.2 molar equivalents to about 0.5 molar equivalents, about 0.2 molar equivalents to about 0.75 molar equivalents, about 0.2 molar equivalents to about 1 molar equivalents, about 0.5 molar equivalents to about 0.75 molar equivalents, about 0.5 molar equivalents to about 1 molar equivalents, or about 0.75 molar equivalents to about 1 molar equivalents. In some embodiments, the pharmaceutically acceptable salt comprises about 0.1 molar equivalents, about 0.2 molar equivalents, about 0.5 molar equivalents, about 0.75 molar equivalents, or about 1 molar equivalents of the unionized pharmaceutical compound. In some embodiments, the pharmaceutically acceptable salt comprises at least about 0.1 molar equivalents, about 0.2 molar equivalents, about 0.5 molar equivalents, or about 0.75 molar equivalents of the unionized pharmaceutical compound. In some embodiments, the pharmaceutically acceptable salt comprises at most about 0.2 molar equivalents, about 0.5 molar equivalents, about 0.75 molar equivalents, or about 1 molar equivalents of the unionized pharmaceutical compound. In some embodiments, the pharmaceutically acceptable salt comprises about 0.5 equivalents to about 5 equivalents of the unionized pharmaceutical compound. In some embodiments, the pharmaceutically acceptable salt comprises about 0.5 equivalents to about 1 equivalents, about 0.5 equivalents to about 2 equivalents, about 0.5 equivalents to about 3 equivalents, about 0.5 equivalents to about 4 equivalents, about 0.5 equivalents to about 5 equivalents, about 1 equivalents to about 2 equivalents, about 1 equivalents to about 3 equivalents, about 1 equivalents to about 4 equivalents, about 1 equivalents to about 5 equivalents, about 2 equivalents to about 3 equivalents, about 2 equivalents to about 4 equivalents, about 2 equivalents to about 5 equivalents, about 3 equivalents to about 4 equivalents, about 3 equivalents to about 5 equivalents, or about 4 equivalents to about 5 equivalents of the unionized pharmaceutical compound. In some embodiments, the pharmaceutically acceptable salt comprises about 0.5 equivalents, about 1 equivalent, about 2 equivalents, about 3 equivalents, about 4 equivalents, or about 5 equivalents of the unionized pharmaceutical compound. In some embodiments, the pharmaceutically acceptable salt comprises at least about 0.5 equivalents, about 1 equivalent, about 2 equivalents, about 3 equivalents, or about 4 equivalents of the unionized pharmaceutical compound. In some embodiments, the pharmaceutically acceptable salt comprises at most about 1 equivalent, about 2 equivalents, about 3 equivalents, about 4 equivalents, or about 5 equivalents of the unionized pharmaceutical compound.

In some embodiments, the pharmaceutically acceptable salt comprises molar equivalents of unionized pharmaceutical compound compared to complexing agent of the complexing agent/protonated pharmaceutical compound salt. In some embodiments, the pharmaceutically acceptable salt comprises about 0.01 molar equivalents to about 10 molar equivalents of unionized pharmaceutical compound. In some embodiments, the pharmaceutically acceptable salt comprises about 0.1 molar equivalents to about 10 molar equivalents of unionized opioid. In some embodiments, the pharmaceutically acceptable salt comprises about 0.1 molar equivalents to about 0.5 molar equivalents, about 0.1 molar equivalents to about 1 molar equivalents, about 0.1 molar equivalents to about 2 molar equivalents, about 0.1 molar equivalents to about 3 molar equivalents, about 0.1 molar equivalents to about 5 molar equivalents, about 0.1 molar equivalents to about 7 molar equivalents, about 0.1 molar equivalents to about 10 molar equivalents, about 0.5 molar equivalents to about 1 molar equivalents, about 0.5 molar equivalents to about 2 molar equivalents, about 0.5 molar equivalents to about 3 molar equivalents, about 0.5 molar equivalents to about 5 molar equivalents, about 0.5 molar equivalents to about 7 molar equivalents, about 0.5 molar equivalents to about 10 molar equivalents, about 1 molar equivalents to about 2 molar equivalents, about 1 molar equivalents to about 3 molar equivalents, about 1 molar equivalents to about 5 molar equivalents, about 1 molar equivalents to about 7 molar equivalents, about 1 molar equivalents to about 10 molar equivalents, about 2 molar equivalents to about 3 molar equivalents, about 2 molar equivalents to about 5 molar equivalents, about 2 molar equivalents to about 7 molar equivalents, about 2 molar equivalents to about 10 molar equivalents, about 3 molar equivalents to about 5 molar equivalents, about 3 molar equivalents to about 7 molar equivalents, about 3 molar equivalents to about 10 molar equivalents, about 5 molar equivalents to about 7 molar equivalents, about 5 molar equivalents to about 10 molar equivalents, or about 7 molar equivalents to about 10 molar equivalents of unionized pharmaceutical compound of unionized pharmaceutical compound. In some embodiments, the pharmaceutically acceptable salt comprises about 0.1 molar equivalents, about 0.5 molar equivalents, about 1 molar equivalents, about 2 molar equivalents, about 3 molar equivalents, about 5 molar equivalents, about 7 molar equivalents, or about 10 molar equivalents of unionized pharmaceutical compound. In some embodiments, the pharmaceutically acceptable salt comprises at least about 0.1 molar equivalents, about 0.5 molar equivalents, about 1 molar equivalents, about 2 molar equivalents, about 3 molar equivalents, about 5 molar equivalents, or about 7 molar equivalents of unionized pharmaceutical compound. In some embodiments, the pharmaceutically acceptable salt comprises at most about 0.5 molar equivalents, about 1 molar equivalents, about 2 molar equivalents, about 3 molar equivalents, about 5 molar equivalents, about 7 molar equivalents, or about 10 molar equivalents of unionized pharmaceutical compound. In some embodiments, the pharmaceutically acceptable salt comprises about 0.01 molar equivalents to about 20 molar equivalents of unionized pharmaceutical compound compared to the complexing agent. In some embodiments, the pharmaceutically acceptable salt comprises about 0.1 molar equivalents to about 20 molar equivalents of unionized pharmaceutical compound compared to the complexing agent. In some embodiments, the pharmaceutical composition comprises about 1 molar equivalent to about 20 molar equivalents of unionized pharmaceutical compound. compared to the complexing agent. In some embodiments, at least a portion of these additional equivalents of the unionized pharmaceutical compound relative to the complexing agent are complexed to the complexing agent. (e.g. up to about 1 molar equivalent of the unionized pharmaceutical compound relative to the moles of complexing agent).

In an aspect, provided herein is a pharmaceutically acceptable salt of an pharmaceutical compound comprising: (i) an pharmaceutical compound, or an enantiomer, a mixture of enantiomers, or an isotopic variant thereof; or a pharmaceutically acceptable salt, solvate or hydrate thereof, wherein the pharmaceutical compound comprises a prodrug comprising an unionized substance conjugated to a chemical entity, wherein the chemical entity comprises a protonated nitrogen atom; and (ii) a conjugate base of a complexing agent, wherein the conjugate base of the complexing agent acts as a counterion of the pharmaceutical compound. In some embodiments, the unionized substance comprises brexanolone. In some embodiments, the chemical entity comprises γ-aminobutyric acid (GABA).

In some embodiments, the prodrug comprises esters of carboxylic acid groups between the unionized substance and the chemical entity. In some embodiments, the prodrug is converted in vivo by the action of biochemicals within the patient's body, such as enzymes, to an active pharmaceutical ingredient. In some embodiments, the esters of carboxylic acid groups can be hydrolyzed by endogenous esterases as are found in the bloodstream of humans and other mammals. Further examples of prodrugs include boronate esters which can be hydrolyzed under physiological conditions to afford the corresponding boronic acid.

In some embodiments, the pharmaceutically acceptable salt provided herein consists essentially of the complexing agent and the pharmaceutical compound. In some embodiments, the pharmaceutically acceptable salt provided herein consists of the complexing agent and the pharmaceutical compound. In some embodiments, the pharmaceutically acceptable salt provided herein consists of the protonated pharmaceutical compound and the deprotonated complexing agent.

In some embodiments, the pharmaceutically acceptable salt is in a solid form. In some embodiments, the solid form is a crystalline form or an amorphous form. In some embodiments, the solid form is an amorphous powder. In some embodiments, the solid form is a lyophilized powder. In some embodiments, the solid form is a crystalline form.

In some embodiments, the pharmaceutically acceptable salt is dissolved or suspended in a liquid medium. In some embodiments, the liquid medium is an aqueous medium, an organic solvent, or a combination thereof. In some embodiments, the liquid medium is an aqueous medium. In some embodiments, the liquid medium is an organic solvent. In some embodiments, the organic solvent comprises acetic acid, acetone, acetonitrile, benzene, tert-butyl alcohol, tert-butyl methyl ether, carbon tetrachloride, chloroform, cyclohexane, 1,2-dichloroethane, dichloromethane, diethyl ether, diglyme, 1,2-dimethoxyethane, dimethyl acetamide, dimethylformamide, dimethyl sulfoxide, dioxane, ethanol, ethyl acetate, ethyl methyl ketone, ethylene glycol, hexanes, hexamethylphosphoramide, methanol, nitromethane, pentanes, 2-propanol, pyridine, tetrahydrofuran, toluene, xylenes, or any combination thereof. In some embodiments, the pharmaceutically acceptable salt is dissolved or suspended in the liquid medium as an intermediate step in its preparation or in the preparation of a pharmaceutical composition comprising the salt.

In some embodiments, the pharmaceutically acceptable salt is substantially free of excess ions. Examples of such ions include other salts that may be left over from the preparation of the salts or byproducts of the production of the salts (e.g. sodium chloride, lithium chloride, potassium chloride, sodium bromide, and the like). In some embodiments, the excess ions are counterions to excess complexing agent or pharmaceutical compound in the salt preparation, such as excess sodium ions occupying the deprotonated acidic sites or chloride ions associated with excess protonated pharmaceutical compound.

In some embodiments, the pharmaceutically acceptable salt may comprise an unionized compound. In some embodiments, the compound is the unionized substance. In some embodiments, the compound is difference from the unionized substance. The presence of unionized compound can have numerous benefits in certain contexts, including increasing the dose per unit weight or volume of the salt when the salt is used in a pharmaceutical composition. Additionally, when used in a pharmaceutical composition, the presence of free base or unionized compound can be used to raise the pH of the composition as it is administered, thus potentially facilitating both bioavailability and tolerability in certain contexts (e. g. when the pKa of the pharmaceutical compound is lower than the pH at which the compound can be comfortably administered to the target tissue). Additionally, many of the complexing agents contemplated herein have an additional coordination site for unionized APIs (e.g. the middle complexing site of a cyclodextrin). Thus, in some embodiments, the complexing agents used herein can offer additional solubilization of compounds beyond that accomplished merely by acid/base chemistry and ion exchange/ion pairing. In some embodiments, the conjugate base of the complexing agent comprises a non-polar region. In some embodiments, the conjugate base of the complexing agent comprises a non-polar pore. In some embodiments, the unionized compound is complexed to the complexing agent through the non-polar region or non-polar pore.

In some embodiments, the pharmaceutically acceptable salt comprises molar equivalents of the pharmaceutical compound. In some embodiments, the molar equivalents are measured as compared to the moles of complexing agent. In some embodiments, the pharmaceutically acceptable salt comprises additional molar equivalents of the pharmaceutical compound. In some embodiments, the additional molar equivalents are measured as compared to the moles of complexing agent. In some embodiments, the molar equivalents are measured as compared to the moles of the pharmaceutical compound which forms a salt with the complexing agent (e.g. the protonated pharmaceutical compound).

In some embodiments, the pharmaceutically acceptable salt comprises molar equivalents of unionized substance compared to the protonated pharmaceutical compound of the complexing agent/protonated pharmaceutical compound salt. In some embodiments, the pharmaceutically acceptable salt comprises about 0.01 molar equivalents to about 10 molar equivalents of the unionized substance. In some embodiments, the pharmaceutically acceptable salt comprises about 0.1 molar equivalents to about 1 molar equivalents of the unionized substance. In some embodiments, the pharmaceutically acceptable salt comprises about 0.1 molar equivalents to about 10 molar equivalents of unionized substance. In some embodiments, the pharmaceutically acceptable salt comprises about 0.1 molar equivalents to about 0.2 molar equivalents, about 0.1 molar equivalents to about 0.5 molar equivalents, about 0.1 molar equivalents to about 0.75 molar equivalents, about 0.1 molar equivalents to about 1 molar equivalents, about 0.2 molar equivalents to about 0.5 molar equivalents, about 0.2 molar equivalents to about 0.75 molar equivalents, about 0.2 molar equivalents to about 1 molar equivalents, about 0.5 molar equivalents to about 0.75 molar equivalents, about 0.5 molar equivalents to about 1 molar equivalents, or about 0.75 molar equivalents to about 1 molar equivalents of the unionized substance. In some embodiments, the pharmaceutically acceptable salt comprises about 0.1 molar equivalents, about 0.2 molar equivalents, about 0.5 molar equivalents, about 0.75 molar equivalents, or about 1 molar equivalents of the unionized pharmaceutical compound. In some embodiments, the pharmaceutically acceptable salt comprises at least about 0.1 molar equivalents, about 0.2 molar equivalents, about 0.5 molar equivalents, or about 0.75 molar equivalents of the unionized substance. In some embodiments, the pharmaceutically acceptable salt comprises at most about 0.2 molar equivalents, about 0.5 molar equivalents, about 0.75 molar equivalents, or about 1 molar equivalents of the unionized substance. In some embodiments, the pharmaceutically acceptable salt comprises about 0.5 equivalents to about 5 equivalents of the unionized substance. In some embodiments, the pharmaceutically acceptable salt comprises about 0.5 equivalents to about 1 equivalents, about 0.5 equivalents to about 2 equivalents, about 0.5 equivalents to about 3 equivalents, about 0.5 equivalents to about 4 equivalents, about 0.5 equivalents to about 5 equivalents, about 1 equivalents to about 2 equivalents, about 1 equivalents to about 3 equivalents, about 1 equivalents to about 4 equivalents, about 1 equivalents to about 5 equivalents, about 2 equivalents to about 3 equivalents, about 2 equivalents to about 4 equivalents, about 2 equivalents to about 5 equivalents, about 3 equivalents to about 4 equivalents, about 3 equivalents to about 5 equivalents, or about 4 equivalents to about 5 equivalents of the unionized substance. In some embodiments, the pharmaceutically acceptable salt comprises about 0.5 equivalents, about 1 equivalent, about 2 equivalents, about 3 equivalents, about 4 equivalents, or about 5 equivalents of the unionized substance. In some embodiments, the pharmaceutically acceptable salt comprises at least about 0.5 equivalents, about 1 equivalent, about 2 equivalents, about 3 equivalents, or about 4 equivalents of the unionized substance. In some embodiments, the pharmaceutically acceptable salt comprises at most about 1 equivalent, about 2 equivalents, about 3 equivalents, about 4 equivalents, or about 5 equivalents of the unionized substance.

In some embodiments, the pharmaceutically acceptable salt comprises molar equivalents of unionized substance compared to complexing agent of the complexing agent/protonated pharmaceutical compound salt. In some embodiments, the pharmaceutically acceptable salt comprises about 0.01 molar equivalents to about 10 molar equivalents of unionized substance. In some embodiments, the pharmaceutically acceptable salt comprises about 0.1 molar equivalents to about 10 molar equivalents of unionized substance. In some embodiments, the pharmaceutically acceptable salt comprises about 0.1 molar equivalents to about 0.5 molar equivalents, about 0.1 molar equivalents to about 1 molar equivalents, about 0.1 molar equivalents to about 2 molar equivalents, about 0.1 molar equivalents to about 3 molar equivalents, about 0.1 molar equivalents to about 5 molar equivalents, about 0.1 molar equivalents to about 7 molar equivalents, about 0.1 molar equivalents to about 10 molar equivalents, about 0.5 molar equivalents to about 1 molar equivalents, about 0.5 molar equivalents to about 2 molar equivalents, about 0.5 molar equivalents to about 3 molar equivalents, about 0.5 molar equivalents to about 5 molar equivalents, about 0.5 molar equivalents to about 7 molar equivalents, about 0.5 molar equivalents to about 10 molar equivalents, about 1 molar equivalents to about 2 molar equivalents, about 1 molar equivalents to about 3 molar equivalents, about 1 molar equivalents to about 5 molar equivalents, about 1 molar equivalents to about 7 molar equivalents, about 1 molar equivalents to about 10 molar equivalents, about 2 molar equivalents to about 3 molar equivalents, about 2 molar equivalents to about 5 molar equivalents, about 2 molar equivalents to about 7 molar equivalents, about 2 molar equivalents to about 10 molar equivalents, about 3 molar equivalents to about 5 molar equivalents, about 3 molar equivalents to about 7 molar equivalents, about 3 molar equivalents to about 10 molar equivalents, about 5 molar equivalents to about 7 molar equivalents, about 5 molar equivalents to about 10 molar equivalents, or about 7 molar equivalents to about 10 molar equivalents of unionized pharmaceutical compound of unionized substance. In some embodiments, the pharmaceutically acceptable salt comprises about 0.1 molar equivalents, about 0.5 molar equivalents, about 1 molar equivalents, about 2 molar equivalents, about 3 molar equivalents, about 5 molar equivalents, about 7 molar equivalents, or about 10 molar equivalents of unionized substance. In some embodiments, the pharmaceutically acceptable salt comprises at least about 0.1 molar equivalents, about 0.5 molar equivalents, about 1 molar equivalents, about 2 molar equivalents, about 3 molar equivalents, about 5 molar equivalents, or about 7 molar equivalents of unionized substance. In some embodiments, the pharmaceutically acceptable salt comprises at most about 0.5 molar equivalents, about 1 molar equivalents, about 2 molar equivalents, about 3 molar equivalents, about 5 molar equivalents, about 7 molar equivalents, or about 10 molar equivalents of unionized substance. In some embodiments, the pharmaceutically acceptable salt comprises about 0.01 molar equivalents to about 20 molar equivalents of unionized substance compared to the complexing agent. In some embodiments, the pharmaceutically acceptable salt comprises about 0.1 molar equivalents to about 20 molar equivalents of unionized substance compared to the complexing agent. In some embodiments, the pharmaceutical composition comprises about 1 molar equivalent to about 20 molar equivalents of unionized substance compared to the complexing agent. In some embodiments, at least a portion of these molar equivalents of the unionized substance relative to the complexing agent are complexed to the complexing agent. (e.g. up to about 1 molar equivalent of the unionized substance relative to the moles of complexing agent). In some embodiments, unionized substance is complexed to the non-polar region or non-polar pore of the complexing agent.

In some embodiments, the solubility of the ununionized substance in the pharmaceutically acceptable salt is higher than (i) the solubility of the unionized substance as a salt; or (ii) the solubility of the unionized substance in a salt comprising the unionized substance in freebase form that is complexing to a non-polar pore of the complexing agent, wherein the unionized substance has the same concentration in the pharmaceutically acceptable salt, the unionized substance as a salt, and the salt comprising the unionized substance in freebase form. In some embodiments, the solubility of the unionized substance in the pharmaceutically acceptable salt is higher than the solubility of the unionized substance in a salt comprising the unionized substance and complexing agent with a higher molar ratio of the unionized substance to the complexing agent, wherein the unionized substance has the same concentration in the pharmaceutically acceptable salt and the salt with a higher molar ratio of the unionized substance to the complexing agent. In some embodiments, the solubility of the unionized substance in the pharmaceutically acceptable salt has an increase of at least 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, or 100-fold compared with (i) the solubility of the unionized substance as a salt; or (ii) the solubility of the unionized substance in a salt comprising the unionized substance in freebase form that is complexing to a non-polar pore of the complexing agent, wherein the unionized substance has the same concentration in the pharmaceutically acceptable salt, the unionized substance as a salt, and the salt comprising the unionized substance in freebase form. In some embodiments, the solubility of the unionized substance in the pharmaceutically acceptable salt has an increase of at least 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, or 100-fold compared with the solubility of the unionized substance in a salt comprising the unionized substance and complexing agent with a higher molar ratio of the unionized substance to the complexing agent, wherein the unionized substance has the same concentration in the pharmaceutically acceptable salt and the salt with a higher molar ratio of the unionized substance to the complexing agent.

In an aspect, provided herein is a pharmaceutically acceptable salt of a compound pharmaceutical comprising: (i) a first pharmaceutical compound, or an enantiomer, a mixture of enantiomers, or an isotopic variant thereof; or a pharmaceutically acceptable salt, solvate or hydrate thereof, wherein the pharmaceutical compound comprises a protonated nitrogen atom; (ii) a conjugate base of a complexing agent comprising a plurality of acidic functional groups, wherein at least one acidic functional group of the plurality of acidic functional groups acts as a counterion of the first pharmaceutical compound; and (iii) a second pharmaceutical compound, or an enantiomer, a mixture of enantiomers, or an isotopic variant thereof; or a pharmaceutically acceptable salt, solvate or hydrate thereof, wherein the second pharmaceutical compound is unionized. In some embodiments, the pharmaceutically acceptable salt has a ratio of the conjugate base of the complexing agent to the first pharmaceutical compound that is from about 1:4 to about 1:10. In some embodiments, the pharmaceutically acceptable salt has a ratio of the conjugate base of the complexing agent to the first pharmaceutical compound that is about 1:4. 1:5, 1:6, 1:7, 1:8, 1:9, or about 1:10, or any ratio therebetween. In some embodiments, the pharmaceutically acceptable salt has a ratio of the conjugate base of the complexing agent to the second pharmaceutical compound that is about 1:1. In some embodiments, the pharmaceutically acceptable salt comprises an additional molar equivalent of the first pharmaceutical compound in unionized form. In some embodiments, the second pharmaceutical compound comprises an unionized form. In some embodiments, the second pharmaceutical compound does not comprise an ionizable nitrogen atom.

In some embodiments, the complexing agent is sulfobutylether-β-cyclodextrin. In some embodiments, the first pharmaceutical compound comprises a dissociative medication compound, a dissociative hallucinogen compound, a dissociative anesthetic compound, an arylcyclo-hexylamine, a 1,2-diarylethylamine, a β-keto-arylcyclohexylamine, or a compound that modulates the NMDA receptor. In some embodiments, the first pharmaceutical compound is ketamine, arylcyclo-hexylamine, 1,2-diarylethylamine, β-keto-arylcyclohexylamine, methoxetamine, deschloroketamine, N-ethyl deschloroketamine (eticyclidone), 3-methoxyphencyclidine, methoxieticyclidine, ephenidine, lanicemine, dextromethorphan, dextrorphan, methoxyketamine, a N,N-dimethyltryptamine, a N,N-diethyltryptamine, a N,N-dipropyltryptamine, a N-Methyl-N-propyltryptamine, a N-methyl-N-isopropyltryptamine, a N,N-diallyltryptamine, a N-methyl-N-allyltryptamine, N-methyl-N-ethyltryptamine, a N,N-Diisopropyltryptamine, 4-hydroxy-N-methyl-N-ethyltryptamine, 5-methoxy-N,N-diisopropyltryptamine, O-acetylpsilocin, methylisopropyllysergamide, ethylisopropyllysergamide, 6-allyl-6-nor-LSD, 6-ethyl-6-nor-lysergic acid diethylamide, 1-acetyl-LSD, 1-propionyl-6-ethyl-6-nor-lysergic acid diethylamide, 1-propionyl-lysergic acid diethylamide, 1-Cyclopropionyl-d-lysergic acid diethylamide, N1-butyryl-lysergic acid diethylamide, 6-propyl-6-nor-Lysergic acid diethylamide, mescaline, 2,5-dimethoxy-4-bromophenethylamine (2C—B), 2-(4-Iodo-2,5-dimethoxyphenyl) ethan-1-amine (2C—I), 2-(4-Chloro-2,5-dimethoxyphenyl) ethan-1-amine (2C—C), 2,5-Dimethoxy-4-iodoamphetamine, 2-[2,5-Dimethoxy-4-(propylsulfanyl)phenyl]ethan-1-amine, 2-(4-iodo-2,5-dimethoxyphenyl)-N-[(2-methoxyphenyl)methyl]ethanamine, racemorphan, levorphanol, racemethorphan, buprenorphine, morphine, loperamide, morphine, codeine, hydrocodone, oxymorphone, buprenorphine, fentanyl, methadone, tramadol, alpha-methyl acetyl fentanyl, alfentanil, butyryl fentanyl, butyrfentanyl, carfentanil, 3-methylcarfentanil, 4-fluorofentanyl, beta-hydroxyfentanyl, alpha-methylfentanyl, cis-3-methylfentanyl, beta-hydroxy-3-methylfentanyl, remifentanil, sufentanil, 3-methylthiofentanyl, naloxone, naltrexone, a cathinone, a 3,4-methylenedioxyamphetamine derivative, an aminoalkyl-substituted benzofuran, a substituted amphetamine, an aminoindane, diphenhydramine, hydroxazine, phenylephrine, dopamine, adrenaline, lidocaine, oxymetazoline, clemastine, chlorpheniramine, or 6-chloro-2-aminotetralin. In some embodiments, the first pharmaceutical compound comprises ketamine.

In some embodiments, the second pharmaceutical compound comprises rapamycin. In some embodiments, the first pharmaceutical compound comprises ketamine and the second pharmaceutical compound comprises rapamycin. In some embodiments, the first pharmaceutical compound comprises no ketamine and the second pharmaceutical compound comprises rapamycin. In some embodiments, the second pharmaceutical compound comprises clonidine. In some embodiments, the first pharmaceutical compound comprises ketamine and the second pharmaceutical compound comprises clonidine. In some embodiments, the first pharmaceutical compound comprises no ketamine and the second pharmaceutical compound comprises clonidine. In some embodiments, the complexing agent comprises a substituted cyclodextrin. In some embodiments, the complexing agent comprises a cyclodextrin substituted with at least one acidic functional group. In some embodiments, the cyclodextrin is substituted with 3 to 8 acidic functional groups. In some embodiments, the cyclodextrin is sulfobutylether-β-cyclodextrin. In some embodiments, the pharmaceutically acceptable salt is formulated for subcutaneous, intramuscular, sublingual, oral, rectal, transvaginal, or intranasal administration. In some embodiments, the complexing agent comprises a non-polar region. In some embodiments, the second pharmaceutical compound is complexed to the complexing agent through the non-polar region.

In some embodiments, the pharmaceutically acceptable salt provided herein consists essentially of the complexing agent and the first and second pharmaceutical compounds. In some embodiments, the pharmaceutically acceptable salt provided herein consists of the complexing agent and the first and second pharmaceutical compounds. In some embodiments, the pharmaceutically acceptable salt provided herein consists of the protonated first pharmaceutical compound and the deprotonated complexing agent.

In some embodiments, the pharmaceutically acceptable salt is in a solid form. In some embodiments, the solid form is a crystalline form or an amorphous form. In some embodiments, the solid form is an amorphous powder. In some embodiments, the solid form is a lyophilized powder. In some embodiments, the solid form is a crystalline form.

In some embodiments, the pharmaceutically acceptable salt provided herein consists essentially of the complexing agent, ketamine and rapamycin. In some embodiments, the pharmaceutically acceptable salt provided herein consists of the complexing agent, ketamine and rapamycin. In some embodiments, the pharmaceutically acceptable salt provided herein consists of the protonated ketamine, unionized rapamycin and the deprotonated complexing agent. In some embodiments, the pharmaceutically acceptable salt provided herein consists essentially of the complexing agent, ketamine and clonidine. In some embodiments, the pharmaceutically acceptable salt provided herein consists of the complexing agent, ketamine and clonidine. In some embodiments, the pharmaceutically acceptable salt provided herein consists of the protonated ketamine, unionized clonidine and the deprotonated complexing agent.

In some embodiments, the pharmaceutically acceptable salt is dissolved or suspended in a liquid medium. In some embodiments, the liquid medium is an aqueous medium, an organic solvent, or a combination thereof. In some embodiments, the liquid medium is an aqueous medium. In some embodiments, the liquid medium is an organic solvent. In some embodiments, the organic solvent comprises acetic acid, acetone, acetonitrile, benzene, tert-butyl alcohol, tert-butyl methyl ether, carbon tetrachloride, chloroform, cyclohexane, 1,2-dichloroethane, dichloromethane, diethyl ether, diglyme, 1,2-dimethoxyethane, dimethyl acetamide, dimethylformamide, dimethyl sulfoxide, dioxane, ethanol, ethyl acetate, ethyl methyl ketone, ethylene glycol, hexanes, hexamethylphosphoramide, methanol, nitromethane, pentanes, 2-propanol, pyridine, tetrahydrofuran, toluene, xylenes, or any combination thereof. In some embodiments, the pharmaceutically acceptable salt is dissolved or suspended in the liquid medium as an intermediate step in its preparation or in the preparation of a pharmaceutical composition comprising the salt.

In some embodiments, the pharmaceutically acceptable salt is substantially free of excess ions. Examples of such ions include other salts that may be left over from the preparation of the salts or byproducts of the production of the salts (e.g. sodium chloride, lithium chloride, potassium chloride, sodium bromide, and the like). In some embodiments, the excess ions are counterions to excess complexing agent or first pharmaceutical compound in the salt preparation, such as excess sodium ions occupying the deprotonated acidic sites or chloride ions associated with excess protonated pharmaceutical compound.

The presence of unionized second pharmaceutical compound can have numerous benefits in certain contexts, including increasing the dose per unit weight or volume of the salt when the salt is used in a pharmaceutical composition. Additionally, when used in a pharmaceutical composition, the presence of free base or unionized pharmaceutical compound can be used to raise the pH of the composition as it is administered, thus potentially facilitating both bioavailability and tolerability in certain contexts (e.g. when the pKa of the pharmaceutical compound is lower than the pH at which the compound can be comfortably administered to the target tissue). Additionally, many of the complexing agents contemplated herein have an additional coordination site for unionized APIs (e.g. the middle complexing site of a cyclodextrin). Thus, in some embodiments, the complexing agents used herein can offer additional solubilization of pharmaceutical compounds beyond that accomplished merely by acid/base chemistry and ion exchange/ion pairing.

In some embodiments, the pharmaceutically acceptable salt comprises additional equivalents of the first or second pharmaceutical compound. In some embodiments, the additional equivalents are measured as compared to the moles of complexing agent. In some embodiments, the pharmaceutically acceptable salt comprises additional molar equivalents of the deprotonated first pharmaceutical compound. In some embodiments, the additional molar equivalents are measured as compared to the moles of complexing agent. In some embodiments, the additional equivalents are measured as compared to the moles of the pharmaceutical compound which forms a salt with the complexing agent (e.g. the protonated pharmaceutical compound).

In some embodiments, the pharmaceutically acceptable salt comprises molar equivalents of unionized second pharmaceutical compound compared to the protonated pharmaceutical compound of the complexing agent/protonated pharmaceutical compound salt. In some embodiments, the pharmaceutically acceptable salt comprises about 0.01 molar equivalents to about 10 molar equivalents of the unionized second pharmaceutical compound. In some embodiments, the pharmaceutically acceptable salt comprises about 0.1 molar equivalents to about 1 molar equivalents of the unionized second pharmaceutical compound. In some embodiments, the pharmaceutically acceptable salt comprises about 0.1 molar equivalents to about 10 molar equivalents of unionized second pharmaceutical compound. In some embodiments, the pharmaceutically acceptable salt comprises about 0.1 molar equivalents to about 0.2 molar equivalents, about 0.1 molar equivalents to about 0.5 molar equivalents, about 0.1 molar equivalents to about 0.75 molar equivalents, about 0.1 molar equivalents to about 1 molar equivalents, about 0.2 molar equivalents to about 0.5 molar equivalents, about 0.2 molar equivalents to about 0.75 molar equivalents, about 0.2 molar equivalents to about 1 molar equivalents, about 0.5 molar equivalents to about 0.75 molar equivalents, about 0.5 molar equivalents to about 1 molar equivalents, or about 0.75 molar equivalents to about 1 molar equivalents. In some embodiments, the pharmaceutically acceptable salt comprises about 0.1 molar equivalents, about 0.2 molar equivalents, about 0.5 molar equivalents, about 0.75 molar equivalents, or about 1 molar equivalents of the unionized second pharmaceutical compound. In some embodiments, the pharmaceutically acceptable salt comprises at least about 0.1 molar equivalents, about 0.2 molar equivalents, about 0.5 molar equivalents, or about 0.75 molar equivalents of the unionized second pharmaceutical compound. In some embodiments, the pharmaceutically acceptable salt comprises at most about 0.2 molar equivalents, about 0.5 molar equivalents, about 0.75 molar equivalents, or about 1 molar equivalents of the unionized second pharmaceutical compound. In some embodiments, the pharmaceutically acceptable salt comprises about 0.5 equivalents to about 5 equivalents of the unionized second pharmaceutical compound. In some embodiments, the pharmaceutically acceptable salt comprises about 0.5 equivalents to about 1 equivalents, about 0.5 equivalents to about 2 equivalents, about 0.5 equivalents to about 3 equivalents, about 0.5 equivalents to about 4 equivalents, about 0.5 equivalents to about 5 equivalents, about 1 equivalents to about 2 equivalents, about 1 equivalents to about 3 equivalents, about 1 equivalents to about 4 equivalents, about 1 equivalents to about 5 equivalents, about 2 equivalents to about 3 equivalents, about 2 equivalents to about 4 equivalents, about 2 equivalents to about 5 equivalents, about 3 equivalents to about 4 equivalents, about 3 equivalents to about 5 equivalents, or about 4 equivalents to about 5 equivalents of the unionized second pharmaceutical compound. In some embodiments, the pharmaceutically acceptable salt comprises about 0.5 equivalents, about 1 equivalents, about 2 equivalents, about 3 equivalents, about 4 equivalents, or about 5 equivalents of the unionized second pharmaceutical compound. In some embodiments, the pharmaceutically acceptable salt comprises at least about 0.5 equivalents, about 1 equivalents, about 2 equivalents, about 3 equivalents, or about 4 equivalents of the unionized second pharmaceutical compound. In some embodiments, the pharmaceutically acceptable salt comprises at most about 1 equivalents, about 2 equivalents, about 3 equivalents, about 4 equivalents, or about 5 equivalents of the unionized second pharmaceutical compound.

In some embodiments, the pharmaceutically acceptable salt comprises molar equivalents of unionized second pharmaceutical compound compared to complexing agent of the complexing agent/protonated pharmaceutical compound salt. In some embodiments, the pharmaceutically acceptable salt comprises about 0.01 molar equivalents to about 10 molar equivalents of unionized second pharmaceutical compound. In some embodiments, the pharmaceutically acceptable salt comprises about 0.1 molar equivalents to about 10 molar equivalents of unionized second pharmaceutical compound. In some embodiments, the pharmaceutically acceptable salt comprises about 0.1 molar equivalents to about 0.5 molar equivalents, about 0.1 molar equivalents to about 1 molar equivalents, about 0.1 molar equivalents to about 2 molar equivalents, about 0.1 molar equivalents to about 3 molar equivalents, about 0.1 molar equivalents to about 5 molar equivalents, about 0.1 molar equivalents to about 7 molar equivalents, about 0.1 molar equivalents to about 10 molar equivalents, about 0.5 molar equivalents to about 1 molar equivalents, about 0.5 molar equivalents to about 2 molar equivalents, about 0.5 molar equivalents to about 3 molar equivalents, about 0.5 molar equivalents to about 5 molar equivalents, about 0.5 molar equivalents to about 7 molar equivalents, about 0.5 molar equivalents to about 10 molar equivalents, about 1 molar equivalents to about 2 molar equivalents, about 1 molar equivalents to about 3 molar equivalents, about 1 molar equivalents to about 5 molar equivalents, about 1 molar equivalents to about 7 molar equivalents, about 1 molar equivalents to about 10 molar equivalents, about 2 molar equivalents to about 3 molar equivalents, about 2 molar equivalents to about 5 molar equivalents, about 2 molar equivalents to about 7 molar equivalents, about 2 molar equivalents to about 10 molar equivalents, about 3 molar equivalents to about 5 molar equivalents, about 3 molar equivalents to about 7 molar equivalents, about 3 molar equivalents to about 10 molar equivalents, about 5 molar equivalents to about 7 molar equivalents, about 5 molar equivalents to about 10 molar equivalents, or about 7 molar equivalents to about 10 molar equivalents of unionized pharmaceutical compound of unionized second pharmaceutical compound. In some embodiments, the pharmaceutically acceptable salt comprises about 0.1 molar equivalents, about 0.5 molar equivalents, about 1 molar equivalents, about 2 molar equivalents, about 3 molar equivalents, about 5 molar equivalents, about 7 molar equivalents, or about 10 molar equivalents of unionized second pharmaceutical compound. In some embodiments, the pharmaceutically acceptable salt comprises at least about 0.1 molar equivalents, about 0.5 molar equivalents, about 1 molar equivalents, about 2 molar equivalents, about 3 molar equivalents, about 5 molar equivalents, or about 7 molar equivalents of unionized second pharmaceutical compound. In some embodiments, the pharmaceutically acceptable salt comprises at most about 0.5 molar equivalents, about 1 molar equivalents, about 2 molar equivalents, about 3 molar equivalents, about 5 molar equivalents, about 7 molar equivalents, or about 10 molar equivalents of unionized second pharmaceutical compound. In some embodiments, the pharmaceutically acceptable salt comprises about 0.01 molar equivalents to about 20 molar equivalents of unionized second pharmaceutical compound compared to the complexing agent. In some embodiments, the pharmaceutically acceptable salt comprises about 0.1 molar equivalents to about 20 molar equivalents of unionized second pharmaceutical compound compared to the complexing agent. In some embodiments, the pharmaceutical composition comprises about 1 molar equivalent to about 20 molar equivalents of unionized second pharmaceutical compound.

In some embodiments, the pharmaceutically acceptable salt comprises additional molar equivalents of unionized first pharmaceutical compound compared to the protonated pharmaceutical compound of the complexing agent/protonated pharmaceutical compound salt. In some embodiments, the unionized first pharmaceutical compound is complexed to the non-polar region or non-polar pore of the complexing agent. In some embodiments, the pharmaceutically acceptable salt comprises about 0.01 molar equivalents to about 10 molar equivalents of the unionized first pharmaceutical compound. In some embodiments, the pharmaceutically acceptable salt comprises about 0.1 molar equivalents to about 1 molar equivalents of the unionized first pharmaceutical compound. In some embodiments, the pharmaceutically acceptable salt comprises about 0.1 molar equivalents to about 10 molar equivalents of unionized first pharmaceutical compound. In some embodiments, the pharmaceutically acceptable salt comprises about 0.1 molar equivalents to about 0.2 molar equivalents, about 0.1 molar equivalents to about 0.5 molar equivalents, about 0.1 molar equivalents to about 0.75 molar equivalents, about 0.1 molar equivalents to about 1 molar equivalents, about 0.2 molar equivalents to about 0.5 molar equivalents, about 0.2 molar equivalents to about 0.75 molar equivalents, about 0.2 molar equivalents to about 1 molar equivalents, about 0.5 molar equivalents to about 0.75 molar equivalents, about 0.5 molar equivalents to about 1 molar equivalents, or about 0.75 molar equivalents to about 1 molar equivalents. In some embodiments, the pharmaceutically acceptable salt comprises about 0.1 molar equivalents, about 0.2 molar equivalents, about 0.5 molar equivalents, about 0.75 molar equivalents, or about 1 molar equivalents of the unionized first pharmaceutical compound. In some embodiments, the pharmaceutically acceptable salt comprises at least about 0.1 molar equivalents, about 0.2 molar equivalents, about 0.5 molar equivalents, or about 0.75 molar equivalents of the unionized first pharmaceutical compound. In some embodiments, the pharmaceutically acceptable salt comprises at most about 0.2 molar equivalents, about 0.5 molar equivalents, about 0.75 molar equivalents, or about 1 molar equivalents of the unionized first pharmaceutical compound. In some embodiments, the pharmaceutically acceptable salt comprises about 0.5 equivalents to about 5 equivalents of the unionized first pharmaceutical compound. In some embodiments, the pharmaceutically acceptable salt comprises about 0.5 equivalents to about 1 equivalents, about 0.5 equivalents to about 2 equivalents, about 0.5 equivalents to about 3 equivalents, about 0.5 equivalents to about 4 equivalents, about 0.5 equivalents to about 5 equivalents, about 1 equivalents to about 2 equivalents, about 1 equivalents to about 3 equivalents, about 1 equivalents to about 4 equivalents, about 1 equivalents to about 5 equivalents, about 2 equivalents to about 3 equivalents, about 2 equivalents to about 4 equivalents, about 2 equivalents to about 5 equivalents, about 3 equivalents to about 4 equivalents, about 3 equivalents to about 5 equivalents, or about 4 equivalents to about 5 equivalents of the unionized first pharmaceutical compound. In some embodiments, the pharmaceutically acceptable salt comprises about 0.5 equivalents, about 1 equivalent, about 2 equivalents, about 3 equivalents, about 4 equivalents, or about 5 equivalents of the unionized first pharmaceutical compound. In some embodiments, the pharmaceutically acceptable salt comprises at least about 0.5 equivalents, about 1 equivalent, about 2 equivalents, about 3 equivalents, or about 4 equivalents of the unionized first pharmaceutical compound. In some embodiments, the pharmaceutically acceptable salt comprises at most about 1 equivalent, about 2 equivalents, about 3 equivalents, about 4 equivalents, or about 5 equivalents of the unionized first pharmaceutical compound.

In some embodiments, the pharmaceutically acceptable salt comprises molar equivalents of unionized first pharmaceutical compound compared to complexing agent of the complexing agent/protonated pharmaceutical compound salt. In some embodiments, the pharmaceutically acceptable salt comprises about 0.01 molar equivalents to about 10 molar equivalents of unionized first pharmaceutical compound. In some embodiments, the pharmaceutically acceptable salt comprises about 0.1 molar equivalents to about 10 molar equivalents of unionized first pharmaceutical compound. In some embodiments, the pharmaceutically acceptable salt comprises about 0.1 molar equivalents to about 0.5 molar equivalents, about 0.1 molar equivalents to about 1 molar equivalents, about 0.1 molar equivalents to about 2 molar equivalents, about 0.1 molar equivalents to about 3 molar equivalents, about 0.1 molar equivalents to about 5 molar equivalents, about 0.1 molar equivalents to about 7 molar equivalents, about 0.1 molar equivalents to about 10 molar equivalents, about 0.5 molar equivalents to about 1 molar equivalents, about 0.5 molar equivalents to about 2 molar equivalents, about 0.5 molar equivalents to about 3 molar equivalents, about 0.5 molar equivalents to about 5 molar equivalents, about 0.5 molar equivalents to about 7 molar equivalents, about 0.5 molar equivalents to about 10 molar equivalents, about 1 molar equivalents to about 2 molar equivalents, about 1 molar equivalents to about 3 molar equivalents, about 1 molar equivalents to about 5 molar equivalents, about 1 molar equivalents to about 7 molar equivalents, about 1 molar equivalents to about 10 molar equivalents, about 2 molar equivalents to about 3 molar equivalents, about 2 molar equivalents to about 5 molar equivalents, about 2 molar equivalents to about 7 molar equivalents, about 2 molar equivalents to about 10 molar equivalents, about 3 molar equivalents to about 5 molar equivalents, about 3 molar equivalents to about 7 molar equivalents, about 3 molar equivalents to about 10 molar equivalents, about 5 molar equivalents to about 7 molar equivalents, about 5 molar equivalents to about 10 molar equivalents, or about 7 molar equivalents to about 10 molar equivalents of unionized pharmaceutical compound of unionized first pharmaceutical compound. In some embodiments, the pharmaceutically acceptable salt comprises about 0.1 molar equivalents, about 0.5 molar equivalents, about 1 molar equivalents, about 2 molar equivalents, about 3 molar equivalents, about 5 molar equivalents, about 7 molar equivalents, or about 10 molar equivalents of unionized first pharmaceutical compound. In some embodiments, the pharmaceutically acceptable salt comprises at least about 0.1 molar equivalents, about 0.5 molar equivalents, about 1 molar equivalents, about 2 molar equivalents, about 3 molar equivalents, about 5 molar equivalents, or about 7 molar equivalents of unionized first pharmaceutical compound. In some embodiments, the pharmaceutically acceptable salt comprises at most about 0.5 molar equivalents, about 1 molar equivalents, about 2 molar equivalents, about 3 molar equivalents, about 5 molar equivalents, about 7 molar equivalents, or about 10 molar equivalents of unionized first pharmaceutical compound. In some embodiments, the pharmaceutically acceptable salt comprises about 0.01 molar equivalents to about 20 molar equivalents of unionized first pharmaceutical compound compared to the complexing agent. In some embodiments, the pharmaceutically acceptable salt comprises about 0.1 molar equivalents to about 20 molar equivalents of unionized first pharmaceutical compound compared to the complexing agent. In some embodiments, the pharmaceutical composition comprises about 1 molar equivalent to about 20 molar equivalents of unionized first pharmaceutical compound.

In some embodiments, the solubility of the second pharmaceutical compound in the pharmaceutically acceptable salt is higher than the solubility of the second pharmaceutical compound as a salt, wherein the second pharmaceutical compound has the same concentration in the pharmaceutically acceptable salt, and the second pharmaceutical compound as a salt. In some embodiments, the solubility of the second pharmaceutical compound in the pharmaceutically acceptable salt has an increase of at least 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, or 100-fold compared with the solubility of the second pharmaceutical compound as a salt, wherein the second pharmaceutical compound has the same concentration in the pharmaceutically acceptable salt, and the second pharmaceutical compound as a salt.

In some embodiments, the first pharmaceutical compound modulates the NMDA receptor. In some embodiments, the first pharmaceutical compound is an NMDA receptor antagonist, an NMDA receptor agonist, a mixed NMDA receptor agonist-antagonist, or an NMDA receptor reverse agonist. In some embodiments, the first pharmaceutical compound modulates the NMDA receptor at the polyamine site, the glycine binding site, the glutamate binding site, the PCP binding site, the ketamine binding site, an allosteric modulation site, the zinc binding site, or the magnesium binding site. In some embodiments, the first pharmaceutical compound is not ketamine.

In some embodiments, the first pharmaceutical compound does not modulate the NMDA receptor. In some embodiments, the first pharmaceutical compound is not an NMDA receptor agonist. In some embodiments, the first pharmaceutical compound is not ketamine.

In some embodiments, the first pharmaceutical compound is methoxetamine, deschloroketamine, N-ethyl deschloroketamine (eticyclidone), 3-methoxyphencyclidine, methoxieticyclidine, ephenidine, lanicemine, dextromethorphan, dextrorphan, or methoxyketamine. In some embodiments, the first pharmaceutical compound is methoxetamine or deschloroketamine. In some embodiments, the first pharmaceutical compound is methoxetamine. In some embodiments, the first pharmaceutical compound is deschloroketamine.

In some embodiments, the first pharmaceutical compound is ketamine, methoxetamine, deschloroketamine, N-ethyl deschloroketamine (eticyclidone), 3-methoxyphencyclidine, methoxieticyclidine, ephenidine, lanicemine, dextromethorphan, dextrorphan, or methoxyketamine. In some embodiments, the first pharmaceutical compound is methoxetamine or deschloroketamine. In some embodiments, the first pharmaceutical compound is ketamine. In some embodiments, the ketamine is racemic ketamine. In some embodiments, the ketamine is stereopure or stereoenhanced ketamine. In some embodiments, the ketamine is (R)-ketamine. In some embodiments, the ketamine is(S)-ketamine.

In some embodiments, the first pharmaceutical compound is tryptamine, phenethylamine, or a lysergamide compound. In some embodiments, the first pharmaceutical compound is a tryptamine, a phenethylamine, or a lysergamide. In some embodiments, the first pharmaceutical compound is an N,N-Dimethyltryptamine, a N,N-diethyltryptamine, a N,N-dipropyltryptamine, a N-Methyl-N-propyltryptamine, a N-methyl-N-isopropyltryptamine, a N,N-diallyltryptamine, a N-methyl-N-allyltryptamine, N-methyl-N-ethyltryptamine, a N,N-Diisopropyltryptamine, an α-ethyltryptamine, or a lysergamide.

In some embodiments, the first pharmaceutical compound is an N,N-dialkyltryptamine. In some embodiments, the pharmaceutical compound is tryptamine selected from an N,N-Dimethyltryptamine, a N,N-diethyltryptamine, a N,N-dipropyltryptamine, a N-Methyl-N-propyltryptamine, a N-methyl-N-isopropyltryptamine, a N,N-diallyltryptamine, a N-methyl-N-allyltryptamine, N-methyl-N-ethyltryptamine, a N,N-Diisopropyltryptamine, wherein the tryptamine is optionally substituted. In some embodiments, the tryptamine is optionally substituted on the tryptamine ring. In some embodiments, the tryptamine is optionally substituted on the tryptamine ring with a substituent selected from hydroxy, acetoxy, alkoxy, halogen, or alkyl. In some embodiments, the tryptamine is optionally substituted at the 4- or 5-position of the tryptamine ring. In some embodiments, the tryptamine is optionally substituted at the 4- or 5-position of the tryptamine ring with a substituent selected from hydroxy, acetoxy, or methoxy.

In some embodiments, the first pharmaceutical compound is a N-methyl-N-ethyltryptamine. In some embodiments, the first pharmaceutical compound is an N,N-dimethyltryptamine. In some embodiments, the first pharmaceutical compound is psilocin, O-acetylpsilocin, or 5-methoxy-N,N-dimethyltryptamine.

In some embodiments, the first pharmaceutical compound is a N,N-diethyltryptamine. In some embodiments, the pharmaceutical compound is N,N-diethyltryptamine, 4-hydroxy-diethyl-tryptamine, 4-acetoxy-N,N-diethyltryptamine, or 5-methoxy-N,N-diethyltryptamine.

In some embodiments, the first pharmaceutical compound is a N,N-dipropyltryptamine. In some embodiments, the first pharmaceutical compound is dipropyltryptamine, 4-hydroxy-dipropyltryptamine, 4-acetoxy-N,N-dipropyltryptamine, or 5-methoxy-N,N-dipropyltryptamine.

In some embodiments, the first pharmaceutical compound is a N-methyl-N-propyltryptamine. In some embodiments, the first pharmaceutical compound is N-methyl-N-propyltryptamine, 4-hydroxy-N-methyl-N-propyltryptamine, 4-acetoxy-N-methyl-N-propyltryptamine, or 5-methoxy-N-methyl-N-propyltryptamine.

In some embodiments, the first pharmaceutical compound is a N-methyl-N-ethyltryptamine. In some embodiments, the first pharmaceutical compound is N-methyl-N-ethyltryptamine, 4-hydroxy-N-methyl-N-ethyltryptamine, 4-acetoxy-N-methyl-N-ethyltryptamine, or 5 methoxy-N-methyl-N-ethyltryptamine.

In some embodiments, the first pharmaceutical compound is a N-methyl-N-isopropyltryptamine. In some embodiments, the first pharmaceutical compound is N-methyl-N-isopropyltryptamine, 4-hydroxy-N-methyl-N-isopropyltryptamine, 4-acetoxy-N-methyl-N-isopropyltryptamine, or 5 methoxy-N-methyl-N-isopropyltryptamine.

In some embodiments, the first pharmaceutical compound is a N,N-diallyltryptamine. In some embodiments, the first pharmaceutical compound is N,N-diallyltryptamine, 4-hydroxy-N,N-diallyltryptamine, 4-acetoxy-N,N-diallyltryptamine, or 5-methoxy-N,N-diallyltryptamine.

In some embodiments, the first pharmaceutical compound is a N-methyl-N-allyl-tryptamine. In some embodiments, the first pharmaceutical compound is N-methyl-N-allyl-tryptamine, 4-hydroxy-N-methyl-N-allyl-tryptamine, 4-acetoxy-N-methyl-N-allyl-tryptamine, or 5-methoxy-N-methyl-N-allyl-tryptamine.

In some embodiments, the first pharmaceutical compound is a N,N-diisopropyltryptamine. In some embodiments, the first pharmaceutical compound is N,N-diisopropyltryptamine, 4-hydroxy-N,N-diisopropyltryptamine, 4-acetoxy-N,N-diisopropyltryptamine, or 5-methoxy-N,N-diisopropyltryptamine. In some embodiments, the pharmaceutical compound is an α-ethyltryptamine.

In some embodiments, the first pharmaceutical compound is a lysergamide. In some embodiments, the first pharmaceutical compound is methylisopropyllysergamide, ethylisopropyllysergamide, 6-allyl-6-nor-LSD, 6-ethyl-6-nor-lysergic acid diethylamide, 1-acetyl-LSD, 1-propionyl-6-ethyl-6-nor-lysergic acid diethylamide, 1-propionyl-lysergic acid diethylamide, 1-Cyclopropionyl-d-lysergic acid diethylamide, N1-butyryl-lysergic acid diethylamide, or 6-propyl-6-nor-Lysergic acid diethylamide.

In some embodiments, the first pharmaceutical compound is a phenethylamine. In some embodiments, the first pharmaceutical compound is mescaline, 2,5-dimethoxy-4-bromophenethylamine (2C—B), 2-(4-Iodo-2,5-dimethoxyphenyl) ethan-1-amine (2C—I), 2-(4-Chloro-2,5-dimethoxyphenyl) ethan-1-amine (2C—C), 2,5-Dimethoxy-4-iodoamphetamine, or 2-(4-iodo-2,5-dimethoxyphenyl)-N-[(2-methoxyphenyl)methyl]ethanamine. In some embodiments, the first pharmaceutical compound is mescaline. In some embodiments, the first pharmaceutical compound is 2,5-dimethoxy-4-bromophenethylamine (2C—B). In some embodiments, the first pharmaceutical compound is 2-(4-Iodo-2,5-dimethoxyphenyl) ethan-1-amine (2C—I).). In some embodiments, the first pharmaceutical compound is 2-[2,5-Dimethoxy-4-(propylsulfanyl)phenyl]ethan-1-amine.

In some embodiments, the first pharmaceutical compound is an opioid. In some embodiments, the opioid is a naturally occurring opioid. In some embodiments, the opioid is a synthetic opioid. In some embodiments, the opioid is an opioid derivative. In some embodiments, the opioid is a morphine derivative. In some embodiments, the opioid is a fentanyl derivative. In some embodiments, the opioid is a semi-synthetic opioid.

In some embodiments, the opioid is racemorphan, levorphanol, racemethorphan, buprenorphine, morphine, loperamide, codeine, hydrocodone, oxymorphone, buprenorphine, fentanyl, methadone, tramadol, alpha-methyl acetyl fentanyl, alfentanil, butyryl fentanyl, butyrfentanyl, carfentanil, 3-methylcarfentanil, 4-fluorofentanyl, beta-hydroxyfentanyl, alpha-methylfentanyl, cis-3-methylfentanyl, beta-hydroxy-3-methylfentanyl, remifentanil, sufentanil, or 3-methylthiofentanyl. In some embodiments, the opioid is racemorphan, levorphanol, or racemethorphan. In some embodiments, the opioid is racemorphan. In some embodiments, the opioid is levorphanol. In some embodiments, the opioid is racemethorphan.

In some embodiments, the opioid has a pKa from about 7 to about 11. In some embodiments, the opioid is morphine, codeine, hydrocodone, oxymorphone, buprenorphine, fentanyl, methadone, tramadol, alpha-methyl acetyl fentanyl, alfentanil, butyryl fentanyl, butyrfentanyl, carfentanil, 3-methylcarfentanil, 4-fluorofentanyl, beta-hydroxyfentanyl, alpha-methylfentanyl, cis-3-methylfentanyl, beta-hydroxy-3-methylfentanyl, remifentanil, sufentanil, or 3-methylthiofentanyl.

In some embodiments, the opioid is an opioid receptor antagonist. In some embodiments, the opioid receptor antagonist is naloxone or naltrexone. In some embodiments, the opioid receptor antagonist does not include naloxone.

In some embodiments, the first pharmaceutical compound is a 3-substituted methcathinone a 3-substituted ethcathinone, a 4-substituted methcathinone, a 4-substituted ethcathinone, methylone, ethylone, or butylone.

In some embodiments, the first pharmaceutical compound is a 3-substituted methcathinone. In some embodiments, the first pharmaceutical compound is 3-methylmethcathinone, 3-ethylmethcathinone, 3-fluoromethcathinone, 3-chloromethcathinone, or 3-bromomethcathinone. In some embodiments, the first pharmaceutical compound is 3-methylmethcathinone.

In some embodiments, the first pharmaceutical compound is a 3-substituted ethcathinone. In some embodiments, the first pharmaceutical compound is 3-methylethcathinone, 3-ethylethcathinone, 3-fluoroethcathinone, 3-chloroethcathinone, or 3-bromoethcathinone.

In some embodiments, the first pharmaceutical compound is a 4-substituted methcathinone. In some embodiments, the first pharmaceutical compound is 4-methyl methcathinone, 4-ethyl methcathinone, 4-fluoromethcathinone, 4-chloromethcathinone, or 4-bromomethcathinone.

In some embodiments, the first pharmaceutical compound is a 4-substituted ethcathinone. In some embodiments, the first pharmaceutical compound is 4-methylethcathinone, 4-ethylethcathinone, 4-fluoroethcathinone, 4-chloroethcathinone, or 4-bromoethcathinone.

In some embodiments, the first pharmaceutical compound is methylone, ethylone, or butylone. In some embodiments, the first pharmaceutical compound is methylone. In some embodiments, the first pharmaceutical compound is ethylone. In some embodiments, the first pharmaceutical compound is butylone.

In some embodiments, the first pharmaceutical compound is a 3,4-methylenedioxyamphetamine derivative. In some embodiments, the first pharmaceutical compound is 1-(1,3-benzodioxol-5-yl)-2-butanamine, 1-(1,3-benzodioxol-5-yl)-N-methyl-2-butanamine, 1-(1,3-benzodioxol-5-yl)-N-ethyl-2-butanamine, 3,4-methylenedioxyamphetamine, 3,4-methylene-dioxy-N-ethyl-amphetamine, S-3,4-methylene-dioxy-N-ethyl-amphetamine, 3,4-methylenedioxy-N-methylamphetamine, 5,6-methylenedioxy-2-aminoindane, 2-amino-(3,4-methylenedioxy) propiophenone, or methylenedioxypyrovalerone.

In some embodiments, the first pharmaceutical compound is an aminoalkyl-substituted benzofuran. In some embodiments, the first pharmaceutical compound is 5-(2-aminopropyl)-benzofuran, 2,3-dihydro isomer of 5-APB, 1-(benzofuran-5-yl)-N-methylpropan-2-amine, 6-(2-aminopropyl)-benzofuran, 2,3-dihydro isomer of 6-APB, or 1-(benzofuran-6-yl)-N-methylpropan-2-amine.

In some embodiments, the first pharmaceutical compound is a substituted amphetamine. In some embodiments, the pharmaceutical compound is 4-fluoroamphetamine, 4-fluoromethamphetamine, 3-fluoroamphetamine, 3-fluoromethamphetamine, 2-fluoroamphetamine, or 2-fluoromethamphetamine.

In some embodiments, the first pharmaceutical compound is an aminoindane. In some embodiments, the first pharmaceutical compound is 5-iodo-2-aminoindane, 5,6-methylenedioxy-2-aminoindane, 5-methoxy-2-aminoindane (MEAI), N-acetyl-MEAI, 5-hydroxy-N-acetyl-AI, or 5-Methoxy-6-methyl-2-aminoindane.

In some embodiments, the first pharmaceutical compound is a stimulant. In some embodiments, the first pharmaceutical compound is ephedrine, pseudoephedrine, amphetamine, (R)-1-Phenyl-N-propylpentan-2-amine, benzofuranylpropylaminopentane, or methylphenidate.

In some embodiments, the first pharmaceutical compound is diphenhydramine, hydroxazine, phenylephrine, dopamine, adrenaline, lidocaine, oxymetazoline, clemastine, chlorpheniramine, or 6-chloro-2-aminotetralin. In some embodiments, the first pharmaceutical compound is diphenhydramine.

### Pharmaceutical Compositions

Provided herein are pharmaceutical formulations of pharmaceutical compounds suitable for dosing or administration by a variety of routes, including subcutaneous injection, intranasal administration, and/or sublingual administration. These pharmaceutical compositions utilize the complexing agent/pharmaceutical compound salts provided herein. Such salts provide numerous advantages in each type of pharmaceutical composition, some of which are unique to the route of administration.

In an aspect provided herein is a pharmaceutical composition, comprising: (i) an pharmaceutical compound, or an enantiomer, a mixture of enantiomers, or an isotopic variant thereof, wherein the pharmaceutical compound comprises a protonated nitrogen atom; and (ii) a complexing agent, wherein the complexing agent is an acid-substituted cyclodextrin comprising a plurality of acidic functional groups, wherein the plurality of acidic functional groups comprises an acidic group which acts as a counterion for the protonated nitrogen atom of the pharmaceutical compound, wherein the pharmaceutical composition has a ratio of the complexing agent to the pharmaceutical compound that is from about 1:1 to about 1:4.

In some embodiments, the pharmaceutical compound is an antiviral compound, an antibacterial compound, an anti-fungal compound, a compound for treatment of a neurological disorder, a compound for treatment of Parkinson's disease, a treatment for migraine headache, a treatment for autoimmune disease, a treatment for cancer, a treatment for lymphoma, a treatment for pancreatitis, a treatment for opioid overdose, a treatment for flu infection, or a treatment for an inflammatory disorder. In some embodiments, the pharmaceutical compound comprises rotigotine, eletriptan, copanlisib, remdesivir, nafamostat (nafamostat mesylate), melevodopa, tigecycline, naloxone, amikacin, 1-amantadine, 2-amantadine, rimantadine, amifampridine, rapamycin, clonidine, or caspofungin.

In some embodiments, the ratio of the complexing agent to the pharmaceutical compound is from about 1:1 to about 1:4. In some embodiments, the ratio of the complexing agent to the pharmaceutical compound is from about 1:1.1 to about 1:3.9. In some embodiments, the ratio of the complexing agent to the pharmaceutical compound is from about 1:1.2 to about 1:3.8. In some embodiments, the ratio of the complexing agent to the pharmaceutical compound is from about 1:1.3 to about 1:3.7. In some embodiments, the ratio of the complexing agent to the pharmaceutical compound is from about 1:1.4 to about 1:3.6. In some embodiments, the ratio of the complexing agent to the pharmaceutical compound is from about 1:1.5 to about 1:3.5. In some embodiments, the ratio of the complexing agent to the pharmaceutical compound is from about 1:1.6 to about 1:3.4. In some embodiments, the ratio of the complexing agent to the pharmaceutical compound is from about 1:1.7 to about 1:3.3. In some embodiments, the ratio of the complexing agent to the pharmaceutical compound is from about 1:1.8 to about 1:3.2. In some embodiments, the ratio of the complexing agent to the pharmaceutical compound is from about 1:1.9 to about 1:3.1. In some embodiments, the ratio of the complexing agent to the pharmaceutical compound is from about 1:2 to about 1:3. In some embodiments, the ratio of the complexing agent to the pharmaceutical compound is from about 1:2.1 to about 1:2.9. In some embodiments, the ratio of the complexing agent to the pharmaceutical compound is from about 1:2.2 to about 1:2.8. In some embodiments, the ratio of the complexing agent to the pharmaceutical compound is from about 1:2.3 to about 1:2.7. In some embodiments, the ratio of the complexing agent to the pharmaceutical compound is from about 1:2.4 to about 1:2.6. In some embodiments, the ratio of the complexing agent to the pharmaceutical compound is from about 1:1 to about 1:4, about 1:1.1, 1:1.2, 1:1.3, 1:1.4, 1:1.5, 1:1.6, 1:1.7, 1:1.8, 1:1.9, 1:2.0, 1:2.1, 1:2.2, 1:2.3, 1:2.4, 1:2.5, 1:2.6, 1:2.7, 1:2.8, 1:2.9, 1:3.0, 1:3.1, 1:3.2, 1:3.3, 1:3.4, 1:3.5, 1:3.6, 1:3.7, 1:3.8, 1:3.9, or about 1:4, or any ratio therebetween.

In some embodiments, the complexing agent acts as the counterion to between 1 to 4 molecules of the pharmaceutical compound, e.g., about 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or about 4 molecules of the pharmaceutical compound, or any number of molecules of the pharmaceutical compound therebetween. In some embodiments, the total number of acidic functional groups of the complexing agent in the pharmaceutical composition is more than the total number of protonated nitrogen atoms of the pharmaceutical compound in the pharmaceutical composition. In some embodiments, a portion, but not all, of the acidic functional groups of the complexing agent neutralize all of the protonated nitrogen atoms of the pharmaceutical compound.

In some embodiments, fewer than 5 acidic functional groups of the complexing agent neutralize all of the protonated nitrogen atoms of the pharmaceutical compound. In some embodiments, fewer than 4 acidic functional groups of the complexing agent neutralize all of the protonated nitrogen atoms of the pharmaceutical compound. In some embodiments, fewer than 3 acidic functional groups of the complexing agent neutralize all of the protonated nitrogen atoms of the pharmaceutical compound. In some embodiments, fewer than 2 acidic functional groups of the complexing agent neutralize all of the protonated nitrogen atoms of the pharmaceutical compound. In some embodiments, fewer than 1 acidic functional groups of the complexing agent neutralize all of the protonated nitrogen atoms of the pharmaceutical compound.

In some embodiments, all of the protonated nitrogen atoms of the pharmaceutical compound are neutralized by 1 to 4 acidic functional groups. In some embodiments, all of the protonated nitrogen atoms of the pharmaceutical compound are neutralized by 1 to 3 acidic functional groups. In some embodiments, all of the protonated nitrogen atoms of the pharmaceutical compound are neutralized by 1 to 2 acidic functional groups. In some embodiments, all of the protonated nitrogen atoms of the pharmaceutical compound are neutralized by 1-4 acidic functional groups, e.g., about 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or about 4.0 acidic functional groups, or any number therebetween.

In some embodiments, the pharmaceutical composition comprises excess ions. In some embodiments, the excess ions comprise a salt, e.g., sodium chloride, lithium chloride, potassium chloride, or sodium bromide. In some embodiments, an excess ion acts as a counterion to an acidic functional group of the complexing agent. In some embodiments, such as an excess sodium ion occupying a deprotonated acidic site.

In some embodiments, the complexing agent further comprises a non-polar region. In some embodiments, the non-polar region is a non-polar pore. In some embodiments, the pharmaceutical compound in the pharmaceutical composition is in an unionized form. In some embodiments, the pharmaceutical compound in the unionized form is complexed to the non-polar region of the complexing agent. In some embodiments, the pharmaceutical compound in the unionized form is complexed to the non-polar pore of the complexing agent.

In some embodiments, the pharmaceutical composition comprises 0.1-20 molar equivalents of the pharmaceutical compound in an unionized form compared to the complexing agent. In some embodiments, the pharmaceutical composition comprises 0.2-15 molar equivalents of the pharmaceutical compound in an unionized form compared to the complexing agent. In some embodiments, the pharmaceutical composition comprises 0.5-10 molar equivalents of the pharmaceutical compound in an unionized form compared to the complexing agent. In some embodiments, the pharmaceutical composition comprises 1-5 molar equivalents of the pharmaceutical compound in an unionized form compared to the complexing agent. In some embodiments, the pharmaceutical composition comprises 0.1-20 molar equivalents of the pharmaceutical compound in an unionized form compared to the complexing agent, e.g., about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, or 20.0 molar equivalents of the pharmaceutical compound in an unionized form, or any amount therebetween. In some embodiments, the pharmaceutical composition comprises 1-2 molar equivalents of the pharmaceutical compound in an unionized form compared to the complexing agent. In some embodiments, the pharmaceutical composition comprises about 1.1, 1.2, 1.3, 1.4, 1.5, 1.6, 1.7, 1.8, 1.9, or about 2 molar equivalents of the pharmaceutical compound in an unionized form compared to the complexing agent.

In some embodiments, the pharmaceutical compound has a solubility of less than a threshold value in a solvent. In some embodiments, the pharmaceutical compound has a solubility of more than a threshold value in a solvent. In some embodiments, the solvent comprises an aqueous medium, e.g., water. In some embodiments, the solvent comprises an organic solvent, such as an organic solvent disclosed herein. In some embodiments, the protonated nitrogen atom of the pharmaceutical compound has a pKa value that is more than a threshold value. In some embodiments, the protonated nitrogen atom of the pharmaceutical compound has a pKa value that is less than a threshold value.

In some embodiments, the pharmaceutical compound has a solubility of more than 100 mg/ml as salt. In some embodiments, the pharmaceutical compound has a solubility of more than 90 mg/ml as salt. In some embodiments, the pharmaceutical compound has a solubility of more than 80 mg/ml as salt. In some embodiments, the pharmaceutical compound has a solubility of more than 70 mg/ml as salt. In some embodiments, the pharmaceutical compound has a solubility of more than 60 mg/ml as salt. In some embodiments, the pharmaceutical compound has a solubility of more than 50 mg/ml as salt. In some embodiments, the pharmaceutical compound has a solubility of more than 45 mg/ml as salt. In some embodiments, the pharmaceutical compound has a solubility of more than 40 mg/ml as salt. In some embodiments, the pharmaceutical compound has a solubility of more than 35 mg/ml as salt. In some embodiments, the pharmaceutical compound has a solubility of more than 30 mg/ml as salt. In some embodiments, the pharmaceutical compound has a solubility of more than 25 mg/ml as salt. In some embodiments, the pharmaceutical compound has a solubility of more than 20 mg/ml as salt. In some embodiments, the pharmaceutical compound has a solubility of more than 10 mg/ml as salt. In some embodiments, the pharmaceutical compound has a solubility of more than 9 mg/ml as salt. In some embodiments, the pharmaceutical compound has a solubility of more than 8 mg/ml as salt. In some embodiments, the pharmaceutical compound has a solubility of more than 7 mg/ml as salt. In some embodiments, the pharmaceutical compound has a solubility of more than 6 mg/ml as salt. In some embodiments, the pharmaceutical compound has a solubility of more than 5 mg/ml as salt. In some embodiments, the pharmaceutical compound has a solubility of more than 4 mg/ml as salt. In some embodiments, the pharmaceutical compound has a solubility of more than 3 mg/ml as salt. In some embodiments, the pharmaceutical compound has a solubility of more 2 mg/ml as salt. In some embodiments, the pharmaceutical compound has a solubility of more than 1 mg/ml as salt. In some embodiments, the pharmaceutical compound has a solubility of more than 0.9 mg/ml as salt. In some embodiments, the pharmaceutical compound has a solubility of more than 0.8 mg/ml as salt. In some embodiments, the pharmaceutical compound has a solubility of more than 0.7 mg/ml as salt. In some embodiments, the pharmaceutical compound has a solubility of more than 0.6 mg/ml as salt. In some embodiments, the pharmaceutical compound has a solubility of more than 0.5 mg/ml as salt. In some embodiments, the pharmaceutical compound has a solubility of more than 0.4 mg/ml as salt. In some embodiments, the pharmaceutical compound has a solubility of more than 0.3 mg/ml as salt. In some embodiments, the pharmaceutical compound has a solubility of more than 0.2 mg/ml as salt. In some embodiments, the pharmaceutical compound has a solubility of more than 0.1 mg/ml as salt. In some embodiments, the pharmaceutical compound has a solubility of more than 0.09 mg/ml as salt. In some embodiments, the pharmaceutical compound has a solubility of more than 0.08 mg/ml as salt. In some embodiments, the pharmaceutical compound has a solubility of more than 0.07 mg/ml as salt. In some embodiments, the pharmaceutical compound has a solubility of more than 0.06 mg/ml as salt. In some embodiments, the pharmaceutical compound has a solubility of more than 0.05 mg/ml as salt. In some embodiments, the pharmaceutical compound has a solubility of more than 0.04 mg/ml as salt. In some embodiments, the pharmaceutical compound has a solubility of more than 0.03 mg/ml as salt. In some embodiments, the pharmaceutical compound has a solubility of more than 0.02 mg/ml as salt. In some embodiments, the pharmaceutical compound has a solubility of more than 0.01 mg/ml as salt. In some embodiments, the pharmaceutical compound has a solubility of more than 0.009 mg/ml as salt. In some embodiments, the pharmaceutical compound has a solubility of more than 0.008 mg/ml as salt. In some embodiments, the pharmaceutical compound has a solubility of more than 0.007 mg/ml as salt. In some embodiments, the pharmaceutical compound has a solubility of more than 0.006 mg/ml as salt. In some embodiments, the pharmaceutical compound has a solubility of more than 0.005 mg/ml as salt. In some embodiments, the pharmaceutical compound has a solubility of more than 0.004 mg/ml as salt. In some embodiments, the pharmaceutical compound has a solubility of more than 0.003 mg/ml as salt. In some embodiments, the pharmaceutical compound has a solubility of more than 0.002 mg/ml as salt. In some embodiments, the pharmaceutical compound has a solubility of more than 0.001 mg/ml as salt. In some embodiments, the pharmaceutical compound has a solubility of more than 0.9 μg/ml as salt. In some embodiments, the pharmaceutical compound has a solubility of more than 0.8 μg/ml as salt. In some embodiments, the pharmaceutical compound has a solubility of more than 0.7 μg/ml as salt. In some embodiments, the pharmaceutical compound has a solubility of more than 0.6 μg/ml as salt. In some embodiments, the pharmaceutical compound has a solubility of more than 0.5 μg/ml as salt. In some embodiments, the pharmaceutical compound has a solubility of more than 0.4 μg/ml as salt. In some embodiments, the pharmaceutical compound has a solubility of more than 0.3 μg/ml as salt. In some embodiments, the pharmaceutical compound has a solubility of more than 0.2 μg/ml as salt. In some embodiments, the pharmaceutical compound has a solubility of more than 0.1 μg/ml as salt. In some embodiments, the pharmaceutical compound has a solubility of more than 0.09 μg/ml as salt. In some embodiments, the pharmaceutical compound has a solubility of more than 0.08 μg/ml as salt. In some embodiments, the pharmaceutical compound has a solubility of more than 0.07 μg/ml as salt. In some embodiments, the pharmaceutical compound has a solubility of more than 0.06 μg/ml as salt. In some embodiments, the pharmaceutical compound has a solubility of more than 0.05 μg/ml as salt. In some embodiments, the pharmaceutical compound has a solubility of more than 0.04 μg/ml as salt. In some embodiments, the pharmaceutical compound has a solubility of more than 0.03 μg/ml as salt. In some embodiments, the pharmaceutical compound has a solubility of more than 0.02 μg/ml as salt. In some embodiments, the pharmaceutical compound has a solubility of more than 0.01 μg/ml as salt. In some embodiments, the pharmaceutical compound has a solubility of more than 0.009 μg/ml as salt. In some embodiments, the pharmaceutical compound has a solubility of more than 0.008 μg/ml as salt.

In some embodiments, the pharmaceutical compound has a solubility of more than 0.007 μg/ml as salt. In some embodiments, the pharmaceutical compound has a solubility of more than 0.006 μg/ml as salt. In some embodiments, the pharmaceutical compound has a solubility of more than 0.005 μg/ml as salt. In some embodiments, the pharmaceutical compound has a solubility of more than 0.004 μg/ml as salt. In some embodiments, the pharmaceutical compound has a solubility of more than 0.003 μg/ml as salt. In some embodiments, the pharmaceutical compound has a solubility of more than 0.002 μg/ml as salt. In some embodiments, the pharmaceutical compound has a solubility of more than 0.001 μg/ml as salt. In some embodiments, the solubility of the pharmaceutical compound as salt is measured in an aqueous medium. In some embodiments, the solubility of the pharmaceutical compound as salt is measured in an organic solvent. In some embodiments, the aqueous medium comprises water. In some embodiments, the salt of the pharmaceutical compound comprises a HCl salt.

In some embodiments, the pharmaceutical compound has a solubility of between about 0.001 mg/ml and 100 mg/ml as salt. In some embodiments, the pharmaceutical compound has a solubility of between about 0.01 mg/ml and 50 mg/ml as salt. In some embodiments, the pharmaceutical compound has a solubility of between about 0.02 mg/ml and 40 mg/ml as salt. In some embodiments, the pharmaceutical compound has a solubility of between about 0.03 mg/ml and 30 mg/ml as salt. In some embodiments, the pharmaceutical compound has a solubility of between about 0.04 mg/ml and 20 mg/ml as salt. In some embodiments, the pharmaceutical compound has a solubility of between about 0.05 mg/ml and 10 mg/ml as salt. In some embodiments, the pharmaceutical compound has a solubility of between about 0.06 mg/ml and 5 mg/ml as salt. In some embodiments, the pharmaceutical compound has a solubility of between about 0.07 mg/ml and 4 mg/ml as salt. In some embodiments, the pharmaceutical compound has a solubility of between about 0.09 mg/ml and 3 mg/ml as salt. In some embodiments, the pharmaceutical compound has a solubility of between about 0.1 mg/ml and 2 mg/ml as salt. In some embodiments, the solubility of the pharmaceutical compound as salt is measured in an aqueous medium. In some embodiments, the solubility of the pharmaceutical compound as salt is measured in an organic solvent. In some embodiments, the aqueous medium comprises water. In some embodiments, the salt of the pharmaceutical compound comprises a HCl salt.

In some embodiments, the pharmaceutical compound has a solubility of less than 100 mg/ml as salt. In some embodiments, the pharmaceutical compound has a solubility of less than 90 mg/ml as salt. In some embodiments, the pharmaceutical compound has a solubility of less than 80 mg/ml as salt. In some embodiments, the pharmaceutical compound has a solubility of less than 70 mg/ml as salt. In some embodiments, the pharmaceutical compound has a solubility of less than 60 mg/ml as salt. In some embodiments, the pharmaceutical compound has a solubility of less than 50 mg/ml as salt. In some embodiments, the pharmaceutical compound has a solubility of less than 45 mg/ml as salt. In some embodiments, the pharmaceutical compound has a solubility of less than 40 mg/ml as salt. In some embodiments, the pharmaceutical compound has a solubility of less than 35 mg/ml as salt. In some embodiments, the pharmaceutical compound has a solubility of less than 30 mg/ml as salt. In some embodiments, the pharmaceutical compound has a solubility of less than 25 mg/ml as salt. In some embodiments, the pharmaceutical compound has a solubility of less than 20 mg/ml as salt. In some embodiments, the pharmaceutical compound has a solubility of less than 10 mg/ml as salt. In some embodiments, the pharmaceutical compound has a solubility of less than 9 mg/ml as salt. In some embodiments, the pharmaceutical compound has a solubility of less than 8 mg/ml as salt. In some embodiments, the pharmaceutical compound has a solubility of less than 7 mg/ml as salt. In some embodiments, the pharmaceutical compound has a solubility of less than 6 mg/ml as salt. In some embodiments, the pharmaceutical compound has a solubility of less than 5 mg/ml as salt. In some embodiments, the pharmaceutical compound has a solubility of less than 4 mg/ml as salt. In some embodiments, the pharmaceutical compound has a solubility of less than 3 mg/ml as salt. In some embodiments, the pharmaceutical compound has a solubility of more 2 mg/ml as salt. In some embodiments, the pharmaceutical compound has a solubility of less than 1 mg/ml as salt. In some embodiments, the pharmaceutical compound has a solubility of less than 0.9 mg/ml as salt. In some embodiments, the pharmaceutical compound has a solubility of less than 0.8 mg/ml as salt. In some embodiments, the pharmaceutical compound has a solubility of less than 0.7 mg/ml as salt. In some embodiments, the pharmaceutical compound has a solubility of less than 0.6 mg/ml as salt. In some embodiments, the pharmaceutical compound has a solubility of less than 0.5 mg/ml as salt. In some embodiments, the pharmaceutical compound has a solubility of less than 0.4 mg/ml as salt. In some embodiments, the pharmaceutical compound has a solubility of less than 0.3 mg/ml as salt. In some embodiments, the pharmaceutical compound has a solubility of less than 0.2 mg/ml as salt. In some embodiments, the pharmaceutical compound has a solubility of less than 0.1 mg/ml as salt. In some embodiments, the pharmaceutical compound has a solubility of less than 0.09 mg/ml as salt. In some embodiments, the pharmaceutical compound has a solubility of less than 0.08 mg/ml as salt. In some embodiments, the pharmaceutical compound has a solubility of less than 0.07 mg/ml as salt. In some embodiments, the pharmaceutical compound has a solubility of less than 0.06 mg/ml as salt. In some embodiments, the pharmaceutical compound has a solubility of less than 0.05 mg/ml as salt. In some embodiments, the pharmaceutical compound has a solubility of less than 0.04 mg/ml as salt. In some embodiments, the pharmaceutical compound has a solubility of less than 0.03 mg/ml as salt. In some embodiments, the pharmaceutical compound has a solubility of less than 0.02 mg/ml as salt. In some embodiments, the pharmaceutical compound has a solubility of less than 0.01 mg/ml as salt. In some embodiments, the pharmaceutical compound has a solubility of less than 0.009 mg/ml as salt. In some embodiments, the pharmaceutical compound has a solubility of less than 0.008 mg/ml as salt. In some embodiments, the pharmaceutical compound has a solubility of less than 0.007 mg/ml as salt. In some embodiments, the pharmaceutical compound has a solubility of less than 0.006 mg/ml as salt. In some embodiments, the pharmaceutical compound has a solubility of less than 0.005 mg/ml as salt. In some embodiments, the pharmaceutical compound has a solubility of less than 0.004 mg/ml as salt. In some embodiments, the pharmaceutical compound has a solubility of less than 0.003 mg/ml as salt. In some embodiments, the pharmaceutical compound has a solubility of less than 0.002 mg/ml as salt. In some embodiments, the pharmaceutical compound has a solubility of less than 0.001 mg/ml as salt. In some embodiments, the pharmaceutical compound has a solubility of less than 0.9 μg/ml as salt. In some embodiments, the pharmaceutical compound has a solubility of less than 0.8 μg/ml as salt. In some embodiments, the pharmaceutical compound has a solubility of less than 0.7 μg/ml as salt. In some embodiments, the pharmaceutical compound has a solubility of less than 0.6 μg/ml as salt. In some embodiments, the pharmaceutical compound has a solubility of less than 0.5 μg/ml as salt. In some embodiments, the pharmaceutical compound has a solubility of less than 0.4 μg/ml as salt. In some embodiments, the pharmaceutical compound has a solubility of less than 0.3 μg/ml as salt. In some embodiments, the pharmaceutical compound has a solubility of less than 0.2 μg/ml as salt. In some embodiments, the pharmaceutical compound has a solubility of less than 0.1 μg/ml as salt. In some embodiments, the pharmaceutical compound has a solubility of less than 0.09 μg/ml as salt. In some embodiments, the pharmaceutical compound has a solubility of less than 0.08 μg/ml as salt. In some embodiments, the pharmaceutical compound has a solubility of less than 0.07 μg/ml as salt. In some embodiments, the pharmaceutical compound has a solubility of less than 0.06 μg/ml as salt. In some embodiments, the pharmaceutical compound has a solubility of less than 0.05 μg/ml as salt. In some embodiments, the pharmaceutical compound has a solubility of less than 0.04 μg/ml as salt. In some embodiments, the pharmaceutical compound has a solubility of less than 0.03 μg/ml as salt. In some embodiments, the pharmaceutical compound has a solubility of less than 0.02 μg/ml as salt. In some embodiments, the pharmaceutical compound has a solubility of less than 0.01 μg/ml as salt. In some embodiments, the pharmaceutical compound has a solubility of less than 0.009 μg/ml as salt. In some embodiments, the pharmaceutical compound has a solubility of less than 0.008 μg/ml as salt. In some embodiments, the pharmaceutical compound has a solubility of less than 0.007 μg/ml as salt. In some embodiments, the pharmaceutical compound has a solubility of less than 0.006 μg/ml as salt. In some embodiments, the pharmaceutical compound has a solubility of less than 0.005 μg/ml as salt. In some embodiments, the pharmaceutical compound has a solubility of less than 0.004 μg/ml as salt. In some embodiments, the pharmaceutical compound has a solubility of less than 0.003 μg/ml as salt. In some embodiments, the pharmaceutical compound has a solubility of less than 0.002 μg/ml as salt. In some embodiments, the pharmaceutical compound has a solubility of less than 0.001 μg/ml as salt. In some embodiments, the solubility of the pharmaceutical compound as salt is measured in an aqueous medium. In some embodiments, the solubility of the pharmaceutical compound as salt is measured in an organic solvent. In some embodiments, the aqueous medium comprises water. In some embodiments, the salt of the pharmaceutical compound comprises a HCl salt.

In some embodiments, the pharmaceutical compound has a solubility of more than 100 mg/ml as freebase. In some embodiments, the pharmaceutical compound has a solubility of more than 90 mg/ml as freebase. In some embodiments, the pharmaceutical compound has a solubility of more than 80 mg/ml as freebase. In some embodiments, the pharmaceutical compound has a solubility of more than 70 mg/ml as freebase. In some embodiments, the pharmaceutical compound has a solubility of more than 60 mg/ml as freebase. In some embodiments, the pharmaceutical compound has a solubility of more than 50 mg/ml as freebase. In some embodiments, the pharmaceutical compound has a solubility of more than 45 mg/ml as freebase. In some embodiments, the pharmaceutical compound has a solubility of more than 40 mg/ml as freebase. In some embodiments, the pharmaceutical compound has a solubility of more than 35 mg/ml as freebase. In some embodiments, the pharmaceutical compound has a solubility of more than 30 mg/ml as freebase. In some embodiments, the pharmaceutical compound has a solubility of more than 25 mg/ml as freebase. In some embodiments, the pharmaceutical compound has a solubility of more than 20 mg/ml as freebase. In some embodiments, the pharmaceutical compound has a solubility of more than 10 mg/ml as freebase. In some embodiments, the pharmaceutical compound has a solubility of more than 9 mg/ml as freebase. In some embodiments, the pharmaceutical compound has a solubility of more than 8 mg/ml as freebase. In some embodiments, the pharmaceutical compound has a solubility of more than 7 mg/ml as freebase. In some embodiments, the pharmaceutical compound has a solubility of more than 6 mg/ml as freebase. In some embodiments, the pharmaceutical compound has a solubility of more than 5 mg/ml as freebase. In some embodiments, the pharmaceutical compound has a solubility of more than 4 mg/ml as freebase. In some embodiments, the pharmaceutical compound has a solubility of more than 3 mg/ml as freebase. In some embodiments, the pharmaceutical compound has a solubility of more 2 mg/ml as freebase. In some embodiments, the pharmaceutical compound has a solubility of more than 1 mg/ml as freebase. In some embodiments, the pharmaceutical compound has a solubility of more than 0.9 mg/ml as freebase. In some embodiments, the pharmaceutical compound has a solubility of more than 0.8 mg/ml as freebase. In some embodiments, the pharmaceutical compound has a solubility of more than 0.7 mg/ml as freebase. In some embodiments, the pharmaceutical compound has a solubility of more than 0.6 mg/ml as freebase. In some embodiments, the pharmaceutical compound has a solubility of more than 0.5 mg/ml as freebase. In some embodiments, the pharmaceutical compound has a solubility of more than 0.4 mg/ml as freebase. In some embodiments, the pharmaceutical compound has a solubility of more than 0.3 mg/ml as freebase. In some embodiments, the pharmaceutical compound has a solubility of more than 0.2 mg/ml as freebase. In some embodiments, the pharmaceutical compound has a solubility of more than 0.1 mg/ml as freebase. In some embodiments, the pharmaceutical compound has a solubility of more than 0.09 mg/ml as freebase. In some embodiments, the pharmaceutical compound has a solubility of more than 0.08 mg/ml as freebase. In some embodiments, the pharmaceutical compound has a solubility of more than 0.07 mg/ml as freebase. In some embodiments, the pharmaceutical compound has a solubility of more than 0.06 mg/ml as freebase. In some embodiments, the pharmaceutical compound has a solubility of more than 0.05 mg/ml as freebase. In some embodiments, the pharmaceutical compound has a solubility of more than 0.04 mg/ml as freebase. In some embodiments, the pharmaceutical compound has a solubility of more than 0.03 mg/ml as freebase. In some embodiments, the pharmaceutical compound has a solubility of more than 0.02 mg/ml as freebase. In some embodiments, the pharmaceutical compound has a solubility of more than 0.01 mg/ml as freebase. In some embodiments, the pharmaceutical compound has a solubility of more than 0.009 mg/ml as freebase. In some embodiments, the pharmaceutical compound has a solubility of more than 0.008 mg/ml as freebase. In some embodiments, the pharmaceutical compound has a solubility of more than 0.007 mg/ml as freebase. In some embodiments, the pharmaceutical compound has a solubility of more than 0.006 mg/ml as freebase. In some embodiments, the pharmaceutical compound has a solubility of more than 0.005 mg/ml as freebase. In some embodiments, the pharmaceutical compound has a solubility of more than 0.004 mg/ml as freebase. In some embodiments, the pharmaceutical compound has a solubility of more than 0.003 mg/ml as freebase. In some embodiments, the pharmaceutical compound has a solubility of more than 0.002 mg/ml as freebase. In some embodiments, the pharmaceutical compound has a solubility of more than 0.001 mg/ml as freebase. In some embodiments, the pharmaceutical compound has a solubility of more than 0.9 μg/ml as freebase. In some embodiments, the pharmaceutical compound has a solubility of more than 0.8 μg/ml as freebase. In some embodiments, the pharmaceutical compound has a solubility of more than 0.7 μg/ml as freebase. In some embodiments, the pharmaceutical compound has a solubility of more than 0.6 μg/ml as freebase. In some embodiments, the pharmaceutical compound has a solubility of more than 0.5 μg/ml as freebase. In some embodiments, the pharmaceutical compound has a solubility of more than 0.4 μg/ml as freebase. In some embodiments, the pharmaceutical compound has a solubility of more than 0.3 μg/ml as freebase. In some embodiments, the pharmaceutical compound has a solubility of more than 0.2 μg/ml as freebase. In some embodiments, the pharmaceutical compound has a solubility of more than 0.1 μg/ml as freebase. In some embodiments, the pharmaceutical compound has a solubility of more than 0.09 μg/ml as freebase. In some embodiments, the pharmaceutical compound has a solubility of more than 0.08 μg/ml as freebase. In some embodiments, the pharmaceutical compound has a solubility of more than 0.07 μg/ml as freebase. In some embodiments, the pharmaceutical compound has a solubility of more than 0.06 μg/ml as freebase. In some embodiments, the pharmaceutical compound has a solubility of more than 0.05 μg/ml as freebase. In some embodiments, the pharmaceutical compound has a solubility of more than 0.04 μg/ml as freebase. In some embodiments, the pharmaceutical compound has a solubility of more than 0.03 μg/ml as freebase. In some embodiments, the pharmaceutical compound has a solubility of more than 0.02 μg/ml as freebase. In some embodiments, the pharmaceutical compound has a solubility of more than 0.01 μg/ml as freebase. In some embodiments, the pharmaceutical compound has a solubility of more than 0.009 μg/ml as freebase. In some embodiments, the pharmaceutical compound has a solubility of more than 0.008 μg/ml as freebase. In some embodiments, the pharmaceutical compound has a solubility of more than 0.007 μg/ml as freebase. In some embodiments, the pharmaceutical compound has a solubility of more than 0.006 μg/ml as freebase. In some embodiments, the pharmaceutical compound has a solubility of more than 0.005 μg/ml as freebase. In some embodiments, the pharmaceutical compound has a solubility of more than 0.004 μg/ml as freebase. In some embodiments, the pharmaceutical compound has a solubility of more than 0.003 μg/ml as freebase. In some embodiments, the pharmaceutical compound has a solubility of more than 0.002 μg/ml as freebase. In some embodiments, the pharmaceutical compound has a solubility of more than 0.001 μg/ml as freebase. In some embodiments, the solubility of the pharmaceutical compound as freebase is measured in an aqueous medium. In some embodiments, the solubility of the pharmaceutical compound as freebase is measured in an organic solvent. In some embodiments, the aqueous medium comprises water.

In some embodiments, the pharmaceutical compound has a solubility of between about 0.001 mg/ml and 100 mg/ml as freebase. In some embodiments, the pharmaceutical compound has a solubility of between about 0.01 mg/ml and 50 mg/ml as freebase. In some embodiments, the pharmaceutical compound has a solubility of between about 0.02 mg/ml and 40 mg/ml as freebase. In some embodiments, the pharmaceutical compound has a solubility of between about 0.03 mg/ml and 30 mg/ml as freebase. In some embodiments, the pharmaceutical compound has a solubility of between about 0.04 mg/ml and 20 mg/ml as freebase. In some embodiments, the pharmaceutical compound has a solubility of between about 0.05 mg/ml and 10 mg/ml as freebase. In some embodiments, the pharmaceutical compound has a solubility of between about 0.06 mg/ml and 5 mg/ml as freebase. In some embodiments, the pharmaceutical compound has a solubility of between about 0.07 mg/ml and 4 mg/ml as freebase. In some embodiments, the pharmaceutical compound has a solubility of between about 0.09 mg/ml and 3 mg/ml as freebase. In some embodiments, the pharmaceutical compound has a solubility of between about 0.1 mg/ml and 2 mg/ml as freebase. In some embodiments, the solubility of the pharmaceutical compound as freebase is measured in an aqueous medium. In some embodiments, the solubility of the pharmaceutical compound as freebase is measured in an organic solvent. In some embodiments, the aqueous medium comprises water.

In some embodiments, the pharmaceutical compound has a solubility of less than 100 mg/ml as freebase. In some embodiments, the pharmaceutical compound has a solubility of less than 90 mg/ml as freebase. In some embodiments, the pharmaceutical compound has a solubility of less than 80 mg/ml as freebase. In some embodiments, the pharmaceutical compound has a solubility of less than 70 mg/ml as freebase. In some embodiments, the pharmaceutical compound has a solubility of less than 60 mg/ml as freebase. In some embodiments, the pharmaceutical compound has a solubility of less than 50 mg/ml as freebase. In some embodiments, the pharmaceutical compound has a solubility of less than 45 mg/ml as freebase. In some embodiments, the pharmaceutical compound has a solubility of less than 40 mg/ml as freebase. In some embodiments, the pharmaceutical compound has a solubility of less than 35 mg/ml as freebase. In some embodiments, the pharmaceutical compound has a solubility of less than 30 mg/ml as freebase. In some embodiments, the pharmaceutical compound has a solubility of less than 25 mg/ml as freebase. In some embodiments, the pharmaceutical compound has a solubility of less than 20 mg/ml as freebase. In some embodiments, the pharmaceutical compound has a solubility of less than 10 mg/ml as freebase. In some embodiments, the pharmaceutical compound has a solubility of less than 9 mg/ml as freebase. In some embodiments, the pharmaceutical compound has a solubility of less than 8 mg/ml as freebase. In some embodiments, the pharmaceutical compound has a solubility of less than 7 mg/ml as freebase. In some embodiments, the pharmaceutical compound has a solubility of less than 6 mg/ml as freebase. In some embodiments, the pharmaceutical compound has a solubility of less than 5 mg/ml as freebase. In some embodiments, the pharmaceutical compound has a solubility of less than 4 mg/ml as freebase. In some embodiments, the pharmaceutical compound has a solubility of less than 3 mg/ml as freebase. In some embodiments, the pharmaceutical compound has a solubility of more 2 mg/ml as freebase. In some embodiments, the pharmaceutical compound has a solubility of less than 1 mg/ml as freebase. In some embodiments, the pharmaceutical compound has a solubility of less than 0.9 mg/ml as freebase. In some embodiments, the pharmaceutical compound has a solubility of less than 0.8 mg/ml as freebase. In some embodiments, the pharmaceutical compound has a solubility of less than 0.7 mg/ml as freebase. In some embodiments, the pharmaceutical compound has a solubility of less than 0.6 mg/ml as freebase. In some embodiments, the pharmaceutical compound has a solubility of less than 0.5 mg/ml as freebase. In some embodiments, the pharmaceutical compound has a solubility of less than 0.4 mg/ml as freebase. In some embodiments, the pharmaceutical compound has a solubility of less than 0.3 mg/ml as freebase. In some embodiments, the pharmaceutical compound has a solubility of less than 0.2 mg/ml as freebase. In some embodiments, the pharmaceutical compound has a solubility of less than 0.1 mg/ml as freebase. In some embodiments, the pharmaceutical compound has a solubility of less than 0.09 mg/ml as freebase. In some embodiments, the pharmaceutical compound has a solubility of less than 0.08 mg/ml as freebase. In some embodiments, the pharmaceutical compound has a solubility of less than 0.07 mg/ml as freebase. In some embodiments, the pharmaceutical compound has a solubility of less than 0.06 mg/ml as freebase. In some embodiments, the pharmaceutical compound has a solubility of less than 0.05 mg/ml as freebase. In some embodiments, the pharmaceutical compound has a solubility of less than 0.04 mg/ml as freebase. In some embodiments, the pharmaceutical compound has a solubility of less than 0.03 mg/ml as freebase. In some embodiments, the pharmaceutical compound has a solubility of less than 0.02 mg/ml as freebase. In some embodiments, the pharmaceutical compound has a solubility of less than 0.01 mg/ml as freebase. In some embodiments, the pharmaceutical compound has a solubility of less than 0.009 mg/ml as freebase. In some embodiments, the pharmaceutical compound has a solubility of less than 0.008 mg/ml as freebase. In some embodiments, the pharmaceutical compound has a solubility of less than 0.007 mg/ml as freebase. In some embodiments, the pharmaceutical compound has a solubility of less than 0.006 mg/ml as freebase. In some embodiments, the pharmaceutical compound has a solubility of less than 0.005 mg/ml as freebase. In some embodiments, the pharmaceutical compound has a solubility of less than 0.004 mg/ml as freebase. In some embodiments, the pharmaceutical compound has a solubility of less than 0.003 mg/ml as freebase. In some embodiments, the pharmaceutical compound has a solubility of less than 0.002 mg/ml as freebase. In some embodiments, the pharmaceutical compound has a solubility of less than 0.001 mg/ml as freebase. In some embodiments, the pharmaceutical compound has a solubility of less than 0.9 μg/ml as freebase. In some embodiments, the pharmaceutical compound has a solubility of less than 0.8 μg/ml as freebase. In some embodiments, the pharmaceutical compound has a solubility of less than 0.7 μg/ml as freebase. In some embodiments, the pharmaceutical compound has a solubility of less than 0.6 μg/ml as freebase. In some embodiments, the pharmaceutical compound has a solubility of less than 0.5 μg/ml as freebase. In some embodiments, the pharmaceutical compound has a solubility of less than 0.4 μg/ml as freebase. In some embodiments, the pharmaceutical compound has a solubility of less than 0.3 μg/ml as freebase. In some embodiments, the pharmaceutical compound has a solubility of less than 0.2 μg/ml as freebase. In some embodiments, the pharmaceutical compound has a solubility of less than 0.1 μg/ml as freebase. In some embodiments, the pharmaceutical compound has a solubility of less than 0.09 μg/ml as freebase. In some embodiments, the pharmaceutical compound has a solubility of less than 0.08 μg/ml as freebase. In some embodiments, the pharmaceutical compound has a solubility of less than 0.07 μg/ml as freebase. In some embodiments, the pharmaceutical compound has a solubility of less than 0.06 μg/ml as freebase. In some embodiments, the pharmaceutical compound has a solubility of less than 0.05 μg/ml as freebase. In some embodiments, the pharmaceutical compound has a solubility of less than 0.04 μg/ml as freebase. In some embodiments, the pharmaceutical compound has a solubility of less than 0.03 μg/ml as freebase. In some embodiments, the pharmaceutical compound has a solubility of less than 0.02 μg/ml as freebase. In some embodiments, the pharmaceutical compound has a solubility of less than 0.01 μg/ml as freebase. In some embodiments, the pharmaceutical compound has a solubility of less than 0.009 μg/ml as freebase. In some embodiments, the pharmaceutical compound has a solubility of less than 0.008 μg/ml as freebase. In some embodiments, the pharmaceutical compound has a solubility of less than 0.007 μg/ml as freebase. In some embodiments, the pharmaceutical compound has a solubility of less than 0.006 μg/ml as freebase. In some embodiments, the pharmaceutical compound has a solubility of less than 0.005 μg/ml as freebase. In some embodiments, the pharmaceutical compound has a solubility of less than 0.004 μg/ml as freebase. In some embodiments, the pharmaceutical compound has a solubility of less than 0.003 μg/ml as freebase. In some embodiments, the pharmaceutical compound has a solubility of less than 0.002 μg/ml as freebase. In some embodiments, the pharmaceutical compound has a solubility of less than 0.001 μg/ml as freebase. In some embodiments, the solubility of the pharmaceutical compound as freebase is measured in an aqueous medium. In some embodiments, the solubility of the pharmaceutical compound as freebase is measured in an organic solvent. In some embodiments, the aqueous medium comprises water.

In some embodiments, a precipitate forms when an amount of the pharmaceutical compound is mixed with the pharmaceutical composition dissolved in a solvent. In some embodiments, a precipitate forms when between 0.1 and 20 molar equivalents of the pharmaceutical compound, compared to the complexing agent in the pharmaceutical composition, is mixed with the pharmaceutical composition dissolved in a solvent. In some embodiments, a precipitate forms when between 0.5 and 15 molar equivalents of the pharmaceutical compound, compared to the complexing agent in the pharmaceutical composition, is mixed with the pharmaceutical composition dissolved in a solvent. In some embodiments, a precipitate forms when between 1 and 10 molar equivalents of the pharmaceutical compound, compared to the complexing agent in the pharmaceutical composition, is mixed with the pharmaceutical composition dissolved in a solvent. In some embodiments, a precipitate forms when between 2 and 5 molar equivalents of the pharmaceutical compound, compared to the complexing agent in the pharmaceutical composition, is mixed with the pharmaceutical composition dissolved in a solvent. In some embodiments, a precipitate forms when between 3 and 4 molar equivalents of the pharmaceutical compound, compared to the complexing agent in the pharmaceutical composition, is mixed with the pharmaceutical composition dissolved in a solvent. In some embodiments, a precipitate forms when an amount of the pharmaceutical compound is mixed with the pharmaceutical composition dissolved in a solvent and the amount of the pharmaceutical compound being added is between 0.1 and 20 molar equivalents compared to the complexing agent in the pharmaceutical composition, e.g., about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, or about 20.0 molar equivalents, or any amount therebetween. In some embodiments, the solvent comprises an organic solvent disclosed herein. In some embodiments, the solvent comprises an aqueous medium. In some embodiments, the solvent comprises water.

In some embodiments, the solubility of the pharmaceutical compound in the pharmaceutical composition is higher than (i) the solubility of the pharmaceutical compound as a salt; or (ii) the solubility of the pharmaceutical compound in a composition comprising the pharmaceutical compound in freebase form that is complexing to a non-polar pore of the complexing agent, wherein the pharmaceutical compound has the same concentration in the pharmaceutical composition, the pharmaceutical compound as a salt, and the a composition comprising the pharmaceutical compound in freebase form. In some embodiments, the solubility of the pharmaceutical compound in the pharmaceutical composition is higher than the solubility of the pharmaceutical compound in a composition comprising the pharmaceutical compound and complexing agent with a higher molar ratio of the pharmaceutical compound to the complexing agent, wherein the pharmaceutical compound has the same concentration in the pharmaceutical composition and the a composition with a higher molar ratio of the pharmaceutical compound to the complexing agent. In some embodiments, the solubility of the pharmaceutical compound in the pharmaceutical composition has an increase of at least 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, or 100-fold compared with (i) the solubility of the pharmaceutical compound as a salt; or (ii) the solubility of the pharmaceutical compound in a composition comprising the pharmaceutical compound in freebase form that is complexing to a non-polar pore of the complexing agent, wherein the pharmaceutical compound has the same concentration in the pharmaceutical composition, the pharmaceutical compound as a salt, and the composition comprising the pharmaceutical compound in freebase form. In some embodiments, the solubility of the pharmaceutical compound in the pharmaceutical composition has an increase of at least 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, or 100-fold compared with the solubility of the pharmaceutical compound in a composition comprising the pharmaceutical compound and complexing agent with a higher molar ratio of the pharmaceutical compound to the complexing agent, wherein the pharmaceutical compound has the same concentration in the pharmaceutical composition and the composition with a higher molar ratio of the pharmaceutical compound to the complexing agent.

In an aspect provided herein is a pharmaceutical composition, comprising: (i) a pharmaceutical compound, or an enantiomer, a mixture of enantiomers, or an isotopic variant thereof, wherein the pharmaceutical compound comprises a protonated nitrogen atom; (ii) a complexing agent, wherein the complexing agent comprises a plurality of acidic functional groups, wherein the plurality of acidic functional groups comprise a conjugate base of an acid which acts as a counterion for the protonated nitrogen atom of the pharmaceutical compound, wherein the pharmaceutical composition has a ratio of the complexing agent to the protonated pharmaceutical compound that is from about 1:1 to about 1:4; and (iii) an additional molar equivalent of the pharmaceutical compound, wherein the additional molar equivalent of the pharmaceutical compound is unionized.

In some embodiments, the pharmaceutical compound is an antiviral compound, an antibacterial compound, an anti-fungal compound, a compound for treatment of a neurological disorder, a compound for treatment of Parkinson's disease, a treatment for migraine headache, a treatment for autoimmune disease, a treatment for cancer, a treatment for lymphoma, a treatment for pancreatitis, a treatment for opioid overdose, a treatment for flu infection, or a treatment for an inflammatory disorder. In some embodiments, the pharmaceutical compound comprises rotigotine, eletriptan, copanlisib, remdesivir, nafamostat (nafamostat mesylate), melevodopa, tigecycline, naloxone, amikacin, 1-amantadine, 2-amantadine, rimantadine, amifampridine, rapamycin, clonidine, or caspofungin.

In some embodiments, the ratio of the complexing agent to the protonated pharmaceutical compound is from about 1:1 to about 1:4. In some embodiments, the ratio of the complexing agent to the protonated pharmaceutical compound is from about 1:1.1 to about 1:3.9. In some embodiments, the ratio of the complexing agent to the protonated pharmaceutical compound is from about 1:1.2 to about 1:3.8. In some embodiments, the ratio of the complexing agent to the protonated pharmaceutical compound is from about 1:1.3 to about 1:3.7. In some embodiments, the ratio of the complexing agent to the protonated pharmaceutical compound is from about 1:1.4 to about 1:3.6. In some embodiments, the ratio of the complexing agent to the protonated pharmaceutical compound is from about 1:1.5 to about 1:3.5. In some embodiments, the ratio of the complexing agent to the protonated pharmaceutical compound is from about 1:1.6 to about 1:3.4. In some embodiments, the ratio of the complexing agent to the protonated pharmaceutical compound is from about 1:1.7 to about 1:3.3. In some embodiments, the ratio of the complexing agent to the protonated pharmaceutical compound is from about 1:1.8 to about 1:3.2. In some embodiments, the ratio of the complexing agent to the protonated pharmaceutical compound is from about 1:1.9 to about 1:3.1. In some embodiments, the ratio of the complexing agent to the protonated pharmaceutical compound is from about 1:2 to about 1:3. In some embodiments, the ratio of the complexing agent to the protonated pharmaceutical compound is from about 1:2.1 to about 1:2.9. In some embodiments, the ratio of the complexing agent to the protonated pharmaceutical compound is from about 1:2.2 to about 1:2.8. In some embodiments, the ratio of the complexing agent to the protonated pharmaceutical compound is from about 1:2.3 to about 1:2.7. In some embodiments, the ratio of the complexing agent to the protonated pharmaceutical compound is from about 1:2.4 to about 1:2.6. In some embodiments, the ratio of the complexing agent to the protonated pharmaceutical compound is from about 1:1 to about 1:4, about 1:1.1, 1:1.2, 1:1.3, 1:1.4, 1:1.5, 1:1.6, 1:1.7, 1:1.8, 1:1.9, 1:2.0, 1:2.1, 1:2.2, 1:2.3, 1:2.4, 1:2.5, 1:2.6, 1:2.7, 1:2.8, 1:2.9, 1:3.0, 1:3.1, 1:3.2, 1:3.3, 1:3.4, 1:3.5, 1:3.6, 1:3.7, 1:3.8, 1:3.9, or about 1:4, or any ratio therebetween.

In some embodiments, the complexing agent acts as the counterion to between 1 to 4 molecules of the protonated pharmaceutical compound, e.g., about 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or about 4 molecules of the protonated pharmaceutical compound, or any number of molecules of the pharmaceutical compound therebetween. In some embodiments, the total number of acidic functional groups of the complexing agent in the pharmaceutical composition is more than the total number of protonated nitrogen atoms of the pharmaceutical compound in the pharmaceutical composition. In some embodiments, a portion, but not all, of the acidic functional groups of the complexing agent neutralize all of the protonated nitrogen atoms of the pharmaceutical compound.

In some embodiments, fewer than 5 acidic functional groups of the complexing agent neutralize all of the protonated nitrogen atoms of the pharmaceutical compound. In some embodiments, fewer than 4 acidic functional groups of the complexing agent neutralize all of the protonated nitrogen atoms of the pharmaceutical compound. In some embodiments, fewer than 3 acidic functional groups of the complexing agent neutralize all of the protonated nitrogen atoms of the pharmaceutical compound. In some embodiments, fewer than 2 acidic functional groups of the complexing agent neutralize all of the protonated nitrogen atoms of the pharmaceutical compound. In some embodiments, fewer than 1 acidic functional groups of the complexing agent neutralize all of the protonated nitrogen atoms of the pharmaceutical compound.

In some embodiments, all of the protonated nitrogen atoms of the pharmaceutical compound are neutralized by 1 to 4 acidic functional groups. In some embodiments, all of the protonated nitrogen atoms of the pharmaceutical compound are neutralized by 1 to 3 acidic functional groups. In some embodiments, all of the protonated nitrogen atoms of the pharmaceutical compound are neutralized by 1 to 2 acidic functional groups. In some embodiments, all of the protonated nitrogen atoms of the pharmaceutical compound are neutralized by 1-4 acidic functional groups, e.g., about 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or about 4.0 acidic functional groups, or any number therebetween.

In some embodiments, the pharmaceutical composition comprises excess ions. In some embodiments, the excess ions comprise a salt, e.g., sodium chloride, lithium chloride, potassium chloride, or sodium bromide. In some embodiments, an excess ion acts as a counterion to an acidic functional group of the complexing agent. In some embodiments, such as an excess sodium ion occupying a deprotonated acidic site.

In some embodiments, the complexing agent further comprises a non-polar region. In some embodiments, the non-polar region is a non-polar pore. In some embodiments, the additional molar equivalent of the pharmaceutical compound in the unionized form is complexed to the non-polar region of the complexing agent. In some embodiments, the additional molar equivalent of the pharmaceutical compound in the unionized form is complexed to the non-polar pore of the complexing agent.

In some embodiments, the pharmaceutical composition comprises 0.1-20 molar equivalents of the pharmaceutical compound in an unionized form compared to the complexing agent. In some embodiments, the pharmaceutical composition comprises 0.2-15 molar equivalents of the pharmaceutical compound in an unionized form compared to the complexing agent. In some embodiments, the pharmaceutical composition comprises 0.5-10 molar equivalents of the pharmaceutical compound in an unionized form compared to the complexing agent. In some embodiments, the pharmaceutical composition comprises 1-5 molar equivalents of the pharmaceutical compound in an unionized form compared to the complexing agent. In some embodiments, the pharmaceutical composition comprises 0.1-20 molar equivalents of the pharmaceutical compound in an unionized form compared to the complexing agent, e.g., about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, or 20.0 molar equivalents of the pharmaceutical compound in an unionized form, or any amount therebetween. In some embodiments, the pharmaceutical composition comprises 1-2 molar equivalents of the pharmaceutical compound in an unionized form compared to the complexing agent. In some embodiments, the pharmaceutical composition comprises about 1.1, 1.2, 1.3, 1.4, 1.5, 1.6, 1.7, 1.8, 1.9, or about 2 molar equivalents of the pharmaceutical compound in an unionized form compared to the complexing agent.

In some embodiments, the pharmaceutical compound has a solubility of less than a threshold value in a solvent. In some embodiments, the pharmaceutical compound has a solubility of more than a threshold value in a solvent. In some embodiments, the solvent comprises an aqueous medium, e.g., water. In some embodiments, the solvent comprises an organic solvent, such as an organic solvent disclosed herein. In some embodiments, the protonated nitrogen atom of the pharmaceutical compound has a pKa value that is more than a threshold value. In some embodiments, the protonated nitrogen atom of the pharmaceutical compound has a pKa value that is less than a threshold value.

In some embodiments, a precipitate forms when an amount of the pharmaceutical compound is mixed with the pharmaceutical composition dissolved in a solvent. In some embodiments, a precipitate forms when between 0.1 and 20 molar equivalents of the pharmaceutical compound, compared to the complexing agent in the pharmaceutical composition, is mixed with the pharmaceutical composition dissolved in a solvent. In some embodiments, a precipitate forms when between 0.5 and 15 molar equivalents of the pharmaceutical compound, compared to the complexing agent in the pharmaceutical composition, is mixed with the pharmaceutical composition dissolved in a solvent. In some embodiments, a precipitate forms when between 1 and 10 molar equivalents of the pharmaceutical compound, compared to the complexing agent in the pharmaceutical composition, is mixed with the pharmaceutical composition dissolved in a solvent. In some embodiments, a precipitate forms when between 2 and 5 molar equivalents of the pharmaceutical compound, compared to the complexing agent in the pharmaceutical composition, is mixed with the pharmaceutical composition dissolved in a solvent. In some embodiments, a precipitate forms when between 3 and 4 molar equivalents of the pharmaceutical compound, compared to the complexing agent in the pharmaceutical composition, is mixed with the pharmaceutical composition dissolved in a solvent. In some embodiments, a precipitate forms when an amount of the pharmaceutical compound is mixed with the pharmaceutical composition dissolved in a solvent and the amount of the pharmaceutical compound being added is between 0.1 and 20 molar equivalents compared to the complexing agent in the pharmaceutical composition, e.g., about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, or about 20.0 molar equivalents, or any amount therebetween. In some embodiments, the solvent comprises an organic solvent disclosed herein. In some embodiments, the solvent comprises an aqueous medium. In some embodiments, the solvent comprises water.

In some embodiments, the pharmaceutical compound has a solubility of more than 100 mg/ml as salt. In some embodiments, the pharmaceutical compound has a solubility of more than 90 mg/ml as salt. In some embodiments, the pharmaceutical compound has a solubility of more than 80 mg/ml as salt. In some embodiments, the pharmaceutical compound has a solubility of more than 70 mg/ml as salt. In some embodiments, the pharmaceutical compound has a solubility of more than 60 mg/ml as salt. In some embodiments, the pharmaceutical compound has a solubility of more than 50 mg/ml as salt. In some embodiments, the pharmaceutical compound has a solubility of more than 45 mg/ml as salt. In some embodiments, the pharmaceutical compound has a solubility of more than 40 mg/ml as salt. In some embodiments, the pharmaceutical compound has a solubility of more than 35 mg/ml as salt. In some embodiments, the pharmaceutical compound has a solubility of more than 30 mg/ml as salt. In some embodiments, the pharmaceutical compound has a solubility of more than 25 mg/ml as salt. In some embodiments, the pharmaceutical compound has a solubility of more than 20 mg/ml as salt. In some embodiments, the pharmaceutical compound has a solubility of more than 10 mg/ml as salt. In some embodiments, the pharmaceutical compound has a solubility of more than 9 mg/ml as salt. In some embodiments, the pharmaceutical compound has a solubility of more than 8 mg/ml as salt. In some embodiments, the pharmaceutical compound has a solubility of more than 7 mg/ml as salt. In some embodiments, the pharmaceutical compound has a solubility of more than 6 mg/ml as salt. In some embodiments, the pharmaceutical compound has a solubility of more than 5 mg/ml as salt. In some embodiments, the pharmaceutical compound has a solubility of more than 4 mg/ml as salt. In some embodiments, the pharmaceutical compound has a solubility of more than 3 mg/ml as salt. In some embodiments, the pharmaceutical compound has a solubility of more 2 mg/ml as salt. In some embodiments, the pharmaceutical compound has a solubility of more than 1 mg/ml as salt. In some embodiments, the pharmaceutical compound has a solubility of more than 0.9 mg/ml as salt. In some embodiments, the pharmaceutical compound has a solubility of more than 0.8 mg/ml as salt. In some embodiments, the pharmaceutical compound has a solubility of more than 0.7 mg/ml as salt. In some embodiments, the pharmaceutical compound has a solubility of more than 0.6 mg/ml as salt. In some embodiments, the pharmaceutical compound has a solubility of more than 0.5 mg/ml as salt. In some embodiments, the pharmaceutical compound has a solubility of more than 0.4 mg/ml as salt. In some embodiments, the pharmaceutical compound has a solubility of more than 0.3 mg/ml as salt. In some embodiments, the pharmaceutical compound has a solubility of more than 0.2 mg/ml as salt. In some embodiments, the pharmaceutical compound has a solubility of more than 0.1 mg/ml as salt. In some embodiments, the pharmaceutical compound has a solubility of more than 0.09 mg/ml as salt. In some embodiments, the pharmaceutical compound has a solubility of more than 0.08 mg/ml as salt. In some embodiments, the pharmaceutical compound has a solubility of more than 0.07 mg/ml as salt. In some embodiments, the pharmaceutical compound has a solubility of more than 0.06 mg/ml as salt. In some embodiments, the pharmaceutical compound has a solubility of more than 0.05 mg/ml as salt. In some embodiments, the pharmaceutical compound has a solubility of more than 0.04 mg/ml as salt. In some embodiments, the pharmaceutical compound has a solubility of more than 0.03 mg/ml as salt. In some embodiments, the pharmaceutical compound has a solubility of more than 0.02 mg/ml as salt. In some embodiments, the pharmaceutical compound has a solubility of more than 0.01 mg/ml as salt. In some embodiments, the pharmaceutical compound has a solubility of more than 0.009 mg/ml as salt. In some embodiments, the pharmaceutical compound has a solubility of more than 0.008 mg/ml as salt. In some embodiments, the pharmaceutical compound has a solubility of more than 0.007 mg/ml as salt. In some embodiments, the pharmaceutical compound has a solubility of more than 0.006 mg/ml as salt. In some embodiments, the pharmaceutical compound has a solubility of more than 0.005 mg/ml as salt. In some embodiments, the pharmaceutical compound has a solubility of more than 0.004 mg/ml as salt. In some embodiments, the pharmaceutical compound has a solubility of more than 0.003 mg/ml as salt. In some embodiments, the pharmaceutical compound has a solubility of more than 0.002 mg/ml as salt. In some embodiments, the pharmaceutical compound has a solubility of more than 0.001 mg/ml as salt. In some embodiments, the pharmaceutical compound has a solubility of more than 0.9 μg/ml as salt. In some embodiments, the pharmaceutical compound has a solubility of more than 0.8 μg/ml as salt. In some embodiments, the pharmaceutical compound has a solubility of more than 0.7 μg/ml as salt. In some embodiments, the pharmaceutical compound has a solubility of more than 0.6 μg/ml as salt. In some embodiments, the pharmaceutical compound has a solubility of more than 0.5 μg/ml as salt. In some embodiments, the pharmaceutical compound has a solubility of more than 0.4 μg/ml as salt. In some embodiments, the pharmaceutical compound has a solubility of more than 0.3 μg/ml as salt. In some embodiments, the pharmaceutical compound has a solubility of more than 0.2 μg/ml as salt. In some embodiments, the pharmaceutical compound has a solubility of more than 0.1 μg/ml as salt. In some embodiments, the pharmaceutical compound has a solubility of more than 0.09 μg/ml as salt. In some embodiments, the pharmaceutical compound has a solubility of more than 0.08 μg/ml as salt. In some embodiments, the pharmaceutical compound has a solubility of more than 0.07 μg/ml as salt. In some embodiments, the pharmaceutical compound has a solubility of more than 0.06 μg/ml as salt. In some embodiments, the pharmaceutical compound has a solubility of more than 0.05 μg/ml as salt. In some embodiments, the pharmaceutical compound has a solubility of more than 0.04 μg/ml as salt. In some embodiments, the pharmaceutical compound has a solubility of more than 0.03 μg/ml as salt. In some embodiments, the pharmaceutical compound has a solubility of more than 0.02 μg/ml as salt. In some embodiments, the pharmaceutical compound has a solubility of more than 0.01 μg/ml as salt. In some embodiments, the pharmaceutical compound has a solubility of more than 0.009 μg/ml as salt. In some embodiments, the pharmaceutical compound has a solubility of more than 0.008 μg/ml as salt. In some embodiments, the pharmaceutical compound has a solubility of more than 0.007 μg/ml as salt. In some embodiments, the pharmaceutical compound has a solubility of more than 0.006 μg/ml as salt. In some embodiments, the pharmaceutical compound has a solubility of more than 0.005 μg/ml as salt. In some embodiments, the pharmaceutical compound has a solubility of more than 0.004 μg/ml as salt. In some embodiments, the pharmaceutical compound has a solubility of more than 0.003 μg/ml as salt. In some embodiments, the pharmaceutical compound has a solubility of more than 0.002 μg/ml as salt. In some embodiments, the pharmaceutical compound has a solubility of more than 0.001 μg/ml as salt. In some embodiments, the solubility of the pharmaceutical compound as salt is measured in an aqueous medium. In some embodiments, the solubility of the pharmaceutical compound as salt is measured in an organic solvent. In some embodiments, the aqueous medium comprises water. In some embodiments, the salt of the pharmaceutical compound comprises a HCl salt.

In some embodiments, the pharmaceutical compound has a solubility of between about 0.001 mg/ml and 100 mg/ml as salt. In some embodiments, the pharmaceutical compound has a solubility of between about 0.01 mg/ml and 50 mg/ml as salt. In some embodiments, the pharmaceutical compound has a solubility of between about 0.02 mg/ml and 40 mg/ml as salt. In some embodiments, the pharmaceutical compound has a solubility of between about 0.03 mg/ml and 30 mg/ml as salt. In some embodiments, the pharmaceutical compound has a solubility of between about 0.04 mg/ml and 20 mg/ml as salt. In some embodiments, the pharmaceutical compound has a solubility of between about 0.05 mg/ml and 10 mg/ml as salt. In some embodiments, the pharmaceutical compound has a solubility of between about 0.06 mg/ml and 5 mg/ml as salt. In some embodiments, the pharmaceutical compound has a solubility of between about 0.07 mg/ml and 4 mg/ml as salt. In some embodiments, the pharmaceutical compound has a solubility of between about 0.09 mg/ml and 3 mg/ml as salt. In some embodiments, the pharmaceutical compound has a solubility of between about 0.1 mg/ml and 2 mg/ml as salt. In some embodiments, the solubility of the pharmaceutical compound as salt is measured in an aqueous medium. In some embodiments, the solubility of the pharmaceutical compound as salt is measured in an organic solvent. In some embodiments, the aqueous medium comprises water. In some embodiments, the salt of the pharmaceutical compound comprises a HCl salt.

In some embodiments, the pharmaceutical compound has a solubility of less than 100 mg/ml as salt. In some embodiments, the pharmaceutical compound has a solubility of less than 90 mg/ml as salt. In some embodiments, the pharmaceutical compound has a solubility of less than 80 mg/ml as salt. In some embodiments, the pharmaceutical compound has a solubility of less than 70 mg/ml as salt. In some embodiments, the pharmaceutical compound has a solubility of less than 60 mg/ml as salt. In some embodiments, the pharmaceutical compound has a solubility of less than 50 mg/ml as salt. In some embodiments, the pharmaceutical compound has a solubility of less than 45 mg/ml as salt. In some embodiments, the pharmaceutical compound has a solubility of less than 40 mg/ml as salt. In some embodiments, the pharmaceutical compound has a solubility of less than 35 mg/ml as salt. In some embodiments, the pharmaceutical compound has a solubility of less than 30 mg/ml as salt. In some embodiments, the pharmaceutical compound has a solubility of less than 25 mg/ml as salt. In some embodiments, the pharmaceutical compound has a solubility of less than 20 mg/ml as salt. In some embodiments, the pharmaceutical compound has a solubility of less than 10 mg/ml as salt. In some embodiments, the pharmaceutical compound has a solubility of less than 9 mg/ml as salt. In some embodiments, the pharmaceutical compound has a solubility of less than 8 mg/ml as salt. In some embodiments, the pharmaceutical compound has a solubility of less than 7 mg/ml as salt. In some embodiments, the pharmaceutical compound has a solubility of less than 6 mg/ml as salt. In some embodiments, the pharmaceutical compound has a solubility of less than 5 mg/ml as salt. In some embodiments, the pharmaceutical compound has a solubility of less than 4 mg/ml as salt. In some embodiments, the pharmaceutical compound has a solubility of less than 3 mg/ml as salt. In some embodiments, the pharmaceutical compound has a solubility of more 2 mg/ml as salt. In some embodiments, the pharmaceutical compound has a solubility of less than 1 mg/ml as salt. In some embodiments, the pharmaceutical compound has a solubility of less than 0.9 mg/ml as salt. In some embodiments, the pharmaceutical compound has a solubility of less than 0.8 mg/ml as salt. In some embodiments, the pharmaceutical compound has a solubility of less than 0.7 mg/ml as salt. In some embodiments, the pharmaceutical compound has a solubility of less than 0.6 mg/ml as salt. In some embodiments, the pharmaceutical compound has a solubility of less than 0.5 mg/ml as salt. In some embodiments, the pharmaceutical compound has a solubility of less than 0.4 mg/ml as salt. In some embodiments, the pharmaceutical compound has a solubility of less than 0.3 mg/ml as salt. In some embodiments, the pharmaceutical compound has a solubility of less than 0.2 mg/ml as salt. In some embodiments, the pharmaceutical compound has a solubility of less than 0.1 mg/ml as salt. In some embodiments, the pharmaceutical compound has a solubility of less than 0.09 mg/ml as salt. In some embodiments, the pharmaceutical compound has a solubility of less than 0.08 mg/ml as salt. In some embodiments, the pharmaceutical compound has a solubility of less than 0.07 mg/ml as salt. In some embodiments, the pharmaceutical compound has a solubility of less than 0.06 mg/ml as salt. In some embodiments, the pharmaceutical compound has a solubility of less than 0.05 mg/ml as salt. In some embodiments, the pharmaceutical compound has a solubility of less than 0.04 mg/ml as salt. In some embodiments, the pharmaceutical compound has a solubility of less than 0.03 mg/ml as salt. In some embodiments, the pharmaceutical compound has a solubility of less than 0.02 mg/ml as salt. In some embodiments, the pharmaceutical compound has a solubility of less than 0.01 mg/ml as salt. In some embodiments, the pharmaceutical compound has a solubility of less than 0.009 mg/ml as salt. In some embodiments, the pharmaceutical compound has a solubility of less than 0.008 mg/ml as salt. In some embodiments, the pharmaceutical compound has a solubility of less than 0.007 mg/ml as salt. In some embodiments, the pharmaceutical compound has a solubility of less than 0.006 mg/ml as salt. In some embodiments, the pharmaceutical compound has a solubility of less than 0.005 mg/ml as salt. In some embodiments, the pharmaceutical compound has a solubility of less than 0.004 mg/ml as salt. In some embodiments, the pharmaceutical compound has a solubility of less than 0.003 mg/ml as salt. In some embodiments, the pharmaceutical compound has a solubility of less than 0.002 mg/ml as salt. In some embodiments, the pharmaceutical compound has a solubility of less than 0.001 mg/ml as salt. In some embodiments, the pharmaceutical compound has a solubility of less than 0.9 μg/ml as salt. In some embodiments, the pharmaceutical compound has a solubility of less than 0.8 μg/ml as salt. In some embodiments, the pharmaceutical compound has a solubility of less than 0.7 μg/ml as salt. In some embodiments, the pharmaceutical compound has a solubility of less than 0.6 μg/ml as salt. In some embodiments, the pharmaceutical compound has a solubility of less than 0.5 μg/ml as salt. In some embodiments, the pharmaceutical compound has a solubility of less than 0.4 μg/ml as salt. In some embodiments, the pharmaceutical compound has a solubility of less than 0.3 μg/ml as salt. In some embodiments, the pharmaceutical compound has a solubility of less than 0.2 μg/ml as salt. In some embodiments, the pharmaceutical compound has a solubility of less than 0.1 μg/ml as salt. In some embodiments, the pharmaceutical compound has a solubility of less than 0.09 μg/ml as salt. In some embodiments, the pharmaceutical compound has a solubility of less than 0.08 μg/ml as salt. In some embodiments, the pharmaceutical compound has a solubility of less than 0.07 μg/ml as salt. In some embodiments, the pharmaceutical compound has a solubility of less than 0.06 μg/ml as salt. In some embodiments, the pharmaceutical compound has a solubility of less than 0.05 μg/ml as salt. In some embodiments, the pharmaceutical compound has a solubility of less than 0.04 μg/ml as salt. In some embodiments, the pharmaceutical compound has a solubility of less than 0.03 μg/ml as salt. In some embodiments, the pharmaceutical compound has a solubility of less than 0.02 μg/ml as salt. In some embodiments, the pharmaceutical compound has a solubility of less than 0.01 μg/ml as salt. In some embodiments, the pharmaceutical compound has a solubility of less than 0.009 μg/ml as salt. In some embodiments, the pharmaceutical compound has a solubility of less than 0.008 μg/ml as salt. In some embodiments, the pharmaceutical compound has a solubility of less than 0.007 μg/ml as salt. In some embodiments, the pharmaceutical compound has a solubility of less than 0.006 μg/ml as salt. In some embodiments, the pharmaceutical compound has a solubility of less than 0.005 μg/ml as salt. In some embodiments, the pharmaceutical compound has a solubility of less than 0.004 μg/ml as salt. In some embodiments, the pharmaceutical compound has a solubility of less than 0.003 μg/ml as salt. In some embodiments, the pharmaceutical compound has a solubility of less than 0.002 μg/ml as salt. In some embodiments, the pharmaceutical compound has a solubility of less than 0.001 μg/ml as salt. In some embodiments, the solubility of the pharmaceutical compound as salt is measured in an aqueous medium. In some embodiments, the solubility of the pharmaceutical compound as salt is measured in an organic solvent. In some embodiments, the aqueous medium comprises water. In some embodiments, the salt of the pharmaceutical compound comprises a HCl salt.

In some embodiments, the pharmaceutical compound has a solubility of more than 100 mg/ml as freebase. In some embodiments, the pharmaceutical compound has a solubility of more than 90 mg/ml as freebase. In some embodiments, the pharmaceutical compound has a solubility of more than 80 mg/ml as freebase. In some embodiments, the pharmaceutical compound has a solubility of more than 70 mg/ml as freebase. In some embodiments, the pharmaceutical compound has a solubility of more than 60 mg/ml as freebase. In some embodiments, the pharmaceutical compound has a solubility of more than 50 mg/ml as freebase. In some embodiments, the pharmaceutical compound has a solubility of more than 45 mg/ml as freebase. In some embodiments, the pharmaceutical compound has a solubility of more than 40 mg/ml as freebase. In some embodiments, the pharmaceutical compound has a solubility of more than 35 mg/ml as freebase. In some embodiments, the pharmaceutical compound has a solubility of more than 30 mg/ml as freebase. In some embodiments, the pharmaceutical compound has a solubility of more than 25 mg/ml as freebase. In some embodiments, the pharmaceutical compound has a solubility of more than 20 mg/ml as freebase. In some embodiments, the pharmaceutical compound has a solubility of more than 10 mg/ml as freebase. In some embodiments, the pharmaceutical compound has a solubility of more than 9 mg/ml as freebase. In some embodiments, the pharmaceutical compound has a solubility of more than 8 mg/ml as freebase. In some embodiments, the pharmaceutical compound has a solubility of more than 7 mg/ml as freebase. In some embodiments, the pharmaceutical compound has a solubility of more than 6 mg/ml as freebase. In some embodiments, the pharmaceutical compound has a solubility of more than 5 mg/ml as freebase. In some embodiments, the pharmaceutical compound has a solubility of more than 4 mg/ml as freebase. In some embodiments, the pharmaceutical compound has a solubility of more than 3 mg/ml as freebase. In some embodiments, the pharmaceutical compound has a solubility of more 2 mg/ml as freebase. In some embodiments, the pharmaceutical compound has a solubility of more than 1 mg/ml as freebase. In some embodiments, the pharmaceutical compound has a solubility of more than 0.9 mg/ml as freebase. In some embodiments, the pharmaceutical compound has a solubility of more than 0.8 mg/ml as freebase. In some embodiments, the pharmaceutical compound has a solubility of more than 0.7 mg/ml as freebase. In some embodiments, the pharmaceutical compound has a solubility of more than 0.6 mg/ml as freebase. In some embodiments, the pharmaceutical compound has a solubility of more than 0.5 mg/ml as freebase. In some embodiments, the pharmaceutical compound has a solubility of more than 0.4 mg/ml as freebase. In some embodiments, the pharmaceutical compound has a solubility of more than 0.3 mg/ml as freebase. In some embodiments, the pharmaceutical compound has a solubility of more than 0.2 mg/ml as freebase. In some embodiments, the pharmaceutical compound has a solubility of more than 0.1 mg/ml as freebase. In some embodiments, the pharmaceutical compound has a solubility of more than 0.09 mg/ml as freebase. In some embodiments, the pharmaceutical compound has a solubility of more than 0.08 mg/ml as freebase. In some embodiments, the pharmaceutical compound has a solubility of more than 0.07 mg/ml as freebase. In some embodiments, the pharmaceutical compound has a solubility of more than 0.06 mg/ml as freebase. In some embodiments, the pharmaceutical compound has a solubility of more than 0.05 mg/ml as freebase. In some embodiments, the pharmaceutical compound has a solubility of more than 0.04 mg/ml as freebase. In some embodiments, the pharmaceutical compound has a solubility of more than 0.03 mg/ml as freebase. In some embodiments, the pharmaceutical compound has a solubility of more than 0.02 mg/ml as freebase. In some embodiments, the pharmaceutical compound has a solubility of more than 0.01 mg/ml as freebase. In some embodiments, the pharmaceutical compound has a solubility of more than 0.009 mg/ml as freebase. In some embodiments, the pharmaceutical compound has a solubility of more than 0.008 mg/ml as freebase. In some embodiments, the pharmaceutical compound has a solubility of more than 0.007 mg/ml as freebase. In some embodiments, the pharmaceutical compound has a solubility of more than 0.006 mg/ml as freebase. In some embodiments, the pharmaceutical compound has a solubility of more than 0.005 mg/ml as freebase. In some embodiments, the pharmaceutical compound has a solubility of more than 0.004 mg/ml as freebase. In some embodiments, the pharmaceutical compound has a solubility of more than 0.003 mg/ml as freebase. In some embodiments, the pharmaceutical compound has a solubility of more than 0.002 mg/ml as freebase. In some embodiments, the pharmaceutical compound has a solubility of more than 0.001 mg/ml as freebase. In some embodiments, the pharmaceutical compound has a solubility of more than 0.9 μg/ml as freebase. In some embodiments, the pharmaceutical compound has a solubility of more than 0.8 μg/ml as freebase. In some embodiments, the pharmaceutical compound has a solubility of more than 0.7 μg/ml as freebase. In some embodiments, the pharmaceutical compound has a solubility of more than 0.6 μg/ml as freebase. In some embodiments, the pharmaceutical compound has a solubility of more than 0.5 μg/ml as freebase. In some embodiments, the pharmaceutical compound has a solubility of more than 0.4 μg/ml as freebase. In some embodiments, the pharmaceutical compound has a solubility of more than 0.3 μg/ml as freebase. In some embodiments, the pharmaceutical compound has a solubility of more than 0.2 μg/ml as freebase. In some embodiments, the pharmaceutical compound has a solubility of more than 0.1 μg/ml as freebase. In some embodiments, the pharmaceutical compound has a solubility of more than 0.09 μg/ml as freebase. In some embodiments, the pharmaceutical compound has a solubility of more than 0.08 μg/ml as freebase. In some embodiments, the pharmaceutical compound has a solubility of more than 0.07 μg/ml as freebase. In some embodiments, the pharmaceutical compound has a solubility of more than 0.06 μg/ml as freebase. In some embodiments, the pharmaceutical compound has a solubility of more than 0.05 μg/ml as freebase. In some embodiments, the pharmaceutical compound has a solubility of more than 0.04 μg/ml as freebase. In some embodiments, the pharmaceutical compound has a solubility of more than 0.03 μg/ml as freebase. In some embodiments, the pharmaceutical compound has a solubility of more than 0.02 μg/ml as freebase. In some embodiments, the pharmaceutical compound has a solubility of more than 0.01 μg/ml as freebase. In some embodiments, the pharmaceutical compound has a solubility of more than 0.009 μg/ml as freebase. In some embodiments, the pharmaceutical compound has a solubility of more than 0.008 μg/ml as freebase. In some embodiments, the pharmaceutical compound has a solubility of more than 0.007 μg/ml as freebase. In some embodiments, the pharmaceutical compound has a solubility of more than 0.006 μg/ml as freebase. In some embodiments, the pharmaceutical compound has a solubility of more than 0.005 μg/ml as freebase. In some embodiments, the pharmaceutical compound has a solubility of more than 0.004 μg/ml as freebase. In some embodiments, the pharmaceutical compound has a solubility of more than 0.003 μg/ml as freebase. In some embodiments, the pharmaceutical compound has a solubility of more than 0.002 μg/ml as freebase. In some embodiments, the pharmaceutical compound has a solubility of more than 0.001 μg/ml as freebase. In some embodiments, the solubility of the pharmaceutical compound as freebase is measured in an aqueous medium. In some embodiments, the solubility of the pharmaceutical compound as freebase is measured in an organic solvent. In some embodiments, the aqueous medium comprises water.

In some embodiments, the pharmaceutical compound has a solubility of between about 0.001 mg/ml and 100 mg/ml as freebase. In some embodiments, the pharmaceutical compound has a solubility of between about 0.01 mg/ml and 50 mg/ml as freebase. In some embodiments, the pharmaceutical compound has a solubility of between about 0.02 mg/ml and 40 mg/ml as freebase. In some embodiments, the pharmaceutical compound has a solubility of between about 0.03 mg/ml and 30 mg/ml as freebase. In some embodiments, the pharmaceutical compound has a solubility of between about 0.04 mg/ml and 20 mg/ml as freebase. In some embodiments, the pharmaceutical compound has a solubility of between about 0.05 mg/ml and 10 mg/ml as freebase. In some embodiments, the pharmaceutical compound has a solubility of between about 0.06 mg/ml and 5 mg/ml as freebase. In some embodiments, the pharmaceutical compound has a solubility of between about 0.07 mg/ml and 4 mg/ml as freebase. In some embodiments, the pharmaceutical compound has a solubility of between about 0.09 mg/ml and 3 mg/ml as freebase. In some embodiments, the pharmaceutical compound has a solubility of between about 0.1 mg/ml and 2 mg/ml as freebase. In some embodiments, the solubility of the pharmaceutical compound as freebase is measured in an aqueous medium. In some embodiments, the solubility of the pharmaceutical compound as freebase is measured in an organic solvent. In some embodiments, the aqueous medium comprises water.

In some embodiments, the pharmaceutical compound has a solubility of less than 100 mg/ml as freebase. In some embodiments, the pharmaceutical compound has a solubility of less than 90 mg/ml as freebase. In some embodiments, the pharmaceutical compound has a solubility of less than 80 mg/ml as freebase. In some embodiments, the pharmaceutical compound has a solubility of less than 70 mg/ml as freebase. In some embodiments, the pharmaceutical compound has a solubility of less than 60 mg/ml as freebase. In some embodiments, the pharmaceutical compound has a solubility of less than 50 mg/ml as freebase. In some embodiments, the pharmaceutical compound has a solubility of less than 45 mg/ml as freebase. In some embodiments, the pharmaceutical compound has a solubility of less than 40 mg/ml as freebase. In some embodiments, the pharmaceutical compound has a solubility of less than 35 mg/ml as freebase. In some embodiments, the pharmaceutical compound has a solubility of less than 30 mg/ml as freebase. In some embodiments, the pharmaceutical compound has a solubility of less than 25 mg/ml as freebase. In some embodiments, the pharmaceutical compound has a solubility of less than 20 mg/ml as freebase. In some embodiments, the pharmaceutical compound has a solubility of less than 10 mg/ml as freebase. In some embodiments, the pharmaceutical compound has a solubility of less than 9 mg/ml as freebase. In some embodiments, the pharmaceutical compound has a solubility of less than 8 mg/ml as freebase. In some embodiments, the pharmaceutical compound has a solubility of less than 7 mg/ml as freebase. In some embodiments, the pharmaceutical compound has a solubility of less than 6 mg/ml as freebase. In some embodiments, the pharmaceutical compound has a solubility of less than 5 mg/ml as freebase. In some embodiments, the pharmaceutical compound has a solubility of less than 4 mg/ml as freebase. In some embodiments, the pharmaceutical compound has a solubility of less than 3 mg/ml as freebase. In some embodiments, the pharmaceutical compound has a solubility of more 2 mg/ml as freebase. In some embodiments, the pharmaceutical compound has a solubility of less than 1 mg/ml as freebase. In some embodiments, the pharmaceutical compound has a solubility of less than 0.9 mg/ml as freebase. In some embodiments, the pharmaceutical compound has a solubility of less than 0.8 mg/ml as freebase. In some embodiments, the pharmaceutical compound has a solubility of less than 0.7 mg/ml as freebase. In some embodiments, the pharmaceutical compound has a solubility of less than 0.6 mg/ml as freebase. In some embodiments, the pharmaceutical compound has a solubility of less than 0.5 mg/ml as freebase. In some embodiments, the pharmaceutical compound has a solubility of less than 0.4 mg/ml as freebase. In some embodiments, the pharmaceutical compound has a solubility of less than 0.3 mg/ml as freebase. In some embodiments, the pharmaceutical compound has a solubility of less than 0.2 mg/ml as freebase. In some embodiments, the pharmaceutical compound has a solubility of less than 0.1 mg/ml as freebase. In some embodiments, the pharmaceutical compound has a solubility of less than 0.09 mg/ml as freebase. In some embodiments, the pharmaceutical compound has a solubility of less than 0.08 mg/ml as freebase. In some embodiments, the pharmaceutical compound has a solubility of less than 0.07 mg/ml as freebase. In some embodiments, the pharmaceutical compound has a solubility of less than 0.06 mg/ml as freebase. In some embodiments, the pharmaceutical compound has a solubility of less than 0.05 mg/ml as freebase. In some embodiments, the pharmaceutical compound has a solubility of less than 0.04 mg/ml as freebase. In some embodiments, the pharmaceutical compound has a solubility of less than 0.03 mg/ml as freebase. In some embodiments, the pharmaceutical compound has a solubility of less than 0.02 mg/ml as freebase. In some embodiments, the pharmaceutical compound has a solubility of less than 0.01 mg/ml as freebase. In some embodiments, the pharmaceutical compound has a solubility of less than 0.009 mg/ml as freebase. In some embodiments, the pharmaceutical compound has a solubility of less than 0.008 mg/ml as freebase. In some embodiments, the pharmaceutical compound has a solubility of less than 0.007 mg/ml as freebase. In some embodiments, the pharmaceutical compound has a solubility of less than 0.006 mg/ml as freebase. In some embodiments, the pharmaceutical compound has a solubility of less than 0.005 mg/ml as freebase. In some embodiments, the pharmaceutical compound has a solubility of less than 0.004 mg/ml as freebase. In some embodiments, the pharmaceutical compound has a solubility of less than 0.003 mg/ml as freebase. In some embodiments, the pharmaceutical compound has a solubility of less than 0.002 mg/ml as freebase. In some embodiments, the pharmaceutical compound has a solubility of less than 0.001 mg/ml as freebase. In some embodiments, the pharmaceutical compound has a solubility of less than 0.9 μg/ml as freebase. In some embodiments, the pharmaceutical compound has a solubility of less than 0.8 μg/ml as freebase. In some embodiments, the pharmaceutical compound has a solubility of less than 0.7 μg/ml as freebase. In some embodiments, the pharmaceutical compound has a solubility of less than 0.6 μg/ml as freebase. In some embodiments, the pharmaceutical compound has a solubility of less than 0.5 μg/ml as freebase. In some embodiments, the pharmaceutical compound has a solubility of less than 0.4 μg/ml as freebase. In some embodiments, the pharmaceutical compound has a solubility of less than 0.3 μg/ml as freebase. In some embodiments, the pharmaceutical compound has a solubility of less than 0.2 μg/ml as freebase. In some embodiments, the pharmaceutical compound has a solubility of less than 0.1 μg/ml as freebase. In some embodiments, the pharmaceutical compound has a solubility of less than 0.09 μg/ml as freebase. In some embodiments, the pharmaceutical compound has a solubility of less than 0.08 μg/ml as freebase. In some embodiments, the pharmaceutical compound has a solubility of less than 0.07 μg/ml as freebase. In some embodiments, the pharmaceutical compound has a solubility of less than 0.06 μg/ml as freebase. In some embodiments, the pharmaceutical compound has a solubility of less than 0.05 μg/ml as freebase. In some embodiments, the pharmaceutical compound has a solubility of less than 0.04 μg/ml as freebase. In some embodiments, the pharmaceutical compound has a solubility of less than 0.03 μg/ml as freebase. In some embodiments, the pharmaceutical compound has a solubility of less than 0.02 μg/ml as freebase. In some embodiments, the pharmaceutical compound has a solubility of less than 0.01 μg/ml as freebase. In some embodiments, the pharmaceutical compound has a solubility of less than 0.009 μg/ml as freebase. In some embodiments, the pharmaceutical compound has a solubility of less than 0.008 μg/ml as freebase. In some embodiments, the pharmaceutical compound has a solubility of less than 0.007 μg/ml as freebase. In some embodiments, the pharmaceutical compound has a solubility of less than 0.006 μg/ml as freebase. In some embodiments, the pharmaceutical compound has a solubility of less than 0.005 μg/ml as freebase. In some embodiments, the pharmaceutical compound has a solubility of less than 0.004 μg/ml as freebase. In some embodiments, the pharmaceutical compound has a solubility of less than 0.003 μg/ml as freebase. In some embodiments, the pharmaceutical compound has a solubility of less than 0.002 μg/ml as freebase. In some embodiments, the pharmaceutical compound has a solubility of less than 0.001 μg/ml as freebase. In some embodiments, the solubility of the pharmaceutical compound as freebase is measured in an aqueous medium. In some embodiments, the solubility of the pharmaceutical compound as freebase is measured in an organic solvent. In some embodiments, the aqueous medium comprises water.

In some embodiments, the solubility of the pharmaceutical compound in the pharmaceutical composition is higher than (i) the solubility of the pharmaceutical compound as a salt; or (ii) the solubility of the pharmaceutical compound in a composition comprising the pharmaceutical compound in freebase form that is complexing to a non-polar pore of the complexing agent, wherein the pharmaceutical compound has the same concentration in the pharmaceutical composition, the pharmaceutical compound as a salt, and the a composition comprising the pharmaceutical compound in freebase form. In some embodiments, the solubility of the pharmaceutical compound in the pharmaceutical composition is higher than the solubility of the pharmaceutical compound in a composition comprising the pharmaceutical compound and complexing agent with a higher molar ratio of the pharmaceutical compound to the complexing agent, wherein the pharmaceutical compound has the same concentration in the pharmaceutical composition and the a composition with a higher molar ratio of the pharmaceutical compound to the complexing agent. In some embodiments, the solubility of the pharmaceutical compound in the pharmaceutical composition has an increase of at least 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, or 100-fold compared with (i) the solubility of the pharmaceutical compound as a salt; or (ii) the solubility of the pharmaceutical compound in a composition comprising the pharmaceutical compound in freebase form that is complexing to a non-polar pore of the complexing agent, wherein the pharmaceutical compound has the same concentration in the pharmaceutical composition, the pharmaceutical compound as a salt, and the composition comprising the pharmaceutical compound in freebase form. In some embodiments, the solubility of the pharmaceutical compound in the pharmaceutical composition has an increase of at least 2-fold, 50-fold, 60-fold, 70-fold, 80-fold, 90-fold, or 100-fold compared with the solubility of the pharmaceutical compound in a composition comprising the pharmaceutical compound and complexing agent with a higher molar ratio of the pharmaceutical compound to the complexing agent, wherein the pharmaceutical compound has the same concentration in the pharmaceutical composition and the composition with a higher molar ratio of the pharmaceutical compound to the complexing agent.

In an aspect, provided herein is a pharmaceutical composition, comprising: (i) a pharmaceutical compound, or an enantiomer, a mixture of enantiomers, or an isotopic variant thereof, wherein the pharmaceutical compound comprises a prodrug comprising an unionized substance conjugated to a chemical entity, wherein the chemical entity comprises a protonated nitrogen atom; and (ii) a complexing agent, wherein the complexing agent is an acid-substituted cyclodextrin comprising a plurality of acidic functional groups, wherein the plurality of acidic functional groups comprise an acidic group which acts as a counterion for the protonated nitrogen atom of the pharmaceutical compound.

In some embodiments, the prodrug comprises esters of carboxylic acid groups between the unionized substance and the chemical entity. In some embodiments, the prodrug is converted in vivo by the action of biochemicals within the patient's body, such as enzymes, to an active pharmaceutical ingredient. In some embodiments, the esters of carboxylic acid groups can be hydrolyzed by endogenous esterases as are found in the bloodstream of humans and other mammals. Further examples of prodrugs include boronate esters which can be hydrolyzed under physiological conditions to afford the corresponding boronic acid. In some embodiments, the unionized substance comprises brexanolone. In some embodiments, the chemical entity comprises γ-aminobutyric acid (GABA).

In some embodiments, the plurality of acidic functional groups comprise an acidic group which acts as a counterion for the protonated nitrogen atom of the chemical entity of the pharmaceutical compound. In some embodiments, the acidic group is the conjugate base of the acidic group. In some embodiments, the acidic group is a carboxylic acid or carboxylate. In some embodiments, the acidic group is a carboxylate. In some embodiments, the acidic group is a sulfonic acid or sulfonate. In some embodiments, the acidic group is a sulfonate. In some embodiments, the conjugate base of the complexing agent acts as the counterion for a plurality of the pharmaceutical compound. In some embodiments, each acidic group of the plurality of acidic functional groups acts as a counterion for a plurality of the pharmaceutical compound. In some embodiments, each acidic group of the plurality of acidic functional groups acts as a counterion for a protonated amine of a plurality of the pharmaceutical compound. In some embodiments, each of the plurality of acidic functional groups acts as a counterion for a pronated amine.

In some embodiments, the complexing agent is a cyclodextrin substituted with the plurality of acidic functional group. In some embodiments, the plurality of acidic functional groups is a carboxylic acid, sulfonic acid, sulfonic acid, phosphonic acid, or any combination thereof. In some embodiments, the cyclodextrin is substituted with at least 1, at least 2, at least 3, at least 4, at least 5, or at least 6 acidic functional groups. In some embodiments, the cyclodextrin is substituted with 3 to 8 acidic functional groups, 3 to 7 acidic functional groups, 4 to 8 acidic functional groups, 4 to 7 acidic functional groups, 5 to 8 acidic functional groups, 6 to 8 acidic functional groups, or 7 to 8 acidic functional groups.

In some embodiments, the complexing agent is a substituted cyclodextrin. In some cases, substituted cyclodextrins provided herein are complex mixtures wherein individual cyclodextrin molecules may comprise different numbers of substituents from other individual cyclodextrin molecules. In such cases, the number of substituents (e.g. the number of acidic functional groups) described as being present on the cyclodextrins provided herein may refer to an average degree of substitution of the mixture. For example, when a cyclodextrin is described as substituted with 3 to 8 acidic functional groups, it is intended that a complex mixture of cyclodextrins having an average degree of substitution from 3 to 8 acidic functional groups is covered. The average degree of substitution need not be an integer value and will often be a decimal value. For example, commercially available SBEBCD has an average degree of substitution of about 6.5.

In some embodiments, the complexing agent is a substituted cyclodextrin. In some embodiments, the substituted cyclodextrin is substituted with one or more acidic functional groups, or a pharmaceutically acceptable salt thereof. In some embodiments, the substituted cyclodextrin is substituted with a plurality of carboxylic acid, sulfonic acid, sulfonic acid, or phosphonic acid functional groups. In some embodiments, the cyclodextrin is substituted with at least 1, at least 2, at least 3, at least 4, at least 5, or at least 6 acidic functional groups. In some embodiments, the cyclodextrin is substituted with 3 to 8 acidic functional groups, 3 to 7 acidic functional groups, 4 to 8 acidic functional groups, 4 to 7 acidic functional groups, 5 to 8 acidic functional groups, 6 to 8 acidic functional groups, or 7 to 8 acidic functional groups.

In some embodiments, the molar ratio of the cyclodextrin to the pharmaceutical compound comprising a protonated nitrogen atom is from about 1:4 to about 1:8. In some embodiments, the molar ratio of the cyclodextrin to the pharmaceutical compound comprising a protonated nitrogen atom is from about 1:4 to about 1:10. In some embodiments, the molar ratio of the cyclodextrin to the pharmaceutical compound comprising a protonated nitrogen atom is from about 1:5 to about 1:7. In some embodiments, the molar ratio of the cyclodextrin to the pharmaceutical compound comprising a protonated nitrogen atom is about 1:4. In some embodiments, the molar ratio of the cyclodextrin to the pharmaceutical compound comprising a protonated nitrogen atom is about 1:5. In some embodiments, the molar ratio of the cyclodextrin to the pharmaceutical compound comprising a protonated nitrogen atom is about 1:6. In some embodiments, the molar ratio of the cyclodextrin to the pharmaceutical compound comprising a protonated nitrogen atom is about 1:7. In some embodiments, the molar ratio of the cyclodextrin to the pharmaceutical compound comprising a protonated nitrogen atom is about 1:8. In some embodiments, the molar ratio of the cyclodextrin to the pharmaceutical compound comprising a protonated nitrogen atom is about 1:9. In some embodiments, the molar ratio of the cyclodextrin to the pharmaceutical compound comprising a protonated nitrogen atom is about 1:10.

In some embodiments, molar ratio of acidic functional groups of the complexing agent to the pharmaceutical compound comprising a protonated nitrogen atom is from about 2:1 to about 1:2. In some embodiments, molar ratio of acidic functional groups of the complexing agent to the pharmaceutical compound comprising a protonated nitrogen atom is from about 1.75:1 to about 1:1.75. In some embodiments, molar ratio of acidic functional groups of the complexing agent to the pharmaceutical compound comprising a protonated nitrogen atom is from about 1.5:1 to about 1:1.5. In some embodiments, molar ratio of acidic functional groups of the complexing agent to the pharmaceutical compound comprising a protonated nitrogen atom is from about 1.4:1 to about 1:1.4. In some embodiments, molar ratio of acidic functional groups of the complexing agent to the pharmaceutical compound comprising a protonated nitrogen atom is from 1.3:1 to about 1:1.3. In some embodiments, molar ratio of acidic functional groups of the complexing agent to the pharmaceutical compound comprising a protonated nitrogen atom is from about 1.25:1 to about 1:1.25. In some embodiments, molar ratio of acidic functional groups of the complexing agent to the pharmaceutical compound comprising a protonated nitrogen atom is from about 1.2:1 to about 1:1.2. In some embodiments, molar ratio of acidic functional groups of the complexing agent to the pharmaceutical compound comprising a protonated nitrogen atom is from about 1.15:1 to about 1:1.15. In some embodiments, molar ratio of acidic functional groups of the complexing agent to the pharmaceutical compound comprising a protonated nitrogen atom is from about 1.1:1 to about 1:1.1. In some embodiments, molar ratio of acidic functional groups of the complexing agent to the pharmaceutical compound comprising a protonated nitrogen atom is from about 1.05:1 to about 1:1.05. In some embodiments, molar ratio of acidic functional groups of the complexing agent to the pharmaceutical compound comprising a protonated nitrogen atom is about 1:1.

In some embodiments, the pharmaceutical composition comprises excess ions. In some embodiments, the excess ions comprise a salt, e.g., sodium chloride, lithium chloride, potassium chloride, or sodium bromide. In some embodiments, an excess ion acts as a counterion to an acidic functional group of the complexing agent. In some embodiments, such as an excess sodium ion occupying a deprotonated acidic site.

In some embodiments, the complexing agent further comprises a non-polar region. In some embodiments, the non-polar region is a non-polar pore. In some embodiments, the pharmaceutical composition comprises an additional molar equivalent of the unionized substance. In some embodiments, the additional molar equivalent of the unionized substance is complexed to the non-polar region of the complexing agent. In some embodiments, the additional molar equivalent of the unionized substance is complexed to the non-polar pore of the complexing agent.

In some embodiments, the pharmaceutical composition comprises 0.1-20 molar equivalents of the unionized substance compared to the complexing agent. In some embodiments, the pharmaceutical composition comprises 0.2-15 molar equivalents of unionized substance compared to the complexing agent. In some embodiments, the pharmaceutical composition comprises 0.5-10 molar equivalents of the unionized substance compared to the complexing agent. In some embodiments, the pharmaceutical composition comprises 1-5 molar equivalents of the unionized substance compared to the complexing agent. In some embodiments, the pharmaceutical composition comprises 0.1-20 molar equivalents of the unionized substance compared to the complexing agent, e.g., about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, or 20.0 molar equivalents of the unionized substance, or any amount therebetween. In some embodiments, the pharmaceutical composition comprises 1-2 molar equivalents of the unionized substance compared to the complexing agent. In some embodiments, the pharmaceutical composition comprises about 1.1, 1.2, 1.3, 1.4, 1.5, 1.6, 1.7, 1.8, 1.9, or about 2 molar equivalents of the unionized substance compared to the complexing agent.

In some embodiments, the solubility of the ununionized substance in the pharmaceutical composition is higher than (i) the solubility of the unionized substance as a salt; or (ii) the solubility of the unionized substance in a composition comprising the unionized substance in freebase form that is complexing to a non-polar pore of the complexing agent, wherein the unionized substance has the same concentration in the pharmaceutical composition, the unionized substance as a salt, and the composition comprising the unionized substance in freebase form. In some embodiments, the solubility of the unionized substance in the pharmaceutical composition is higher than the solubility of the unionized substance in a composition comprising the unionized substance and complexing agent with a higher molar ratio of the unionized substance to the complexing agent, wherein the unionized substance has the same concentration in the pharmaceutical composition and the composition with a higher molar ratio of the unionized substance to the complexing agent. In some embodiments, the solubility of the unionized substance in the pharmaceutical composition has an increase of at least 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, or 100-fold compared with (i) the solubility of the unionized substance as a salt; or (ii) the solubility of the unionized substance in a composition comprising the unionized substance in freebase form that is complexing to a non-polar pore of the complexing agent, wherein the unionized substance has the same concentration in the pharmaceutical composition, the unionized substance as a salt, and the composition comprising the unionized substance in freebase form. In some embodiments, the solubility of the unionized substance in the pharmaceutical composition has an increase of at least 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, or 100-fold compared with the solubility of the unionized substance in a composition comprising the unionized substance and complexing agent with a higher molar ratio of the unionized substance to the complexing agent, wherein the unionized substance has the same concentration in the pharmaceutical composition and the composition with a higher molar ratio of the unionized substance to the complexing agent.

In an aspect, provided herein is a pharmaceutical composition, comprising (i) a pharmaceutical compound, or an enantiomer, a mixture of enantiomers, or an isotopic variant thereof, wherein the pharmaceutical compound comprises a prodrug comprising an unionized substance conjugated to a chemical entity, wherein the chemical entity comprises a protonated nitrogen atom; (ii) a complexing agent, wherein the complexing agent is an acid-substituted cyclodextrin comprising a plurality of acidic functional groups, wherein the plurality of acidic functional groups comprise an acidic group which acts as a counterion for the protonated nitrogen atom of the pharmaceutical compound; and (iii) an additional molar equivalent of the unionized substance compared to the complexing agent.

In some embodiments, the prodrug comprises esters of carboxylic acid groups between the unionized substance and the chemical entity. In some embodiments, the prodrug is converted in vivo by the action of biochemicals within the patient's body, such as enzymes, to an active pharmaceutical ingredient. In some embodiments, the esters of carboxylic acid groups can be hydrolyzed by endogenous esterases as are found in the bloodstream of humans and other mammals. Further examples of prodrugs include boronate esters which can be hydrolyzed under physiological conditions to afford the corresponding boronic acid. In some embodiments, the unionized substance comprises brexanolone. In some embodiments, the chemical entity comprises γ-aminobutyric acid (GABA).

In some embodiments, the complexing agent further comprises a non-polar region. In some embodiments, the non-polar region is a non-polar pore. In some embodiments, the additional molar equivalent of the unionized substance is complexed to the non-polar region of the complexing agent. In some embodiments, the additional molar equivalent of the unionized substance is complexed to the non-polar pore of the complexing agent.

In some embodiments, the pharmaceutical composition comprises 0.1-20 molar equivalents of the unionized substance compared to the complexing agent. In some embodiments, the pharmaceutical composition comprises 0.2-15 molar equivalents of unionized substance compared to the complexing agent. In some embodiments, the pharmaceutical composition comprises 0.5-10 molar equivalents of the unionized substance compared to the complexing agent. In some embodiments, the pharmaceutical composition comprises 1-5 molar equivalents of the unionized substance compared to the complexing agent. In some embodiments, the pharmaceutical composition comprises 0.1-20 molar equivalents of the unionized substance compared to the complexing agent, e.g., about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, or 20.0 molar equivalents of the unionized substance, or any amount therebetween. In some embodiments, the pharmaceutical composition comprises 1-2 molar equivalents of the unionized substance compared to the complexing agent. In some embodiments, the pharmaceutical composition comprises about 1.1, 1.2, 1.3, 1.4, 1.5, 1.6, 1.7, 1.8, 1.9, or about 2 molar equivalents of the unionized substance compared to the complexing agent.

In some embodiments, the molar ratio of the cyclodextrin to the pharmaceutical compound comprising a protonated nitrogen atom is from about 1:4 to about 1:8. In some embodiments, the molar ratio of the cyclodextrin to the pharmaceutical compound comprising a protonated nitrogen atom is from about 1:4 to about 1:10. In some embodiments, the molar ratio of the cyclodextrin to the pharmaceutical compound comprising a protonated nitrogen atom is from about 1:5 to about 1:7. In some embodiments, the molar ratio of the cyclodextrin to the pharmaceutical compound comprising a protonated nitrogen atom is about 1:4. In some embodiments, the molar ratio of the cyclodextrin to the pharmaceutical compound comprising a protonated nitrogen atom is about 1:5. In some embodiments, the molar ratio of the cyclodextrin to the pharmaceutical compound comprising a protonated nitrogen atom is about 1:6. In some embodiments, the molar ratio of the cyclodextrin to the pharmaceutical compound comprising a protonated nitrogen atom is about 1:7. In some embodiments, the molar ratio of the cyclodextrin to the pharmaceutical compound comprising a protonated nitrogen atom is about 1:8. In some embodiments, the molar ratio of the cyclodextrin to the pharmaceutical compound comprising a protonated nitrogen atom is about 1:9. In some embodiments, the molar ratio of the cyclodextrin to the pharmaceutical compound comprising a protonated nitrogen atom is about 1:10.

In some embodiments, molar ratio of acidic functional groups of the complexing agent to the pharmaceutical compound comprising a protonated nitrogen atom is from about 2:1 to about 1:2. In some embodiments, molar ratio of acidic functional groups of the complexing agent to the pharmaceutical compound comprising a protonated nitrogen atom is from about 1.75:1 to about 1:1.75. In some embodiments, molar ratio of acidic functional groups of the complexing agent to the pharmaceutical compound comprising a protonated nitrogen atom is from about 1.5:1 to about 1:1.5. In some embodiments, molar ratio of acidic functional groups of the complexing agent to the pharmaceutical compound comprising a protonated nitrogen atom is from about 1.4:1 to about 1:1.4. In some embodiments, molar ratio of acidic functional groups of the complexing agent to the pharmaceutical compound comprising a protonated nitrogen atom is from 1.3:1 to about 1:1.3. In some embodiments, molar ratio of acidic functional groups of the complexing agent to the pharmaceutical compound comprising a protonated nitrogen atom is from about 1.25:1 to about 1:1.25. In some embodiments, molar ratio of acidic functional groups of the complexing agent to the pharmaceutical compound comprising a protonated nitrogen atom is from about 1.2:1 to about 1:1.2. In some embodiments, molar ratio of acidic functional groups of the complexing agent to the pharmaceutical compound comprising a protonated nitrogen atom is from about 1.15:1 to about 1:1.15. In some embodiments, molar ratio of acidic functional groups of the complexing agent to the pharmaceutical compound comprising a protonated nitrogen atom is from about 1.1:1 to about 1:1.1. In some embodiments, molar ratio of acidic functional groups of the complexing agent to the pharmaceutical compound comprising a protonated nitrogen atom is from about 1.05:1 to about 1:1.05. In some embodiments, molar ratio of acidic functional groups of the complexing agent to the pharmaceutical compound comprising a protonated nitrogen atom is about 1:1.

In some embodiments, the solubility of the ununionized substance in the pharmaceutical composition is higher than (i) the solubility of the unionized substance as a salt; or (ii) the solubility of the unionized substance in a composition comprising the unionized substance in freebase form that is complexing to a non-polar pore of the complexing agent, wherein the unionized substance has the same concentration in the pharmaceutical composition, the unionized substance as a salt, and the composition comprising the unionized substance in freebase form. In some embodiments, the solubility of the unionized substance in the pharmaceutical composition is higher than the solubility of the unionized substance in a composition comprising the unionized substance and complexing agent with a higher molar ratio of the unionized substance to the complexing agent, wherein the unionized substance has the same concentration in the pharmaceutical composition and the composition with a higher molar ratio of the unionized substance to the complexing agent. In some embodiments, the solubility of the unionized substance in the pharmaceutical composition has an increase of at least 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, or 100-fold compared with (i) the solubility of the unionized substance as a salt; or (ii) the solubility of the unionized substance in a composition comprising the unionized substance in freebase form that is complexing to a non-polar pore of the complexing agent, wherein the unionized substance has the same concentration in the pharmaceutical composition, the unionized substance as a salt, and the composition comprising the unionized substance in freebase form. In some embodiments, the solubility of the unionized substance in the pharmaceutical composition has an increase of at least 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, or 100-fold compared with the solubility of the unionized substance in a composition comprising the unionized substance and complexing agent with a higher molar ratio of the unionized substance to the complexing agent, wherein the unionized substance has the same concentration in the pharmaceutical composition and the composition with a higher molar ratio of the unionized substance to the complexing agent.

In an aspect, provided herein is a pharmaceutical composition, comprising (i) a pharmaceutical compound, or an enantiomer, a mixture of enantiomers, or an isotopic variant thereof, wherein the pharmaceutical compound comprises a prodrug comprising an unionized substance conjugated to a chemical entity, wherein the chemical entity comprises a protonated nitrogen atom; (ii) a complexing agent, wherein the complexing agent is an acid-substituted cyclodextrin comprising a plurality of acidic functional groups, wherein the plurality of acidic functional groups comprise an acidic group which acts as a counterion for the protonated nitrogen atom of the pharmaceutical compound, wherein the pharmaceutical composition has a ratio of the pharmaceutical compound to the complexing agent that is from about 1:1 to 1:4.

In an aspect, provided herein is a pharmaceutical composition, comprising (i) a pharmaceutical compound, or an enantiomer, a mixture of enantiomers, or an isotopic variant thereof, wherein the pharmaceutical compound comprises a prodrug comprising an unionized substance conjugated to a chemical entity, wherein the chemical entity comprises a protonated nitrogen atom; (ii) a complexing agent, wherein the complexing agent is an acid-substituted cyclodextrin comprising a plurality of acidic functional groups, wherein the plurality of acidic functional groups comprise an acidic group which acts as a counterion for the protonated nitrogen atom of the pharmaceutical compound, wherein the pharmaceutical composition has a ratio of the pharmaceutical compound to the complexing agent that is from about 1:1 to 1:4; and (iii) an additional molar equivalent of the unionized substance compared to the complexing agent.

In some embodiments, the prodrug comprises esters of carboxylic acid groups between the unionized substance and the chemical entity. In some embodiments, the prodrug is converted in vivo by the action of biochemicals within the patient's body, such as enzymes, to an active pharmaceutical ingredient. In some embodiments, the esters of carboxylic acid groups can be hydrolyzed by endogenous esterases as are found in the bloodstream of humans and other mammals. Further examples of prodrugs include boronate esters which can be hydrolyzed under physiological conditions to afford the corresponding boronic acid. In some embodiments, the unionized substance comprises brexanolone. In some embodiments, the chemical entity comprises γ-aminobutyric acid (GABA).

In some embodiments, the molar ratio of the cyclodextrin to the pharmaceutical compound comprising a protonated nitrogen atom is from about 1:4 to about 1:8. In some embodiments, the molar ratio of the cyclodextrin to the pharmaceutical compound comprising a protonated nitrogen atom is from about 1:4 to about 1:10. In some embodiments, the molar ratio of the cyclodextrin to the pharmaceutical compound comprising a protonated nitrogen atom is from about 1:5 to about 1:7. In some embodiments, the molar ratio of the cyclodextrin to the pharmaceutical compound comprising a protonated nitrogen atom is about 1:4. In some embodiments, the molar ratio of the cyclodextrin to the pharmaceutical compound comprising a protonated nitrogen atom is about 1:5. In some embodiments, the molar ratio of the cyclodextrin to the pharmaceutical compound comprising a protonated nitrogen atom is about 1:6. In some embodiments, the molar ratio of the cyclodextrin to the pharmaceutical compound comprising a protonated nitrogen atom is about 1:7. In some embodiments, the molar ratio of the cyclodextrin to the pharmaceutical compound comprising a protonated nitrogen atom is about 1:8. In some embodiments, the molar ratio of the cyclodextrin to the pharmaceutical compound comprising a protonated nitrogen atom is about 1:9. In some embodiments, the molar ratio of the cyclodextrin to the pharmaceutical compound comprising a protonated nitrogen atom is about 1:10.

In some embodiments, molar ratio of acidic functional groups of the complexing agent to the pharmaceutical compound comprising a protonated nitrogen atom is from about 2:1 to about 1:2. In some embodiments, molar ratio of acidic functional groups of the complexing agent to the pharmaceutical compound comprising a protonated nitrogen atom is from about 1.75:1 to about 1:1.75. In some embodiments, molar ratio of acidic functional groups of the complexing agent to the pharmaceutical compound comprising a protonated nitrogen atom is from about 1.5:1 to about 1:1.5. In some embodiments, molar ratio of acidic functional groups of the complexing agent to the pharmaceutical compound comprising a protonated nitrogen atom is from about 1.4:1 to about 1:1.4. In some embodiments, molar ratio of acidic functional groups of the complexing agent to the pharmaceutical compound comprising a protonated nitrogen atom is from 1.3:1 to about 1:1.3. In some embodiments, molar ratio of acidic functional groups of the complexing agent to the pharmaceutical compound comprising a protonated nitrogen atom is from about 1.25:1 to about 1:1.25. In some embodiments, molar ratio of acidic functional groups of the complexing agent to the pharmaceutical compound comprising a protonated nitrogen atom is from about 1.2:1 to about 1:1.2. In some embodiments, molar ratio of acidic functional groups of the complexing agent to the pharmaceutical compound comprising a protonated nitrogen atom is from about 1.15:1 to about 1:1.15. In some embodiments, molar ratio of acidic functional groups of the complexing agent to the pharmaceutical compound comprising a protonated nitrogen atom is from about 1.1:1 to about 1:1.1. In some embodiments, molar ratio of acidic functional groups of the complexing agent to the pharmaceutical compound comprising a protonated nitrogen atom is from about 1.05:1 to about 1:1.05. In some embodiments, molar ratio of acidic functional groups of the complexing agent to the pharmaceutical compound comprising a protonated nitrogen atom is about 1:1.

In some embodiments, the complexing agent further comprises a non-polar region. In some embodiments, the non-polar region is a non-polar pore. In some embodiments, the additional molar equivalent of the unionized substance is complexed to the non-polar region of the complexing agent. In some embodiments, the additional molar equivalent of the unionized substance is complexed to the non-polar pore of the complexing agent.

In some embodiments, the pharmaceutical composition comprises 0.1-20 molar equivalents of the unionized substance compared to the complexing agent. In some embodiments, the pharmaceutical composition comprises 0.2-15 molar equivalents of unionized substance compared to the complexing agent. In some embodiments, the pharmaceutical composition comprises 0.5-10 molar equivalents of the unionized substance compared to the complexing agent. In some embodiments, the pharmaceutical composition comprises 1-5 molar equivalents of the unionized substance compared to the complexing agent. In some embodiments, the pharmaceutical composition comprises 0.1-20 molar equivalents of the unionized substance compared to the complexing agent, e.g., about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, or 20.0 molar equivalents of the unionized substance, or any amount therebetween. In some embodiments, the pharmaceutical composition comprises 1-2 molar equivalents of the unionized substance compared to the complexing agent. In some embodiments, the pharmaceutical composition comprises about 1.1, 1.2, 1.3, 1.4, 1.5, 1.6, 1.7, 1.8, 1.9, or about 2 molar equivalents of the unionized substance compared to the complexing agent.

In some embodiments, the solubility of the ununionized substance in the pharmaceutical composition is higher than (i) the solubility of the unionized substance as a salt; or (ii) the solubility of the unionized substance in a composition comprising the unionized substance in freebase form that is complexing to a non-polar pore of the complexing agent, wherein the unionized substance has the same concentration in the pharmaceutical composition, the unionized substance as a salt, and the composition comprising the unionized substance in freebase form. In some embodiments, the solubility of the unionized substance in the pharmaceutical composition is higher than the solubility of the unionized substance in a composition comprising the unionized substance and complexing agent with a higher molar ratio of the unionized substance to the complexing agent, wherein the unionized substance has the same concentration in the pharmaceutical composition and the composition with a higher molar ratio of the unionized substance to the complexing agent. In some embodiments, the solubility of the unionized substance in the pharmaceutical composition has an increase of at least 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, or 100-fold compared with (i) the solubility of the unionized substance as a salt; or (ii) the solubility of the unionized substance in a composition comprising the unionized substance in freebase form that is complexing to a non-polar pore of the complexing agent, wherein the unionized substance has the same concentration in the pharmaceutical composition, the unionized substance as a salt, and the composition comprising the unionized substance in freebase form. In some embodiments, the solubility of the unionized substance in the pharmaceutical composition has an increase of at least 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, or 100-fold compared with the solubility of the unionized substance in a composition comprising the unionized substance and complexing agent with a higher molar ratio of the unionized substance to the complexing agent, wherein the unionized substance has the same concentration in the pharmaceutical composition and the composition with a higher molar ratio of the unionized substance to the complexing agent.

In an aspect, provided herein is a pharmaceutical composition comprising (i) a first pharmaceutical compound, or an enantiomer, a mixture of enantiomers, or an isotopic variant thereof, wherein the first pharmaceutical compound comprises a protonated nitrogen atom; (ii) a complexing agent, wherein the complexing agent comprises a plurality of acidic functional groups, wherein the plurality of acidic functional groups comprise a conjugate base of an acid which acts as a counterion for the protonated nitrogen atom of the first pharmaceutical compound; and (iii) a second pharmaceutical compound, wherein the second pharmaceutical compound is unionized.

In some embodiments, the complexing agent is sulfobutylether-β-cyclodextrin. In some embodiments, the first pharmaceutical compound comprises a dissociative medication compound, a dissociative hallucinogen compound, a dissociative anesthetic compound, an arylcyclo-hexylamine, a 1,2-diarylethylamine, a β-keto-arylcyclohexylamine, or a compound that modulates the NMDA receptor. In some embodiments, the first pharmaceutical compound is ketamine, arylcyclo-hexylamine, 1,2-diarylethylamine, β-keto-arylcyclohexylamine, methoxetamine, deschloroketamine, N-ethyl deschloroketamine (eticyclidone), 3-methoxyphencyclidine, methoxieticyclidine, ephenidine, lanicemine, dextromethorphan, dextrorphan, methoxyketamine, a N,N-dimethyltryptamine, a N,N-diethyltryptamine, a N,N-dipropyltryptamine, a N-Methyl-N-propyltryptamine, a N-methyl-N-isopropyltryptamine, a N,N-diallyltryptamine, a N-methyl-N-allyltryptamine, N-methyl-N-ethyltryptamine, a N,N-Diisopropyltryptamine, 4-hydroxy-N-methyl-N-ethyltryptamine, 5-methoxy-N,N-diisopropyltryptamine, O-acetylpsilocin, methylisopropyllysergamide, ethylisopropyllysergamide, 6-allyl-6-nor-LSD, 6-ethyl-6-nor-lysergic acid diethylamide, 1-acetyl-LSD, 1-propionyl-6-ethyl-6-nor-lysergic acid diethylamide, 1-propionyl-lysergic acid diethylamide, 1-Cyclopropionyl-d-lysergic acid diethylamide, N1-butyryl-lysergic acid diethylamide, 6-propyl-6-nor-Lysergic acid diethylamide, mescaline, 2,5-dimethoxy-4-bromophenethylamine (2C—B), 2-(4-Iodo-2,5-dimethoxyphenyl) ethan-1-amine (2C—I), 2-(4-Chloro-2,5-dimethoxyphenyl) ethan-1-amine (2C—C), 2,5-Dimethoxy-4-iodoamphetamine, 2-[2,5-Dimethoxy-4-(propylsulfanyl)phenyl]ethan-1-amine, 2-(4-iodo-2,5-dimethoxyphenyl)-N-[(2-methoxyphenyl)methyl]ethanamine, racemorphan, levorphanol, racemethorphan, buprenorphine, morphine, loperamide, morphine, codeine, hydrocodone, oxymorphone, buprenorphine, fentanyl, methadone, tramadol, alpha-methyl acetyl fentanyl, alfentanil, butyryl fentanyl, butyrfentanyl, carfentanil, 3-methylcarfentanil, 4-fluorofentanyl, beta-hydroxyfentanyl, alpha-methylfentanyl, cis-3-methylfentanyl, beta-hydroxy-3-methylfentanyl, remifentanil, sufentanil, 3-methylthiofentanyl, naloxone, naltrexone, a cathinone, a 3,4-methylenedioxyamphetamine derivative, an aminoalkyl-substituted benzofuran, a substituted amphetamine, an aminoindane, diphenhydramine, hydroxazine, phenylephrine, dopamine, adrenaline, lidocaine, oxymetazoline, clemastine, chlorpheniramine, or 6-chloro-2-aminotetralin. In some embodiments, the first pharmaceutical compound comprises ketamine. In some embodiments, the second pharmaceutical compound comprises rapamycin. In some embodiments, the first pharmaceutical compound comprises ketamine and the second pharmaceutical compound comprises rapamycin. In some embodiments, the first pharmaceutical compound comprises no ketamine and the second pharmaceutical compound comprises rapamycin. In some embodiments, the second pharmaceutical compound comprises clonidine. In some embodiments, the first pharmaceutical compound comprises ketamine and the second pharmaceutical compound comprises clonidine. In some embodiments, the first pharmaceutical compound comprises no ketamine and the second pharmaceutical compound comprises clonidine. In some embodiments, the pharmaceutical composition comprises and additional molar equivalent of the first pharmaceutical compound in an unionized form. In some embodiments, the second pharmaceutical compound is unionized. In some embodiments, the second pharmaceutical compound does not comprise an ionizable nitrogen atom.

In some embodiments, the plurality of acidic functional groups comprise an acidic group which acts as a counterion for the protonated amine of the first pharmaceutical compound. In some embodiments, the acidic group is the conjugate base of the acidic group. In some embodiments, the acidic group is a carboxylic acid or carboxylate. In some embodiments, the acidic group is a carboxylate. In some embodiments, the acidic group is a sulfonic acid or sulfonate. In some embodiments, the acidic group is a sulfonate. In some embodiments, the conjugate base of the complexing agent acts as the counterion for a plurality of the first pharmaceutical compound. In some embodiments, each acidic group of the plurality of acidic functional groups acts as a counterion for a plurality of the first pharmaceutical compound. In some embodiments, each acidic group of the plurality of acidic functional groups acts as a counterion for a protonated amine of a plurality of the first pharmaceutical compound. In some embodiments, each of the plurality of acidic functional groups acts as a counterion for a pronated amine and/or pronated nitrogen atom.

In some embodiments, the pharmaceutically composition comprises a cyclodextrin substituted with at least one acidic functional group. In some embodiments, the at least one acidic functional group is a carboxylic acid, sulfonic acid, sulfonic acid, phosphonic acid, or any combination thereof. In some embodiments, the cyclodextrin is substituted with at least 1, at least 2, at least 3, at least 4, at least 5, or at least 6 acidic functional groups. In some embodiments, the cyclodextrin is substituted with 3 to 8 acidic functional groups, 3 to 7 acidic functional groups, 4 to 8 acidic functional groups, 4 to 7 acidic functional groups, 5 to 8 acidic functional groups, 6 to 8 acidic functional groups, or 7 to 8 acidic functional groups.

In some embodiments, the complexing agent is a substituted or unsubstituted cyclodextrin. In some cases, substituted cyclodextrins provided herein are complex mixtures wherein individual cyclodextrin molecules may comprise different numbers of substituents from other individual cyclodextrin molecules. In such cases, the number of substituents (e.g. the number of acidic functional groups) described as being present on the cyclodextrins provided herein may refer to an average degree of substitution of the mixture. For example, when a cyclodextrin is described as substituted with 3 to 8 acidic functional groups, it is intended that a complex mixture of cyclodextrins having an average degree of substitution from 3 to 8 acidic functional groups is covered. The average degree of substitution need not be an integer value and will often be a decimal value. For example, commercially available SBEBCD has an average degree of substitution of about 6.5.

In some embodiments, the complexing agent is a substituted cyclodextrin. In some embodiments, the substituted cyclodextrin is substituted with one or more acidic functional groups, or a pharmaceutically acceptable salt thereof. In some embodiments, the substituted cyclodextrin is substituted with one or more carboxylic acid, sulfonic acid, sulfonic acid, or phosphonic acid. In some embodiments, the cyclodextrin is substituted with at least 1, at least 2, at least 3, at least 4, at least 5, or at least 6 acidic functional groups. In some embodiments, the cyclodextrin is substituted with 3 to 8 acidic functional groups, 3 to 7 acidic functional groups, 4 to 8 acidic functional groups, 4 to 7 acidic functional groups, 5 to 8 acidic functional groups, 6 to 8 acidic functional groups, or 7 to 8 acidic functional groups.

In some embodiments, the molar ratio of the cyclodextrin to first the pharmaceutical compound comprising a protonated nitrogen atom is from about 1:4 to about 1:8. In some embodiments, the molar ratio of the cyclodextrin to the first pharmaceutical compound comprising a protonated nitrogen atom is from about 1:4 to about 1:10. In some embodiments, the molar ratio of the cyclodextrin to the first pharmaceutical compound comprising a protonated nitrogen atom is from about 1:5 to about 1:7. In some embodiments, the molar ratio of the cyclodextrin to the first pharmaceutical compound comprising a protonated nitrogen atom is about 1:4. In some embodiments, the molar ratio of the cyclodextrin to the first pharmaceutical compound comprising a protonated nitrogen atom is about 1:5. In some embodiments, the molar ratio of the cyclodextrin to the first pharmaceutical compound comprising a protonated nitrogen atom is about 1:6. In some embodiments, the molar ratio of the cyclodextrin to the first pharmaceutical compound comprising a protonated nitrogen atom is about 1:7. In some embodiments, the molar ratio of the cyclodextrin to the first pharmaceutical compound comprising a protonated nitrogen atom is about 1:8. In some embodiments, the molar ratio of the cyclodextrin to the first pharmaceutical compound comprising a protonated nitrogen atom is about 1:9. In some embodiments, the molar ratio of the cyclodextrin to the first pharmaceutical compound comprising a protonated nitrogen atom is about 1:10. In some embodiments, the molar ratio of the cyclodextrin to the first pharmaceutical compound comprising a protonated nitrogen atom is from about 1:4 to about 1:10. In some embodiments, the molar ratio of the cyclodextrin to the first pharmaceutical compound comprising a protonated nitrogen atom is from about 1:5 to about 1:7. In some embodiments, the molar ratio of the cyclodextrin to the first pharmaceutical compound comprising a protonated nitrogen atom is about 1:4. In some embodiments, the molar ratio of the cyclodextrin to the first pharmaceutical compound comprising a protonated nitrogen atom is about 1:5. In some embodiments, the molar ratio of the cyclodextrin to the first pharmaceutical compound comprising a protonated nitrogen atom is about 1:6. In some embodiments, the molar ratio of the cyclodextrin to the first pharmaceutical compound comprising a protonated nitrogen is about 1:7.

In some embodiments, molar ratio of acidic functional groups of the complexing agent to the first pharmaceutical compound comprising a protonated nitrogen atom is from about 2:1 to about 1:2. In some embodiments, molar ratio of acidic functional groups of the complexing agent to the first pharmaceutical compound comprising a protonated nitrogen atom is from about 1.75:1 to about 1:1.75. In some embodiments, molar ratio of acidic functional groups of the complexing agent to the first pharmaceutical compound comprising a protonated nitrogen atom is from about 1.5:1 to about 1:1.5. In some embodiments, molar ratio of acidic functional groups of the complexing agent to the first pharmaceutical compound comprising a protonated nitrogen atom is from about 1.4:1 to about 1:1.4. In some embodiments, molar ratio of acidic functional groups of the complexing agent to the first pharmaceutical compound comprising a protonated nitrogen atom is from 1.3:1 to about 1:1.3. In some embodiments, molar ratio of acidic functional groups of the complexing agent to the first pharmaceutical compound comprising a protonated nitrogen atom is from about 1.25:1 to about 1:1.25. In some embodiments, molar ratio of acidic functional groups of the complexing agent to the first pharmaceutical compound comprising a protonated nitrogen atom is from about 1.2:1 to about 1:1.2. In some embodiments, molar ratio of acidic functional groups of the complexing agent to the first pharmaceutical compound comprising a protonated nitrogen atom is from about 1.15:1 to about 1:1.15. In some embodiments, molar ratio of acidic functional groups of the complexing agent to the first pharmaceutical compound comprising a protonated nitrogen atom is from about 1.1:1 to about 1:1.1. In some embodiments, molar ratio of acidic functional groups of the complexing agent to the first pharmaceutical compound comprising a protonated nitrogen atom is from about 1.05:1 to about 1:1.05. In some embodiments, molar ratio of acidic functional groups of the complexing agent to the first pharmaceutical compound comprising a protonated nitrogen atom is about 1:1.

In some embodiments, molar ratio of acidic functional groups of the complexing agent to the first pharmaceutical compound comprising a protonated nitrogen atom is from about 2:1 to about 1:2. In some embodiments, molar ratio of acidic functional groups of the complexing agent to the first pharmaceutical compound comprising a protonated nitrogen atom is from about 1.75:1 to about 1:1.75. In some embodiments, molar ratio of acidic functional groups of the complexing agent to the first pharmaceutical compound comprising a protonated nitrogen is from about 1.5:1 to about 1:1.5. In some embodiments, molar ratio of acidic functional groups of the complexing agent to the first pharmaceutical compound comprising a protonated nitrogen atom is from about 1.4:1 to about 1:1.4. In some embodiments, molar ratio of acidic functional groups of the complexing agent to the first pharmaceutical compound comprising a protonated nitrogen atom is from about 1.3:1 to about 1:1.3. In some embodiments, molar ratio of acidic functional groups of the complexing agent to the first pharmaceutical compound comprising a protonated nitrogen atom is from about 1.25:1 to about 1:1.25. In some embodiments, molar ratio of acidic functional groups of the complexing agent to the first pharmaceutical compound comprising a protonated nitrogen atom is from about 1.2:1 to about 1:1.2. In some embodiments, molar ratio of acidic functional groups of the complexing agent to the first pharmaceutical compound comprising a protonated nitrogen atom is from about 1.15:1 to about 1:1.15. In some embodiments, molar ratio of acidic functional groups of the complexing agent to the first pharmaceutical compound comprising a protonated nitrogen atom is from about 1.1:1 to about 1:1.1. In some embodiments, molar ratio of acidic functional groups of the complexing agent to the first pharmaceutical compound comprising a protonated nitrogen atom is from about 1.05:1 to about 1:1.05. In some embodiments, molar ratio of acidic functional groups of the complexing agent to the first pharmaceutical compound comprising a protonated nitrogen is about 1:1.

In some embodiments, the complexing agent further comprises a non-polar region. In some embodiments, the non-polar region is a non-polar pore. In some embodiments, the unionized second pharmaceutical compound is complexed to the non-polar region of the complexing agent. In some embodiments, the unionized second pharmaceutical compound is complexed to the non-polar pore of the complexing agent. In some embodiments, the additional molar equivalent of the unionized first pharmaceutical compound is complexed to the non-polar region of the complexing agent. In some embodiments, the additional molar equivalent of the unionized first pharmaceutical compound is complexed to the non-polar pore of the complexing agent.

In some embodiments, the pharmaceutical composition comprises 0.1-20 molar equivalents of the second pharmaceutical compound compared to the complexing agent. In some embodiments, the pharmaceutical composition comprises 0.2-15 molar equivalents of second pharmaceutical compound compared to the complexing agent. In some embodiments, the pharmaceutical composition comprises 0.5-10 molar equivalents of the second pharmaceutical compound compared to the complexing agent. In some embodiments, the pharmaceutical composition comprises 1-5 molar equivalents of the second pharmaceutical compound compared to the complexing agent. In some embodiments, the pharmaceutical composition comprises 0.1-20 molar equivalents of the second pharmaceutical compound compared to the complexing agent, e.g., about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, or 20.0 molar equivalents of the second pharmaceutical compound, or any amount therebetween. In some embodiments, the pharmaceutical composition comprises 1-2 molar equivalents of the second pharmaceutical compound compared to the complexing agent. In some embodiments, the pharmaceutical composition comprises about 1.1, 1.2, 1.3, 1.4, 1.5, 1.6, 1.7, 1.8, 1.9, or about 2 molar equivalents of the second pharmaceutical compound compared to the complexing agent.

In some embodiments, the pharmaceutical composition comprises 0.1-20 molar equivalents of the unionized first pharmaceutical compound compared to the complexing agent. In some embodiments, the pharmaceutical composition comprises 0.2-15 molar equivalents of the unionized first pharmaceutical compound compared to the complexing agent. In some embodiments, the pharmaceutical composition comprises 0.5-10 molar equivalents of the unionized first pharmaceutical compound compared to the complexing agent. In some embodiments, the pharmaceutical composition comprises 1-5 molar equivalents of the unionized first pharmaceutical compound compared to the complexing agent. In some embodiments, the pharmaceutical composition comprises 0.1-20 molar equivalents of the unionized first pharmaceutical compound compared to the complexing agent, e.g., about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, or 20.0 molar equivalents of the unionized first pharmaceutical compound, or any amount therebetween. In some embodiments, the pharmaceutical composition comprises 1-2 molar equivalents of the unionized first pharmaceutical compound compared to the complexing agent. In some embodiments, the pharmaceutical composition comprises about 1.1, 1.2, 1.3, 1.4, 1.5, 1.6, 1.7, 1.8, 1.9, or about 2 molar equivalents of the unionized first pharmaceutical compound compared to the complexing agent.

The presence of unionized first or second pharmaceutical compound can have numerous benefits in certain contexts, including increasing the dose per unit weight or volume of the pharmaceutical composition. Additionally, when used in a pharmaceutical composition, the presence of free base or unionized pharmaceutical compound can be used to raise the pH of the composition as it is administered, thus potentially facilitating both bioavailability and tolerability in certain contexts (e.g. when the pKa of the pharmaceutical compound is lower than the pH at which the compound can be comfortably administered to the target tissue). Additionally, many of the complexing agents contemplated herein have an additional coordination site for unionized APIs (e.g. the middle complexing site of a cyclodextrin). Thus, in some embodiments, the complexing agents used herein can offer additional solubilization of pharmaceutical compounds beyond that accomplished merely by acid/base chemistry and ion exchange/ion pairing.

In some embodiments, the pharmaceutical composition comprises additional equivalents of the first or second pharmaceutical compound. In some embodiments, the additional equivalents are measured as compared to the moles of complexing agent. In some embodiments, the pharmaceutical composition comprises additional molar equivalents of the deprotonated first pharmaceutical compound. In some embodiments, the additional molar equivalents are measured as compared to the moles of complexing agent. In some embodiments, the additional equivalents are measured as compared to the moles of the pharmaceutical compound which forms a salt with the complexing agent (e.g. the protonated pharmaceutical compound).

In some embodiments, the solubility of the second pharmaceutical compound in the pharmaceutical composition is higher than the solubility of the second pharmaceutical compound as a salt, wherein the second pharmaceutical compound has the same concentration in the pharmaceutical composition, and the second pharmaceutical compound as a salt. In some embodiments, the solubility of the second pharmaceutical compound in the pharmaceutical composition has an increase of at least 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, or 100-fold compared with the solubility of the second pharmaceutical compound as a salt, wherein the second pharmaceutical compound has the same concentration in the pharmaceutical composition, and the second pharmaceutical compound as a salt.

In some embodiments, the cyclodextrin disclosed herein is a compound of Formula (I):

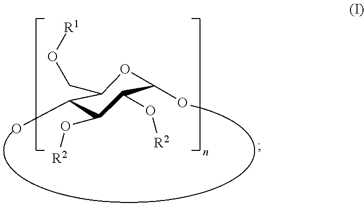

wherein:
each R¹ is independently H or optionally substituted alkyl;
each R² is independently H or optionally substituted alkyl; and
n is 6, 7, or 8;
   or a stereoisomer, a mixture of stereoisomers, or an isotopic variant thereof; or a pharmaceutically acceptable salt, solvate or hydrate thereof.

In some embodiments, each R¹ is independently H or alkyl optionally substituted with a polar functional group. In some embodiments, the polar functional group is an amido functional group, an acidic functional group, an ester functional group, a hydroxyl functional group, an alkoxy functional group, or a poly(alkylene oxide) functional group. In some embodiments, each R¹ is independently H or alkyl optionally substituted with an acidic functional group or a hydroxyl functional group.

In some embodiments, each R¹ is independently H or alkyl optionally substituted with an acidic functional group. In some embodiments, each R¹ is independently H or alkyl substituted with an acidic functional group. In some embodiments, each R¹ is independently H or C₁-C₆ alkyl substituted with an acidic functional group. In some embodiments, each R¹ is independently H or C₁-C₆ alkyl substituted with an acidic functional group selected from a carboxylic acid, a sulfonic acid, a sulfonic acid, a phosphonic acid, or a phosphonic acid. In some embodiments, each R¹ is independently H,

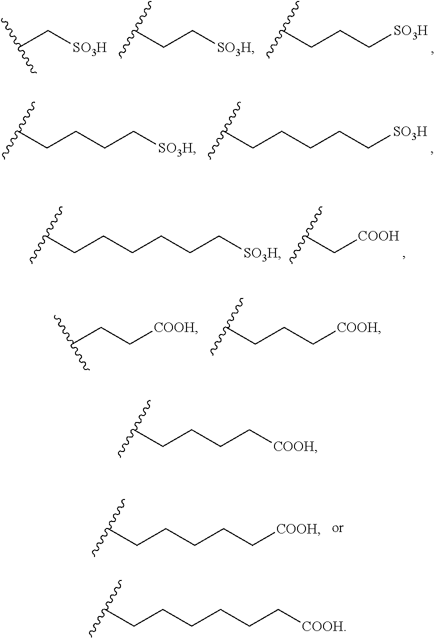

In some embodiments, each R¹ is independently H,

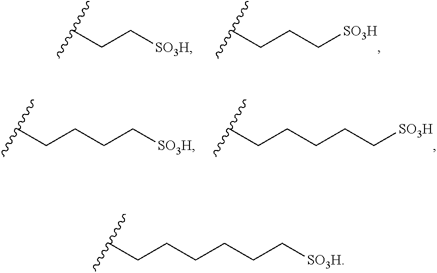 [SMILES: *CCS(=O)(=O)O ; *CCCS(=O)(=O)O ; *CCCCS(=O)(=O)O ; *CCCCCS(=O)(=O)O ; *CCCCCCS(=O)(=O)O]

In some embodiments wherein R¹ comprises an acidic functional group, each R² is H or acetyl. In some embodiments wherein R¹ comprises an acidic functional group, each R² is H.

In some embodiments, each R¹ is independently H or alkyl optionally substituted with a hydroxyl functional group. In some embodiments, each R¹ is independently H or alkyl substituted with a hydroxyl functional group. In some embodiments, each R¹ is independently H or C₁-C₆ alkyl substituted with a hydroxyl functional group. In some embodiments, each R¹ is independently H or hydroxypropyl, hydroxybutyl, hydroxypentyl, or hydroxyhexyl. In some embodiments, each R¹ and R² is independently H or hydroxypropyl, hydroxybutyl, hydroxypentyl, or hydroxyhexyl.

In some embodiments, each R² is independently H or alkyl optionally substituted with a polar functional group. In some embodiments, each R² is independently H or alkyl optionally substituted with a hydroxyl functional group. In some embodiments, each R² is independently H or alkyl substituted with a hydroxyl functional group. In some embodiments, each R² is independently H or C₁-C₆ alkyl substituted with a hydroxyl functional group. In some embodiments, each R² is independently H or hydroxypropyl, hydroxybutyl, hydroxypentyl, or hydroxyhexyl. In some embodiments, each R² is H. In some embodiments, each R² is H or acetyl.

In some embodiments, each R² is independently H or alkyl optionally substituted with an acidic functional group. In some embodiments, each R² is independently H or C₁-C₆ alkyl optionally substituted with an acidic functional group. In some embodiments, each R² is independently H or C₁-C₆ alkyl optionally substituted with a sulfonic acid or carboxylic acid functional group.

In some embodiments, n is 6 or 7. In some embodiments, n is 7 or 8. In some embodiments, n is 6. In some embodiments, n is 7. In some embodiments, n is 8. In some embodiments, the cyclodextrin is a sulfobutyl-ether-beta-cyclodextrin (SBEBCD) or a hydroxypropyl-beta-cyclodextrin (HPBCD). In some embodiments, the cyclodextrin is a SBEBCD. In some embodiments, the SBEBCD is the free acid form of SBEBCD.

In some embodiments, the molar ratio of SBEBCD to the pharmaceutical compound comprising a protonated nitrogen atom is from about 1:1 to about 1:4. In some embodiments, the molar ratio of SBEBCD to the pharmaceutical compound comprising a protonated nitrogen atom is from about 1:4 to about 1:8. In some embodiments, the molar ratio of SBEBCD to the pharmaceutical compound comprising a protonated nitrogen atom is from about 1:4 to about 1:10. In some embodiments, the molar ratio of SBEBCD to the pharmaceutical compound comprising a protonated nitrogen atom is from about 1:4 to about 1:8. In some embodiments, the molar ratio of SBEBCD to the pharmaceutical compound comprising a protonated nitrogen atom is from about 1:4 to about 1:10. In some embodiments, the molar ratio of SBEBCD to the pharmaceutical compound comprising a protonated nitrogen atom is from about 1:5 to about 1:7. In some embodiments, the molar ratio of SBEBCD to the pharmaceutical compound comprising a protonated nitrogen atom is about 1:4. In some embodiments, the molar ratio of SBEBCD to the pharmaceutical compound comprising a protonated nitrogen atom is about 1:5. In some embodiments, the molar ratio of SBEBCD to the pharmaceutical compound comprising a protonated nitrogen atom is about 1:6. In some embodiments, the molar ratio of SBEBCD to the pharmaceutical compound comprising a protonated nitrogen atom is about 1:7. In some embodiments, the molar ratio of SBEBCD to the pharmaceutical compound comprising a protonated nitrogen atom is about 1:8. In some embodiments, the molar ratio of SBEBCD to the pharmaceutical compound comprising a protonated nitrogen atom is about 1:9. In some embodiments, the molar ratio of SBEBCD to the pharmaceutical compound comprising a protonated nitrogen atom is about 1:10. In some embodiments, the molar ratio of SBEBCD to the pharmaceutical compound comprising a protonated nitrogen atom is about 1:1 to about 1:10. In some embodiments, the molar ratio of SBEBCD to the pharmaceutical compound comprising a protonated nitrogen atom is about 1:1 to about 1:2, about 1:2 to about 1:3, about 1:2 to about 1:4, about 1:2 to about 1:5, about 1:2 to about 1:6, about 1:2 to about 1:7, about 1:2 to about 1:8, about 1:2 to about 1:9, about 1:2 to about 1:10, about 1:3 to about 1:4, about 1:3 to about 1:5, about 1:3 to about 1:6, about 1:3 to about 1:7, about 1:3 to about 1:8, about 1:3 to about 1:9, about 1:3 to about 1:10, about 1:4 to about 1:5, about 1:4 to about 1:6, about 1:4 to about 1:7, about 1:4 to about 1:8, about 1:4 to about 1:9, about 1:4 to about 1:10, about 1:5 to about 1:6, about 1:5 to about 1:7, about 1:5 to about 1:8, about 1:5 to about 1:9, about 1:5 to about 1:10, about 1:6 to about 1:7, about 1:6 to about 1:8, about 1:6 to about 1:9, about 1:6 to about 1:10, about 1:7 to about 1:8, about 1:7 to about 1:9, about 1:7 to about 1:10, about 1:8 to about 1:9, about 1:8 to about 1:10, or about 1:9 to about 1:10. In some embodiments, the molar ratio of SBEBCD to the pharmaceutical compound comprising a protonated nitrogen atom is about 1:1, 1:2, about 1:3, about 1:4, about 1:5, about 1:6, about 1:7, about 1:8, about 1:9, or about 1:10. In some embodiments, the molar ratio of SBEBCD to the pharmaceutical compound comprising a protonated nitrogen atom is at least about 1:2, about 1:3, about 1:4, about 1:5, about 1:6, about 1:7, about 1:8, or about 1:9. In some embodiments, the molar ratio of SBEBCD to the pharmaceutical compound comprising a protonated nitrogen atom is at most about 1:3, about 1:4, about 1:5, about 1:6, about 1:7, about 1:8, about 1:9, or about 1:10.

In some embodiments, the pharmaceutically acceptable salt or pharmaceutical composition provided herein consists essentially of the complexing agent and the pharmaceutical compound. In some embodiments, the pharmaceutically acceptable salt or pharmaceutical composition provided herein consists of the complexing agent and the pharmaceutical compound. In some embodiments, the pharmaceutically acceptable salt or pharmaceutical composition provided herein consists of the protonated pharmaceutical compound and the deprotonated complexing agent.

In some embodiments, the pharmaceutical composition further comprises a base, a buffer, or a combination thereof.

In some embodiments, the pharmaceutical composition does not comprise a base, a buffer, or a combination thereof.

In some embodiments, the co-solvent is ethanol, propylene glycol, tween 20, tween 80, glycerin, or a combination thereof.

In some embodiments, the complexing agent is a substituted or unsubstituted cyclodextrin. In some cases, substituted cyclodextrins provided herein are complex mixtures wherein individual cyclodextrin molecules may comprise different numbers of substituents from other individual cyclodextrin molecules. In such cases, the number of substituents (e.g. the number of acidic functional groups) described as being present on the cyclodextrins provided herein may refer to an average degree of substitution of the mixture. For example, when a cyclodextrin is described as substituted with 3 to 8 acidic functional groups, it is intended that a complex mixture of cyclodextrins having an average degree of substitution from 3 to 8 acidic functional groups is covered.

In some embodiments, the complexing agent is a substituted cyclodextrin. In some embodiments, the substituted cyclodextrin is substituted with one or more acidic functional groups, or a pharmaceutically acceptable salt thereof. In some embodiments, the substituted cyclodextrin is substituted with one or more carboxylic acid, sulfonic acid, sulfonic acid, phosphonic acid, or phosphonic acid. In some embodiments, the cyclodextrin is substituted with at least 1, at least 2, at least 3, at least 4, at least 5, or at least 6 acidic functional groups. In some embodiments, the cyclodextrin is substituted with 3 to 8 acidic functional groups, 3 to 7 acidic functional groups, 4 to 8 acidic functional groups, 4 to 7 acidic functional groups, 5 to 8 acidic functional groups, 6 to 8 acidic functional groups, or 7 to 8 acidic functional groups.

In some embodiments, the cyclodextrin is a compound of Formula (I).

In some embodiments, the cyclodextrin is a sulfobutyl-ether-beta-cyclodextrin (SBEBCD). In some embodiments, the SBEBCD is the free acid form of SBEBCD.

In some embodiments, the pharmaceutically acceptable salt is formulated in an aqueous medium, either as a solution or a suspension. Such solutions or suspensions can be used in a variety of formulations, such as formulations for subcutaneous administration, intranasal administration, or sublingual administration. In some embodiments, when formulated in an aqueous medium, the pharmaceutical compositions provided herein will have lowered osmolality compared to other formulations having the same concentration of complexing agent and pharmaceutical compound which utilize salts of the complexing agent, the pharmaceutical compound, or both.

In some embodiments, a complexing agent disclosed herein can form any of the pharmaceutical compositions disclosed herein. In some embodiments, a complexing agent disclosed herein can form any of the pharmaceutically acceptable salts disclosed herein. In some embodiments, a complexing agent is used in any of the methods disclosed herein.

In some embodiments, the pharmaceutical composition disclosed herein has lower osmolality than a composition comprising a salt of the pharmaceutical compound disclosed herein and a salt of the complexing agent disclosed herein. In some embodiments, the pharmaceutical composition disclosed herein has lower osmolality than a composition comprising a salt of the pharmaceutical compound disclosed herein. In some embodiments, the pharmaceutical composition disclosed herein has lower osmolality than a composition comprising a salt of the complexing agent disclosed herein (e.g. a sodium salt), the pharmaceutical composition disclosed herein has substantially the same osmolality as a solution of the same concentration of a salt of the complexing agent disclosed herein. In some embodiments, the pharmaceutical composition disclosed herein has substantially the same osmolality as a solution of the same concentration of a sodium salt of the complexing agent disclosed herein. In some embodiments, the comparison of osmolality of the pharmaceutical composition disclosed herein is compared to one in which the concentration of the pharmaceutical compound disclosed herein and the complexing agent disclosed herein is the same. In some embodiments, the salt of the complexing agent disclosed herein used for the comparison is the sodium salt. In some embodiments, the salt of the pharmaceutical compound is the HCl salt. In some embodiments, the pharmaceutical composition has lower osmolality than (i) a composition comprising a salt of the pharmaceutical compound; or (ii) a composition comprising the pharmaceutical compound in freebase form that is complexing to a non-polar pore of the complexing agent, wherein the pharmaceutical compound has the same concentration in the pharmaceutical composition, the composition comprising the salt of the pharmaceutical compound, and the composition comprising the pharmaceutical compound in freebase form.

In some embodiments, the pharmaceutical composition disclosed herein has an osmolality that is about 10% to about 50% less than a corresponding pharmaceutical composition prepared from a salt of the complexing agent disclosed herein. In some embodiments, the pharmaceutical composition disclosed herein has an osmolality that is about 10% to about 20%, about 10% to about 30%, about 10% to about 40%, about 10% to about 50%, about 20% to about 30%, about 20% to about 40%, about 20% to about 50%, about 30% to about 40%, about 30% to about 50%, or about 40% to about 50% less than a corresponding pharmaceutical composition prepared from a salt of the complexing agent disclosed herein. In some embodiments, the pharmaceutical composition disclosed herein has an osmolality that is about 10%, about 20%, about 30%, about 40%, or about 50% less than a corresponding pharmaceutical composition prepared from a salt of the complexing agent disclosed herein. In some embodiments, the pharmaceutical composition disclosed herein has an osmolality that is at least about 10%, about 20%, about 30%, or about 40% less than a corresponding pharmaceutical composition prepared from a salt of the complexing agent disclosed herein.

In some embodiments, the pharmaceutical composition disclosed herein has an osmolality that is about 10% to about 50% less than a corresponding pharmaceutical composition prepared from a salt of the pharmaceutical compound disclosed herein. In some embodiments, the pharmaceutical composition has an osmolality that is about 10% to about 20%, about 10% to about 30%, about 10% to about 40%, about 10% to about 50%, about 20% to about 30%, about 20% to about 40%, about 20% to about 50%, about 30% to about 40%, about 30% to about 50%, or about 40% to about 50% less than a corresponding pharmaceutical composition prepared from a salt of the pharmaceutical compound disclosed herein. In some embodiments, the pharmaceutical composition disclosed herein has an osmolality that is about 10%, about 20%, about 30%, about 40%, or about 50% less than a corresponding pharmaceutical composition prepared from a salt of the pharmaceutical compound disclosed herein. In some embodiments, the pharmaceutical composition disclosed herein has an osmolality that is at least about 10%, about 20%, about 30%, or about 40% less than a corresponding pharmaceutical composition prepared from a salt of the pharmaceutical compound disclosed herein.

In some embodiments, the pharmaceutical composition disclosed herein has an osmolality that is about 50 mOsm/kg to about 500 mOsm/kg less than a corresponding pharmaceutical composition prepared from a salt of the complexing agent disclosed herein. In some embodiments, the pharmaceutical composition disclosed herein has an osmolality that is about 50 mOsm/kg to about 100 mOsm/kg, about 50 mOsm/kg to about 150 mOsm/kg, about 50 mOsm/kg to about 200 mOsm/kg, about 50 mOsm/kg to about 250 mOsm/kg, about 50 mOsm/kg to about 300 mOsm/kg, about 50 mOsm/kg to about 400 mOsm/kg, about 50 mOsm/kg to about 500 mOsm/kg, about 100 mOsm/kg to about 150 mOsm/kg, about 100 mOsm/kg to about 200 mOsm/kg, about 100 mOsm/kg to about 250 mOsm/kg, about 100 mOsm/kg to about 300 mOsm/kg, about 100 mOsm/kg to about 400 mOsm/kg, about 100 mOsm/kg to about 500 mOsm/kg, about 150 mOsm/kg to about 200 mOsm/kg, about 150 mOsm/kg to about 250 mOsm/kg, about 150 mOsm/kg to about 300 mOsm/kg, about 150 mOsm/kg to about 400 mOsm/kg, about 150 mOsm/kg to about 500 mOsm/kg, about 200 mOsm/kg to about 250 mOsm/kg, about 200 mOsm/kg to about 300 mOsm/kg, about 200 mOsm/kg to about 400 mOsm/kg, about 200 mOsm/kg to about 500 mOsm/kg, about 250 mOsm/kg to about 300 mOsm/kg, about 250 mOsm/kg to about 400 mOsm/kg, about 250 mOsm/kg to about 500 mOsm/kg, about 300 mOsm/kg to about 400 mOsm/kg, about 300 mOsm/kg to about 500 mOsm/kg, or about 400 mOsm/kg to about 500 mOsm/kg less than a corresponding pharmaceutical composition prepared from a salt of the complexing agent disclosed herein. In some embodiments, the pharmaceutical composition disclosed herein has an osmolality that is about 50 mOsm/kg, about 100 mOsm/kg, about 150 mOsm/kg, about 200 mOsm/kg, about 250 mOsm/kg, about 300 mOsm/kg, about 400 mOsm/kg, or about 500 mOsm/kg less than a corresponding pharmaceutical composition prepared from a salt of the complexing agent disclosed herein. In some embodiments, the pharmaceutical composition has an osmolality that is at least about 50 mOsm/kg, about 100 mOsm/kg, about 150 mOsm/kg, about 200 mOsm/kg, about 250 mOsm/kg, about 300 mOsm/kg, or about 400 mOsm/kg less than a corresponding pharmaceutical composition disclosed herein prepared from a salt of the complexing agent disclosed herein.

In some embodiments, the pharmaceutical composition disclosed herein has an osmolality that is about 50 mOsm/kg to about 500 mOsm/kg less than a corresponding pharmaceutical composition prepared from a salt of the pharmaceutical compound disclosed herein. In some embodiments, the pharmaceutical composition disclosed herein has an osmolality that is about 50 mOsm/kg to about 100 mOsm/kg, about 50 mOsm/kg to about 150 mOsm/kg, about 50 mOsm/kg to about 200 mOsm/kg, about 50 mOsm/kg to about 250 mOsm/kg, about 50 mOsm/kg to about 300 mOsm/kg, about 50 mOsm/kg to about 400 mOsm/kg, about 50 mOsm/kg to about 500 mOsm/kg, about 100 mOsm/kg to about 150 mOsm/kg, about 100 mOsm/kg to about 200 mOsm/kg, about 100 mOsm/kg to about 250 mOsm/kg, about 100 mOsm/kg to about 300 mOsm/kg, about 100 mOsm/kg to about 400 mOsm/kg, about 100 mOsm/kg to about 500 mOsm/kg, about 150 mOsm/kg to about 200 mOsm/kg, about 150 mOsm/kg to about 250 mOsm/kg, about 150 mOsm/kg to about 300 mOsm/kg, about 150 mOsm/kg to about 400 mOsm/kg, about 150 mOsm/kg to about 500 mOsm/kg, about 200 mOsm/kg to about 250 mOsm/kg, about 200 mOsm/kg to about 300 mOsm/kg, about 200 mOsm/kg to about 400 mOsm/kg, about 200 mOsm/kg to about 500 mOsm/kg, about 250 mOsm/kg to about 300 mOsm/kg, about 250 mOsm/kg to about 400 mOsm/kg, about 250 mOsm/kg to about 500 mOsm/kg, about 300 mOsm/kg to about 400 mOsm/kg, about 300 mOsm/kg to about 500 mOsm/kg, or about 400 mOsm/kg to about 500 mOsm/kg less than a corresponding pharmaceutical composition prepared from a salt of the pharmaceutical compound disclosed herein. In some embodiments, the pharmaceutical composition has an osmolality that is about 50 mOsm/kg, about 100 mOsm/kg, about 150 mOsm/kg, about 200 mOsm/kg, about 250 mOsm/kg, about 300 mOsm/kg, about 400 mOsm/kg, or about 500 mOsm/kg less than a corresponding pharmaceutical composition prepared from a salt of the pharmaceutical compound disclosed herein. In some embodiments, the pharmaceutical composition disclosed herein has an osmolality that is at least about 50 mOsm/kg, about 100 mOsm/kg, about 150 mOsm/kg, about 200 mOsm/kg, about 250 mOsm/kg, about 300 mOsm/kg, or about 400 mOsm/kg less than a corresponding pharmaceutical composition prepared from a salt of the pharmaceutical compound disclosed herein.

In some embodiments, the pharmaceutical composition disclosed herein has an osmolality that is about 50 mOsm/kg to about 500 mOsm/kg less than a corresponding pharmaceutical composition prepared from a salt of the pharmaceutical compound and a salt of the complexing agent disclosed herein. In some embodiments, the pharmaceutical composition disclosed herein has an osmolality that is about 50 mOsm/kg to about 100 mOsm/kg, about 50 mOsm/kg to about 150 mOsm/kg, about 50 mOsm/kg to about 200 mOsm/kg, about 50 mOsm/kg to about 250 mOsm/kg, about 50 mOsm/kg to about 300 mOsm/kg, about 50 mOsm/kg to about 400 mOsm/kg, about 50 mOsm/kg to about 500 mOsm/kg, about 100 mOsm/kg to about 150 mOsm/kg, about 100 mOsm/kg to about 200 mOsm/kg, about 100 mOsm/kg to about 250 mOsm/kg, about 100 mOsm/kg to about 300 mOsm/kg, about 100 mOsm/kg to about 400 mOsm/kg, about 100 mOsm/kg to about 500 mOsm/kg, about 150 mOsm/kg to about 200 mOsm/kg, about 150 mOsm/kg to about 250 mOsm/kg, about 150 mOsm/kg to about 300 mOsm/kg, about 150 mOsm/kg to about 400 mOsm/kg, about 150 mOsm/kg to about 500 mOsm/kg, about 200 mOsm/kg to about 250 mOsm/kg, about 200 mOsm/kg to about 300 mOsm/kg, about 200 mOsm/kg to about 400 mOsm/kg, about 200 mOsm/kg to about 500 mOsm/kg, about 250 mOsm/kg to about 300 mOsm/kg, about 250 mOsm/kg to about 400 mOsm/kg, about 250 mOsm/kg to about 500 mOsm/kg, about 300 mOsm/kg to about 400 mOsm/kg, about 300 mOsm/kg to about 500 mOsm/kg, or about 400 mOsm/kg to about 500 mOsm/kg less than a corresponding pharmaceutical composition prepared from a salt of the pharmaceutical compound disclosed herein and a salt of the complexing agent disclosed herein. In some embodiments, the pharmaceutical composition disclosed herein has an osmolality that is about 50 mOsm/kg, about 100 mOsm/kg, about 150 mOsm/kg, about 200 mOsm/kg, about 250 mOsm/kg, about 300 mOsm/kg, about 400 mOsm/kg, or about 500 mOsm/kg less than a corresponding pharmaceutical composition prepared from a salt of the pharmaceutical compound disclosed herein and a salt of the complexing agent disclosed herein. In some embodiments, the pharmaceutical composition has an osmolality that is at least about 50 mOsm/kg, about 100 mOsm/kg, about 150 mOsm/kg, about 200 mOsm/kg, about 250 mOsm/kg, about 300 mOsm/kg, or about 400 mOsm/kg less than a corresponding pharmaceutical composition prepared from a salt of the pharmaceutical compound disclosed herein and a salt of the complexing agent disclosed herein.

In some embodiments, the osmolarity values and ranges disclosed above are applicable to any of the pharmaceutical compositions disclosed herein. In some embodiments, the osmolarity values and ranges disclosed above are applicable to a solution prepared from any of the pharmaceutically acceptable salts disclosed herein. In some embodiments, the osmolarity values and ranges disclosed above are applicable to a pharmaceutical composition prepared from any of the methods disclosed herein.

### Subcutaneous Formulations

In some aspects, the pharmaceutical composition provided herein is formulated for subcutaneous administration. Subcutaneously deliverable compounds have the advantage over other forms of compounds (e.g. IV or IM delivery) in that it can be used outside of a hospital or clinical setting, such as at home by the subject. Other formulations of compounds suitable for at home use, such as oral or nasal delivery formulations, tend to require higher doses to achieve comparable clinical effects, which carries risks, including bladder dysfunction due to the higher dosing and dissociative effects. Additionally, oral or sublingual administration is sometimes unreliable due to the presence of food or chyme in the stomach or proximal small intestines and substantial first pass metabolism. Intranasal administration can cause allergic or irritation rhinitis, epistaxis (nosebleeds), or bacterial or viral sinusitis in certain contexts.

In some embodiments, the pharmaceutical formulation provided herein are able to combine a high concentration of compound (e.g. >20 mg/mL) with additional characteristics of the formulation making it ideally suited to subcutaneous injection. These additional characteristics may include osmolality and pH closer to physiological levels than is possible with a composition prepared exclusively or partially with a salt of the compound and/or complexing agent while still maintaining stability of the formulation and solubility of compound. In some embodiments, these desired properties are achieved through use of a complexing agent, particularly cyclodextrins, which act enhance the solubility of the compound at elevated pHs (e.g. pHs as high as about 5.5, or another pH near the low end of the buffering capacity of the particular compound of interest). In some embodiments, these attributes are further enhanced through the use modified cyclodextrins, particularly cyclodextrins modified by sulfonate functional groups (e.g. a sulfobutyl-ether-beta-cyclodextrin (SBEBCD). In some embodiments, use of a cyclodextrin modified to replace the sodium in the sodium sulfonate salt functional groups to form sulfonic acid functional groups (e.g. SBEBCD) is particularly advantageous, as the sulfonic acidic functional groups can act as the counter-anion to protonate the non-ionized or freebase form of the compound. In such embodiments, a low osmolality in a high concentration pharmaceutical compound formulation is achieved because additional salts and counterions can be omitted from the formulation. Thus, high concentrations of compounds at pH levels compatible with subcutaneous injection can be achieved at osmolalities comparable to physiological levels (˜300 mOsm/kg), thus enabling the subcutaneous administration of compounds without side effects such as pain or injection site irritation.

In some embodiments, the pharmaceutical composition is a solution. In some embodiments, the pharmaceutical composition is a solid. In some embodiments, the pharmaceutical composition has a pH >about 4.

In some embodiments, the pharmaceutical composition has a pH of about 4 to about 7. In some embodiments, the pharmaceutical composition has a pH of about 4 to about 7. In some embodiments, the pharmaceutical composition has a pH of about 4 to about 4.5, about 4 to about 5, about 4 to about 5.5, about 4 to about 6, about 4 to about 6.5, about 4 to about 7, about 4.5 to about 5, about 4.5 to about 5.5, about 4.5 to about 6, about 4.5 to about 6.5, about 4.5 to about 7, about 5 to about 5.5, about 5 to about 6, about 5 to about 6.5, about 5 to about 7, about 5.5 to about 6, about 5.5 to about 6.5, about 5.5 to about 7, about 6 to about 6.5, about 6 to about 7, or about 6.5 to about 7. In some embodiments, the pharmaceutical composition has a pH of about 4, about 4.5, about 5, about 5.5, about 6, about 6.5, or about 7. In some embodiments, the pharmaceutical composition has a pH of at least about 4, about 4.5, about 5, about 5.5, about 6, or about 6.5. In some embodiments, the pharmaceutical composition has a pH of at most about 4.5, about 5, about 5.5, about 6, about 6.5, or about 7.

In some embodiments, the pharmaceutical composition has a pH of about 4.5 to about 5.5.

In some embodiments, the pharmaceutical composition has an osmolality of from about 250 mOsm/kg to about 850 mOsm/kg. In some embodiments, the pharmaceutical composition has an osmolality of from about 275 mOsm/kg to about 850 mOsm/kg. In some embodiments, the pharmaceutical composition has an osmolality of from about 300 mOsm/kg to about 850 mOsm/kg. In some embodiments, the pharmaceutical composition has an osmolality of from about 325 mOsm/kg to about 850 mOsm/kg. In some embodiments, the pharmaceutical composition has an osmolality of from about 350 mOsm/kg to about 850 mOsm/kg. In some embodiments, the pharmaceutical composition has an osmolality of from about 375 mOsm/kg to about 850 mOsm/kg. In some embodiments, the pharmaceutical composition has an osmolality of from about 400 mOsm/kg to about 850 mOsm/kg. In some embodiments, the pharmaceutical composition has an osmolality of from about 300 mOsm/kg to about 450 mOsm/kg. In some embodiments, the pharmaceutical composition has an osmolality of from about 475 mOsm/kg to about 850 mOsm/kg. In some embodiments, the pharmaceutical composition has an osmolality of from about 500 mOsm/kg to about 850 mOsm/kg.

In some embodiments, the pharmaceutical composition has an osmolality of at least about 250 mOsm/kg. In some embodiments, the pharmaceutical composition has an osmolality of at least about 275 mOsm/kg. In some embodiments, the pharmaceutical composition has an osmolality of at least about 300 mOsm/kg. In some embodiments, the pharmaceutical composition has an osmolality of at least about 325 mOsm/kg. In some embodiments, the pharmaceutical composition has an osmolality of at least about 350 mOsm/kg. In some embodiments, the pharmaceutical composition has an osmolality of at least about 375 mOsm/kg. In some embodiments, the pharmaceutical composition has an osmolality of at least about 400 mOsm/kg. In some embodiments, the pharmaceutical composition has an osmolality of at least about 425 mOsm/kg. In some embodiments, the pharmaceutical composition has an osmolality of at least about 450 mOsm/kg. In some embodiments, the pharmaceutical composition has an osmolality of at least about 475 mOsm/kg. In some embodiments, the pharmaceutical composition has an osmolality of at least about 500 mOsm/kg.

In some embodiments, the pharmaceutical composition has an osmolality of about <850 mOsm/kg. In some embodiments, the pharmaceutical composition has an osmolality of about <825 mOsm/kg. In some embodiments, the pharmaceutical composition has an osmolality of about <800 mOsm/kg. In some embodiments, the pharmaceutical composition has an osmolality of about <775 mOsm/kg. In some embodiments, the pharmaceutical composition has an osmolality of about <750 mOsm/kg. In some embodiments, the pharmaceutical composition has an osmolality of about <725 mOsm/kg. In some embodiments, the pharmaceutical composition has an osmolality of about <700 mOsm/kg. In some embodiments, the pharmaceutical composition has an osmolality of about <675 mOsm/kg. In some embodiments, the pharmaceutical composition has an osmolality of about <650 mOsm/kg. In some embodiments, the pharmaceutical composition has an osmolality of about <625 mOsm/kg. In some embodiments, the pharmaceutical composition has an osmolality of about <600 mOsm/kg. In some embodiments, the pharmaceutical composition has an osmolality of about <575 mOsm/kg. In some embodiments, the pharmaceutical composition has an osmolality of about <550 mOsm/kg. In some embodiments, the pharmaceutical composition has an osmolality of about <525 mOsm/kg. In some embodiments, the pharmaceutical composition has an osmolality of about <500 mOsm/kg. In some embodiments, the pharmaceutical composition has an osmolality of about <450 mOsm/kg. In some embodiments, the pharmaceutical composition has an osmolality of about <400 mOsm/kg. In some embodiments, the pharmaceutical composition has an osmolality of about <350 mOsm/kg.

In some embodiments, the pharmaceutical composition has an osmolality of about 300 mOsm/kg to about 850 mOsm/kg. In some embodiments, the pharmaceutical composition has an osmolality of about 300 mOsm/kg to about 350 mOsm/kg, about 300 mOsm/kg to about 400 mOsm/kg, about 300 mOsm/kg to about 450 mOsm/kg, about 300 mOsm/kg to about 500 mOsm/kg, about 300 mOsm/kg to about 550 mOsm/kg, about 300 mOsm/kg to about 600 mOsm/kg, about 300 mOsm/kg to about 650 mOsm/kg, about 300 mOsm/kg to about 700 mOsm/kg, about 300 mOsm/kg to about 750 mOsm/kg, about 300 mOsm/kg to about 800 mOsm/kg, about 300 mOsm/kg to about 850 mOsm/kg, about 350 mOsm/kg to about 400 mOsm/kg, about 350 mOsm/kg to about 450 mOsm/kg, about 350 mOsm/kg to about 500 mOsm/kg, about 350 mOsm/kg to about 550 mOsm/kg, about 350 mOsm/kg to about 600 mOsm/kg, about 350 mOsm/kg to about 650 mOsm/kg, about 350 mOsm/kg to about 700 mOsm/kg, about 350 mOsm/kg to about 750 mOsm/kg, about 350 mOsm/kg to about 800 mOsm/kg, about 350 mOsm/kg to about 850 mOsm/kg, about 400 mOsm/kg to about 450 mOsm/kg, about 400 mOsm/kg to about 500 mOsm/kg, about 400 mOsm/kg to about 550 mOsm/kg, about 400 mOsm/kg to about 600 mOsm/kg, about 400 mOsm/kg to about 650 mOsm/kg, about 400 mOsm/kg to about 700 mOsm/kg, about 400 mOsm/kg to about 750 mOsm/kg, about 400 mOsm/kg to about 800 mOsm/kg, about 400 mOsm/kg to about 850 mOsm/kg, about 450 mOsm/kg to about 500 mOsm/kg, about 450 mOsm/kg to about 550 mOsm/kg, about 450 mOsm/kg to about 600 mOsm/kg, about 450 mOsm/kg to about 650 mOsm/kg, about 450 mOsm/kg to about 700 mOsm/kg, about 450 mOsm/kg to about 750 mOsm/kg, about 450 mOsm/kg to about 800 mOsm/kg, about 450 mOsm/kg to about 850 mOsm/kg, about 500 mOsm/kg to about 550 mOsm/kg, about 500 mOsm/kg to about 600 mOsm/kg, about 500 mOsm/kg to about 650 mOsm/kg, about 500 mOsm/kg to about 700 mOsm/kg, about 500 mOsm/kg to about 750 mOsm/kg, about 500 mOsm/kg to about 800 mOsm/kg, about 500 mOsm/kg to about 850 mOsm/kg, about 550 mOsm/kg to about 600 mOsm/kg, about 550 mOsm/kg to about 650 mOsm/kg, about 550 mOsm/kg to about 700 mOsm/kg, about 550 mOsm/kg to about 750 mOsm/kg, about 550 mOsm/kg to about 800 mOsm/kg, about 550 mOsm/kg to about 850 mOsm/kg, about 600 mOsm/kg to about 650 mOsm/kg, about 600 mOsm/kg to about 700 mOsm/kg, about 600 mOsm/kg to about 750 mOsm/kg, about 600 mOsm/kg to about 800 mOsm/kg, about 600 mOsm/kg to about 850 mOsm/kg, about 650 mOsm/kg to about 700 mOsm/kg, about 650 mOsm/kg to about 750 mOsm/kg, about 650 mOsm/kg to about 800 mOsm/kg, about 650 mOsm/kg to about 850 mOsm/kg, about 700 mOsm/kg to about 750 mOsm/kg, about 700 mOsm/kg to about 800 mOsm/kg, about 700 mOsm/kg to about 850 mOsm/kg, about 750 mOsm/kg to about 800 mOsm/kg, about 750 mOsm/kg to about 850 mOsm/kg, or about 800 mOsm/kg to about 850 mOsm/kg. In some embodiments, the pharmaceutical composition has an osmolality of about 300 mOsm/kg, about 350 mOsm/kg, about 400 mOsm/kg, about 450 mOsm/kg, about 500 mOsm/kg, about 550 mOsm/kg, about 600 mOsm/kg, about 650 mOsm/kg, about 700 mOsm/kg, about 750 mOsm/kg, about 800 mOsm/kg, or about 850 mOsm/kg. In some embodiments, the pharmaceutical composition has an osmolality of at least about 300 mOsm/kg about 350 mOsm/kg, about 400 mOsm/kg, about 450 mOsm/kg, about 500 mOsm/kg, about 550 mOsm/kg, about 600 mOsm/kg, about 650 mOsm/kg, about 700 mOsm/kg, about 750 mOsm/kg, or about 800 mOsm/kg. In some embodiments, the pharmaceutical composition has an osmolality of at most about 350 mOsm/kg, about 400 mOsm/kg, about 450 mOsm/kg, about 500 mOsm/kg, about 550 mOsm/kg, about 600 mOsm/kg, about 650 mOsm/kg, about 700 mOsm/kg, about 750 mOsm/kg, about 800 mOsm/kg, or about 850 mOsm/kg.

In some embodiments, the pharmaceutical composition is isotonic.

In some embodiments, the pharmaceutical composition has an osmolality of about 500 mOsm/kg.

In some embodiments, the pharmaceutical compound or an enantiomer, a mixture of enantiomers, or an isotopic variant thereof; or solvate or hydrate thereof, has a concentration of about 20 mg/mL to about 150 mg/mL. In some embodiments, the pharmaceutical compound or an enantiomer, a mixture of enantiomers, or an isotopic variant thereof; or solvate or hydrate thereof, has a concentration up to about 150 mg/mL. In some embodiments, the pharmaceutical compound or an enantiomer, a mixture of enantiomers, or an isotopic variant thereof; or solvate or hydrate thereof, has a concentration at least about 20 mg/mL.

In some embodiments, the pharmaceutical compound, or an enantiomer, a mixture of enantiomers, or an isotopic variant thereof; or solvate or hydrate thereof, has a concentration of about 80 mg/mL to about 120 mg/mL.

In some embodiments, the pharmaceutical compound, or an enantiomer, a mixture of enantiomers, or an isotopic variant thereof; or solvate or hydrate thereof, has a concentration of about 95 mg/mL to about 105 mg/mL.

In some embodiments, the pharmaceutical compound, or an enantiomer, a mixture of enantiomers, or an isotopic variant thereof; or solvate or hydrate thereof, has a concentration of about 20 mg/mL. In some embodiments, the pharmaceutical compound, or an enantiomer, a mixture of enantiomers, or an isotopic variant thereof; or solvate or hydrate thereof, has a concentration of about 25 mg/mL. In some embodiments, the pharmaceutical compound, or an enantiomer, a mixture of enantiomers, or an isotopic variant thereof, or solvate or hydrate thereof, has a concentration of about 30 mg/mL. In some embodiments, the pharmaceutical compound, or an enantiomer, a mixture of enantiomers, or an isotopic variant thereof; or solvate or hydrate thereof, has a concentration of about 35 mg/mL. In some embodiments, the pharmaceutical compound, or an enantiomer, a mixture of enantiomers, or an isotopic variant thereof; or solvate or hydrate thereof, has a concentration of about 40 mg/mL. In some embodiments, the pharmaceutical compound, or an enantiomer, a mixture of enantiomers, or an isotopic variant thereof; or solvate or hydrate thereof, has a concentration of about 45 mg/mL. In some embodiments, the pharmaceutical compound, or an enantiomer, a mixture of enantiomers, or an isotopic variant thereof; or solvate or hydrate thereof, has a concentration of about 50 mg/mL. In some embodiments, the pharmaceutical compound, or an enantiomer, a mixture of enantiomers, or an isotopic variant thereof; or solvate or hydrate thereof, has a concentration of about 55 mg/mL. In some embodiments, the pharmaceutical compound, or an enantiomer, a mixture of enantiomers, or an isotopic variant thereof; or solvate or hydrate thereof, has a concentration of about 60 mg/mL. In some embodiments, the pharmaceutical compound, or an enantiomer, a mixture of enantiomers, or an isotopic variant thereof; or solvate or hydrate thereof, has a concentration of about 65 mg/mL. In some embodiments, the pharmaceutical compound, or an enantiomer, a mixture of enantiomers, or an isotopic variant thereof; or solvate or hydrate thereof, has a concentration of about 70 mg/mL. In some embodiments, the pharmaceutical compound, or an enantiomer, a mixture of enantiomers, or an isotopic variant thereof; or solvate or hydrate thereof, has a concentration of about 75 mg/mL. In some embodiments, the pharmaceutical compound, or an enantiomer, a mixture of enantiomers, or an isotopic variant thereof; or solvate or hydrate thereof, has a concentration of about 80 mg/mL. In some embodiments, the pharmaceutical compound, or an enantiomer, a mixture of enantiomers, or an isotopic variant thereof; or solvate or hydrate thereof, has a concentration of about 85 mg/mL. In some embodiments, the pharmaceutical compound, or an enantiomer, a mixture of enantiomers, or an isotopic variant thereof; or solvate or hydrate thereof, has a concentration of about 90 mg/mL. In some embodiments, the pharmaceutical compound, or an enantiomer, a mixture of enantiomers, or an isotopic variant thereof; or solvate or hydrate thereof, has a concentration of about 95 mg/mL. In some embodiments, the pharmaceutical compound, or an enantiomer, a mixture of enantiomers, or an isotopic variant thereof, or solvate or hydrate thereof, has a concentration of about 100 mg/mL. In some embodiments, the pharmaceutical compound, or an enantiomer, a mixture of enantiomers, or an isotopic variant thereof; or solvate or hydrate thereof, has a concentration of about 105 mg/mL. In some embodiments, the pharmaceutical compound, or an enantiomer, a mixture of enantiomers, or an isotopic variant thereof; or solvate or hydrate thereof, has a concentration of about 110 mg/mL. In some embodiments, the pharmaceutical compound, or an enantiomer, a mixture of enantiomers, or an isotopic variant thereof; or solvate or hydrate thereof, has a concentration of about 115 mg/mL. In some embodiments, the pharmaceutical compound, or an enantiomer, a mixture of enantiomers, or an isotopic variant thereof; or solvate or hydrate thereof, has a concentration of about 120 mg/mL. In some embodiments, the pharmaceutical compound, or an enantiomer, a mixture of enantiomers, or an isotopic variant thereof; or solvate or hydrate thereof, has a concentration of about 125 mg/mL. In some embodiments, the pharmaceutical compound, or an enantiomer, a mixture of enantiomers, or an isotopic variant thereof; or solvate or hydrate thereof, has a concentration of about 130 mg/mL. In some embodiments, the pharmaceutical compound, or an enantiomer, a mixture of enantiomers, or an isotopic variant thereof; or solvate or hydrate thereof, has a concentration of about 135 mg/mL. In some embodiments, the pharmaceutical compound, or an enantiomer, a mixture of enantiomers, or an isotopic variant thereof; or solvate or hydrate thereof, has a concentration of about 140 mg/mL. In some embodiments, the pharmaceutical compound, or an enantiomer, a mixture of enantiomers, or an isotopic variant thereof; or solvate or hydrate thereof, has a concentration of about 145 mg/mL. In some embodiments, the pharmaceutical compound, or an enantiomer, a mixture of enantiomers, or an isotopic variant thereof; or solvate or hydrate thereof, has a concentration of about 150 mg/mL.

In some embodiments, the pharmaceutical compound, or an enantiomer, a mixture of enantiomers, or an isotopic variant thereof; or solvate or hydrate thereof, has a concentration of about 95 mg/mL, about 96 mg/mL, about 97 mg/mL, about 98 mg/mL, about 99 mg/mL, about 100 mg/mL, about 101 mg/mL, 102 mg/mL, about 103 mg/mL, about 104 mg/mL, or about 105 mg/mL.

In some embodiments, the pharmaceutical compound or an enantiomer, a mixture of enantiomers, or an isotopic variant thereof; or solvate or hydrate thereof, has a concentration of about 20 mg/mL to about 150 mg/mL. In some embodiments, the pharmaceutical compound or an enantiomer, a mixture of enantiomers, or an isotopic variant thereof, or solvate or hydrate thereof, has a concentration of about 20 mg/mL to about 40 mg/mL, about 20 mg/mL to about 60 mg/mL, about 20 mg/mL to about 80 mg/mL, about 20 mg/mL to about 100 mg/mL, about 20 mg/mL to about 120 mg/mL, about 20 mg/mL to about 140 mg/mL, about 20 mg/mL to about 150 mg/mL, about 40 mg/mL to about 60 mg/mL, about 40 mg/mL to about 80 mg/mL, about 40 mg/mL to about 100 mg/mL, about 40 mg/mL to about 120 mg/mL, about 40 mg/mL to about 140 mg/mL, about 40 mg/mL to about 150 mg/mL, about 60 mg/mL to about 80 mg/mL, about 60 mg/mL to about 100 mg/mL, about 60 mg/mL to about 120 mg/mL, about 60 mg/mL to about 140 mg/mL, about 60 mg/mL to about 150 mg/mL, about 80 mg/mL to about 100 mg/mL, about 80 mg/mL to about 120 mg/mL, about 80 mg/mL to about 140 mg/mL, about 80 mg/mL to about 150 mg/mL, about 100 mg/mL to about 120 mg/mL, about 100 mg/mL to about 140 mg/mL, about 100 mg/mL to about 150 mg/mL, about 120 mg/mL to about 140 mg/mL, about 120 mg/mL to about 150 mg/mL, or about 140 mg/mL to about 150 mg/mL. In some embodiments, the pharmaceutical compound or an enantiomer, a mixture of enantiomers, or an isotopic variant thereof; or solvate or hydrate thereof, has a concentration of about 20 mg/mL, about 40 mg/mL, about 60 mg/mL, about 80 mg/mL, about 100 mg/mL, about 120 mg/mL, about 140 mg/mL, or about 150 mg/mL. In some embodiments, the pharmaceutical compound or an enantiomer, a mixture of enantiomers, or an isotopic variant thereof; or solvate or hydrate thereof, has a concentration of at least about 20 mg/mL, about 40 mg/mL, about 60 mg/mL, about 80 mg/mL, about 100 mg/mL, about 120 mg/mL, or about 140 mg/mL. In some embodiments, the pharmaceutical compound or an enantiomer, a mixture of enantiomers, or an isotopic variant thereof; or solvate or hydrate thereof, has a concentration of at most about 40 mg/mL, about 60 mg/mL, about 80 mg/mL, about 100 mg/mL, about 120 mg/mL, about 140 mg/mL, or about 150 mg/mL.

In some embodiments, the cyclodextrin is present in an amount of about 50 mg/mL to about 600 mg/mL. In some embodiments, the cyclodextrin is present in an amount of about 10 mg/mL to about 600 mg/mL. In some embodiments, the cyclodextrin is present in an amount of about 50 mg/mL to about 600 mg/mL. In some embodiments, the cyclodextrin is present in an amount of about 50 mg/mL to about 100 mg/mL, about 50 mg/mL to about 200 mg/mL, about 50 mg/mL to about 300 mg/mL, about 50 mg/mL to about 400 mg/mL, about 50 mg/mL to about 500 mg/mL, about 50 mg/mL to about 600 mg/mL, about 100 mg/mL to about 200 mg/mL, about 100 mg/mL to about 300 mg/mL, about 100 mg/mL to about 400 mg/mL, about 100 mg/mL to about 500 mg/mL, about 100 mg/mL to about 600 mg/mL, about 200 mg/mL to about 300 mg/mL, about 200 mg/mL to about 400 mg/mL, about 200 mg/mL to about 500 mg/mL, about 200 mg/mL to about 600 mg/mL, about 300 mg/mL to about 400 mg/mL, about 300 mg/mL to about 500 mg/mL, about 300 mg/mL to about 600 mg/mL, about 400 mg/mL to about 500 mg/mL, about 400 mg/mL to about 600 mg/mL, or about 500 mg/mL to about 600 mg/mL. In some embodiments, the cyclodextrin is present in an amount of about 50 mg/mL, about 100 mg/mL, about 200 mg/mL, about 300 mg/mL, about 400 mg/mL, about 500 mg/mL, or about 600 mg/mL. In some embodiments, the cyclodextrin is present in an amount of at least about 50 mg/mL, about 100 mg/mL, about 200 mg/mL, about 300 mg/mL, about 400 mg/mL, or about 500 mg/mL. In some embodiments, the cyclodextrin is present in an amount of at most about 100 mg/mL, about 200 mg/mL, about 300 mg/mL, about 400 mg/mL, about 500 mg/mL, or about 600 mg/mL.

In some embodiments, the pharmaceutical composition further comprises a preservative. In some embodiments, the preservative is benzethonium chloride. In some embodiments, the benzethonium chloride is present in an amount of about 0.1 mg/mL to about 0.5 mg/mL. In some embodiments, the preservative is benzethonium chloride, benzalkonium chloride, or chloroxylenol. Other preservatives include benzyl alcohol, methyl parabens, ethyl or n-propyl, and p-hydroxybenzoate. In some embodiments, preservatives are antimicrobial agents, including, but not limited to: Phenol, Meta-cresol, Benzyl alcohol, parabens (methyl, propyl, or butyl), benzalkonium chloride, benzethonium chloride, chlorobutanol, Myristyl gamma picolinium chloride, 2-phenoxyethanol, Phenethyl alcohol, Sorbates (sorbic acid, sodium sorbate), Ethanol, and/or Propylene glycol. In some embodiments, the preservative is present in an amount of about 0.1 mg/mL to about 1 mg/mL. In some embodiments, the preservative is present in an amount of about 0.1 mg/mL to about 0.2 mg/mL, about 0.1 mg/mL to about 0.3 mg/mL, about 0.1 mg/mL to about 0.4 mg/mL, about 0.1 mg/mL to about 0.5 mg/mL, about 0.1 mg/mL to about 0.6 mg/mL, about 0.1 mg/mL to about 0.7 mg/mL, about 0.1 mg/mL to about 0.8 mg/mL, about 0.1 mg/mL to about 0.9 mg/mL, about 0.1 mg/mL to about 1 mg/mL, about 0.2 mg/mL to about 0.3 mg/mL, about 0.2 mg/mL to about 0.4 mg/mL, about 0.2 mg/mL to about 0.5 mg/mL, about 0.2 mg/mL to about 0.6 mg/mL, about 0.2 mg/mL to about 0.7 mg/mL, about 0.2 mg/mL to about 0.8 mg/mL, about 0.2 mg/mL to about 0.9 mg/mL, about 0.2 mg/mL to about 1 mg/mL, about 0.3 mg/mL to about 0.4 mg/mL, about 0.3 mg/mL to about 0.5 mg/mL, about 0.3 mg/mL to about 0.6 mg/mL, about 0.3 mg/mL to about 0.7 mg/mL, about 0.3 mg/mL to about 0.8 mg/mL, about 0.3 mg/mL to about 0.9 mg/mL, about 0.3 mg/mL to about 1 mg/mL, about 0.4 mg/mL to about 0.5 mg/mL, about 0.4 mg/mL to about 0.6 mg/mL, about 0.4 mg/mL to about 0.7 mg/mL, about 0.4 mg/mL to about 0.8 mg/mL, about 0.4 mg/mL to about 0.9 mg/mL, about 0.4 mg/mL to about 1 mg/mL, about 0.5 mg/mL to about 0.6 mg/mL, about 0.5 mg/mL to about 0.7 mg/mL, about 0.5 mg/mL to about 0.8 mg/mL, about 0.5 mg/mL to about 0.9 mg/mL, about 0.5 mg/mL to about 1 mg/mL, about 0.6 mg/mL to about 0.7 mg/mL, about 0.6 mg/mL to about 0.8 mg/mL, about 0.6 mg/mL to about 0.9 mg/mL, about 0.6 mg/mL to about 1 mg/mL, about 0.7 mg/mL to about 0.8 mg/mL, about 0.7 mg/mL to about 0.9 mg/mL, about 0.7 mg/mL to about 1 mg/mL, about 0.8 mg/mL to about 0.9 mg/mL, about 0.8 mg/mL to about 1 mg/mL, or about 0.9 mg/mL to about 1 mg/mL. In some embodiments, the preservative is present in an amount of about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, or about 1 mg/mL. In some embodiments, the preservative is present in an amount of about at least about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, or about 0.9 mg/mL. In some embodiments, the preservative is present in an amount of about at most about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, or about 1 mg/mL.

In some embodiments of the pharmaceutical compositions disclosed herein, the form is a subcutaneous (e.g., infusion or bolus) dosage form. In some embodiments of the pharmaceutical composition, wherein the form is a subcutaneous dosage form, the pH is from about 3.0 to about 7.0. In some embodiments of the pharmaceutical composition, wherein the form is a subcutaneous dosage form, the pH is from about 4.0 to about 5.0. In some embodiments of the pharmaceutical composition, wherein the form is a subcutaneous dosage form, the pH is from about 4.5 to about 5.5. In some embodiments of the pharmaceutical composition, wherein the form is a subcutaneous dosage form, the pH is from about 5.0 to about 6.0. In some embodiments of the pharmaceutical composition, wherein the form is a subcutaneous dosage form, the pH is from about 5.5 to about 6.5. In some embodiments of the pharmaceutical composition, wherein the form is a subcutaneous dosage form, the pH is from about 6.0 to about 7.0. In some embodiments of the pharmaceutical composition, wherein the form is a subcutaneous dosage form, the pH is about 3.0. In some embodiments of the pharmaceutical composition, wherein the form is a subcutaneous dosage form, the pH is about 3.5. In some embodiments of the pharmaceutical composition, wherein the form is a subcutaneous dosage form, the pH is about 4.0. In some embodiments of the pharmaceutical composition, wherein the form is a subcutaneous dosage form, the pH is about 4.5. In some embodiments of the pharmaceutical composition, wherein the form is a subcutaneous dosage form, the pH is about 5.0. In some embodiments of the pharmaceutical composition, wherein the form is a subcutaneous dosage form, the pH is about 5.1. In some embodiments of the pharmaceutical composition, wherein the form is a subcutaneous dosage form, the pH is about 5.2. In some embodiments of the pharmaceutical composition, wherein the form is a subcutaneous dosage form, the pH is about 5.3. In some embodiments of the pharmaceutical composition, wherein the form is a subcutaneous dosage form, the pH is about 5.4. In some embodiments of the pharmaceutical composition, wherein the form is a subcutaneous dosage form, the pH is about 5.5. In some embodiments of the pharmaceutical composition, wherein the form is a subcutaneous dosage form, the pH is about 5.6. In some embodiments of the pharmaceutical composition, wherein the form is a subcutaneous dosage form, the pH is about 5.7. In some embodiments of the pharmaceutical composition, wherein the form is a subcutaneous dosage form, the pH is about 5.8. In some embodiments of the pharmaceutical composition, wherein the form is a subcutaneous dosage form, the pH is about 5.9. In some embodiments of the pharmaceutical composition, wherein the form is a subcutaneous dosage form, the pH is about 6.0. In some embodiments of the pharmaceutical composition, wherein the form is a subcutaneous dosage form, the pH is about 6.5. In some embodiments of the pharmaceutical composition, wherein the form is a subcutaneous dosage form, the pH is about 7.0.

### Intranasal Formulations

In some aspects, the pharmaceutical composition comprising the pharmaceutical compound provided herein is formulated for intranasal administration. While many traditional approaches to intranasal administration of pharmaceutical compounds face distinct challenges due to poor tissue tolerability (e.g. burning, nosebleeds, infections), bioavailability, and rate of tissue uptake, the pharmaceutical compositions comprising the complexing agent salts provided herein overcome many of these difficulties. In some cases, the complexing agent/pharmaceutical compound salts allow for delivery of the selected compound at a targeted pH to match natural tissue conditions while maintaining an osmolality that is also compatible with the nasal tissue. Additionally, in some cases, the presence of the complexing agent ionically associated with the pharmaceutical compound helps to solubilize the non-ionized compound component as the pharmaceutical composition is deposited in the nasal tissue and ions and other compounds exchange with the components of nasal mucosa to allow uptake of the compounds. In some cases, the presence of the complexing agent in the formulation also allows enhanced solubility of the pharmaceutical compound at the pH of the surrounding nasal mucosa after delivery.

Additionally, in some embodiments, a pharmaceutical composition comprising the complexing agent/pharmaceutical compound salts provided herein comprise additional equivalents of the pharmaceutical compound that is not ionically associated with the complexing agent, such as unionized pharmaceutical compound or ionized with a different counterion. When additional unionized pharmaceutical compound is used, the complexing agent (e.g., a cyclodextrin such as SBEBCD) may complex with this additional pharmaceutical compound through non-ionic interactions. Additionally, the pharmaceutical composition may further comprise additional equivalents unionized pharmaceutical compound relative to the amount complexing agent present (e.g. up to 20 or more molar equivalents of free base pharmaceutical compound). When a substantial excess of pharmaceutical compound is present, the cyclodextrin can deliver and solubilize one equivalent of the pharmaceutical compound at a time through complexation interactions with the pore of the cyclodextrin, and can further act in a shuttle-like mechanism to further solubilize the additional molar equivalents of free base pharmaceutical compound which may also be present. When such a formulation is administered, bioavailability and tolerability may be increased due to the presence of an amount of unionized pharmaceutical compound. This may occur for multiple reasons, including that free base (unionized) pharmaceutical compound is more readily taken up by the pertinent cells and distributed to the targeted tissue in the body (e.g. by passive diffusion). Additionally, the presence of additional unionized pharmaceutical compound can act as a buffer to aid in achieving the desired pH of the localized tissue (depending on the amount of unionized pharmaceutical compound added and the pKa of the pharmaceutical compound). In some embodiments, the desired pH can thus be achieved without the presence of additional base or buffer.

In some embodiments, the pharmaceutical composition comprising the complexing agent/pharmaceutical compound salt is formulated for intranasal administration. In some embodiments, the pharmaceutical composition is formulated as an inhalable powder for intranasal administration. In some embodiments, the pharmaceutical composition is formulated as a liquid suspension for intranasal administration. In some embodiments, the pharmaceutical composition is formulated as a liquid solution for intranasal administration. In some embodiments, the liquid suspension is an aqueous suspension. In some embodiments, the liquid solution is an aqueous solution.

In some embodiments, the pharmaceutical composition comprises additional equivalents of the pharmaceutical compound. In some embodiments, the pharmaceutical composition comprises additional molar equivalents of the pharmaceutical compound. In some embodiments, the additional equivalents are measured as compared to the moles of complexing agent. In some embodiments, the additional equivalents are measured as compared to the moles of the pharmaceutical compound which forms a salt with the complexing agent.

In some embodiments, the pharmaceutical composition comprises additional equivalents of unionized pharmaceutical compound compared to the protonated pharmaceutical compound of the complexing agent/protonated pharmaceutical compound salt. In some embodiments, the pharmaceutical composition comprises about 0.01 molar equivalents to about 10 molar equivalents of the unionized pharmaceutical compound. In some embodiments, the pharmaceutical composition comprises about 0.1 molar equivalents to about 1 molar equivalents of the unionized pharmaceutical compound. In some embodiments, the pharmaceutical composition comprises about 0.01 molar equivalents to about 1 molar equivalents of the unionized pharmaceutical compound. In some embodiments, the pharmaceutical composition comprises about 0.1 molar equivalents to about 0.2 molar equivalents, about 0.1 molar equivalents to about 0.5 molar equivalents, about 0.1 molar equivalents to about 0.75 molar equivalents, about 0.1 molar equivalents to about 1 molar equivalents, about 0.2 molar equivalents to about 0.5 molar equivalents, about 0.2 molar equivalents to about 0.75 molar equivalents, about 0.2 molar equivalents to about 1 molar equivalents, about 0.5 molar equivalents to about 0.75 molar equivalents, about 0.5 molar equivalents to about 1 molar equivalents, or about 0.75 molar equivalents to about 1 molar equivalents of the unionized pharmaceutical compound. In some embodiments, the pharmaceutical composition comprises about 0.1 molar equivalents, about 0.2 molar equivalents, about 0.5 molar equivalents, about 0.75 molar equivalents, or about 1 molar equivalents of the unionized pharmaceutical compound. In some embodiments, the pharmaceutical composition comprises at least about 0.1 molar equivalents, about 0.2 molar equivalents, about 0.5 molar equivalents, or about 0.75 molar equivalents of the unionized pharmaceutical compound. In some embodiments, the pharmaceutical composition comprises at most about 0.2 molar equivalents, about 0.5 molar equivalents, about 0.75 molar equivalents, or about 1 molar equivalents of the unionized pharmaceutical compound. In some embodiments, the pharmaceutical composition comprises about 0.5 equivalents to about 5 equivalents of the unionized pharmaceutical compound. In some embodiments, the pharmaceutical composition comprises about 0.5 equivalents to about 1 equivalents, about 0.5 equivalents to about 2 equivalents, about 0.5 equivalents to about 3 equivalents, about 0.5 equivalents to about 4 equivalents, about 0.5 equivalents to about 5 equivalents, about 1 equivalents to about 2 equivalents, about 1 equivalents to about 3 equivalents, about 1 equivalents to about 4 equivalents, about 1 equivalents to about 5 equivalents, about 2 equivalents to about 3 equivalents, about 2 equivalents to about 4 equivalents, about 2 equivalents to about 5 equivalents, about 3 equivalents to about 4 equivalents, about 3 equivalents to about 5 equivalents, or about 4 equivalents to about 5 equivalents of the unionized pharmaceutical compound. In some embodiments, the pharmaceutical composition comprises about 0.5 equivalents, about 1 equivalent, about 2 equivalents, about 3 equivalents, about 4 equivalents, or about 5 equivalents of the unionized pharmaceutical compound. In some embodiments, the pharmaceutical composition comprises at least about 0.5 equivalents, about 1 equivalent, about 2 equivalents, about 3 equivalents, or about 4 equivalents of the unionized pharmaceutical compound. In some embodiments, the pharmaceutical composition comprises at most about 1 equivalent, about 2 equivalents, about 3 equivalents, about 4 equivalents, or about 5 equivalents of the unionized pharmaceutical compound.

In some embodiments, the pharmaceutical composition comprises a sufficient amount of unionized pharmaceutical compound to form a buffering system with the ionized pharmaceutical compound in the composition. In some embodiments, the buffering system is at a desired pH. In some embodiments, the buffering system results in a desired pH when administered to nasal tissue.

In some embodiments, the buffering system results in a pH within a target range of the pKa value of the pharmaceutical compound. In some embodiments, the target pH is within about 0.2 pH units to about 2 pH units of the pKa value. In some embodiments, the target pH is within about 0.2 pH units to about 0.5 pH units, about 0.2 pH units to about 1 pH units, about 0.2 pH units to about 1.5 pH units, about 0.2 pH units to about 2 pH units, about 0.5 pH units to about 1 pH units, about 0.5 pH units to about 1.5 pH units, about 0.5 pH units to about 2 pH units, about 1 pH units to about 1.5 pH units, about 1 pH units to about 2 pH units, or about 1.5 pH units to about 2 pH units of the pKa value. In some embodiments, the target pH is within about 0.2 pH units, about 0.5 pH units, about 1 pH units, about 1.5 pH units, or about 2 pH units of the pKa value. In some embodiments, the target pH is within at least about 0.2 pH units, about 0.5 pH units, about 1 pH units, or about 1.5 pH units of the pKa value. In some embodiments, the target pH is within at most about 0.5 pH units, about 1 pH units, about 1.5 pH units, or about 2 pH units of the pKa value.

In some embodiments, the buffering system results in a pH wherein a certain percentage of the pharmaceutical compound is ionized upon administration. In some embodiments, the buffering system results in a pH wherein about 1% to about 99% of the pharmaceutical compound is ionized. In some embodiments, the buffering system results in a pH wherein about 1% to about 5%, about 1% to about 10%, about 1% to about 25%, about 1% to about 50%, about 1% to about 70%, about 1% to about 90%, about 1% to about 95%, about 1% to about 99%, about 5% to about 10%, about 5% to about 25%, about 5% to about 50%, about 5% to about 70%, about 5% to about 90%, about 5% to about 95%, about 5% to about 99%, about 10% to about 25%, about 10% to about 50%, about 10% to about 70%, about 10% to about 90%, about 10% to about 95%, about 10% to about 99%, about 25% to about 50%, about 25% to about 70%, about 25% to about 90%, about 25% to about 95%, about 25% to about 99%, about 50% to about 70%, about 50% to about 90%, about 50% to about 95%, about 50% to about 99%, about 70% to about 90%, about 70% to about 95%, about 70% to about 99%, about 90% to about 95%, about 90% to about 99%, or about 95% to about 99% of the pharmaceutical compound is ionized. In some embodiments, the buffering system results in a pH wherein about 1%, about 5%, about 10%, about 25%, about 50%, about 70%, about 90%, about 95%, or about 99% of the pharmaceutical compound is ionized. In some embodiments, the buffering system results in a pH wherein at least about 1%, about 5%, about 10%, about 25%, about 50%, about 70%, about 90%, or about 95% of the pharmaceutical compound is ionized. In some embodiments, the buffering system results in a pH wherein at most about 5%, about 10%, about 25%, about 50%, about 70%, about 90%, about 95%, or about 99% of the pharmaceutical compound is ionized. In some embodiments, the percent ionization is measured immediately after administration.

In some embodiments, the pharmaceutical composition comprises additional molar equivalents of unionized pharmaceutical compound compared to complexing agent of the complexing agent/protonated pharmaceutical compound salt. In some embodiments, the pharmaceutical composition comprises about 0.01 molar equivalents to about 10 molar equivalents of unionized pharmaceutical compound compared to the complexing agent. In some embodiments, the pharmaceutical composition comprises about 0.1 molar equivalents to about 10 molar equivalents of unionized pharmaceutical compound. In some embodiments, the pharmaceutical composition comprises about 0.01 molar equivalents to about 1 molar equivalents of the unionized pharmaceutical compound. In some embodiments, the pharmaceutical composition comprises about 0.1 molar equivalents to about 0.5 molar equivalents, about 0.1 molar equivalents to about 1 molar equivalents, about 0.1 molar equivalents to about 2 molar equivalents, about 0.1 molar equivalents to about 3 molar equivalents, about 0.1 molar equivalents to about 5 molar equivalents, about 0.1 molar equivalents to about 7 molar equivalents, about 0.1 molar equivalents to about 10 molar equivalents, about 0.5 molar equivalents to about 1 molar equivalents, about 0.5 molar equivalents to about 2 molar equivalents, about 0.5 molar equivalents to about 3 molar equivalents, about 0.5 molar equivalents to about 5 molar equivalents, about 0.5 molar equivalents to about 7 molar equivalents, about 0.5 molar equivalents to about 10 molar equivalents, about 1 molar equivalents to about 2 molar equivalents, about 1 molar equivalents to about 3 molar equivalents, about 1 molar equivalents to about 5 molar equivalents, about 1 molar equivalents to about 7 molar equivalents, about 1 molar equivalents to about 10 molar equivalents, about 2 molar equivalents to about 3 molar equivalents, about 2 molar equivalents to about 5 molar equivalents, about 2 molar equivalents to about 7 molar equivalents, about 2 molar equivalents to about 10 molar equivalents, about 3 molar equivalents to about 5 molar equivalents, about 3 molar equivalents to about 7 molar equivalents, about 3 molar equivalents to about 10 molar equivalents, about 5 molar equivalents to about 7 molar equivalents, about 5 molar equivalents to about 10 molar equivalents, or about 7 molar equivalents to about 10 molar equivalents of unionized pharmaceutical compound of unionized pharmaceutical compound. In some embodiments, the pharmaceutical composition comprises about 0.1 molar equivalents, about 0.5 molar equivalents, about 1 molar equivalents, about 2 molar equivalents, about 3 molar equivalents, about 5 molar equivalents, about 7 molar equivalents, or about 10 molar equivalents of unionized pharmaceutical compound. In some embodiments, the pharmaceutical composition comprises at least about 0.1 molar equivalents, about 0.5 molar equivalents, about 1 molar equivalents, about 2 molar equivalents, about 3 molar equivalents, about 5 molar equivalents, or about 7 molar equivalents of unionized pharmaceutical compound. In some embodiments, the pharmaceutical composition comprises at most about 0.5 molar equivalents, about 1 molar equivalents, about 2 molar equivalents, about 3 molar equivalents, about 5 molar equivalents, about 7 molar equivalents, or about 10 molar equivalents of unionized pharmaceutical compound. In some embodiments, the pharmaceutical composition comprises about 0.01 molar equivalents to about 20 molar equivalents of unionized pharmaceutical compound compared to the complexing agent. In some embodiments, the pharmaceutical composition comprises about 1 molar equivalent to about 20 molar equivalents of unionized pharmaceutical compound compared to the complexing agent. In some embodiments, at least a portion these additional equivalents of the unionized pharmaceutical compound relative to the complexing agent are complexed to the complexing agent (e.g. up to about 1 molar equivalent of the unionized pharmaceutical compound).

In some embodiments, the pharmaceutical composition comprises a suitable carrier for intranasal administration. Carriers are usually inert and frequently function as a diluent for dispensing the therapeutic agent into a storage container like a capsule or in a device, or aid in the intranasal administration of the pharmaceutical compound. In some embodiments, a pharmaceutically acceptable carrier for the present compositions includes but are not limited to amino acids, peptides, proteins, non-biological polymers, biological polymers, simple sugars, carbohydrates, gums, inorganic salts and metal compounds which may be present singularly or in combination. In some embodiments, the pharmaceutically acceptable carrier comprises native, derivatized, modified forms, or combinations thereof.

In some embodiments, useful proteins include, but are not limited to, gelatin or albumin. In some embodiments, useful sugars that can serve as pharmaceutically acceptable carriers include, but are not limited to fructose, galactose, glucose, lactitol, lactose, maltitol, maltose, mannitol, melezitose, myoinositol, palatinite, raffinose, stachyose, sucrose, trehalose, xylitol, hydrates thereof, and combinations of thereof.

In some embodiments, useful carbohydrates that can serve as pharmaceutically acceptable carriers include, but are not limited to starches such as corn starch, potato starch, amylose, amylopectin, pectin, hydroxypropyl starch, carboxymethyl starch, and cross-linked starch. In other embodiments, useful carbohydrates that can serve as pharmaceutically acceptable carriers include, but are not limited to cellulose, crystalline cellulose, microcrystalline cellulose, α-cellulose, methylcellulose, hydroxypropyl cellulose, carboxymethyl cellulose, ethyl cellulose, hydroxypropyl methyl cellulose, and cellulose acetate.

In another embodiment, useful inorganic salts or metal compounds include, but are not limited to aluminum, calcium, magnesium, silicon, and zinc salts. In some embodiments, the aluminum salts include for example, aluminum hydroxychloride, aluminum magnesium hydroxide, aluminum hydroxide, aluminum sulfate, aluminum stearate, aluminum monostearate and potassium aluminum sulfate. In other embodiments, the calcium salts include for example, apatite, hydroxyapatite, calcium carbonate, calcium chloride, calcium citrate, calcium silicate, calcium oxide, calcium hydroxide, calcium stearate, calcium phosphate tribasic, calcium lactate, calcium oleate, calcium palmirate, calcium hydrogenphosphate, calcium primary phosphate, calcium acetate, and calcium sulfate. In some embodiments, the magnesium compounds include, for example, magnesium chloride, magnesium aluminate silicate, magnesium silicate, magnesium oxide, magnesium hydroxide, magnesium stearate, magnesium carbonate, magnesium sulfate, and sodium magnesium silicate.

In some embodiments, the carrier is substantially water insoluble. In further embodiments, the substantially water insoluble carrier is selected from the group consisting of peptides, proteins, non-biological polymers, biological polymers, carbohydrates, gums, inorganic salts and metal compounds. In some embodiments, substantially water insoluble carbohydrates include cellulose, crystalline cellulose, and microcrystalline cellulose.

In some embodiments, the carrier is substantially water soluble. In further embodiments, the substantially water soluble carrier is selected from the group consisting of polysaccharides, sugars, salts, peptides, proteins, carbohydrates, non-biological polymers, biological polymers, gums, inorganic salts and metal compounds. In some embodiments, the substantially water soluble polysaccharide is cellulose. In some embodiments, the cellulose is hydroxypropyl cellulose, carboxymethyl cellulose, ethyl cellulose, hydroxypropyl methyl cellulose, or cellulose acetate. In other embodiments, the substantially water soluble polysaccharide is a starch. In some embodiments, the substantially water soluble starch is hydroxypropyl starch, carboxymethyl starch, cross-linked starch, amylose, amylopectin, or pectin and combinations of thereof. In some embodiments, the substantially water soluble sugar includes fructose, galactose, glucose, lactitol, lactose, maltitol, maltose, mannitol, melezitose, myoinositol, palatinite, raffinose, stachyose, sucrose, trehalose, xylitol, hydrates thereof, and combinations of thereof.

Usually, carriers have a mean particle size and/or particle size distribution that is substantially larger than that of the drug. The small particle size of therapeutic agents frequently exhibit very poor flow properties that compromise the filling accuracy of the dispensed agent when it is loaded into storage containers like capsules or into devices. The same poor flow properties will also impede aerosolisation or spray characteristics and compromise the intended amount of therapeutic agent to be delivered to the patient. By blending a microtine therapeutic agent with an excess of carrier that has a substantially larger median particle size, the flow properties of the composition will essentially determine the properties of the carrier thereby improving the handling characteristics required for accurate dispensing and administration.

For some formulations herein, the carrier particles have a mean particle size of at least 1 μm, at least 2 μm, at least 3 μm, at least 4 μm, at least 5 μm, at least 10 μm, at least 15 μm, at least 20 μm, at least, 25 μm, at least 30 μm, at least 40 μm, at least 50 μm, at least 60 μm, at least 70 μm, at least 80 μm, at least 90 μm, at least 100 μm, at least 150 μm, at least 200 μm, or at least 250 μm.

The intranasal pharmaceutical compositions may also comprise any of the other excipients provided herein, including without limitation any lubricant, fluidizer, surfactant, buffer, preservative, anti-oxidant, wetting agent, or any other such suitable excipient which is compatible with an intranasal formulation.

### Sublingual Formulations

In some aspects, the pharmaceutical composition provided herein is formulated for sublingual administration. In some embodiments, pharmaceutical compounds with low bioavailability, slow onset of physiological effect, and compounds with substantial non-target effects on the gastrointestinal tract or liver are ideal compounds for use with sublingual formulations comprising the complexing agent/pharmaceutical compound salts as provided herein. As with the intranasal formulations provided herein, traditional sublingual approaches to formulations of pharmaceutical compounds suffer from drawbacks of poor bioavailability (in some cases due, at least in part, to poor solubility) as well as tissue irritation (e.g. burning or stinging sensations) in the mouth. The instant salts remedy this by enhancing solubility of the compounds at relatively high doses at tolerable pHs and concentrations without a substantial increase in osmolality compared to formulations made from salts of the complexing agents or the compounds, thus minimizing tissue irritation while also maximizing bioavailability and absorption.

Additionally, in some embodiments, a pharmaceutical composition comprising the complexing agent/pharmaceutical compound salts provided herein comprise additional equivalents of the pharmaceutical compound that is not ionically associated with the complexing agent, such as unionized pharmaceutical compound or ionized with a different counterion. When additional unionized pharmaceutical compound is used, the complexing agent (e.g., a cyclodextrin such as SBEBCD) may complex with this additional pharmaceutical compound through non-ionic interactions. When such a formulation is administered, bioavailability and tolerability may be increased due to the presence of an amount of unionized pharmaceutical compound. This may occur for multiple reasons, including that free base (unionized) pharmaceutical compound is more readily taken up by the pertinent cells and distributed to the targeted tissue in the body (e.g. by passive diffusion). Additionally, the presence of additional unionized pharmaceutical compound, even beyond a 1:1 molar ratio of pharmaceutical compound:complexing agent (e.g., a ratio up to 20:1 or even higher) that would be expected to be complexed inside the complexing agent (e.g., a cyclodextrin such as SBEBCD) can act to improve the sublingual formulation. For example, the additional equivalents of free base compound can simultaneously act as a buffer, as an aid in achieving the desired pH of the localized tissue (depending on the amount of unionized pharmaceutical compound added and the pKa of the pharmaceutical compound), and as a source of unionized or free base drug product for absorption and update by the tissue (e.g. by mucosal absorption). In some embodiments, the desired pH can thus be achieved without the presence of additional base or buffer.

In some embodiments, the pharmaceutical composition comprising the complexing agent/pharmaceutical compound salt is formulated for sublingual administration. In some embodiments, the pharmaceutical composition is formulated as a sublingual tablet, a sublingual strip, a sublingual drop, a sublingual spray, a sublingual troche, or a lozenge.

In some embodiments, the pharmaceutical composition comprises additional equivalents of unionized pharmaceutical compound compared to the protonated pharmaceutical compound of the complexing agent/protonated pharmaceutical compound salt. In some embodiments, the pharmaceutical composition comprises about 0.01 molar equivalents to about 5 molar equivalents of the unionized pharmaceutical compound. In some embodiments, the pharmaceutical composition comprises about 0.1 molar equivalents to about 1 molar equivalents of the unionized pharmaceutical compound. In some embodiments, the pharmaceutical composition comprises about 0.1 molar equivalents to about 0.2 molar equivalents, about 0.1 molar equivalents to about 0.5 molar equivalents, about 0.1 molar equivalents to about 0.75 molar equivalents, about 0.1 molar equivalents to about 1 molar equivalents, about 0.2 molar equivalents to about 0.5 molar equivalents, about 0.2 molar equivalents to about 0.75 molar equivalents, about 0.2 molar equivalents to about 1 molar equivalents, about 0.5 molar equivalents to about 0.75 molar equivalents, about 0.5 molar equivalents to about 1 molar equivalents, or about 0.75 molar equivalents to about 1 molar equivalents. In some embodiments, the pharmaceutical composition comprises about 0.1 molar equivalents, about 0.2 molar equivalents, about 0.5 molar equivalents, about 0.75 molar equivalents, or about 1 molar equivalents of the unionized pharmaceutical compound. In some embodiments, the pharmaceutical composition comprises at least about 0.1 molar equivalents, about 0.2 molar equivalents, about 0.5 molar equivalents, or about 0.75 molar equivalents of the unionized pharmaceutical compound. In some embodiments, the pharmaceutical composition comprises at most about 0.2 molar equivalents, about 0.5 molar equivalents, about 0.75 molar equivalents, or about 1 molar equivalents of the unionized pharmaceutical compound. In some embodiments, the pharmaceutical composition comprises about 0.5 equivalents to about 5 equivalents of the unionized pharmaceutical compound. In some embodiments, the pharmaceutical composition comprises about 0.5 equivalents to about 1 equivalents, about 0.5 equivalents to about 2 equivalents, about 0.5 equivalents to about 3 equivalents, about 0.5 equivalents to about 4 equivalents, about 0.5 equivalents to about 5 equivalents, about 1 equivalents to about 2 equivalents, about 1 equivalents to about 3 equivalents, about 1 equivalents to about 4 equivalents, about 1 equivalents to about 5 equivalents, about 2 equivalents to about 3 equivalents, about 2 equivalents to about 4 equivalents, about 2 equivalents to about 5 equivalents, about 3 equivalents to about 4 equivalents, about 3 equivalents to about 5 equivalents, or about 4 equivalents to about 5 equivalents of the unionized pharmaceutical compound. In some embodiments, the pharmaceutical composition comprises about 0.5 equivalents, about 1 equivalents, about 2 equivalents, about 3 equivalents, about 4 equivalents, or about 5 equivalents of the unionized pharmaceutical compound. In some embodiments, the pharmaceutical composition comprises at least about 0.5 equivalents, about 1 equivalents, about 2 equivalents, about 3 equivalents, or about 4 equivalents of the unionized pharmaceutical compound. In some embodiments, the pharmaceutical composition comprises at most about 1 equivalents, about 2 equivalents, about 3 equivalents, about 4 equivalents, or about 5 equivalents of the unionized pharmaceutical compound.

In some embodiments, the pharmaceutical composition comprises a sufficient amount of unionized pharmaceutical compound to form a buffering system with the ionized pharmaceutical compound in the composition. In some embodiments, the buffering system is at a desired pH. In some embodiments, the buffering system results in a desired pH when administered to sublingual tissue.

In some embodiments, the buffering system results in a pH within a target range of the pKa value of the pharmaceutical compound. In some embodiments, the target pH is within about 0.2 pH units to about 2 pH units of the pKa value. In some embodiments, the target pH is within about 0.2 pH units to about 0.5 pH units, about 0.2 pH units to about 1 pH units, about 0.2 pH units to about 1.5 pH units, about 0.2 pH units to about 2 pH units, about 0.5 pH units to about 1 pH units, about 0.5 pH units to about 1.5 pH units, about 0.5 pH units to about 2 pH units, about 1 pH units to about 1.5 pH units, about 1 pH units to about 2 pH units, or about 1.5 pH units to about 2 pH units of the pKa value. In some embodiments, the target pH is within about 0.2 pH units, about 0.5 pH units, about 1 pH units, about 1.5 pH units, or about 2 pH units of the pKa value. In some embodiments, the target pH is within at least about 0.2 pH units, about 0.5 pH units, about 1 pH units, or about 1.5 pH units of the pKa value. In some embodiments, the target pH is within at most about 0.5 pH units, about 1 pH units, about 1.5 pH units, or about 2 pH units of the pKa value.

In some embodiments, the buffering system results in a pH wherein a certain percentage of the pharmaceutical compound is ionized upon administration. In some embodiments, the buffering system results in a pH wherein about 1% to about 99% of the pharmaceutical compound is ionized. In some embodiments, the buffering system results in a pH wherein about 1% to about 5%, about 1% to about 10%, about 1% to about 25%, about 1% to about 50%, about 1% to about 70%, about 1% to about 90%, about 1% to about 95%, about 1% to about 99%, about 5% to about 10%, about 5% to about 25%, about 5% to about 50%, about 5% to about 70%, about 5% to about 90%, about 5% to about 95%, about 5% to about 99%, about 10% to about 25%, about 10% to about 50%, about 10% to about 70%, about 10% to about 90%, about 10% to about 95%, about 10% to about 99%, about 25% to about 50%, about 25% to about 70%, about 25% to about 90%, about 25% to about 95%, about 25% to about 99%, about 50% to about 70%, about 50% to about 90%, about 50% to about 95%, about 50% to about 99%, about 70% to about 90%, about 70% to about 95%, about 70% to about 99%, about 90% to about 95%, about 90% to about 99%, or about 95% to about 99% of the pharmaceutical compound is ionized. In some embodiments, the buffering system results in a pH wherein about 1%, about 5%, about 10%, about 25%, about 50%, about 70%, about 90%, about 95%, or about 99% of the pharmaceutical compound is ionized. In some embodiments, the buffering system results in a pH wherein at least about 1%, about 5%, about 10%, about 25%, about 50%, about 70%, about 90%, or about 95% of the pharmaceutical compound is ionized. In some embodiments, the buffering system results in a pH wherein at most about 5%, about 10%, about 25%, about 50%, about 70%, about 90%, about 95%, or about 99% of the pharmaceutical compound is ionized. In some embodiments, the percent ionization is measured immediately after administration.

In some embodiments, the pharmaceutical composition comprises additional molar equivalents of unionized pharmaceutical compound compared to complexing agent of the complexing agent/protonated pharmaceutical compound salt. In some embodiments, the additional molar equivalents of unionized pharmaceutical compound comprise the same pharmaceutical compound as the ionized pharmaceutical compound in the pharmaceutical composition which comprises a protonated nitrogen atom to provide counterion to the acidic functional groups of the complexing agent. In some embodiments, the additional molar equivalents of unionized pharmaceutical compound comprise a pharmaceutical compound different from the ionized pharmaceutical compound in the pharmaceutical composition which comprises a protonated nitrogen atom to provide counterion to the acidic functional groups of the complexing agent. In some embodiments, the pharmaceutical composition comprises about 0.01 molar equivalents to about 10 molar equivalents of unionized pharmaceutical compound. In some embodiments, the pharmaceutical composition comprises about 0.1 molar equivalents to about 10 molar equivalents of unionized pharmaceutical compound. In some embodiments, the pharmaceutical composition comprises about 0.1 molar equivalents to about 0.5 molar equivalents, about 0.1 molar equivalents to about 1 molar equivalents, about 0.1 molar equivalents to about 2 molar equivalents, about 0.1 molar equivalents to about 3 molar equivalents, about 0.1 molar equivalents to about 5 molar equivalents, about 0.1 molar equivalents to about 7 molar equivalents, about 0.1 molar equivalents to about 10 molar equivalents, about 0.5 molar equivalents to about 1 molar equivalents, about 0.5 molar equivalents to about 2 molar equivalents, about 0.5 molar equivalents to about 3 molar equivalents, about 0.5 molar equivalents to about 5 molar equivalents, about 0.5 molar equivalents to about 7 molar equivalents, about 0.5 molar equivalents to about 10 molar equivalents, about 1 molar equivalents to about 2 molar equivalents, about 1 molar equivalents to about 3 molar equivalents, about 1 molar equivalents to about 5 molar equivalents, about 1 molar equivalents to about 7 molar equivalents, about 1 molar equivalents to about 10 molar equivalents, about 2 molar equivalents to about 3 molar equivalents, about 2 molar equivalents to about 5 molar equivalents, about 2 molar equivalents to about 7 molar equivalents, about 2 molar equivalents to about 10 molar equivalents, about 3 molar equivalents to ab out 5 molar equivalents, about 3 molar equivalents to about 7 molar equivalents, about 3 molar equivalents to about 10 molar equivalents, about 5 molar equivalents to about 7 molar equivalents, about 5 molar equivalents to about 10 molar equivalents, or about 7 molar equivalents to about 10 molar equivalents of unionized pharmaceutical compound of unionized pharmaceutical compound. In some embodiments, the pharmaceutical composition comprises about 0.1 molar equivalents, about 0.5 molar equivalents, about 1 molar equivalents, about 2 molar equivalents, about 3 molar equivalents, about 5 molar equivalents, about 7 molar equivalents, or about 10 molar equivalents of unionized pharmaceutical compound. In some embodiments, the pharmaceutical composition comprises at least about 0.1 molar equivalents, about 0.5 molar equivalents, about 1 molar equivalents, about 2 molar equivalents, about 3 molar equivalents, about 5 molar equivalents, or about 7 molar equivalents of unionized pharmaceutical compound. In some embodiments, the pharmaceutical composition comprises at most about 0.5 molar equivalents, about 1 molar equivalents, about 2 molar equivalents, about 3 molar equivalents, about 5 molar equivalents, about 7 molar equivalents, or about 10 molar equivalents of unionized pharmaceutical compound. In some embodiments, the pharmaceutical composition comprises about 0.01 molar equivalents to about 20 molar equivalents of unionized pharmaceutical compound compared to the complexing agent. In some embodiments, the pharmaceutical composition comprises about 1 molar equivalent to about 20 molar equivalents of unionized pharmaceutical compound compared to the complexing agent. In some embodiments, at least a portion these additional equivalents of the unionized pharmaceutical compound relative to the complexing agent are complexed to the complexing agent (e.g. up to about 1 molar equivalent of the unionized pharmaceutical compound).

In some embodiments, the pharmaceutical composition comprises additional components to improve properties specific to sublingual administration. Non-limiting examples of such additional excipients or components include permeation enhancers, stabilizers, lyophilization excipients, disintegrants, masking agents, flavors, binders, sweeteners, bittering agents, texturing agents, wetting agents, dispersing agents, additional buffers, and other such excipients.

### Additional Formulation Components

The compounds (e.g., rotigotine, eletriptan, copanlisib, remdesivir, nafamostat (nafamostat mesylate), melevodopa, tigecycline, naloxone, amikacin, 1-amantadine, 2-amantadine, rimantadine, amifampridine, caspofungin, rapamycin, clonidine, ketamine, methoxetamine, deschloroketamine, mescaline, tryptamines, phenethylamines, lysergamides, racemorphan, levorphanol, racemethorphan, 3-metylmethcathinone, ethylone, diphenhydramine, etc.) of the present disclosure may be in the form of compositions suitable for administration to a subject. In general, such compositions are “pharmaceutical compositions” comprising a compound (e.g., rotigotine, eletriptan, copanlisib, remdesivir, nafamostat (nafamostat mesylate), melevodopa, tigecycline, naloxone, amikacin, 1-amantadine, 2-amantadine, rimantadine, amifampridine, caspofungin, rapamycin, clonidine, ketamine, methoxetamine, deschloroketamine, mescaline, tryptamines, phenethylamines, lysergamides, racemorphan, levorphanol, racemethorphan, 3-metylmethcathinone, ethylone, diphenhydramine, etc.) and one or more pharmaceutically acceptable or physiologically acceptable diluents, carriers or excipients. In some embodiments, the compounds (e.g., ketamine, methoxetamine, deschloroketamine, mescaline, tryptamines, phenethylamines, lysergamides, racemorphan, levorphanol, racemethorphan, 3-metylmethcathinone, ethylone, diphenhydramine, etc.) are present in a therapeutically acceptable amount. The pharmaceutical compositions may be used in the methods of the present disclosure; thus, for example, the pharmaceutical compositions can be administered ex vivo or in vivo to a subject in order to practice the therapeutic and prophylactic methods and uses described herein.

The pharmaceutical compositions of the present disclosure can be formulated to be compatible with the intended method or route of administration; exemplary routes of administration are set forth herein.

In certain embodiments of the pharmaceutical compositions described herein, the co-solvent comprises PEG200, PEG300, PEG400, PEG600, propylene glycol, ethanol, poly sorbate 20, polysorbate 80, cremephor, glycerin, benzyl alcohol, dimethylacetamide (DMA), N-methyl-2-pyrrolidone (NMP), tert-butanol, or combinations thereof.

In certain embodiments, the dosage form or pharmaceutical composition comprises a surface-active agent.

In certain embodiments of the pharmaceutical compositions described herein, the surface-active agent comprises polyoxyethylene sorbitan monooleate (Tween 80), sorbitan monooleate, polyoxyethylene sorbitan monolaurate (Tween 20), lecithin, polyoxyethylene-polyoxypropylene copolymers (Pluronics1), or combinations thereof.

In certain embodiments, the dosage form or pharmaceutical composition comprises a non-ionic surfactant.

In certain embodiments of the pharmaceutical compositions described herein, the non-ionic surfactant comprises Cremophor RH40, Cremophor RH60, d-alpha-topopherol polyethylene glycol 1000 succinate, polysorbate 20, polysorbate 80, Solutol HS 15, sorbitan monooleate, poloxamer 407, Labrafil M-1944CS, Labrafil M-2125CS, Labrasol, Gellucire 44/14, Softigen 767, or combinations thereof.

In some embodiments, the pharmaceutical composition comprises one or more co-solvents, solubilization/solubilizing agents, stabilization agents, antioxidants, preservatives, cryoprotectants, lyoprotectants, bulking agents, tonicity-adjusting agents, or antimicrobial agents. In some embodiments, the pharmaceutical composition comprises at least one co-solvent. In some embodiments, the pharmaceutical composition comprises at least one solubilizing agent. In some embodiments, the pharmaceutical composition comprises at least one stabilization agent. In some embodiments, the pharmaceutical composition comprises at least one antioxidant. In some embodiments, the pharmaceutical composition comprises at least one preservative. In some embodiments, the pharmaceutical composition comprises at least one cryoprotectant. In some embodiments, the pharmaceutical composition comprises at least one lyoprotectant. In some embodiments, the pharmaceutical composition comprises at least one bulking agent. In some embodiments, the pharmaceutical composition comprises at least one tonicity-adjusting agent. In some embodiments, the pharmaceutical composition comprises at least one antimicrobial agent.

In some embodiments, the formulation or pharmaceutical composition is a pharmaceutical composition. In some embodiments, the formulation is in the form of a sterile injectable aqueous or oleaginous suspension. This suspension may be formulated according to the known art using those suitable dispersing or wetting agents and suspending agents mentioned herein. The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution, Cremophor® EL (BASF, Parsippany, NJ) or phosphate buffered saline (PBS), ethanol, polyol (e.g., glycerol, propylene glycol, and liquid polyethylene glycol), and suitable mixtures thereof. In addition, sterile fixed oils are conventionally employed as a solvent or suspending medium; for this purpose, any bland fixed oil may be employed, including synthetic mono- or diglycerides. Moreover, fatty acids, such as oleic acid, find use in the preparation of injectables. Prolonged absorption of particular injectable formulations can be achieved by including an agent that delays absorption (e.g., aluminum monostearate or gelatin). In some embodiments, the formulation comprises a co-solvent. In some embodiments, a suitable co-solvent is propylene glycol, glycerin, ethanol, polyethylene glycol (300 and 400), Sorbitol, dimethylacetamide, Cremophor EL, or N-methyl-2-pyrrolidone, or dimethylsulfoxide.

In some embodiments, the formulation or pharmaceutical composition is an aqueous suspension. Aqueous suspensions contain active materials in admixture with excipients suitable for the manufacture thereof. Such excipients can be suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives (e.g. benzethonium chloride).

In some embodiments, the formulation or pharmaceutical composition comprises a stabilization agent. In some embodiments, the formulation comprises a surface-active solubilization agent. Surface-active solubilization agents include, but are not limited to: polyoxyethylene sorbitan monooleate (Tween 80), sorbitan monooleate, polyoxyethylene sorbitan monolaurate (Tween 20), lecithin, and Polyoxyethylene-polyoxypropylene copolymers (Pluronics1). In some embodiments, the formulation comprises a non-ionic surfactant solubilization agent. Non-ionic surfactants include, but are not limited: Cremophor RH 40, Cremophor RH 60, d-alpha-tocopherol polyethylene glycol 1000 succinate, polysorbate 20, polysorbate 80, Solutol HS 1, sorbitan monooleate, poloxamer 407, Labrafil M-1944CS, Labrafil M-2125CS, Labrasol, Gellucire 44/14, Softigen 767, and mono-fatty esters and di-fatty acid esters of PEG 300, 400, and 1750. In some embodiments, the formulation comprises a phospholipid solubilizing agent such as, hydrogenated soy phosphatidylcholine, phosphatidylcholine, distearoylphosphatidylglycerol, L-alpha-dimyristoylphosphatidylcholine, or L-alpha-dimyristoylphosphatidylglycerol.

In some embodiments, the formulation or pharmaceutical composition comprises a complexation agent. In some embodiments, the complexation agent is hydroxypropyl-b-cyclodextrin, bulfobutylether-b-cyclodextrin (Captisol1), or polyvinylpyrrolidone. In some embodiments, the complexation agent is an amino acid such as, arginine, lysine, or histidine. In some embodiments, the formulation or pharmaceutical composition comprises a cyclodextrin excipient. Cyclodextrin excipients are used to enhance the stability, tolerability and absorption of compounds in parenteral aqueous solutions. Common cyclodextrin excipients include but are not limited to: alpha-Cyclodextrin (alpha-CD), beta-Cyclodextrin (beta-CD), gamma-Cyclodextrin (gamma-CD), Diethyl-ethyl-beta-cyclodextrin (DE-beta-CD), Dimethyl-ethyl-beta-cyclodextrin (DM-beta-CD), Hydroxypropyl-beta-cyclodextrin (HP-beta-CD), Hydroxypropyl-gamma-cyclodextrin (HP-gamma-CD), Methyl-b-cyclodextrin (M-beta-CD), Sulfobutylether-beta-cyclodextrin (SBE-beta-CD), Randomly methylated-beta-CD (RM-beta-CD), Maltosyl-beta-CD (mal-beta-CD), Hydroxypropyl-alpha-CD.

The formulations or pharmaceutical compositions of the present disclosure may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

The formulation or pharmaceutical composition typically comprises a therapeutically effective amount of an active compound (e.g., ketamine, methoxetamine, deschloroketamine, mescaline, tryptamines, phenethylamines, lysergamides, racemorphan, levorphanol, or racemethorphan, 3-metylmethcathinone, ethylone, diphenhydramine, etc.), or a hydrate, solvate, tautomer, or pharmaceutically acceptable salt thereof, and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate-buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a triethanolamine (Tris) buffer, histidine, bicarbonate; N-(2-Hydroxyethyl) piperazine-N′-(2-ethanesulfonic acid) (HEPES); 2-(N-Morpholino) ethanesulfonic acid (MES); 2-(N-Morpholino) ethanesulfonic acid sodium salt (MES); 3-(N-Morpholino) propanesulfonic acid (MOPS); and N-tris [Hydroxymethyl]methyl-3-aminopropanesulfonic acid (TAPS).

Many active pharmaceutical ingredients (APIs), including pharmaceutical compounds, are weak acids or weak bases. Weak acids or weak bases can exist in an un-ionized form or as an ionized complex prepared by the addition of a base or acid respectively. The resultant complex is stabilized by ionic interactions and is known as a salt. This complex exists via an ionic bond between an ionized API and an oppositely charged counterion. Salts offer a number of advantages over their un-ionized counterparts. The choice of counterion can have a large influence on the salts properties and the use of a given salt form of a given API in a pharmaceutical product is influenced and guided by a number of factors for example stability (photo, hydrolytic and thermal), solubility, physicochemical properties, solid state properties (crystallinity, polymorphism, particle size, crystal morphology, melting point, compactability), production considerations (e.g., ease of handling and processing), dissolution rate, modulation of drug release, compatibility with excipients and containers, ease and consistency of production, desired route of administration, and organoleptic factors (e.g., taste). Furthermore, with respect to injection, salt can influence pain and irritation at the injection site.

APIs that are weak acids or weak bases can act as their own buffers at pH's near the pKa of the API. For example, a compound which comprises an amino functionality with a pKa of ˜7.5, and can thus serve as a buffer in the region of about ±2 pH units from the pKa (e.g. from pHs of about 5.5 to about 9.5). When the formulation has a target pH within this range, an additional buffer may not be required. In some embodiments, the pharmaceutical composition provided herein does not comprise an additional buffer.

With regard to cyclodextrin solubilization, specific salts of various APIs have been found to form multicomponent complexes/systems or ternary systems which can have distinct desirable properties as compared to their standard binary complexes/systems counterparts prepared between the cyclodextrin and the un-ionized API, as well as compared to other multicomponent ternary complexes/systems involving different salt forms of that API. These multicomponent complexes/systems can thus dramatically influence solubility of the API in aqueous solutions, dissolution rates, can influence product stability, and pharmacokinetic properties of the pharmaceutical preparation.

After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form. In some embodiments, the pharmaceutical composition is provided in a single-use container (e.g., a single-use vial, ampule, syringe, or autoinjector (similar to, e.g., an EpiPen®)), whereas a multi-use container (e.g., a multi-use vial) is provided in other embodiments.

Formulations or pharmaceutical compositions can also include carriers to protect the composition against rapid degradation or elimination from the body, such as a controlled release formulation, including liposomes, hydrogels, prodrugs and microencapsulated delivery systems. For example, a time-delay material such as glyceryl monostearate or glyceryl stearate alone, or in combination with a wax, may be employed. Any drug delivery apparatus may be used to deliver a pharmaceutical compound, including implants (e.g., implantable pumps) and catheter systems, slow injection pumps and devices, all of which are well known to the skilled artisan.

In some embodiments, the formulation or pharmaceutical composition is stored in a reservoir of the drug delivery device. In some embodiments, the formulation is stored in a cartridge that is insertable and/or attachable to the drug delivery device. In some embodiments, the cartridge and/or drug delivery device comprises a product label for intramuscular injection. In some embodiments, the cartridge and/or drug delivery device comprises a product label for subcutaneous injection. In some embodiments, the cartridge and/or drug delivery device comprises a product label for intravenous injection. In some embodiments, disclosed herein is a kit comprising a product label for intramuscular injection. In some embodiments, disclosed herein is a kit comprising a product label for subcutaneous injection. In some embodiments, disclosed herein is a kit comprising a product label for intravenous injection.

In some embodiments, the formulation or pharmaceutical composition is a liquid formulation comprising a pharmaceutical compound.

It is frequently beneficial to improve one of more physical properties of the treatment modalities disclosed herein and/or the manner in which they are administered. Improvements of physical properties include, for example, methods of increasing water solubility, bioavailability, serum half-life, and/or therapeutic half-life; and/or modulating biological activity. Modifications known in the art include pegylation, Fc-fusion and albumin fusion. Although generally associated with large molecule agents (e.g., polypeptides), such modifications have recently been evaluated with particular small molecules. By way of example, Chiang, M. et al. (J. Am. Chem. Soc., 2014, 136 (9): 3370-73) describe a small molecule agonist of the adenosine 2a receptor conjugated to the immunoglobulin Fc domain. The small molecule-Fc conjugate retained potent Fc receptor and adenosine 2a receptor interactions and showed superior properties compared to the unconjugated small molecule. Covalent attachment of PEG molecules to small molecule therapeutics has also been described (Li, W. et al., Progress in Polymer Science, 2013 38:421-44).

The pharmaceutical compound of the present disclosure may be administered to a subject in an amount that is dependent upon, for example, the goal of administration (e.g., the degree of resolution desired); the age, weight, sex, and health and physical condition of the subject to which the formulation is being administered; the route of administration; and the nature of the disease, disorder, condition or symptom thereof. The dosing regimen may also take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan.

In general, dosing parameters dictate that the dosage amount be less than an amount that could be irreversibly toxic to the subject (the maximum tolerated dose (MTD) and not less than an amount required to produce a measurable effect on the subject. Such amounts are determined by, for example, the pharmacokinetic and pharmacodynamic parameters associated with ADME, taking into consideration the route of administration and other factors.

An effective dose (ED) is the dose or amount of an agent that produces a therapeutic response or desired effect in some fraction of the subjects taking it. The “median effective dose” or ED₅₀ of an agent is the dose or amount of an agent that produces a therapeutic response or desired effect in 50% of the population to which it is administered. Although the ED₅₀ is commonly used as a measure of reasonable expectance of an agent's effect, it is not necessarily the dose that a clinician might deem appropriate taking into consideration all relevant factors. Thus, in some situations the effective amount is more than the calculated ED₅₀, in other situations the effective amount is less than the calculated ED₅₀, and in still other situations the effective amount is the same as the calculated ED₅₀.

In addition, an effective dose of the compound of the present disclosure may be an amount that, when administered in one or more doses to a subject, produces a desired result relative to a healthy subject. For example, for a subject experiencing a particular disorder, an effective dose may be one that improves a diagnostic parameter, measure, marker and the like of that disorder by at least about 5%, at least about 10%, at least about 20%, at least about 25%, at least about 30%, at least about 40%, at least about 50%, at least about 60%, at least about 70%, at least about 80%, at least about 90%, or more than 90%, where 100% is defined as the diagnostic parameter, measure, marker and the like exhibited by a normal subject.

In embodiments, the dosage of the compound is contained in a “unit dosage form.” The phrase “unit dosage form” refers to physically discrete units, each unit including a predetermined amount of the compound (e.g., ketamine, methoxetamine, deschloroketamine, mescaline, tryptamines, phenethylamines, lysergamides, racemorphan, levorphanol, or racemethorphan, 3-metylmethcathinone, ethylone, diphenhydramine, etc.), or a hydrate, solvate, or pharmaceutically acceptable salt thereof), sufficient to produce the desired effect. It will be appreciated that the parameters of a unit dosage form will depend on the particular agent and the effect to be achieved.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents, such as those set forth above, and flavoring agents may be added to provide a palatable oral preparation.

Dispersible powders and granules suitable for preparation of an aqueous suspension by the addition of water provide the active ingredient in admixture with a dispersing or wetting agent, and optionally one or more suspending agents and/or preservatives. Suitable dispersing or wetting agents and suspending agents are exemplified herein.

Depot injections, which are generally administered subcutaneously or intramuscularly, may also be utilized to release the compound (e.g., ketamine, methoxetamine, deschloroketamine, mescaline, tryptamines, phenethylamines, lysergamides, racemorphan, levorphanol, or racemethorphan, 3-metylmethcathinone, ethylone, diphenhydramine, etc.) disclosed herein over a defined period of time. Depot injections are usually either solid-or oil-based and generally comprise at least one of the formulation components set forth herein. One of ordinary skill in the art is familiar with possible formulations and uses of depot injections.

Some formulations include one or more stabilization agents. Potential stabilization agents that are contemplated include buffers: Acetate, Citrate, Sodium Citrate, Tartrate, Phosphate, histidine, bicarbonate, Triethanolamine (TRIS) and their salts. In some formulations, the potential stabilization agents might include antioxidants and preservatives such as: Ascorbic acid, Acetylcysteine (NAC), Sulfurous acid salts (bisulfite, metabisulfite), Monothioglyercol. Butylated hydroxyanisole (BHA), Butylated hydroxytoluene (BHT), Tert-butylhydroquinone (TBHQ), 2′,4′,5′-Trihydroxybutyrophenone phenylhydrazone (THBP), Ethylenediaminetetraacetic acid (EDTA), Sodium formaldehyde sulfoxylate (SFS), Tocopherol (Vitamin E), Ascorbyl palmitate, Gallates (e.g., propyl gallate, octyl gallate, lauryl gallate), Cysteine ethyl ether, Tartaric acid, Phosphoric acid, Thiourea, Sodium thioglycolate, Nitrogen, and/or Argon.

In some formulations, the potential stabilization agents might include bulking agents, cryoprotectants, and lyoprotectants. Agents that were considered include: Mannitol, Glycine, Sucrose, Lactose, Trehalose, Dextran, Povidone, Sorbitol and/or Polydextrose. In some formulations potential stabilization agents might include tonicity-adjusting agents. Agents that were considered include: sodium chloride, Glycerin, Mannitol, Dextrose, and/or glycerol. In some formulations the potential stabilization agents might include antimicrobial agents including, but not limited to: Phenol, Meta-cresol, Benzyl alcohol, parabens (methyl, propyl, or butyl), benzalkonium chloride, benzethonium chloride, chlorobutanol, Myristyl gamma picolinium chloride, 2-phenoxyethanol, Phenethyl alcohol, Sorbates (sorbic acid, sodium sorbate), Ethanol, and/or Propylene glycol.

In some formulations, soothing agents might include topical analgesics such as: lidocaine, benzocaine, tetracaine, bupivicaine, ropivacaine, and/or levobupivacaine.

In some formulations, emulsion stabilizers include hydroxyethyl cellulose, hydroxypropylcellulose, and/or hydroxypropyl methyl cellulose (hypromellose).

The compound (e.g., ketamine, methoxetamine, deschloroketamine) contemplated by the present disclosure may be in the form of any other suitable pharmaceutical composition currently known or developed in the future.

### III. Methods

### Methods of Treatment Using Pharmaceutical Compositions

In an aspect, provided herein is a method of treating a disease or condition in a subject, the method comprising administering to the subject a pharmaceutical composition provided herein. The disease or condition will depend on the particular compound selected in the pharmaceutical composition. In some embodiments, the method comprises administering the pharmaceutical composition according to any of the modes of administration described herein. The mode of administration can include subcutaneous injection or intramuscular injection, which may be carried out using a pump or other injection device. As an example, a subcutaneous pump can be used to provide continuous infusion of an opioid and/or dissociative drug.

### Anti-Infection Compounds and Others

Generally, the anti-viral, anti-bacterial, or anti-fungal compounds provided herein (e.g. remdesivir, nafamostat, tigecycline, caspofungin, etc.) will be useful for a variety of indications. In some embodiments, formulations and methods disclosed herein are used to treat infections. Examples of infection and infectious diseases include hepatitis C, Ebola virus disease, Marburg virus infections, COVID-19, pancreatitis, pancreatic cancer, complicated skin and structure infections, complicated intra-abdominal infections, bacterial pneumonia, joint infections, intra-abdominal infections, meningitis, pneumonia, sepsis, and urinary tract infections, flu infections, aspergillosis intra-abdominal abscesses, peritonitis, pleural cavity infections, and esophagitis. Other compounds described herein (e.g., rotigotine, eletriptan, copanlisib, rapamycin, clonidine, amifampridine, melevodopa, naloxone, etc.) may be used to treat other disorders including Parkinson's disease (PD), restless legs syndrome (RLS), migraine headaches, follicular lymphoma, opioid overdose, organ transplant rejection, lymphangioleiomyomatosis, perivascular epithelioid cell tumor (PEComa), opioid dependence and detoxification, pain including but not limited to neuropathic pain, complex regional pain syndrome and post herpetic neuralgia, attention deficit hyperactivity disorder, high blood pressure, diarrhea, menopausal flushing, drug withdrawal (alcohol, opioids, or nicotine), and spasticity.

### Dissociative Compounds

Generally, the dissociative compounds provided herein (e.g. ketamine or analogs or derivatives thereof) will be useful for all of the listed indications. In some embodiments, formulations and methods disclosed herein are used to treat pain or a pain disorder. In some embodiments, chronic pain refers to pain having a duration of greater than 3 months. Examples of pain and pain disorders include pain that is not otherwise specified (NOS) such as acute pain, body aches, buttock muscular pain, lower back pain, chronic back pain, chronic coccygeal pain, chronic low back pain, chronic malignant pain, chronic neck pain, chronic nonmalignant pain, chronic pain, and generalized pain. In some embodiments, the pain can include pain crisis, pain in buttocks, pain of coccyx (chronic or acute), or neoplasm related pain (chronic or acute). In some embodiments, the pain is chronic pain. In some embodiments, the pain is acute pain.

In some embodiments, the pain is chronic post-procedural and/or post-surgical pain. Examples of post-procedural pain include chronic pain due to bilateral total hip arthroplasty, chronic pain due to bilateral total knee arthroplasty, chronic pain due to left total hip arthroplasty, chronic pain due to left total knee replacement, chronic pain due to right total hip arthroplasty, chronic pain due to right total knee replacement, chronic pain following bilateral partial hip arthroplasty, chronic pain following bilateral partial knee arthroplasty, chronic pain following left partial hip arthroplasty, chronic pain following left partial knee arthroplasty, chronic pain following right partial hip arthroplasty, chronic pain following right partial knee arthroplasty, pain due to bilateral total hip arthroplasty, pain due to bilateral total knee arthroplasty, pain due to left total hip arthroplasty, pain due to left total knee replacement, pain due to right total hip arthroplasty, pain due to right total knee replacement, pain following bilateral partial hip arthroplasty, pain following bilateral partial knee arthroplasty, pain following left partial hip arthroplasty, pain following left partial knee arthroplasty, pain following right partial hip arthroplasty, pain following right partial knee arthroplasty, chronic post-mastectomy pain, chronic post-mastectomy pain, and chronic postoperative pain.

In some embodiments, the pain is chronic pain due to trauma or injury. In some embodiments, the pain is a chronic pain syndrome, also referred to as chronic pain associated with psychosocial dysfunction or psychosocial dysfunction due to chronic pain. In some embodiments, the pain is a neoplasm related pain or pain due to neoplastic disease (chronic or acute). In some embodiments, the pain is causalgia (lower limb and/or upper limb).

In some embodiments, the pain is central pain syndrome, complex regional pain syndrome I, complex regional pain syndrome II (lower limb), or complex regional pain syndrome II (upper limb).

In some embodiments, the disease or disorder is a psychiatric disorder. In some embodiments, the psychiatric disorder is major depressive disorder, treatment resistant major depressive disorder, suicidality, suicidal ideation, dysthymia or persistent depressive disorder, bipolar depressive disorder type I, bipolar depressive disorder type II, chronic pain, eating disorder NOS, pain disorder NOS, panic disorder, post-traumatic stress disorder, obsessive-compulsive disorder, complex regional pain syndrome, reflex sympathetic dystrophy, or any combination thereof.

In some embodiments, the disease or disorder is a cognitive or neurological disorder. In some embodiments, the cognitive or neurological disorder is Huntington's disease, Parkinson's disease, frontotemporal dementia, dementia, Alzheimer's disease, amyotrophic lateral sclerosis, spinal cord trauma, stroke, diffuse traumatic brain injury, HIV-associated dementia, epilepsy, Rett syndrome, dyskinesia, unspecified dystonia, or pseudobulbar affect.

In some embodiments, formulations and methods disclosed herein are used to treat one or more personality disorders. Examples of personality disorders include avoidant personality disorder, dependent personality disorder, antisocial personality disorder, histrionic personality disorder, borderline personality disorder, obsessive-compulsive personality disorder, cyclothymic personality disorder, obsessive compulsive disorder, and impulse control disorder (NOS).

In some embodiments, formulations and methods disclosed herein are used to treat one or more of major depressive disorder, treatment resistant major depressive disorder, suicidality, suicidal ideation, dysthymia, bipolar disorder (Type I-Depressed), bipolar disorder (Type II-Depressed), post-traumatic stress disorder (PTSD), panic disorder, generalized anxiety disorder, and substance abuse induced mood disorder.

In some embodiments, formulations and methods disclosed herein are used to treat drug dependence. Examples of drug dependence include opiate dependence, benzodiazepine dependence, sedative (hypnotic or anxiolytic) dependence, alcohol dependence, stimulant dependence, cocaine dependence, cannabis detoxification, opiate dependence (with withdrawal), benzodiazepine dependence (with withdrawal), sedative (with withdrawal) dependence, alcohol dependence (with withdrawal), stimulant dependence (with withdrawal), cocaine dependence (with withdrawal), and cannabis detoxification (with withdrawal).

In another aspect is provided a method of treating, preventing, or ameliorating at least one symptom of a disorder, disease, or condition with the pharmaceutical compositions disclosed herein, including embodiments, wherein the disorder, disease, or condition is a mental or psychiatric disorder, a mood disorder, a neurological condition or disorder, type 2 diabetes mellitus and/or complications thereof, endometriosis, glaucoma, pain, or an inflammatory disorder.

### Psychedelic Compounds

Generally, the psychedelic compounds provided herein (e.g. mescaline, etc.) will be useful for all of the listed indications.

In some embodiments, the disease or disorder is a psychiatric disorder. In some embodiments, the psychiatric disorder is major depressive disorder, treatment resistant major depressive disorder, dysthymia, suicidality, suicidal ideation, dysthymia or persistent depressive disorder, bipolar depressive disorder type I, bipolar depressive disorder type II, chronic pain, eating disorder NOS, pain disorder NOS, panic disorder, post-traumatic stress disorder, obsessive-compulsive disorder, personality disorders, complex regional pain syndrome, reflex sympathetic dystrophy, post-concussive memory disorders and cognitive disorders, traumatic brain injury, post-chemotherapy cognitive dysfunction and memory disorders, inflammatory disorders, cognitive disorders, memory disorders, dementia NOS, fatigue or any combination thereof. In some embodiments, the psychiatric disorder is major depressive disorder, treatment resistant major depressive disorder, Suicidality, Suicidal Ideation, dysthymia, bipolar I disorder, bipolar II disorder, post-traumatic stress disorder (PTSD), complex trauma, anorexia nervosa, bulimia nervosa, eating disorder NOS, obsessive compulsive disorder, a substance-related disorder (e.g., cannabis dependence or withdrawal, barbiturate dependence or withdrawal, benzodiazepine dependence or withdrawal, amphetamine dependence or withdrawal, opioid dependence or withdrawal, alcohol dependence or withdrawal, cocaine dependence or withdrawal).

In some embodiments, formulations and methods disclosed herein are used to treat one or more personality disorders. Examples of personality disorders include avoidant personality disorder, dependent personality disorder, antisocial personality disorder, histrionic personality disorder, borderline personality disorder, obsessive-compulsive personality disorder, cyclothymic personality disorder, obsessive compulsive disorder, and impulse control disorder (NOS).

In some embodiments, formulations and methods disclosed herein are used to treat one or more of major depressive disorder, treatment resistant major depressive disorder, suicidality, suicidal ideation, dysthymia, bipolar disorder (Type I-Depressed), bipolar disorder (Type II-Depressed), post-traumatic stress disorder (PTSD), panic disorder, generalized anxiety disorder, and substance abuse induced mood disorder.

In some embodiments, formulations and methods disclosed herein are used to treat drug dependence. Examples of drug dependence include opiate dependence, benzodiazepine dependence, sedative (hypnotic or anxiolytic) dependence, alcohol dependence, stimulant dependence, cocaine dependence, cannabis detoxification, opiate dependence (with withdrawal), benzodiazepine dependence (with withdrawal), sedative (with withdrawal) dependence, alcohol dependence (with withdrawal), stimulant dependence (with withdrawal), cocaine dependence (with withdrawal), and cannabis detoxification (with withdrawal).

In some embodiments, formulations and methods disclosed herein are used to treat pain or a pain disorder. In some embodiments, when the embodiments, formulations, and methods provided herein are used to treat pain, the pharmaceutical compounds provided herein are administered as microdoses (e.g., doses below the threshold which induce psychedelic effects). In some embodiments, chronic pain refers to pain having a duration of greater than 3 months. Examples of pain and pain disorders include pain that is not otherwise specified (NOS) such as acute pain, body aches, buttock muscular pain, lower back pain, chronic back pain, chronic coccygeal pain, chronic low back pain, chronic malignant pain, chronic neck pain, chronic nonmalignant pain, chronic pain, and generalized pain. In some embodiments, the pain can include pain crisis, pain in buttocks, pain of coccyx (chronic or acute), or neoplasm related pain (chronic or acute). In some embodiments, the pain is chronic pain. In some embodiments, the pain is acute pain.

In some embodiments, the pain is chronic post-procedural and/or post-surgical pain. Examples of post-procedural pain include chronic pain due to bilateral total hip arthroplasty, chronic pain due to bilateral total knee arthroplasty, chronic pain due to left total hip arthroplasty, chronic pain due to left total knee replacement, chronic pain due to right total hip arthroplasty, chronic pain due to right total knee replacement, chronic pain following bilateral partial hip arthroplasty, chronic pain following bilateral partial knee arthroplasty, chronic pain following left partial hip arthroplasty, chronic pain following left partial knee arthroplasty, chronic pain following right partial hip arthroplasty, chronic pain following right partial knee arthroplasty, pain due to bilateral total hip arthroplasty, pain due to bilateral total knee arthroplasty, pain due to left total hip arthroplasty, pain due to left total knee replacement, pain due to right total hip arthroplasty, pain due to right total knee replacement, pain following bilateral partial hip arthroplasty, pain following bilateral partial knee arthroplasty, pain following left partial hip arthroplasty, pain following left partial knee arthroplasty, pain following right partial hip arthroplasty, pain following right partial knee arthroplasty, chronic post-mastectomy pain, chronic post-mastectomy pain, and chronic postoperative pain.

In some embodiments, the pain is chronic pain due to trauma or injury. In some embodiments, the pain is a chronic pain syndrome, also referred to as chronic pain associated with psychosocial dysfunction or psychosocial dysfunction due to chronic pain. In some embodiments, the pain is a neoplasm related pain or pain due to neoplastic disease (chronic or acute). In some embodiments, the pain is causalgia (lower limb and/or upper limb).

In some embodiments, the pain is central pain syndrome, complex regional pain syndrome I, complex regional pain syndrome II (lower limb), or complex regional pain syndrome II (upper limb).

In some embodiments, the disease or disorder is an inflammatory disorder or disease associated with inflammation. Non-limiting examples of inflammatory disorders and diseases associated with inflammation include asthma, atherosclerosis, autoimmune diseases, autoinflammatory diseases, celiac disease, chronic prostatis, colitis, diverticulitis, glomerulonephritis, inflammatory bowel disease, interstitial cystitis, mastocytosis, pelvic inflammatory disease, reperfusion injury, rheumatic fever, rheumatoid arthritis, rhinitis,

### Opioids

In an aspect, provided herein is a method of treating pain in a subject, the method comprising administering to the subject a pharmaceutical composition provided herein.

In some embodiments, the pain is acute or chronic pain. In some embodiments, the pain is chronic pain.

In some embodiments, the pain is complex regional pain syndrome, central pain syndrome, chronic pain, acute pain, or phantom limb syndrome with pain. In some embodiments, the pain is acute pain. In some embodiments, the pain is post-operative pain. In some embodiments, the pain is from a traumatic injury, such as a battlefield wound. In some embodiments, the pain is cancer pain.

In some embodiments, formulations and methods disclosed herein are used to treat pain or a pain disorder. In some embodiments, chronic pain refers to pain having a duration of greater than 3 months. Examples of pain and pain disorders include pain that is not otherwise specified (NOS) such as acute pain, body aches, buttock muscular pain, lower back pain, chronic back pain, chronic coccygeal pain, chronic low back pain, chronic malignant pain, chronic neck pain, chronic nonmalignant pain, chronic pain, and generalized pain. In some embodiments, the pain can include pain crisis, pain in buttocks, pain of coccyx (chronic or acute), or neoplasm related pain (chronic or acute). In some embodiments, the pain is chronic pain. In some embodiments, the pain is acute pain.

In some embodiments, the pain is chronic post-procedural and/or post-surgical pain. Examples of post-procedural pain include chronic pain due to bilateral total hip arthroplasty, chronic pain due to bilateral total knee arthroplasty, chronic pain due to left total hip arthroplasty, chronic pain due to left total knee replacement, chronic pain due to right total hip arthroplasty, chronic pain due to right total knee replacement, chronic pain following bilateral partial hip arthroplasty, chronic pain following bilateral partial knee arthroplasty, chronic pain following left partial hip arthroplasty, chronic pain following left partial knee arthroplasty, chronic pain following right partial hip arthroplasty, chronic pain following right partial knee arthroplasty, pain due to bilateral total hip arthroplasty, pain due to bilateral total knee arthroplasty, pain due to left total hip arthroplasty, pain due to left total knee replacement, pain due to right total hip arthroplasty, pain due to right total knee replacement, pain following bilateral partial hip arthroplasty, pain following bilateral partial knee arthroplasty, pain following left partial hip arthroplasty, pain following left partial knee arthroplasty, pain following right partial hip arthroplasty, pain following right parti al knee arthroplasty, chronic post-mastectomy pain, chronic post-mastectomy pain, and chronic postoperative pain.

In some embodiments, the pain is chronic pain due to trauma or injury. In some embodiments, the pain is a chronic pain syndrome, also referred to as chronic pain associated with psychosocial dysfunction or psychosocial dysfunction due to chronic pain. In some embodiments, the pain is a neoplasm related pain or pain due to neoplastic disease (chronic or acute). In some embodiments, the pain is causalgia (lower limb and/or upper limb).

In some embodiments, the pain is central pain syndrome, complex regional pain syndrome I, complex regional pain syndrome II (lower limb), or complex regional pain syndrome II (upper limb).

In an aspect, provided herein, is a method of treating a disease or condition in a subject, the method comprising administering to the subject a pharmaceutical composition provided herein, wherein the disease or condition is depression or opioid overdose. In some embodiments, the disease or condition is opioid overdose and the opioid of the pharmaceutical composition is an opioid receptor antagonist, such as naloxone or naltrexone.

### Empathogenic and Entactogenic Compounds

In an aspect, provided herein is a method of treating a disease or disorder in a subject, the method comprising administering to the subject a pharmaceutical composition provided herein.

In some embodiments, the disease or disorder is acute pain or chronic pain. In some embodiments, the pain is acute pain. In some embodiments, the pain is post-operative pain. In some embodiments, the pain is from a traumatic injury, such as a battlefield wound. In some embodiments, the pain is cancer pain.

In some embodiments, formulations and methods disclosed herein are used to treat pain or a pain disorder. In some embodiments, chronic pain refers to pain having a duration of greater than 3 months. Examples of pain and pain disorders include pain that is not otherwise specified (NOS) such as acute pain, body aches, buttock muscular pain, lower back pain, chronic back pain, chronic coccygeal pain, chronic low back pain, chronic malignant pain, chronic neck pain, chronic nonmalignant pain, chronic pain, and generalized pain. In some embodiments, the pain can include pain crisis, pain in buttocks, pain of coccyx (chronic or acute), or neoplasm related pain (chronic or acute).

In some embodiments, the disease or disorder is a sleep disorder.

In some embodiments, the disease or disorder is an inflammatory disorder or disease associated with inflammation. Non-limiting examples of inflammatory disorders and diseases associated with inflammation include asthma, atherosclerosis, autoimmune diseases, autoinflammatory diseases, celiac disease, chronic prostatis, colitis, diverticulitis, glomerulonephritis, inflammatory bowel disease, interstitial cystitis, mastocytosis, pelvic inflammatory disease, reperfusion injury, rheumatic fever, rheumatoid arthritis, rhinitis, sarcoidosis, transplant rejection, and vasculitis.

In some embodiments, the disease or disorder is a psychiatric disorder. In some embodiments, the psychiatric disorder is major depressive disorder, treatment resistant major depressive disorder, suicidality, suicidal ideation, complex trauma, dysthymia or persistent depressive disorder, bipolar depressive disorder type I, bipolar depressive disorder type II, chronic pain, eating disorder NOS, pain disorder NOS, panic disorder, post-traumatic stress disorder, obsessive-compulsive disorder, complex regional pain syndrome, reflex sympathetic dystrophy, intermittent explosive disorder, or any combination thereof.

In some embodiments, the disease or disorder is a cognitive disorder or a neurological disorder. In some embodiments, the cognitive disorder, or a neurological disorder is Huntington's disease, Parkinson's disease, frontotemporal dementia, dementia, Alzheimer's disease, amyotrophic lateral sclerosis, spinal cord trauma, stroke, diffuse traumatic brain injury, HIV-associated dementia, epilepsy, Rett syndrome, dyskinesia, unspecified dystonia, or pseudobulbar affect.

In some embodiments, formulations and methods disclosed herein are used to treat one or more personality disorders. Examples of personality disorders include avoidant personality disorder, dependent personality disorder, antisocial personality disorder, histrionic personality disorder, borderline personality disorder, obsessive-compulsive personality disorder, cyclothymic personality disorder, obsessive compulsive disorder, and impulse control disorder (NOS).

In some embodiments, formulations and methods disclosed herein are used to treat one or more eating disorders. Examples of eating disorders include anorexia nervosa and bulimia disorder, as well as other eating disorders not otherwise specified (NOS).

In some embodiments, formulations and methods disclosed herein are used to treat one or more of major depressive disorder, treatment resistant major depressive disorder, suicidality, suicidal ideation, dysthymia, bipolar disorder (Type I-Depressed), bipolar disorder (Type II-Depressed), post-traumatic stress disorder (PTSD), panic disorder, generalized anxiety disorder, and substance abuse induced mood disorder.

In some embodiments, formulations and methods disclosed herein are used to treat a cognitive or neurological disorder or condition such as Huntington's disease, Parkinson's disease, frontotemporal dementia, dementia, Alzheimer's disease, amyotrophic lateral sclerosis, spinal cord trauma, stroke, diffuse traumatic brain injury, HIV-associated dementia, epilepsy, suicidal ideation, Rett syndrome, dyskinesia, dystonia (unspecified), or pseudobulbar affect.

In some embodiments, formulations and methods disclosed herein are used to treat drug dependence. Examples of drug dependence include opiate dependence, benzodiazepine dependence, sedative (hypnotic or anxiolytic) dependence, alcohol dependence, stimulant dependence, cocaine dependence, cannabis detoxification, opiate dependence (with withdrawal), benzodiazepine dependence (with withdrawal), sedative (with withdrawal) dependence, alcohol dependence (with withdrawal), stimulant dependence (with withdrawal), cocaine dependence (with withdrawal), and cannabis detoxification (with withdrawal).

### Antibiotics and Anti-Fungals

In an aspect, provided herein is a method of treating or preventing an illness or condition in a subject, the method comprising administering to the subject a pharmaceutical composition provided herein.

In some embodiments, the illness or condition is an infection. In some embodiments, the infection is an acute infection or a chronic infection. In some embodiments, the infection is a bacterial infection. In some embodiments, the infection is a fungal infection.

In some embodiments, formulations and methods disclosed herein are used to reduce or eliminate a bacterial and/or fungal infection. In some embodiments, the formulations and methods disclosed herein provide reduce at least one adverse effect of the infection. Examples of adverse effect include symptoms of an infection such as fever, inflammation, fatigue, weight loss, loss of appetite, night sweats, chills, rash, coughing, and acute or chronic pain.

### Summary of Pharmaceutical Compositions and Salts and Methods of Use

Formulations with Low Molar Ratios

Some of the pharmaceutical compositions and salts have lower molar ratios of complexing agent to APIs, e.g., ratio values of 1:1 to 1:4, than the usual 1:4 to 1:7. The 1:1 ratio of complexing agent to API would produce improved molar solubility compared to normal use of Captisol, wherein the API solubilizes by hiding within the non-polar inner pore, rather than by forming a salt with the polar, ionized sulfobutylether substitutions. Solubility curves utilizing the non-polar complexing pore on the complexing agent molecule have shown that 1:1 ratio is essentially impossible, given that an excess of CD is required to solubilize a give molar equivalent of API. The improved solubility achieved and described herein is via an entirely different mechanism allowing a 1:1 ratio at a minimum. In a standard protocol using off the shelf Captisol, poorly soluble API or non-ionized API, the API is added to Captisol in ratio where the API molar value is lower than that for the Captisol (e.g., 1:0.8 of 1:0.5). This is due to the fact that each Captisol has only one non-polar binding. In reality, because all sites are not likely to be occupied it is essentially impossible to achieve a perfect 1:1 ratio using the complexing sites alone. Though the API may in fact have some inherent solubility in solution that would contribute to the solution strength, these molecules do not fully occupy every pore.

To illustrate, with the API eletriptan, it is disclosed herein that a stable 1:1 and 1:2 molar ratio formulation of Complexing Agent:API through salt formation with ionized API, yet when using the non-polar complexing pore to solubilize non-ionized API the stoichiometric ratio of CD:API is different and the same with rotigotine and copanlisib.

The compositions and salts disclosed herein solubilize a highly non-soluble molecule by making a salt instead from the ionized API, the ionization of the API being performed by the excess protons present in the CAP-Acid solution. To solubilize API via complexation through binding to the non-polar pore of the complexing agent, a large excess of the cyclodextrin relative to the API is needed and the process depends on the solubilizing power of the cyclodextrin on the API. By forming a salt, the solubilizing power of the cyclodextrin is being used. Thus, stoichiometry equivalent to 1:1 or greater quantities of the basic nitrogen containing API relative to the cyclodextrin can be obtained. Ability to complex is these cases, is not necessary, however could influence the property of any resulting formulations—with respect to stability and, inherent buffering capacity produced by the non-ionized API depending on inherent pKa properties and the pH of the produced solution.

In addition, because some poorly soluble molecules are too large to fit within the non-polar pore represented in Captisol, as well as the pores present in gamma and alpha cyclodextrins, standard Captisol and cyclodextrin complexing can sometimes be a non-viable technique. Thus, the salt formulation at low ratio is solution, even if one were not able to increase the ratio of API to the cyclodextrin above 1:1 in so far as that other solubilizing agents and solubilizing techniques would not be necessary. In the case of subcutaneous route of delivery, current techniques have drawbacks in subcutaneous formulations due to their bulk, irritant qualities and other noxious qualities. As such, the new formulations disclosed herein allow APIs that exhibit low solubility to be solubilized instead through salt formation at a specific stochiometric ratio of the API to the Captisol or other cyclodextrin. While a large excess of cyclodextrin is generally used to achieve solubilization of low solubility APIs, the formulations disclosed herein can enhance solubility without requiring this excess of cyclodextrin. In some embodiments, the formulations disclosed herein provide improved solubility at certain ratios of ratio range of the complexing agent. For example, eletriptan and rotigotine have been shown to have improved solubility at lower API:cyclodextrin ratios. For example, eletriptan at 2:1 ratio to captisol achieves superior solubility compared to 4:1 ratio which shows precipitation. As another example, rotigotine at a 1:1 ratio to captisol achieves superior solubility compared to a 2:1 ratio which shows precipitation. In some cases, the solubility is at least 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, or 100-fold improved relative to the API:cyclodextrin salt at a higher stoichiometric ratio and/or relative to a conventional formulation formed by simply combining the API and cyclodextrin (i.e., does not form the API-cyclodextrin salt).

The formulations disclosed here are soluble at API:Complexing Agent ratios higher than 1:1 and are a valuable solubilizing technique and protocol capable of more highly concentrated solutions than normal Captisol complexing. Some new potential API:CD salts arise from API that normally would be unable to make use of the complexing agent non-polar complexing pore site due to its size or other non-ideal qualities such as steric interference. Thus, the method disclosed herein can allow solubilization of poorly soluble amines in cases where higher stoichiometric salts (e.g., 7:1-resulting from the full replacement of the Captisol Na+ cations with ammonium API cations) are not possible. The salt formulations and methods allow solubilization power greater than that achievable by complexation.

### Prodrug Formulations

There are many existing API and potential future medications that are poorly soluble and/or exhibit low oral bioavailability. The prodrug formulations and methods disclosed herein can make them available for subcutaneous delivery or delivery through other routes of administration that might benefit from improved solubility by adding to the parent compound a moiety that can be cleaved off in natural processes of metabolism occurring in the liver during first or subsequent passes or also by enzymes and catabolic processes occurring in peripheral tissues. The prodrug formulations and methods disclosed herein are advantageous to create prodrugs with an ionizable nitrogen that would provide a counter cation to the anionic sulfobutylether substitutions, as demonstrated using Brexanolone in the following Scheme 1.

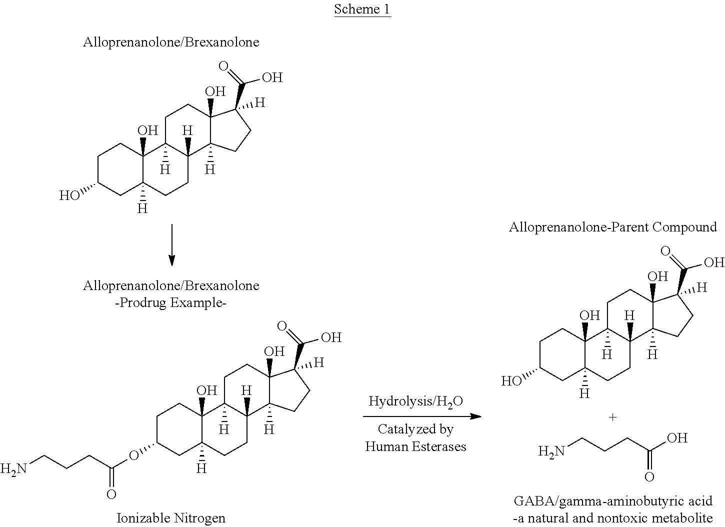

Brexanolone has poor oral bioavailability and very low water solubility. Using the methods and formulations disclosed herein, Brexanolone was formulated with Captisol® (Betadex Sulfobutyl Ether Sodium USP/NF), referred to as sulfobutyl ether betacyclodextrin (SBECD), a solubilizing excipient.

There is no need of nitrogen located on the molecule for application of the disclosed methods, and no other ionizable sites are needed. A prodrug formulation that had an ionizable nitrogen capable of forming a multivalent salt have advantages in a formulation compared to the parent compound formulation including increased potency, decrease in required milligrams of betadex sulfobutyl ether sodium, and improved pH and osmolality. All of these factors allow delivery of this medication to graduate from intravenous only administration to subcutaneous delivery with a wearable, subcutaneous patch pump. This graduation will have substantial benefits in medical economics by increasing the breadth of appropriate venues for medication delivery (e.g., psychiatric ward, standard medical wards, skilled nursing facilities) and decreasing the procedural burden of long period of intravenous infusion.

The methods disclosed herein produce prodrugs using gamma-aminobutyric acid (GABA) attached to the parent molecule's loan hydroxyl molecule in an ester bond. Once delivered, naturally occurring human esterase enzymes will cleave off the GABA through hydrolysis leaving the parent compound to circulate and producing the safe, natural molecule GABA as the cleavage product. A prodrug formulation disclosed herein is stable in liquid formulation for extended shelf life and as lyophilized API-Complexing Agent prodrug salt produced for reconstitution as a liquid formulation for use. The prodrug salts disclosed herein are agnostic to route of delivery, can be delivered through multiple established mechanisms including intravenous, intramuscular, sublingual, subcutaneous, intradermal, intranasal, transmucosal, per rectal, transvaginal, intrathecal and oral delivery.

A formulation disclosed herein can be delivered through a wearable pump with small volume cartridges to be viable clinically by adding a small, but clinically meaningful amount of non-ionized, non-pro-drug version of the active API in the non-polar pore of the complexing agent. The prodrug disclosed herein may form the salt and the parent compound will solubilize in the non-polar pore of the complexing agent. This could produce novel ratio or drug and prodrug that would have novel utility due to the strength of the solution, the onset of the pro-drug and non-prodrug versions of the API and chemical characteristics such as pH and osmolality benefits.

Formulations with Non-Ionizable APIs

A specific combined application of the API-CD salt techniques disclosed herein with the standard complexing technique utilizing the non-polar complexing site pore for non-ionized API can be utilized in numerous variations to produce valuable formulations by combining two or more API, potentially with quite variable properties that would normally make combination in single liquid formulation very difficult.

One of the formulations disclosed herein combine a poorly soluble and nonionizable API, such as rapamycin or clonidine, and an ionizable API, such as ketamine. For example, the medication rapamycin, also known as sirolimus, is a medication for inhibiting T-lymphocyte activation and proliferation stimulated by antigens and cytokines such as interleukin (IL)-2, IL-4, and IL-15. As such it has broad application in disorders wherein autoimmune and inflammatory mechanisms contribute to the pathophysiology of disease. However, none of existing formulations of rapamycin can be delivered concurrently with an intravenous, intramuscular or subcutaneous infusion or bolus of ketamine in treatment of various disorders. No multidose vial of intravenous rapamycin currently exists.

The value of concurrent administration of rapamycin with ketamine includes improvement in interpatient variability in achieving target blood levels and receptor occupancy effects, and treatment of mental health disorders. Temporal relation of delivery of rapamycin to ketamine is important in producing a synergistic clinical benefit. Thus the formulations disclosed herein provide potential for new delivery profiles.

Rapamycin is a poorly water soluble medication with reported aqueous solubility of 0.00173 mg/mL. It does not contain an easily ionizable nitrogen and the reported strongest acidic pKa is 9.96 and strongest basic pKa is −3. A single formulation disclosed herein can deliver appropriate doses of both ketamine (including racemic ketamine, arketamine, esketamine, or any stoichiometric ratio of ketamine enantiomers) and rapamycin.

Another application is combination of ketamine and clonidine in a single liquid formulation for use in treatment of various conditions, including but not limited to management of opioid dependence and detoxification, a variety of different pain etiologies including but not limited to neuropathic pain, complex regional pain syndrome and post herpetic neuralgia.

With regard to detoxification in the treatment of opioid dependence, ketamine delivered by subcutaneous pump in low steady state infusion will have beneficial effects in the context of opioid taper occurring over days or weeks due to not homeostatic resetting properties. The addition of clonidine within the formulation will permit steady state dosing of this medication as well, which is difficult to achieve without using patch forms of the drug that can take days to establish steady state. The short time frames of the disclosed formulation will provide value and have potential new clinical value. Clonidine is an agent for opioid detoxification and can be used for its anti-sympathetic and anti-norepinephrine effects, through blocking alpha 2 receptors and down-regulating norepinephrine release. Clonidine also has anti-dissociation effects when used in conjunction with ketamine, which may have particular value in this case by allowing larger doses of ketamine to be delivered than would normally be tolerated. Thus, the combination of clonidine with low dose steady state ketamine in a formulation in combination with a wearable patch pump designed to mitigate withdrawal symptoms from opioid taper occurring over days or weeks will potentially provide an all-in-one product in a medical treatment arena in urgent need of solutions and improvement in protocols, automation and convenience.

With regarding to pain management, norepinephrine and epinephrine signaling can increase pain signaling and can foster devastating processes that enhances acute pain over time to create long-term severe chronic pain. Clonidine can be used in treatment of different types of pain, but its short half-life makes it difficult to use without using a sustained release patch formulation, which makes it not the right fit for acute pain management. A combination product disclosed herein in acute pain circumstances, like shingles-related pain, will be notably helpful in treating the pain directly but also in decreasing the sympathetic signaling that participates in graduating the acute pain of a shingles outbreak into the serious chronic pain problem known as post-herpetic neuralgia. In addition, the disclosed combination formulation will also help resolve the chronic pain problem just as well, by treating the pain directly but also by decreasing the hyperalgesia central to the syndrome via the pharmacodynamic effects of both ketamine and clonidine.

The combination product uses the non-polar complexing pore to solubilize a potent but highly non-soluble API molecule, such as rapamycin and clonidine, and the acidic substitutions to solubilize a less potent and ionizable API, such as ketamine, to create a valuable synergy of effects in a single product.

### Super-Complexing

The super-complexing phenomena was discovered for certain formulations disclosed herein, which has the benefit of further reducing the osmolality of solutions, or other formulations targeted to intranasal, sublingual, intramuscular, intravenous, per rectal, transvaginal, or oral routes of administration. Osmolality for a number of Captisol Acid:API formulations was substantially lower than the calculated osmolarity for the APIs, and these data were documented in the examples. Super complexing of specific API-Captisol salt formulations disclosed herein, which exhibit osmolalities further lowered from the theoretically calculated osmolarity. In other words, the experimentally measured osmolalities were notably below what would be expected from stochiometric ratios based upon the average number of substitutions present on the Captisol ring and the additional molar equivalent of the API being added.

### Methods of Formulating Pharmaceutical Compounds and Preparing Complexing Agent Salts of Pharmaceutical Compounds

In one aspect, provided herein is a method of preparing a pharmaceutical composition, comprising: mixing a free acid form of a complexing agent comprising a plurality of acidic functional groups and a freebase form of a pharmaceutical compound, wherein the pharmaceutical compound comprises a basic nitrogen atom, wherein the molar ratio of the complexing agent to the pharmaceutical compound is from about 2:1 to about 1:4. In some embodiments, the pharmaceutical compound is an antiviral compound, an antibacterial compound, an anti-fungal compound, a compound for treatment of a neurological disorder, a compound for treatment of Parkinson's disease, a treatment for migraine headache, a treatment for autoimmune disease, a treatment for cancer, a treatment for lymphoma, a treatment for pancreatitis, a treatment for opioid overdose, a treatment for flu infection, or a treatment for an inflammatory disorder. In some embodiments, the pharmaceutical compound comprises an antibiotic (e.g., tigecycline, amikacin), an antifungal (e.g., caspofungin), an antiviral (e.g., nafamostat, remdesivir, an adamantane such as 1-amantadine), a dopaminergic (e.g., rotigotine, melevodopa), a 5-HT receptor agonist (e.g., eletriptan), cancer/anti-neoplastic medication (e.g., the PI3-kinase inhibitor copanlisib). In some embodiments, the pharmaceutical compound comprises a anti-cancer drug (e.g., rapamycin). In some embodiments, the pharmaceutical compound comprises a drug for treatment of variety disorders, including but not limited to high blood pressure, attention deficit hyperactivity disorder, drug withdrawal (alcohol, opioids, or nicotine), menopausal flushing, diarrhea, spasticity (e.g., clonidine). In some embodiments, the pharmaceutical compound comprises rotigotine, eletriptan, copanlisib, remdesivir, nafamostat (nafamostat mesylate), melevodopa, tigecycline, naloxone, amikacin, 1-amantadine, 2-amantadine, rimantadine, amifampridine, rapamycin, clonidine, or caspofungin.

In some embodiments, a precipitate forms when an additional molar equivalent of the pharmaceutical compound is added to the pharmaceutical composition and the additional molar equivalent is ionized after addition. In some embodiments, the method further comprises subjecting the pharmaceutical composition through an ion exchange process to generate a conjugate acid form of the complexing agent. In some embodiments, the ion exchange process comprises a resin ion exchange process. In some embodiments, the conjugate acid form of the complexing agent comprises a sodium ion occupying a deprotonated acidic site. In some embodiments, the method comprises selecting the pharmaceutical compound if the pharmaceutical compound has a solubility in a solvent less than a threshold value.

In some embodiments, the molar ratio of the complexing agent to the pharmaceutical compound is from about 2:1 to about 1:4, in which all or substantially all of the pharmaceutical compound is protonated and there is a molar excess of acidic sites of the pharmaceutical compound, in which the molar excess of acidic sites of the pharmaceutical compound becomes deprotonated and occupied with a sodium ion.

In some embodiments, the method comprises selecting the pharmaceutical compound if the solubility of the pharmaceutical compound is more than a threshold value. In some embodiments, the method comprises selecting the pharmaceutical compound if the solubility of the pharmaceutical compound is less than a threshold value.

In some embodiments, the pharmaceutical compound has a solubility of more than 100 mg/ml as salt. In some embodiments, the pharmaceutical compound has a solubility of more than 90 mg/ml as salt. In some embodiments, the pharmaceutical compound has a solubility of more than 80 mg/ml as salt. In some embodiments, the pharmaceutical compound has a solubility of more than 70 mg/ml as salt. In some embodiments, the pharmaceutical compound has a solubility of more than 60 mg/ml as salt. In some embodiments, the pharmaceutical compound has a solubility of more than 50 mg/ml as salt. In some embodiments, the pharmaceutical compound has a solubility of more than 45 mg/ml as salt. In some embodiments, the pharmaceutical compound has a solubility of more than 40 mg/ml as salt. In some embodiments, the pharmaceutical compound has a solubility of more than 35 mg/ml as salt. In some embodiments, the pharmaceutical compound has a solubility of more than 30 mg/ml as salt. In some embodiments, the pharmaceutical compound has a solubility of more than 25 mg/ml as salt. In some embodiments, the pharmaceutical compound has a solubility of more than 20 mg/ml as salt. In some embodiments, the pharmaceutical compound has a solubility of more than 10 mg/ml as salt. In some embodiments, the pharmaceutical compound has a solubility of more than 9 mg/ml as salt. In some embodiments, the pharmaceutical compound has a solubility of more than 8 mg/ml as salt. In some embodiments, the pharmaceutical compound has a solubility of more than 7 mg/ml as salt. In some embodiments, the pharmaceutical compound has a solubility of more than 6 mg/ml as salt. In some embodiments, the pharmaceutical compound has a solubility of more than 5 mg/ml as salt. In some embodiments, the pharmaceutical compound has a solubility of more than 4 mg/ml as salt. In some embodiments, the pharmaceutical compound has a solubility of more than 3 mg/ml as salt. In some embodiments, the pharmaceutical compound has a solubility of more 2 mg/ml as salt. In some embodiments, the pharmaceutical compound has a solubility of more than 1 mg/ml as salt. In some embodiments, the pharmaceutical compound has a solubility of more than 0.9 mg/ml as salt. In some embodiments, the pharmaceutical compound has a solubility of more than 0.8 mg/ml as salt. In some embodiments, the pharmaceutical compound has a solubility of more than 0.7 mg/ml as salt. In some embodiments, the pharmaceutical compound has a solubility of more than 0.6 mg/ml as salt. In some embodiments, the pharmaceutical compound has a solubility of more than 0.5 mg/ml as salt. In some embodiments, the pharmaceutical compound has a solubility of more than 0.4 mg/ml as salt. In some embodiments, the pharmaceutical compound has a solubility of more than 0.3 mg/ml as salt. In some embodiments, the pharmaceutical compound has a solubility of more than 0.2 mg/ml as salt. In some embodiments, the pharmaceutical compound has a solubility of more than 0.1 mg/ml as salt. In some embodiments, the pharmaceutical compound has a solubility of more than 0.09 mg/ml as salt. In some embodiments, the pharmaceutical compound has a solubility of more than 0.08 mg/ml as salt. In some embodiments, the pharmaceutical compound has a solubility of more than 0.07 mg/ml as salt. In some embodiments, the pharmaceutical compound has a solubility of more than 0.06 mg/ml as salt. In some embodiments, the pharmaceutical compound has a solubility of more than 0.05 mg/ml as salt. In some embodiments, the pharmaceutical compound has a solubility of more than 0.04 mg/ml as salt. In some embodiments, the pharmaceutical compound has a solubility of more than 0.03 mg/ml as salt. In some embodiments, the pharmaceutical compound has a solubility of more than 0.02 mg/ml as salt. In some embodiments, the pharmaceutical compound has a solubility of more than 0.01 mg/ml as salt. In some embodiments, the pharmaceutical compound has a solubility of more than 0.009 mg/ml as salt. In some embodiments, the pharmaceutical compound has a solubility of more than 0.008 mg/ml as salt. In some embodiments, the pharmaceutical compound has a solubility of more than 0.007 mg/ml as salt. In some embodiments, the pharmaceutical compound has a solubility of more than 0.006 mg/ml as salt. In some embodiments, the pharmaceutical compound has a solubility of more than 0.005 mg/ml as salt. In some embodiments, the pharmaceutical compound has a solubility of more than 0.004 mg/ml as salt. In some embodiments, the pharmaceutical compound has a solubility of more than 0.003 mg/ml as salt. In some embodiments, the pharmaceutical compound has a solubility of more than 0.002 mg/ml as salt. In some embodiments, the pharmaceutical compound has a solubility of more than 0.001 mg/ml as salt. In some embodiments, the pharmaceutical compound has a solubility of more than 0.9 μg/ml as salt. In some embodiments, the pharmaceutical compound has a solubility of more than 0.8 μg/ml as salt. In some embodiments, the pharmaceutical compound has a solubility of more than 0.7 μg/ml as salt. In some embodiments, the pharmaceutical compound has a solubility of more than 0.6 μg/ml as salt. In some embodiments, the pharmaceutical compound has a solubility of more than 0.5 μg/ml as salt. In some embodiments, the pharmaceutical compound has a solubility of more than 0.4 μg/ml as salt. In some embodiments, the pharmaceutical compound has a solubility of more than 0.3 μg/ml as salt. In some embodiments, the pharmaceutical compound has a solubility of more than 0.2 μg/ml as salt. In some embodiments, the pharmaceutical compound has a solubility of more than 0.1 μg/ml as salt. In some embodiments, the pharmaceutical compound has a solubility of more than 0.09 μg/ml as salt. In some embodiments, the pharmaceutical compound has a solubility of more than 0.08 μg/ml as salt. In some embodiments, the pharmaceutical compound has a solubility of more than 0.07 μg/ml as salt. In some embodiments, the pharmaceutical compound has a solubility of more than 0.06 μg/ml as salt. In some embodiments, the pharmaceutical compound has a solubility of more than 0.05 μg/ml as salt. In some embodiments, the pharmaceutical compound has a solubility of more than 0.04 μg/ml as salt. In some embodiments, the pharmaceutical compound has a solubility of more than 0.03 μg/ml as salt. In some embodiments, the pharmaceutical compound has a solubility of more than 0.02 μg/ml as salt. In some embodiments, the pharmaceutical compound has a solubility of more than 0.01 μg/ml as salt. In some embodiments, the pharmaceutical compound has a solubility of more than 0.009 μg/ml as salt. In some embodiments, the pharmaceutical compound has a solubility of more than 0.008 μg/ml as salt. In some embodiments, the pharmaceutical compound has a solubility of more than 0.007 μg/ml as salt. In some embodiments, the pharmaceutical compound has a solubility of more than 0.006 μg/ml as salt. In some embodiments, the pharmaceutical compound has a solubility of more than 0.005 μg/ml as salt. In some embodiments, the pharmaceutical compound has a solubility of more than 0.004 μg/ml as salt. In some embodiments, the pharmaceutical compound has a solubility of more than 0.003 μg/ml as salt. In some embodiments, the pharmaceutical compound has a solubility of more than 0.002 μg/ml as salt. In some embodiments, the pharmaceutical compound has a solubility of more than 0.001 μg/ml as salt. In some embodiments, the solubility of the pharmaceutical compound as salt is measured in an aqueous medium. In some embodiments, the solubility of the pharmaceutical compound as salt is measured in an organic solvent. In some embodiments, the aqueous medium comprises water. In some embodiments, the salt of the pharmaceutical compound comprises a HCl salt.

In some embodiments, the pharmaceutical compound has a solubility of between about 0.001 mg/ml and 100 mg/ml as salt. In some embodiments, the pharmaceutical compound has a solubility of between about 0.01 mg/ml and 50 mg/ml as salt. In some embodiments, the pharmaceutical compound has a solubility of between about 0.02 mg/ml and 40 mg/ml as salt. In some embodiments, the pharmaceutical compound has a solubility of between about 0.03 mg/ml and 30 mg/ml as salt. In some embodiments, the pharmaceutical compound has a solubility of between about 0.04 mg/ml and 20 mg/ml as salt. In some embodiments, the pharmaceutical compound has a solubility of between about 0.05 mg/ml and 10 mg/ml as salt. In some embodiments, the pharmaceutical compound has a solubility of between about 0.06 mg/ml and 5 mg/ml as salt. In some embodiments, the pharmaceutical compound has a solubility of between about 0.07 mg/ml and 4 mg/ml as salt. In some embodiments, the pharmaceutical compound has a solubility of between about 0.09 mg/ml and 3 mg/ml as salt. In some embodiments, the pharmaceutical compound has a solubility of between about 0.1 mg/ml and 2 mg/ml as salt. In some embodiments, the solubility of the pharmaceutical compound as salt is measured in an aqueous medium. In some embodiments, the solubility of the pharmaceutical compound as salt is measured in an organic solvent. In some embodiments, the aqueous medium comprises water. In some embodiments, the salt of the pharmaceutical compound comprises a HCl salt.

In some embodiments, the pharmaceutical compound has a solubility of less than 100 mg/ml as salt. In some embodiments, the pharmaceutical compound has a solubility of less than 90 mg/ml as salt. In some embodiments, the pharmaceutical compound has a solubility of less than 80 mg/ml as salt. In some embodiments, the pharmaceutical compound has a solubility of less than 70 mg/ml as salt. In some embodiments, the pharmaceutical compound has a solubility of less than 60 mg/ml as salt. In some embodiments, the pharmaceutical compound has a solubility of less than 50 mg/ml as salt. In some embodiments, the pharmaceutical compound has a solubility of less than 45 mg/ml as salt. In some embodiments, the pharmaceutical compound has a solubility of less than 40 mg/ml as salt. In some embodiments, the pharmaceutical compound has a solubility of less than 35 mg/ml as salt. In some embodiments, the pharmaceutical compound has a solubility of less than 30 mg/ml as salt. In some embodiments, the pharmaceutical compound has a solubility of less than 25 mg/ml as salt. In some embodiments, the pharmaceutical compound has a solubility of less than 20 mg/ml as salt. In some embodiments, the pharmaceutical compound has a solubility of less than 10 mg/ml as salt. In some embodiments, the pharmaceutical compound has a solubility of less than 9 mg/ml as salt. In some embodiments, the pharmaceutical compound has a solubility of less than 8 mg/ml as salt. In some embodiments, the pharmaceutical compound has a solubility of less than 7 mg/ml as salt. In some embodiments, the pharmaceutical compound has a solubility of less than 6 mg/ml as salt. In some embodiments, the pharmaceutical compound has a solubility of less than 5 mg/ml as salt. In some embodiments, the pharmaceutical compound has a solubility of less than 4 mg/ml as salt. In some embodiments, the pharmaceutical compound has a solubility of less than 3 mg/ml as salt. In some embodiments, the pharmaceutical compound has a solubility of more 2 mg/ml as salt. In some embodiments, the pharmaceutical compound has a solubility of less than 1 mg/ml as salt. In some embodiments, the pharmaceutical compound has a solubility of less than 0.9 mg/ml as salt. In some embodiments, the pharmaceutical compound has a solubility of less than 0.8 mg/ml as salt. In some embodiments, the pharmaceutical compound has a solubility of less than 0.7 mg/ml as salt. In some embodiments, the pharmaceutical compound has a solubility of less than 0.6 mg/ml as salt. In some embodiments, the pharmaceutical compound has a solubility of less than 0.5 mg/ml as salt. In some embodiments, the pharmaceutical compound has a solubility of less than 0.4 mg/ml as salt. In some embodiments, the pharmaceutical compound has a solubility of less than 0.3 mg/ml as salt. In some embodiments, the pharmaceutical compound has a solubility of less than 0.2 mg/ml as salt. In some embodiments, the pharmaceutical compound has a solubility of less than 0.1 mg/ml as salt. In some embodiments, the pharmaceutical compound has a solubility of less than 0.09 mg/ml as salt. In some embodiments, the pharmaceutical compound has a solubility of less than 0.08 mg/ml as salt. In some embodiments, the pharmaceutical compound has a solubility of less than 0.07 mg/ml as salt. In some embodiments, the pharmaceutical compound has a solubility of less than 0.06 mg/ml as salt. In some embodiments, the pharmaceutical compound has a solubility of less than 0.05 mg/ml as salt. In some embodiments, the pharmaceutical compound has a solubility of less than 0.04 mg/ml as salt. In some embodiments, the pharmaceutical compound has a solubility of less than 0.03 mg/ml as salt. In some embodiments, the pharmaceutical compound has a solubility of less than 0.02 mg/ml as salt. In some embodiments, the pharmaceutical compound has a solubility of less than 0.01 mg/ml as salt. In some embodiments, the pharmaceutical compound has a solubility of less than 0.009 mg/ml as salt. In some embodiments, the pharmaceutical compound has a solubility of less than 0.008 mg/ml as salt. In some embodiments, the pharmaceutical compound has a solubility of less than 0.007 mg/ml as salt. In some embodiments, the pharmaceutical compound has a solubility of less than 0.006 mg/ml as salt. In some embodiments, the pharmaceutical compound has a solubility of less than 0.005 mg/ml as salt. In some embodiments, the pharmaceutical compound has a solubility of less than 0.004 mg/ml as salt. In some embodiments, the pharmaceutical compound has a solubility of less than 0.003 mg/ml as salt. In some embodiments, the pharmaceutical compound has a solubility of less than 0.002 mg/ml as salt. In some embodiments, the pharmaceutical compound has a solubility of less than 0.001 mg/ml as salt. In some embodiments, the pharmaceutical compound has a solubility of less than 0.9 μg/ml as salt. In some embodiments, the pharmaceutical compound has a solubility of less than 0.8 μg/ml as salt. In some embodiments, the pharmaceutical compound has a solubility of less than 0.7 μg/ml as salt. In some embodiments, the pharmaceutical compound has a solubility of less than 0.6 μg/ml as salt. In some embodiments, the pharmaceutical compound has a solubility of less than 0.5 μg/ml as salt. In some embodiments, the pharmaceutical compound has a solubility of less than 0.4 μg/ml as salt. In some embodiments, the pharmaceutical compound has a solubility of less than 0.3 μg/ml as salt. In some embodiments, the pharmaceutical compound has a solubility of less than 0.2 μg/ml as salt. In some embodiments, the pharmaceutical compound has a solubility of less than 0.1 μg/ml as salt. In some embodiments, the pharmaceutical compound has a solubility of less than 0.09 μg/ml as salt. In some embodiments, the pharmaceutical compound has a solubility of less than 0.08 μg/ml as salt. In some embodiments, the pharmaceutical compound has a solubility of less than 0.07 μg/ml as salt. In some embodiments, the pharmaceutical compound has a solubility of less than 0.06 μg/ml as salt. In some embodiments, the pharmaceutical compound has a solubility of less than 0.05 μg/ml as salt. In some embodiments, the pharmaceutical compound has a solubility of less than 0.04 μg/ml as salt. In some embodiments, the pharmaceutical compound has a solubility of less than 0.03 μg/ml as salt. In some embodiments, the pharmaceutical compound has a solubility of less than 0.02 μg/ml as salt. In some embodiments, the pharmaceutical compound has a solubility of less than 0.01 μg/ml as salt. In some embodiments, the pharmaceutical compound has a solubility of less than 0.009 μg/ml as salt. In some embodiments, the pharmaceutical compound has a solubility of less than 0.008 μg/ml as salt. In some embodiments, the pharmaceutical compound has a solubility of less than 0.007 μg/ml as salt. In some embodiments, the pharmaceutical compound has a solubility of less than 0.006 μg/ml as salt. In some embodiments, the pharmaceutical compound has a solubility of less than 0.005 μg/ml as salt. In some embodiments, the pharmaceutical compound has a solubility of less than 0.004 μg/ml as salt. In some embodiments, the pharmaceutical compound has a solubility of less than 0.003 μg/ml as salt. In some embodiments, the pharmaceutical compound has a solubility of less than 0.002 μg/ml as salt. In some embodiments, the pharmaceutical compound has a solubility of less than 0.001 μg/ml as salt. In some embodiments, the solubility of the pharmaceutical compound as salt is measured in an aqueous medium. In some embodiments, the solubility of the pharmaceutical compound as salt is measured in an organic solvent. In some embodiments, the aqueous medium comprises water. In some embodiments, the salt of the pharmaceutical compound comprises a HCl salt.

In some embodiments, the pharmaceutical compound has a solubility of more than 100 mg/ml as freebase. In some embodiments, the pharmaceutical compound has a solubility of more than 90 mg/ml as freebase. In some embodiments, the pharmaceutical compound has a solubility of more than 80 mg/ml as freebase. In some embodiments, the pharmaceutical compound has a solubility of more than 70 mg/ml as freebase. In some embodiments, the pharmaceutical compound has a solubility of more than 60 mg/ml as freebase. In some embodiments, the pharmaceutical compound has a solubility of more than 50 mg/ml as freebase. In some embodiments, the pharmaceutical compound has a solubility of more than 45 mg/ml as freebase. In some embodiments, the pharmaceutical compound has a solubility of more than 40 mg/ml as freebase. In some embodiments, the pharmaceutical compound has a solubility of more than 35 mg/ml as freebase. In some embodiments, the pharmaceutical compound has a solubility of more than 30 mg/ml as freebase. In some embodiments, the pharmaceutical compound has a solubility of more than 25 mg/ml as freebase. In some embodiments, the pharmaceutical compound has a solubility of more than 20 mg/ml as freebase. In some embodiments, the pharmaceutical compound has a solubility of more than 10 mg/ml as freebase. In some embodiments, the pharmaceutical compound has a solubility of more than 9 mg/ml as freebase. In some embodiments, the pharmaceutical compound has a solubility of more than 8 mg/ml as freebase. In some embodiments, the pharmaceutical compound has a solubility of more than 7 mg/ml as freebase. In some embodiments, the pharmaceutical compound has a solubility of more than 6 mg/ml as freebase. In some embodiments, the pharmaceutical compound has a solubility of more than 5 mg/ml as freebase. In some embodiments, the pharmaceutical compound has a solubility of more than 4 mg/ml as freebase. In some embodiments, the pharmaceutical compound has a solubility of more than 3 mg/ml as freebase. In some embodiments, the pharmaceutical compound has a solubility of more 2 mg/ml as freebase. In some embodiments, the pharmaceutical compound has a solubility of more than 1 mg/ml as freebase. In some embodiments, the pharmaceutical compound has a solubility of more than 0.9 mg/ml as freebase. In some embodiments, the pharmaceutical compound has a solubility of more than 0.8 mg/ml as freebase. In some embodiments, the pharmaceutical compound has a solubility of more than 0.7 mg/ml as freebase. In some embodiments, the pharmaceutical compound has a solubility of more than 0.6 mg/ml as freebase. In some embodiments, the pharmaceutical compound has a solubility of more than 0.5 mg/ml as freebase. In some embodiments, the pharmaceutical compound has a solubility of more than 0.4 mg/ml as freebase. In some embodiments, the pharmaceutical compound has a solubility of more than 0.3 mg/ml as freebase. In some embodiments, the pharmaceutical compound has a solubility of more than 0.2 mg/ml as freebase. In some embodiments, the pharmaceutical compound has a solubility of more than 0.1 mg/ml as freebase. In some embodiments, the pharmaceutical compound has a solubility of more than 0.09 mg/ml as freebase. In some embodiments, the pharmaceutical compound has a solubility of more than 0.08 mg/ml as freebase. In some embodiments, the pharmaceutical compound has a solubility of more than 0.07 mg/ml as freebase. In some embodiments, the pharmaceutical compound has a solubility of more than 0.06 mg/ml as freebase. In some embodiments, the pharmaceutical compound has a solubility of more than 0.05 mg/ml as freebase. In some embodiments, the pharmaceutical compound has a solubility of more than 0.04 mg/ml as freebase. In some embodiments, the pharmaceutical compound has a solubility of more than 0.03 mg/ml as freebase. In some embodiments, the pharmaceutical compound has a solubility of more than 0.02 mg/ml as freebase. In some embodiments, the pharmaceutical compound has a solubility of more than 0.01 mg/ml as freebase. In some embodiments, the pharmaceutical compound has a solubility of more than 0.009 mg/ml as freebase. In some embodiments, the pharmaceutical compound has a solubility of more than 0.008 mg/ml as freebase. In some embodiments, the pharmaceutical compound has a solubility of more than 0.007 mg/ml as freebase. In some embodiments, the pharmaceutical compound has a solubility of more than 0.006 mg/ml as freebase. In some embodiments, the pharmaceutical compound has a solubility of more than 0.005 mg/ml as freebase. In some embodiments, the pharmaceutical compound has a solubility of more than 0.004 mg/ml as freebase. In some embodiments, the pharmaceutical compound has a solubility of more than 0.003 mg/ml as freebase. In some embodiments, the pharmaceutical compound has a solubility of more than 0.002 mg/ml as freebase. In some embodiments, the pharmaceutical compound has a solubility of more than 0.001 mg/ml as freebase. In some embodiments, the pharmaceutical compound has a solubility of more than 0.9 μg/ml as freebase. In some embodiments, the pharmaceutical compound has a solubility of more than 0.8 μg/ml as freebase. In some embodiments, the pharmaceutical compound has a solubility of more than 0.7 μg/ml as freebase. In some embodiments, the pharmaceutical compound has a solubility of more than 0.6 μg/ml as freebase. In some embodiments, the pharmaceutical compound has a solubility of more than 0.5 μg/ml as freebase. In some embodiments, the pharmaceutical compound has a solubility of more than 0.4 μg/ml as freebase. In some embodiments, the pharmaceutical compound has a solubility of more than 0.3 μg/ml as freebase. In some embodiments, the pharmaceutical compound has a solubility of more than 0.2 μg/ml as freebase. In some embodiments, the pharmaceutical compound has a solubility of more than 0.1 μg/ml as freebase. In some embodiments, the pharmaceutical compound has a solubility of more than 0.09 μg/ml as freebase. In some embodiments, the pharmaceutical compound has a solubility of more than 0.08 μg/ml as freebase. In some embodiments, the pharmaceutical compound has a solubility of more than 0.07 μg/ml as freebase. In some embodiments, the pharmaceutical compound has a solubility of more than 0.06 μg/ml as freebase. In some embodiments, the pharmaceutical compound has a solubility of more than 0.05 μg/ml as freebase. In some embodiments, the pharmaceutical compound has a solubility of more than 0.04 μg/ml as freebase. In some embodiments, the pharmaceutical compound has a solubility of more than 0.03 μg/ml as freebase. In some embodiments, the pharmaceutical compound has a solubility of more than 0.02 μg/ml as freebase. In some embodiments, the pharmaceutical compound has a solubility of more than 0.01 μg/ml as freebase. In some embodiments, the pharmaceutical compound has a solubility of more than 0.009 μg/ml as freebase. In some embodiments, the pharmaceutical compound has a solubility of more than 0.008 μg/ml as freebase. In some embodiments, the pharmaceutical compound has a solubility of more than 0.007 μg/ml as freebase. In some embodiments, the pharmaceutical compound has a solubility of more than 0.006 μg/ml as freebase. In some embodiments, the pharmaceutical compound has a solubility of more than 0.005 μg/ml as freebase. In some embodiments, the pharmaceutical compound has a solubility of more than 0.004 μg/ml as freebase. In some embodiments, the pharmaceutical compound has a solubility of more than 0.003 μg/ml as freebase. In some embodiments, the pharmaceutical compound has a solubility of more than 0.002 μg/ml as freebase. In some embodiments, the pharmaceutical compound has a solubility of more than 0.001 μg/ml as freebase. In some embodiments, the solubility of the pharmaceutical compound as freebase is measured in an aqueous medium. In some embodiments, the solubility of the pharmaceutical compound as freebase is measured in an organic solvent. In some embodiments, the aqueous medium comprises water.

In some embodiments, the pharmaceutical compound has a solubility of between about 0.001 mg/ml and 100 mg/ml as freebase. In some embodiments, the pharmaceutical compound has a solubility of between about 0.01 mg/ml and 50 mg/ml as freebase. In some embodiments, the pharmaceutical compound has a solubility of between about 0.02 mg/ml and 40 mg/ml as freebase. In some embodiments, the pharmaceutical compound has a solubility of between about 0.03 mg/ml and 30 mg/ml as freebase. In some embodiments, the pharmaceutical compound has a solubility of between about 0.04 mg/ml and 20 mg/ml as freebase. In some embodiments, the pharmaceutical compound has a solubility of between about 0.05 mg/ml and 10 mg/ml as freebase. In some embodiments, the pharmaceutical compound has a solubility of between about 0.06 mg/ml and 5 mg/ml as freebase. In some embodiments, the pharmaceutical compound has a solubility of between about 0.07 mg/ml and 4 mg/ml as freebase. In some embodiments, the pharmaceutical compound has a solubility of between about 0.09 mg/ml and 3 mg/ml as freebase. In some embodiments, the pharmaceutical compound has a solubility of between about 0.1 mg/ml and 2 mg/ml as freebase. In some embodiments, the solubility of the pharmaceutical compound as freebase is measured in an aqueous medium. In some embodiments, the solubility of the pharmaceutical compound as freebase is measured in an organic solvent. In some embodiments, the aqueous medium comprises water.

In some embodiments, the pharmaceutical compound has a solubility of less than 100 mg/ml as freebase. In some embodiments, the pharmaceutical compound has a solubility of less than 90 mg/ml as freebase. In some embodiments, the pharmaceutical compound has a solubility of less than 80 mg/ml as freebase. In some embodiments, the pharmaceutical compound has a solubility of less than 70 mg/ml as freebase. In some embodiments, the pharmaceutical compound has a solubility of less than 60 mg/ml as freebase. In some embodiments, the pharmaceutical compound has a solubility of less than 50 mg/ml as freebase. In some embodiments, the pharmaceutical compound has a solubility of less than 45 mg/ml as freebase. In some embodiments, the pharmaceutical compound has a solubility of less than 40 mg/ml as freebase. In some embodiments, the pharmaceutical compound has a solubility of less than 35 mg/ml as freebase. In some embodiments, the pharmaceutical compound has a solubility of less than 30 mg/ml as freebase. In some embodiments, the pharmaceutical compound has a solubility of less than 25 mg/ml as freebase. In some embodiments, the pharmaceutical compound has a solubility of less than 20 mg/ml as freebase. In some embodiments, the pharmaceutical compound has a solubility of less than 10 mg/ml as freebase. In some embodiments, the pharmaceutical compound has a solubility of less than 9 mg/ml as freebase. In some embodiments, the pharmaceutical compound has a solubility of less than 8 mg/ml as freebase. In some embodiments, the pharmaceutical compound has a solubility of less than 7 mg/ml as freebase. In some embodiments, the pharmaceutical compound has a solubility of less than 6 mg/ml as freebase. In some embodiments, the pharmaceutical compound has a solubility of less than 5 mg/ml as freebase. In some embodiments, the pharmaceutical compound has a solubility of less than 4 mg/ml as freebase. In some embodiments, the pharmaceutical compound has a solubility of less than 3 mg/ml as freebase. In some embodiments, the pharmaceutical compound has a solubility of more 2 mg/ml as freebase. In some embodiments, the pharmaceutical compound has a solubility of less than 1 mg/ml as freebase. In some embodiments, the pharmaceutical compound has a solubility of less than 0.9 mg/ml as freebase. In some embodiments, the pharmaceutical compound has a solubility of less than 0.8 mg/ml as freebase. In some embodiments, the pharmaceutical compound has a solubility of less than 0.7 mg/ml as freebase. In some embodiments, the pharmaceutical compound has a solubility of less than 0.6 mg/ml as freebase. In some embodiments, the pharmaceutical compound has a solubility of less than 0.5 mg/ml as freebase. In some embodiments, the pharmaceutical compound has a solubility of less than 0.4 mg/ml as freebase. In some embodiments, the pharmaceutical compound has a solubility of less than 0.3 mg/ml as freebase. In some embodiments, the pharmaceutical compound has a solubility of less than 0.2 mg/ml as freebase. In some embodiments, the pharmaceutical compound has a solubility of less than 0.1 mg/ml as freebase. In some embodiments, the pharmaceutical compound has a solubility of less than 0.09 mg/ml as freebase. In some embodiments, the pharmaceutical compound has a solubility of less than 0.08 mg/ml as freebase. In some embodiments, the pharmaceutical compound has a solubility of less than 0.07 mg/ml as freebase. In some embodiments, the pharmaceutical compound has a solubility of less than 0.06 mg/ml as freebase. In some embodiments, the pharmaceutical compound has a solubility of less than 0.05 mg/ml as freebase. In some embodiments, the pharmaceutical compound has a solubility of less than 0.04 mg/ml as freebase. In some embodiments, the pharmaceutical compound has a solubility of less than 0.03 mg/ml as freebase. In some embodiments, the pharmaceutical compound has a solubility of less than 0.02 mg/ml as freebase. In some embodiments, the pharmaceutical compound has a solubility of less than 0.01 mg/ml as freebase. In some embodiments, the pharmaceutical compound has a solubility of less than 0.009 mg/ml as freebase. In some embodiments, the pharmaceutical compound has a solubility of less than 0.008 mg/ml as freebase. In some embodiments, the pharmaceutical compound has a solubility of less than 0.007 mg/ml as freebase. In some embodiments, the pharmaceutical compound has a solubility of less than 0.006 mg/ml as freebase. In some embodiments, the pharmaceutical compound has a solubility of less than 0.005 mg/ml as freebase. In some embodiments, the pharmaceutical compound has a solubility of less than 0.004 mg/ml as freebase. In some embodiments, the pharmaceutical compound has a solubility of less than 0.003 mg/ml as freebase. In some embodiments, the pharmaceutical compound has a solubility of less than 0.002 mg/ml as freebase. In some embodiments, the pharmaceutical compound has a solubility of less than 0.001 mg/ml as freebase. In some embodiments, the pharmaceutical compound has a solubility of less than 0.9 μg/ml as freebase. In some embodiments, the pharmaceutical compound has a solubility of less than 0.8 μg/ml as freebase. In some embodiments, the pharmaceutical compound has a solubility of less than 0.7 μg/ml as freebase. In some embodiments, the pharmaceutical compound has a solubility of less than 0.6 μg/ml as freebase. In some embodiments, the pharmaceutical compound has a solubility of less than 0.5 μg/ml as freebase. In some embodiments, the pharmaceutical compound has a solubility of less than 0.4 μg/ml as freebase. In some embodiments, the pharmaceutical compound has a solubility of less than 0.3 μg/ml as freebase. In some embodiments, the pharmaceutical compound has a solubility of less than 0.2 μg/ml as freebase. In some embodiments, the pharmaceutical compound has a solubility of less than 0.1 μg/ml as freebase. In some embodiments, the pharmaceutical compound has a solubility of less than 0.09 μg/ml as freebase. In some embodiments, the pharmaceutical compound has a solubility of less than 0.08 μg/ml as freebase. In some embodiments, the pharmaceutical compound has a solubility of less than 0.07 μg/ml as freebase. In some embodiments, the pharmaceutical compound has a solubility of less than 0.06 μg/ml as freebase. In some embodiments, the pharmaceutical compound has a solubility of less than 0.05 μg/ml as freebase. In some embodiments, the pharmaceutical compound has a solubility of less than 0.04 μg/ml as freebase. In some embodiments, the pharmaceutical compound has a solubility of less than 0.03 μg/ml as freebase. In some embodiments, the pharmaceutical compound has a solubility of less than 0.02 μg/ml as freebase. In some embodiments, the pharmaceutical compound has a solubility of less than 0.01 μg/ml as freebase. In some embodiments, the pharmaceutical compound has a solubility of less than 0.009 μg/ml as freebase. In some embodiments, the pharmaceutical compound has a solubility of less than 0.008 μg/ml as freebase. In some embodiments, the pharmaceutical compound has a solubility of less than 0.007 μg/ml as freebase. In some embodiments, the pharmaceutical compound has a solubility of less than 0.006 μg/ml as freebase. In some embodiments, the pharmaceutical compound has a solubility of less than 0.005 μg/ml as freebase. In some embodiments, the pharmaceutical compound has a solubility of less than 0.004 μg/ml as freebase. In some embodiments, the pharmaceutical compound has a solubility of less than 0.003 μg/ml as freebase. In some embodiments, the pharmaceutical compound has a solubility of less than 0.002 μg/ml as freebase. In some embodiments, the pharmaceutical compound has a solubility of less than 0.001 μg/ml as freebase. In some embodiments, the solubility of the pharmaceutical compound as freebase is measured in an aqueous medium. In some embodiments, the solubility of the pharmaceutical compound as freebase is measured in an organic solvent. In some embodiments, the aqueous medium comprises water.

In some embodiments, the solubility of the pharmaceutical compound in the pharmaceutical composition is higher than (i) the solubility of the pharmaceutical compound as a salt; or (ii) the solubility of the pharmaceutical compound in a composition comprising the pharmaceutical compound in freebase form that is complexing to a non-polar pore of the complexing agent, wherein the pharmaceutical compound has the same concentration in the pharmaceutical composition, the pharmaceutical compound as a salt, and the a composition comprising the pharmaceutical compound in freebase form. In some embodiments, the solubility of the pharmaceutical compound in the pharmaceutical composition is higher than the solubility of the pharmaceutical compound in a composition comprising the pharmaceutical compound and complexing agent with a higher molar ratio of the pharmaceutical compound to the complexing agent, wherein the pharmaceutical compound has the same concentration in the pharmaceutical composition and the a composition with a higher molar ratio of the pharmaceutical compound to the complexing agent. In some embodiments, the solubility of the pharmaceutical compound in the pharmaceutical composition has an increase of at least 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, or 100-fold compared with (i) the solubility of the pharmaceutical compound as a salt; or (ii) the solubility of the pharmaceutical compound in a composition comprising the pharmaceutical compound in freebase form that is complexing to a non-polar pore of the complexing agent, wherein the pharmaceutical compound has the same concentration in the pharmaceutical composition, the pharmaceutical compound as a salt, and the composition comprising the pharmaceutical compound in freebase form. In some embodiments, the solubility of the pharmaceutical compound in the pharmaceutical composition has an increase of at least 2-fold, 50-fold, 60-fold, 70-fold, 80-fold, 90-fold, or 100-fold compared with the solubility of the pharmaceutical compound in a composition comprising the pharmaceutical compound and complexing agent with a higher molar ratio of the pharmaceutical compound to the complexing agent, wherein the pharmaceutical compound has the same concentration in the pharmaceutical composition and the composition with a higher molar ratio of the pharmaceutical compound to the complexing agent.

In another aspect, provided herein is a method of preparing a pharmaceutical composition, comprising mixing a free acid form of a complexing agent comprising a plurality of acidic functional groups and a freebase form of a pharmaceutical compound, wherein the pharmaceutical compound comprises a basic nitrogen atom, wherein the molar ratio of the complexing agent to the pharmaceutical compound is from about 2:1 to about 1:4, and adding an additional molar equivalent of the pharmaceutical compound, wherein the additional molar equivalent is unionized and does not ionize after addition. In some embodiments, the pharmaceutical compound is an antiviral compound, an antibacterial compound, an anti-fungal compound, a compound for treatment of a neurological disorder, a compound for treatment of Parkinson's disease, a treatment for migraine headache, a treatment for autoimmune disease, a treatment for cancer, a treatment for lymphoma, a treatment for pancreatitis, a treatment for opioid overdose, a treatment for flu infection, a treatment for cancer, a treatment for high blood pressure, attention deficit hyperactivity disorder, drug withdrawal (alcohol, opioids, or nicotine), menopausal flushing, diarrhea, or spasticity, or a treatment for an inflammatory disorder. In some embodiments, the pharmaceutical compound comprises rotigotine, eletriptan, copanlisib, remdesivir, nafamostat (nafamostat mesylate), melevodopa, tigecycline, naloxone, amikacin, 1-amantadine, 2-amantadine, rimantadine, amifampridine, rapamycin, clonidine, or caspofungin.

In some embodiments, a precipitate forms when an additional molar equivalent of the pharmaceutical compound is added to the pharmaceutical composition and the additional molar equivalent is ionized after addition. In some embodiments, the method further comprises subjecting the pharmaceutical composition through an ion exchange process to generate a conjugate acid form of the complexing agent. In some embodiments, the ion exchange process comprises a resin ion exchange process. In some embodiments, the conjugate acid form of the complexing agent comprises a sodium ion occupying a deprotonated acidic site. In some embodiments, the method comprises selecting the pharmaceutical compound if the pharmaceutical compound has a solubility in a solvent less than a threshold value.

In some embodiments, the molar ratio of the complexing agent to the pharmaceutical compound is from about 2:1 to about 1:4, in which all or substantially all of the pharmaceutical compound is protonated and there is a molar excess of acidic sites of the pharmaceutical compound, in which the molar excess of acidic sites of the pharmaceutical compound becomes deprotonated and occupied with a sodium ion.

In some embodiments, all or a portion of the pharmaceutical compound becomes protonated in the liquid medium upon addition of the free acid form of the complexing agent. At this point, in some embodiments, a molar excess of the free base form of the pharmaceutical compound is then added. In some embodiments, the liquid medium is first removed by a suitable method (e.g. evaporation, lyophilization, etc.) before the molar excess of the free base form of the pharmaceutical compound is added. In such cases, any of the ratios of molar equivalents of unionized pharmaceutical compound provided herein may be used.

In some embodiments, the method comprises selecting the pharmaceutical compound if the solubility of the pharmaceutical compound is more than a threshold value. In some embodiments, the method comprises selecting the pharmaceutical compound if the solubility of the pharmaceutical compound is less than a threshold value.

In some embodiments, the pharmaceutical compound has a solubility of more than 100 mg/ml as salt. In some embodiments, the pharmaceutical compound has a solubility of more than 90 mg/ml as salt. In some embodiments, the pharmaceutical compound has a solubility of more than 80 mg/ml as salt. In some embodiments, the pharmaceutical compound has a solubility of more than 70 mg/ml as salt. In some embodiments, the pharmaceutical compound has a solubility of more than 60 mg/ml as salt. In some embodiments, the pharmaceutical compound has a solubility of more than 50 mg/ml as salt. In some embodiments, the pharmaceutical compound has a solubility of more than 45 mg/ml as salt. In some embodiments, the pharmaceutical compound has a solubility of more than 40 mg/ml as salt. In some embodiments, the pharmaceutical compound has a solubility of more than 35 mg/ml as salt. In some embodiments, the pharmaceutical compound has a solubility of more than 30 mg/ml as salt. In some embodiments, the pharmaceutical compound has a solubility of more than 25 mg/ml as salt. In some embodiments, the pharmaceutical compound has a solubility of more than 20 mg/ml as salt. In some embodiments, the pharmaceutical compound has a solubility of more than 10 mg/ml as salt. In some embodiments, the pharmaceutical compound has a solubility of more than 9 mg/ml as salt. In some embodiments, the pharmaceutical compound has a solubility of more than 8 mg/ml as salt. In some embodiments, the pharmaceutical compound has a solubility of more than 7 mg/ml as salt. In some embodiments, the pharmaceutical compound has a solubility of more than 6 mg/ml as salt. In some embodiments, the pharmaceutical compound has a solubility of more than 5 mg/ml as salt. In some embodiments, the pharmaceutical compound has a solubility of more than 4 mg/ml as salt. In some embodiments, the pharmaceutical compound has a solubility of more than 3 mg/ml as salt. In some embodiments, the pharmaceutical compound has a solubility of more 2 mg/ml as salt. In some embodiments, the pharmaceutical compound has a solubility of more than 1 mg/ml as salt. In some embodiments, the pharmaceutical compound has a solubility of more than 0.9 mg/ml as salt. In some embodiments, the pharmaceutical compound has a solubility of more than 0.8 mg/ml as salt. In some embodiments, the pharmaceutical compound has a solubility of more than 0.7 mg/ml as salt. In some embodiments, the pharmaceutical compound has a solubility of more than 0.6 mg/ml as salt. In some embodiments, the pharmaceutical compound has a solubility of more than 0.5 mg/ml as salt. In some embodiments, the pharmaceutical compound has a solubility of more than 0.4 mg/ml as salt. In some embodiments, the pharmaceutical compound has a solubility of more than 0.3 mg/ml as salt. In some embodiments, the pharmaceutical compound has a solubility of more than 0.2 mg/ml as salt. In some embodiments, the pharmaceutical compound has a solubility of more than 0.1 mg/ml as salt. In some embodiments, the pharmaceutical compound has a solubility of more than 0.09 mg/ml as salt. In some embodiments, the pharmaceutical compound has a solubility of more than 0.08 mg/ml as salt. In some embodiments, the pharmaceutical compound has a solubility of more than 0.07 mg/ml as salt. In some embodiments, the pharmaceutical compound has a solubility of more than 0.06 mg/ml as salt. In some embodiments, the pharmaceutical compound has a solubility of more than 0.05 mg/ml as salt. In some embodiments, the pharmaceutical compound has a solubility of more than 0.04 mg/ml as salt. In some embodiments, the pharmaceutical compound has a solubility of more than 0.03 mg/ml as salt. In some embodiments, the pharmaceutical compound has a solubility of more than 0.02 mg/ml as salt. In some embodiments, the pharmaceutical compound has a solubility of more than 0.01 mg/ml as salt. In some embodiments, the pharmaceutical compound has a solubility of more than 0.009 mg/ml as salt. In some embodiments, the pharmaceutical compound has a solubility of more than 0.008 mg/ml as salt. In some embodiments, the pharmaceutical compound has a solubility of more than 0.007 mg/ml as salt. In some embodiments, the pharmaceutical compound has a solubility of more than 0.006 mg/ml as salt. In some embodiments, the pharmaceutical compound has a solubility of more than 0.005 mg/ml as salt. In some embodiments, the pharmaceutical compound has a solubility of more than 0.004 mg/ml as salt. In some embodiments, the pharmaceutical compound has a solubility of more than 0.003 mg/ml as salt. In some embodiments, the pharmaceutical compound has a solubility of more than 0.002 mg/ml as salt. In some embodiments, the pharmaceutical compound has a solubility of more than 0.001 mg/ml as salt. In some embodiments, the pharmaceutical compound has a solubility of more than 0.9 μg/ml as salt. In some embodiments, the pharmaceutical compound has a solubility of more than 0.8 μg/ml as salt. In some embodiments, the pharmaceutical compound has a solubility of more than 0.7 μg/ml as salt. In some embodiments, the pharmaceutical compound has a solubility of more than 0.6 μg/ml as salt. In some embodiments, the pharmaceutical compound has a solubility of more than 0.5 μg/ml as salt. In some embodiments, the pharmaceutical compound has a solubility of more than 0.4 μg/ml as salt. In some embodiments, the pharmaceutical compound has a solubility of more than 0.3 μg/ml as salt. In some embodiments, the pharmaceutical compound has a solubility of more than 0.2 μg/ml as salt. In some embodiments, the pharmaceutical compound has a solubility of more than 0.1 μg/ml as salt. In some embodiments, the pharmaceutical compound has a solubility of more than 0.09 μg/ml as salt. In some embodiments, the pharmaceutical compound has a solubility of more than 0.08 μg/ml as salt. In some embodiments, the pharmaceutical compound has a solubility of more than 0.07 μg/ml as salt. In some embodiments, the pharmaceutical compound has a solubility of more than 0.06 μg/ml as salt. In some embodiments, the pharmaceutical compound has a solubility of more than 0.05 μg/ml as salt. In some embodiments, the pharmaceutical compound has a solubility of more than 0.04 μg/ml as salt. In some embodiments, the pharmaceutical compound has a solubility of more than 0.03 μg/ml as salt. In some embodiments, the pharmaceutical compound has a solubility of more than 0.02 μg/ml as salt. In some embodiments, the pharmaceutical compound has a solubility of more than 0.01 μg/ml as salt. In some embodiments, the pharmaceutical compound has a solubility of more than 0.009 μg/ml as salt. In some embodiments, the pharmaceutical compound has a solubility of more than 0.008 μg/ml as salt. In some embodiments, the pharmaceutical compound has a solubility of more than 0.007 μg/ml as salt. In some embodiments, the pharmaceutical compound has a solubility of more than 0.006 μg/ml as salt. In some embodiments, the pharmaceutical compound has a solubility of more than 0.005 μg/ml as salt. In some embodiments, the pharmaceutical compound has a solubility of more than 0.004 μg/ml as salt. In some embodiments, the pharmaceutical compound has a solubility of more than 0.003 μg/ml as salt. In some embodiments, the pharmaceutical compound has a solubility of more than 0.002 μg/ml as salt. In some embodiments, the pharmaceutical compound has a solubility of more than 0.001 μg/ml as salt. In some embodiments, the solubility of the pharmaceutical compound as salt is measured in an aqueous medium. In some embodiments, the solubility of the pharmaceutical compound as salt is measured in an organic solvent. In some embodiments, the aqueous medium comprises water. In some embodiments, the salt of the pharmaceutical compound comprises a HCl salt.

In some embodiments, the pharmaceutical compound has a solubility of between about 0.001 mg/ml and 100 mg/ml as salt. In some embodiments, the pharmaceutical compound has a solubility of between about 0.01 mg/ml and 50 mg/ml as salt. In some embodiments, the pharmaceutical compound has a solubility of between about 0.02 mg/ml and 40 mg/ml as salt. In some embodiments, the pharmaceutical compound has a solubility of between about 0.03 mg/ml and 30 mg/ml as salt. In some embodiments, the pharmaceutical compound has a solubility of between about 0.04 mg/ml and 20 mg/ml as salt. In some embodiments, the pharmaceutical compound has a solubility of between about 0.05 mg/ml and 10 mg/ml as salt. In some embodiments, the pharmaceutical compound has a solubility of between about 0.06 mg/ml and 5 mg/ml as salt. In some embodiments, the pharmaceutical compound has a solubility of between about 0.07 mg/ml and 4 mg/ml as salt. In some embodiments, the pharmaceutical compound has a solubility of between about 0.09 mg/ml and 3 mg/ml as salt. In some embodiments, the pharmaceutical compound has a solubility of between about 0.1 mg/ml and 2 mg/ml as salt. In some embodiments, the solubility of the pharmaceutical compound as salt is measured in an aqueous medium. In some embodiments, the solubility of the pharmaceutical compound as salt is measured in an organic solvent. In some embodiments, the aqueous medium comprises water. In some embodiments, the salt of the pharmaceutical compound comprises a HCl salt.

In some embodiments, the pharmaceutical compound has a solubility of less than 100 mg/ml as salt. In some embodiments, the pharmaceutical compound has a solubility of less than 90 mg/ml as salt. In some embodiments, the pharmaceutical compound has a solubility of less than 80 mg/ml as salt. In some embodiments, the pharmaceutical compound has a solubility of less than 70 mg/ml as salt. In some embodiments, the pharmaceutical compound has a solubility of less than 60 mg/ml as salt. In some embodiments, the pharmaceutical compound has a solubility of less than 50 mg/ml as salt. In some embodiments, the pharmaceutical compound has a solubility of less than 45 mg/ml as salt. In some embodiments, the pharmaceutical compound has a solubility of less than 40 mg/ml as salt. In some embodiments, the pharmaceutical compound has a solubility of less than 35 mg/ml as salt. In some embodiments, the pharmaceutical compound has a solubility of less than 30 mg/ml as salt. In some embodiments, the pharmaceutical compound has a solubility of less than 25 mg/ml as salt. In some embodiments, the pharmaceutical compound has a solubility of less than 20 mg/ml as salt. In some embodiments, the pharmaceutical compound has a solubility of less than 10 mg/ml as salt. In some embodiments, the pharmaceutical compound has a solubility of less than 9 mg/ml as salt. In some embodiments, the pharmaceutical compound has a solubility of less than 8 mg/ml as salt. In some embodiments, the pharmaceutical compound has a solubility of less than 7 mg/ml as salt. In some embodiments, the pharmaceutical compound has a solubility of less than 6 mg/ml as salt. In some embodiments, the pharmaceutical compound has a solubility of less than 5 mg/ml as salt. In some embodiments, the pharmaceutical compound has a solubility of less than 4 mg/ml as salt. In some embodiments, the pharmaceutical compound has a solubility of less than 3 mg/ml as salt. In some embodiments, the pharmaceutical compound has a solubility of more 2 mg/ml as salt. In some embodiments, the pharmaceutical compound has a solubility of less than 1 mg/ml as salt. In some embodiments, the pharmaceutical compound has a solubility of less than 0.9 mg/ml as salt. In some embodiments, the pharmaceutical compound has a solubility of less than 0.8 mg/ml as salt. In some embodiments, the pharmaceutical compound has a solubility of less than 0.7 mg/ml as salt. In some embodiments, the pharmaceutical compound has a solubility of less than 0.6 mg/ml as salt. In some embodiments, the pharmaceutical compound has a solubility of less than 0.5 mg/ml as salt. In some embodiments, the pharmaceutical compound has a solubility of less than 0.4 mg/ml as salt. In some embodiments, the pharmaceutical compound has a solubility of less than 0.3 mg/ml as salt. In some embodiments, the pharmaceutical compound has a solubility of less than 0.2 mg/ml as salt. In some embodiments, the pharmaceutical compound has a solubility of less than 0.1 mg/ml as salt. In some embodiments, the pharmaceutical compound has a solubility of less than 0.09 mg/ml as salt. In some embodiments, the pharmaceutical compound has a solubility of less than 0.08 mg/ml as salt. In some embodiments, the pharmaceutical compound has a solubility of less than 0.07 mg/ml as salt. In some embodiments, the pharmaceutical compound has a solubility of less than 0.06 mg/ml as salt. In some embodiments, the pharmaceutical compound has a solubility of less than 0.05 mg/ml as salt. In some embodiments, the pharmaceutical compound has a solubility of less than 0.04 mg/ml as salt. In some embodiments, the pharmaceutical compound has a solubility of less than 0.03 mg/ml as salt. In some embodiments, the pharmaceutical compound has a solubility of less than 0.02 mg/ml as salt. In some embodiments, the pharmaceutical compound has a solubility of less than 0.01 mg/ml as salt. In some embodiments, the pharmaceutical compound has a solubility of less than 0.009 mg/ml as salt. In some embodiments, the pharmaceutical compound has a solubility of less than 0.008 mg/ml as salt. In some embodiments, the pharmaceutical compound has a solubility of less than 0.007 mg/ml as salt. In some embodiments, the pharmaceutical compound has a solubility of less than 0.006 mg/ml as salt. In some embodiments, the pharmaceutical compound has a solubility of less than 0.005 mg/ml as salt. In some embodiments, the pharmaceutical compound has a solubility of less than 0.004 mg/ml as salt. In some embodiments, the pharmaceutical compound has a solubility of less than 0.003 mg/ml as salt. In some embodiments, the pharmaceutical compound has a solubility of less than 0.002 mg/ml as salt. In some embodiments, the pharmaceutical compound has a solubility of less than 0.001 mg/ml as salt. In some embodiments, the pharmaceutical compound has a solubility of less than 0.9 μg/ml as salt. In some embodiments, the pharmaceutical compound has a solubility of less than 0.8 μg/ml as salt. In some embodiments, the pharmaceutical compound has a solubility of less than 0.7 μg/ml as salt. In some embodiments, the pharmaceutical compound has a solubility of less than 0.6 μg/ml as salt. In some embodiments, the pharmaceutical compound has a solubility of less than 0.5 μg/ml as salt. In some embodiments, the pharmaceutical compound has a solubility of less than 0.4 μg/ml as salt. In some embodiments, the pharmaceutical compound has a solubility of less than 0.3 μg/ml as salt. In some embodiments, the pharmaceutical compound has a solubility of less than 0.2 μg/ml as salt. In some embodiments, the pharmaceutical compound has a solubility of less than 0.1 μg/ml as salt. In some embodiments, the pharmaceutical compound has a solubility of less than 0.09 μg/ml as salt. In some embodiments, the pharmaceutical compound has a solubility of less than 0.08 μg/ml as salt. In some embodiments, the pharmaceutical compound has a solubility of less than 0.07 μg/ml as salt. In some embodiments, the pharmaceutical compound has a solubility of less than 0.06 μg/ml as salt. In some embodiments, the pharmaceutical compound has a solubility of less than 0.05 μg/ml as salt. In some embodiments, the pharmaceutical compound has a solubility of less than 0.04 μg/ml as salt. In some embodiments, the pharmaceutical compound has a solubility of less than 0.03 μg/ml as salt. In some embodiments, the pharmaceutical compound has a solubility of less than 0.02 μg/ml as salt. In some embodiments, the pharmaceutical compound has a solubility of less than 0.01 μg/ml as salt. In some embodiments, the pharmaceutical compound has a solubility of less than 0.009 μg/ml as salt. In some embodiments, the pharmaceutical compound has a solubility of less than 0.008 μg/ml as salt. In some embodiments, the pharmaceutical compound has a solubility of less than 0.007 μg/ml as salt. In some embodiments, the pharmaceutical compound has a solubility of less than 0.006 μg/ml as salt. In some embodiments, the pharmaceutical compound has a solubility of less than 0.005 μg/ml as salt. In some embodiments, the pharmaceutical compound has a solubility of less than 0.004 μg/ml as salt. In some embodiments, the pharmaceutical compound has a solubility of less than 0.003 μg/ml as salt. In some embodiments, the pharmaceutical compound has a solubility of less than 0.002 μg/ml as salt. In some embodiments, the pharmaceutical compound has a solubility of less than 0.001 μg/ml as salt. In some embodiments, the solubility of the pharmaceutical compound as salt is measured in an aqueous medium. In some embodiments, the solubility of the pharmaceutical compound as salt is measured in an organic solvent. In some embodiments, the aqueous medium comprises water. In some embodiments, the salt of the pharmaceutical compound comprises a HCl salt.

In some embodiments, the pharmaceutical compound has a solubility of more than 100 mg/ml as freebase. In some embodiments, the pharmaceutical compound has a solubility of more than 90 mg/ml as freebase. In some embodiments, the pharmaceutical compound has a solubility of more than 80 mg/ml as freebase. In some embodiments, the pharmaceutical compound has a solubility of more than 70 mg/ml as freebase. In some embodiments, the pharmaceutical compound has a solubility of more than 60 mg/ml as freebase. In some embodiments, the pharmaceutical compound has a solubility of more than 50 mg/ml as freebase. In some embodiments, the pharmaceutical compound has a solubility of more than 45 mg/ml as freebase. In some embodiments, the pharmaceutical compound has a solubility of more than 40 mg/ml as freebase. In some embodiments, the pharmaceutical compound has a solubility of more than 35 mg/ml as freebase. In some embodiments, the pharmaceutical compound has a solubility of more than 30 mg/ml as freebase. In some embodiments, the pharmaceutical compound has a solubility of more than 25 mg/ml as freebase. In some embodiments, the pharmaceutical compound has a solubility of more than 20 mg/ml as freebase. In some embodiments, the pharmaceutical compound has a solubility of more than 10 mg/ml as freebase. In some embodiments, the pharmaceutical compound has a solubility of more than 9 mg/ml as freebase. In some embodiments, the pharmaceutical compound has a solubility of more than 8 mg/ml as freebase. In some embodiments, the pharmaceutical compound has a solubility of more than 7 mg/ml as freebase. In some embodiments, the pharmaceutical compound has a solubility of more than 6 mg/ml as freebase. In some embodiments, the pharmaceutical compound has a solubility of more than 5 mg/ml as freebase. In some embodiments, the pharmaceutical compound has a solubility of more than 4 mg/ml as freebase. In some embodiments, the pharmaceutical compound has a solubility of more than 3 mg/ml as freebase. In some embodiments, the pharmaceutical compound has a solubility of more 2 mg/ml as freebase. In some embodiments, the pharmaceutical compound has a solubility of more than 1 mg/ml as freebase. In some embodiments, the pharmaceutical compound has a solubility of more than 0.9 mg/ml as freebase. In some embodiments, the pharmaceutical compound has a solubility of more than 0.8 mg/ml as freebase. In some embodiments, the pharmaceutical compound has a solubility of more than 0.7 mg/ml as freebase. In some embodiments, the pharmaceutical compound has a solubility of more than 0.6 mg/ml as freebase. In some embodiments, the pharmaceutical compound has a solubility of more than 0.5 mg/ml as freebase. In some embodiments, the pharmaceutical compound has a solubility of more than 0.4 mg/ml as freebase. In some embodiments, the pharmaceutical compound has a solubility of more than 0.3 mg/ml as freebase. In some embodiments, the pharmaceutical compound has a solubility of more than 0.2 mg/ml as freebase. In some embodiments, the pharmaceutical compound has a solubility of more than 0.1 mg/ml as freebase. In some embodiments, the pharmaceutical compound has a solubility of more than 0.09 mg/ml as freebase. In some embodiments, the pharmaceutical compound has a solubility of more than 0.08 mg/ml as freebase. In some embodiments, the pharmaceutical compound has a solubility of more than 0.07 mg/ml as freebase. In some embodiments, the pharmaceutical compound has a solubility of more than 0.06 mg/ml as freebase. In some embodiments, the pharmaceutical compound has a solubility of more than 0.05 mg/ml as freebase. In some embodiments, the pharmaceutical compound has a solubility of more than 0.04 mg/ml as freebase. In some embodiments, the pharmaceutical compound has a solubility of more than 0.03 mg/ml as freebase. In some embodiments, the pharmaceutical compound has a solubility of more than 0.02 mg/ml as freebase. In some embodiments, the pharmaceutical compound has a solubility of more than 0.01 mg/ml as freebase. In some embodiments, the pharmaceutical compound has a solubility of more than 0.009 mg/ml as freebase. In some embodiments, the pharmaceutical compound has a solubility of more than 0.008 mg/ml as freebase. In some embodiments, the pharmaceutical compound has a solubility of more than 0.007 mg/ml as freebase. In some embodiments, the pharmaceutical compound has a solubility of more than 0.006 mg/ml as freebase. In some embodiments, the pharmaceutical compound has a solubility of more than 0.005 mg/ml as freebase. In some embodiments, the pharmaceutical compound has a solubility of more than 0.004 mg/ml as freebase. In some embodiments, the pharmaceutical compound has a solubility of more than 0.003 mg/ml as freebase. In some embodiments, the pharmaceutical compound has a solubility of more than 0.002 mg/ml as freebase. In some embodiments, the pharmaceutical compound has a solubility of more than 0.001 mg/ml as freebase. In some embodiments, the pharmaceutical compound has a solubility of more than 0.9 μg/ml as freebase. In some embodiments, the pharmaceutical compound has a solubility of more than 0.8 μg/ml as freebase. In some embodiments, the pharmaceutical compound has a solubility of more than 0.7 μg/ml as freebase. In some embodiments, the pharmaceutical compound has a solubility of more than 0.6 μg/ml as freebase. In some embodiments, the pharmaceutical compound has a solubility of more than 0.5 μg/ml as freebase. In some embodiments, the pharmaceutical compound has a solubility of more than 0.4 μg/ml as freebase. In some embodiments, the pharmaceutical compound has a solubility of more than 0.3 μg/ml as freebase. In some embodiments, the pharmaceutical compound has a solubility of more than 0.2 μg/ml as freebase. In some embodiments, the pharmaceutical compound has a solubility of more than 0.1 μg/ml as freebase. In some embodiments, the pharmaceutical compound has a solubility of more than 0.09 μg/ml as freebase. In some embodiments, the pharmaceutical compound has a solubility of more than 0.08 μg/ml as freebase. In some embodiments, the pharmaceutical compound has a solubility of more than 0.07 μg/ml as freebase. In some embodiments, the pharmaceutical compound has a solubility of more than 0.06 μg/ml as freebase. In some embodiments, the pharmaceutical compound has a solubility of more than 0.05 μg/ml as freebase. In some embodiments, the pharmaceutical compound has a solubility of more than 0.04 μg/ml as freebase. In some embodiments, the pharmaceutical compound has a solubility of more than 0.03 μg/ml as freebase. In some embodiments, the pharmaceutical compound has a solubility of more than 0.02 μg/ml as freebase. In some embodiments, the pharmaceutical compound has a solubility of more than 0.01 μg/ml as freebase. In some embodiments, the pharmaceutical compound has a solubility of more than 0.009 μg/ml as freebase. In some embodiments, the pharmaceutical compound has a solubility of more than 0.008 μg/ml as freebase. In some embodiments, the pharmaceutical compound has a solubility of more than 0.007 μg/ml as freebase. In some embodiments, the pharmaceutical compound has a solubility of more than 0.006 μg/ml as freebase. In some embodiments, the pharmaceutical compound has a solubility of more than 0.005 μg/ml as freebase. In some embodiments, the pharmaceutical compound has a solubility of more than 0.004 μg/ml as freebase. In some embodiments, the pharmaceutical compound has a solubility of more than 0.003 μg/ml as freebase. In some embodiments, the pharmaceutical compound has a solubility of more than 0.002 μg/ml as freebase. In some embodiments, the pharmaceutical compound has a solubility of more than 0.001 μg/ml as freebase. In some embodiments, the solubility of the pharmaceutical compound as freebase is measured in an aqueous medium. In some embodiments, the solubility of the pharmaceutical compound as freebase is measured in an organic solvent. In some embodiments, the aqueous medium comprises water.

In some embodiments, the pharmaceutical compound has a solubility of between about 0.001 mg/ml and 100 mg/ml as freebase. In some embodiments, the pharmaceutical compound has a solubility of between about 0.01 mg/ml and 50 mg/ml as freebase. In some embodiments, the pharmaceutical compound has a solubility of between about 0.02 mg/ml and 40 mg/ml as freebase. In some embodiments, the pharmaceutical compound has a solubility of between about 0.03 mg/ml and 30 mg/ml as freebase. In some embodiments, the pharmaceutical compound has a solubility of between about 0.04 mg/ml and 20 mg/ml as freebase. In some embodiments, the pharmaceutical compound has a solubility of between about 0.05 mg/ml and 10 mg/ml as freebase. In some embodiments, the pharmaceutical compound has a solubility of between about 0.06 mg/ml and 5 mg/ml as freebase. In some embodiments, the pharmaceutical compound has a solubility of between about 0.07 mg/ml and 4 mg/ml as freebase. In some embodiments, the pharmaceutical compound has a solubility of between about 0.09 mg/ml and 3 mg/ml as freebase. In some embodiments, the pharmaceutical compound has a solubility of between about 0.1 mg/ml and 2 mg/ml as freebase. In some embodiments, the solubility of the pharmaceutical compound as freebase is measured in an aqueous medium. In some embodiments, the solubility of the pharmaceutical compound as freebase is measured in an organic solvent. In some embodiments, the aqueous medium comprises water.

In some embodiments, the pharmaceutical compound has a solubility of less than 100 mg/ml as freebase. In some embodiments, the pharmaceutical compound has a solubility of less than 90 mg/ml as freebase. In some embodiments, the pharmaceutical compound has a solubility of less than 80 mg/ml as freebase. In some embodiments, the pharmaceutical compound has a solubility of less than 70 mg/ml as freebase. In some embodiments, the pharmaceutical compound has a solubility of less than 60 mg/ml as freebase. In some embodiments, the pharmaceutical compound has a solubility of less than 50 mg/ml as freebase. In some embodiments, the pharmaceutical compound has a solubility of less than 45 mg/ml as freebase. In some embodiments, the pharmaceutical compound has a solubility of less than 40 mg/ml as freebase. In some embodiments, the pharmaceutical compound has a solubility of less than 35 mg/ml as freebase. In some embodiments, the pharmaceutical compound has a solubility of less than 30 mg/ml as freebase. In some embodiments, the pharmaceutical compound has a solubility of less than 25 mg/ml as freebase. In some embodiments, the pharmaceutical compound has a solubility of less than 20 mg/ml as freebase. In some embodiments, the pharmaceutical compound has a solubility of less than 10 mg/ml as freebase. In some embodiments, the pharmaceutical compound has a solubility of less than 9 mg/ml as freebase. In some embodiments, the pharmaceutical compound has a solubility of less than 8 mg/ml as freebase. In some embodiments, the pharmaceutical compound has a solubility of less than 7 mg/ml as freebase. In some embodiments, the pharmaceutical compound has a solubility of less than 6 mg/ml as freebase. In some embodiments, the pharmaceutical compound has a solubility of less than 5 mg/ml as freebase. In some embodiments, the pharmaceutical compound has a solubility of less than 4 mg/ml as freebase. In some embodiments, the pharmaceutical compound has a solubility of less than 3 mg/ml as freebase. In some embodiments, the pharmaceutical compound has a solubility of more 2 mg/ml as freebase. In some embodiments, the pharmaceutical compound has a solubility of less than 1 mg/ml as freebase. In some embodiments, the pharmaceutical compound has a solubility of less than 0.9 mg/ml as freebase. In some embodiments, the pharmaceutical compound has a solubility of less than 0.8 mg/ml as freebase. In some embodiments, the pharmaceutical compound has a solubility of less than 0.7 mg/ml as freebase. In some embodiments, the pharmaceutical compound has a solubility of less than 0.6 mg/ml as freebase. In some embodiments, the pharmaceutical compound has a solubility of less than 0.5 mg/ml as freebase. In some embodiments, the pharmaceutical compound has a solubility of less than 0.4 mg/ml as freebase. In some embodiments, the pharmaceutical compound has a solubility of less than 0.3 mg/ml as freebase. In some embodiments, the pharmaceutical compound has a solubility of less than 0.2 mg/ml as freebase. In some embodiments, the pharmaceutical compound has a solubility of less than 0.1 mg/ml as freebase. In some embodiments, the pharmaceutical compound has a solubility of less than 0.09 mg/ml as freebase. In some embodiments, the pharmaceutical compound has a solubility of less than 0.08 mg/ml as freebase. In some embodiments, the pharmaceutical compound has a solubility of less than 0.07 mg/ml as freebase. In some embodiments, the pharmaceutical compound has a solubility of less than 0.06 mg/ml as freebase. In some embodiments, the pharmaceutical compound has a solubility of less than 0.05 mg/ml as freebase. In some embodiments, the pharmaceutical compound has a solubility of less than 0.04 mg/ml as freebase. In some embodiments, the pharmaceutical compound has a solubility of less than 0.03 mg/ml as freebase. In some embodiments, the pharmaceutical compound has a solubility of less than 0.02 mg/ml as freebase. In some embodiments, the pharmaceutical compound has a solubility of less than 0.01 mg/ml as freebase. In some embodiments, the pharmaceutical compound has a solubility of less than 0.009 mg/ml as freebase. In some embodiments, the pharmaceutical compound has a solubility of less than 0.008 mg/ml as freebase. In some embodiments, the pharmaceutical compound has a solubility of less than 0.007 mg/ml as freebase. In some embodiments, the pharmaceutical compound has a solubility of less than 0.006 mg/ml as freebase. In some embodiments, the pharmaceutical compound has a solubility of less than 0.005 mg/ml as freebase. In some embodiments, the pharmaceutical compound has a solubility of less than 0.004 mg/ml as freebase. In some embodiments, the pharmaceutical compound has a solubility of less than 0.003 mg/ml as freebase. In some embodiments, the pharmaceutical compound has a solubility of less than 0.002 mg/ml as freebase. In some embodiments, the pharmaceutical compound has a solubility of less than 0.001 mg/ml as freebase. In some embodiments, the pharmaceutical compound has a solubility of less than 0.9 μg/ml as freebase. In some embodiments, the pharmaceutical compound has a solubility of less than 0.8 μg/ml as freebase. In some embodiments, the pharmaceutical compound has a solubility of less than 0.7 μg/ml as freebase. In some embodiments, the pharmaceutical compound has a solubility of less than 0.6 μg/ml as freebase. In some embodiments, the pharmaceutical compound has a solubility of less than 0.5 μg/ml as freebase. In some embodiments, the pharmaceutical compound has a solubility of less than 0.4 μg/ml as freebase. In some embodiments, the pharmaceutical compound has a solubility of less than 0.3 μg/ml as freebase. In some embodiments, the pharmaceutical compound has a solubility of less than 0.2 μg/ml as freebase. In some embodiments, the pharmaceutical compound has a solubility of less than 0.1 μg/ml as freebase.

In some embodiments, the pharmaceutical compound has a solubility of less than 0.09 μg/ml as freebase. In some embodiments, the pharmaceutical compound has a solubility of less than 0.08 μg/ml as freebase. In some embodiments, the pharmaceutical compound has a solubility of less than 0.07 μg/ml as freebase. In some embodiments, the pharmaceutical compound has a solubility of less than 0.06 μg/ml as freebase. In some embodiments, the pharmaceutical compound has a solubility of less than 0.05 μg/ml as freebase. In some embodiments, the pharmaceutical compound has a solubility of less than 0.04 μg/ml as freebase. In some embodiments, the pharmaceutical compound has a solubility of less than 0.03 μg/ml as freebase. In some embodiments, the pharmaceutical compound has a solubility of less than 0.02 μg/ml as freebase. In some embodiments, the pharmaceutical compound has a solubility of less than 0.01 μg/ml as freebase. In some embodiments, the pharmaceutical compound has a solubility of less than 0.009 μg/ml as freebase. In some embodiments, the pharmaceutical compound has a solubility of less than 0.008 μg/ml as freebase. In some embodiments, the pharmaceutical compound has a solubility of less than 0.007 μg/ml as freebase. In some embodiments, the pharmaceutical compound has a solubility of less than 0.006 μg/ml as freebase. In some embodiments, the pharmaceutical compound has a solubility of less than 0.005 μg/ml as freebase. In some embodiments, the pharmaceutical compound has a solubility of less than 0.004 μg/ml as freebase. In some embodiments, the pharmaceutical compound has a solubility of less than 0.003 μg/ml as freebase. In some embodiments, the pharmaceutical compound has a solubility of less than 0.002 μg/ml as freebase. In some embodiments, the pharmaceutical compound has a solubility of less than 0.001 μg/ml as freebase. In some embodiments, the solubility of the pharmaceutical compound as freebase is measured in an aqueous medium. In some embodiments, the solubility of the pharmaceutical compound as freebase is measured in an organic solvent. In some embodiments, the aqueous medium comprises water.

In one aspect, provided herein is a method of preparing a pharmaceutical composition, comprising: mixing a free acid form of a complexing agent comprising a plurality of acidic functional groups and a freebase form of a pharmaceutical compound, wherein the pharmaceutical compound comprises a prodrug comprising an unionized substance conjugated to a chemical entity, wherein the chemical entity comprises a basic nitrogen atom. In embodiments, the unionized substance comprises brexanolone. In some embodiments, the chemical entity comprises γ-aminobutyric acid (GABA).

In some embodiments, the molar ratio of the pharmaceutical compound comprising the prodrug to the number of moles of the acidic functional groups of the free acid form of the complexing agent is about 1:1, in which case all or substantially all of the pharmaceutical compound comprising the prodrug becomes protonated. In some embodiments, the molar ratio of the additional unionized substance being added to the mixture to the number of moles of the free acid form of the complexing agent is about 1:1, in which the resulting pharmaceutical formulation comprises the unionized substance. In some embodiments, the molar ratio of the pharmaceutical compound comprising the prodrug to the number of moles of acidic functional groups of the complexing agent is greater than 1:1 (e.g., 2:1, 3:1, 4:1, 5:1, 6:1, 7:1, 8:1, 9:1, or 10:1 or greater), in which case a portion of the pharmaceutical compound will remain unionized.

In one aspect, provided herein is a method of preparing a pharmaceutical composition, comprising: mixing a free acid form of a complexing agent comprising a plurality of acidic functional groups and a freebase form of a pharmaceutical compound, wherein the pharmaceutical compound comprises a prodrug comprising an unionized substance conjugated to a chemical entity, wherein the chemical entity comprises a basic nitrogen atom, and adding an additional molar equivalent of the unionized substance, wherein the additional molar equivalent is unionized and does not ionize after addition. In embodiments, the unionized substance comprises brexanolone. In some embodiments, the chemical entity comprises γ-aminobutyric acid (GABA).

In some embodiments, the molar ratio of the pharmaceutical compound comprising the prodrug to the number of moles of the acidic functional groups of the free acid form of the complexing agent is about 1:1, in which case all or substantially all of the pharmaceutical compound comprising the prodrug becomes protonated. In some embodiments, the molar ratio of the additional unionized substance being added to the mixture to the number of moles of the free acid form of the complexing agent is about 1:1, in which the resulting pharmaceutical formulation comprises the unionized substance. In some embodiments, the molar ratio of the pharmaceutical compound comprising the prodrug to the number of moles of acidic functional groups of the complexing agent is greater than 1:1 (e.g., 2:1, 3:1, 4:1, 5:1, 6:1, 7:1, 8:1, 9:1, or 10:1 or greater), in which case a portion of the pharmaceutical compound will remain unionized.

In some embodiments, all or a portion of the pharmaceutical compound comprising the prodrug becomes protonated in the liquid medium upon addition of the free acid form of the complexing agent. At this point, in some embodiments, the unionized substance is then added. In some embodiments, the liquid medium is first removed by a suitable method (e.g. evaporation, lyophilization, etc.) before the unionized substance is added. In such cases, any of the ratios of molar equivalents of unionized substance provided herein may be used.

In some embodiments, the solubility of the ununionized substance in the pharmaceutical composition is higher than (i) the solubility of the unionized substance as a salt; or (ii) the solubility of the unionized substance in a composition comprising the unionized substance in freebase form that is complexing to a non-polar pore of the complexing agent, wherein the unionized substance has the same concentration in the pharmaceutical composition, the unionized substance as a salt, and the composition comprising the unionized substance in freebase form. In some embodiments, the solubility of the unionized substance in the pharmaceutical composition is higher than the solubility of the unionized substance in a composition comprising the unionized substance and complexing agent with a higher molar ratio of the unionized substance to the complexing agent, wherein the unionized substance has the same concentration in the pharmaceutical composition and the composition with a higher molar ratio of the unionized substance to the complexing agent. In some embodiments, the solubility of the unionized substance in the pharmaceutical composition has an increase of at least 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, or 100-fold compared with (i) the solubility of the unionized substance as a salt; or (ii) the solubility of the unionized substance in a composition comprising the unionized substance in freebase form that is complexing to a non-polar pore of the complexing agent, wherein the unionized substance has the same concentration in the pharmaceutical composition, the unionized substance as a salt, and the composition comprising the unionized substance in freebase form. In some embodiments, the solubility of the unionized substance in the pharmaceutical composition has an increase of at least 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, or 100-fold compared with the solubility of the unionized substance in a composition comprising the unionized substance and complexing agent with a higher molar ratio of the unionized substance to the complexing agent, wherein the unionized substance has the same concentration in the pharmaceutical composition and the composition with a higher molar ratio of the unionized substance to the complexing agent.

In one aspect, provided herein is a method of preparing a pharmaceutical composition, comprising: mixing a free acid form of a complexing agent comprising a plurality of acidic functional groups and a freebase form of a first pharmaceutical compound, wherein the first pharmaceutical compound comprises a basic nitrogen atom; and adding a second pharmaceutical compound, wherein the second pharmaceutical compound is unionized after addition.

In some embodiments, the first pharmaceutical compound comprises ketamine. In some embodiments, the second pharmaceutical compound comprises rapamycin. In some embodiments, the first pharmaceutical compound comprises ketamine and the second pharmaceutical compound comprises rapamycin. In some embodiments, the first pharmaceutical compound comprises no ketamine and the second pharmaceutical compound comprises rapamycin. In some embodiments, the second pharmaceutical compound comprises clonidine. In some embodiments, the first pharmaceutical compound comprises ketamine and the second pharmaceutical compound comprises clonidine. In some embodiments, the first pharmaceutical compound comprises no ketamine and the second pharmaceutical compound comprises clonidine. In some embodiments, the second pharmaceutical compound comprises no protonated nitrogen atom.

In some embodiments, the molar ratio of the first pharmaceutical compound to the number of moles of acidic functional groups of the complexing agent is about 1:1 and the molar ratio of the second pharmaceutical compound to the number of moles of the complexing agent is about 1:1, in which case all or substantially all of the first pharmaceutical compound becomes protonated and the resulting pharmaceutical composition comprises the unionized second pharmaceutical compound. In some embodiments, the molar ratio of the first pharmaceutical compound to the number of moles of acidic functional groups of the complexing agent is greater than 1:1 (e.g., 2:1, 3:1, 4:1, 5:1, 6:1, 7:1, 8:1, 9:1, or 10:1 or greater), in which case a portion of the first pharmaceutical compound will remain unionized.

In some embodiments, all or a portion of the first pharmaceutical compound becomes protonated in the liquid medium upon addition of the free acid form of the complexing agent. At this point, in some embodiments, the second pharmaceutical compound is then added. In some embodiments, the liquid medium is first removed by a suitable method (e.g. evaporation, lyophilization, etc.) before the unionized second pharmaceutical compound is added. In such cases, any of the ratios of molar equivalents of unionized second pharmaceutical compound provided herein may be used.

In some embodiments, the solubility of the second pharmaceutical compound in the pharmaceutical composition is higher than the solubility of the second pharmaceutical compound as a salt, wherein the second pharmaceutical compound has the same concentration in the pharmaceutical composition, and the second pharmaceutical compound as a salt. In some embodiments, the solubility of the second pharmaceutical compound in the pharmaceutical composition has an increase of at least 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, or 100-fold compared with the solubility of the second pharmaceutical compound as a salt, wherein the second pharmaceutical compound has the same concentration in the pharmaceutical composition, and the second pharmaceutical compound as a salt.

In some embodiments, the mixing occurs in a suitable medium. In some embodiments, the suitable medium is an aqueous medium. In some embodiments, the suitable medium is an organic solvent. In some embodiments, the mixing occurs in a solution. In some embodiments, the mixing occurs when the complexing agent and the pharmaceutical compound are in powder form.

In some embodiments, the mixing occurs by portion-wise addition of one of the reagents. In some embodiments, the pharmaceutical compound is added portion wise to a solution comprising the complexing agent.

In some embodiments, the pharmaceutical composition is in a form for dosing or administration by subcutaneous injection.

In some embodiments, the complexing agent comprising at least one acidic functional group is a cyclodextrin. Any of the cyclodextrins provided in the “Compositions” section can be used. The cyclodextrin or other complexing agent may also be added at any amount provided in the “Compositions” section or at any molar ratio provided therein, including ratios of acidic functional groups to the pharmaceutical compound.

In some embodiments, a method disclosed herein further comprises adjusting the pH of the pharmaceutical composition. In some embodiments, only a minimal adjustment of the pH is necessary. In some embodiments, the pH is adjusted with a strong base. In some embodiments, the pH is adjusted with sodium hydroxide, potassium hydroxide, barium hydroxide, cesium hydroxide, strontium hydroxide magnesium hydroxide, calcium hydroxide, lithium hydroxide, or rubidium hydroxide. In some embodiments, the pH is adjusted with sodium hydroxide. In some embodiments, the pH is adjusted to a desired pH. In some embodiments, the desired pH is any of the pH values provided herein in the “Compositions” section.

In some embodiments, the pharmaceutical composition has a pH >about 4. The pH of the pharmaceutical composition can be any of the pH values provided in the “Compositions” section, such as a pH from about 4 to about 7, or any other of the pH values or ranges provided therein.

In some embodiments, the method further comprises adding a preservative to the composition. The preservative may be added to the composition at any time or in any order. In some embodiments, the preservative is added after the complexing agent and the pharmaceutical compound have been added. In some embodiments, the preservative is added prior to adding the pharmaceutical compound. In some embodiments, the preservative is benzethonium chloride. The preservative may also be any of the preservatives provided in the “Compositions” section and may be added at any concentration provided therein.

In some embodiments, the method further comprises adding a base, a buffer, an emulsifying agent, a surfactant, a solubilizing agent, an emulsifying agent, a co-solvent, or any combination thereof. The method may also further comprise adding any of the additional components provided herein in the “Compositions” section, including without limitation bases, buffers, co-solvents, preservatives, surface-active agents, surfactants, solubilizing agents, stabilization agents, antioxidants, cryoprotectants, lyoprotectants, bulking agents, tonicity-adjusting agents, antimicrobial agents, diluents, soothing agents, and/or emulsion stabilizers.

In some embodiments, the method further comprises adjusting the osmolality of the pharmaceutical composition. In some embodiments, the adjusting the osmolality of the pharmaceutical comprises diluting the pharmaceutical composition. Diluting the pharmaceutical composition may comprise diluting with water or other physiologically acceptable buffer, such as phosphate buffered saline, or any of the buffers provided in the “Compositions” section. In some embodiments, adjusting the osmolality of the pharmaceutical composition comprises adding a tonicity modifying agent. The tonicity modifying reagent may be any pharmaceutically acceptable reagent, such as sodium chloride, or any of the reagents provided in the “Compositions” section. In some embodiments, there is no need to adjust the osmolality of the pharmaceutical composition after mixing the pharmaceutical compound or the compound of structural Formula (I) and the complexing agent.

The final osmolality of the pharmaceutical composition may be any of the osmolalities provided in the “Compositions” section, such an osmolality from about 250 mOsm/kg to about 850 mOsm/kg, or any other range or value provided therein.

The final concentration of the compound of structural Formula (I) of the pharmaceutical composition may be any of the concentrations provided in the “Compositions” section, including values over 10 or 20 mg/mL or any of the other values or ranges provided therein.

Also provided herein are methods of generating the free acid form of complexing agents comprising at least one acidic functional group. In some cases, complexing agents comprising acidic functional groups are commercially available only as salts of the complexing agents, such as sodium salts. For example, SBEBCD is sold commercially as the sodium salt.

The free acid form of such complexing agents can be generated by any number of methods. For example, the salts of complexing agents comprising acidic functional groups can be bound to a suitable acidic cation exchange resin (e.g. Amberlite® IR120 Hydrogen form resin, available from commercial vendors such as Sigma Aldrich) and then eluted to yield the desired free acid form of the complexing agent.

An additional method potentially suitable for this purpose could involve treating the sodium salt of the complexing agent (e.g. SBEBCD) with a hydrochloric acid or other suitable acid in a suitable organic solvent. Ideally, the organic solvent is selected such the resulting sodium chloride will precipitate out of solution and leave the complexing agent free acid in solution. The sodium chloride salt could then be removed by filtration, and the filtrate could then be concentrated or the solvent removed to yield the free acid. Conversely, the filtrate could be solvent exchanged with water for injection using standard azeotropic distillation under vacuum. Alternatively, the filtrate could be used to prepare the formulation as is and removed at a later stage.

### EXAMPLES

### Example 1. Preparation of Complexing Agent Salt Formulations of Compounds at Low

### Stoichiometric Ratios

This example illustrates the preparation of various complexing agent salt formulations of compounds at low stoichiometric ratios.

### Chemicals and Reagents

Solvents (water and acetonitrile) were HPLC Plus grade and obtained from Sigma Aldrich (St. Louis, MO). The following chemicals were obtained and used without further purification: ammonium formate (≥99.9%, Sigma Aldrich, St. Louis, MO), formic acid (≥98%, Sigma Aldrich, St. Louis, MO), Amberlite IRC120 H hydrogen form resin (Sigma Aldrich, St. Louis, MO), ethanol (200 proof, Pharmco, Greenfield Global, Toronto, Canada), chloroform-d (≥99.8% atom D, Sigma Aldrich, St. Louis, MO), dimethylsulfoxide-d6 (99.9% atom D, Sigma-Aldrich, St. Louis, MO), deuterium oxide (MagniSolv™, Sigma-Aldrich, St. Louis, MO), sodium hydroxide (≥97%, Sigma Aldrich, St. Louis, MO), potassium hydroxide (≥85%, Sigma Aldrich, St. Louis, MO), sodium chloride (Certified ACS crystalline, Fisher Scientific, Fair Lawn, NJ), 0.9% saline solution (AddiPak, Teleflex Inc, USA), sodium tartrate (Aqualine™ Primary Standard, Fisher Scientific, Fair Lawn, NJ), Benzethonium chloride (≥99%, Sigma Aldrich, St. Louis, MO), Captisol® (Lot #NC-06C-20050, Cydex Pharmaceuticals, Lawrence, KS), Amantadine hydrochloride (Alfa Aesar, Ward Hill, MA), Rimantadine hydrochloride (MedChemExpress, Monmouth Junction, NJ), Nafamostat (2HBiochem, Inc., Edison, NJ), Remdesivir (AmBeed, Arlington Heights, IL), Copanlisib (Arctom Scientific, Westlake Village, CA), Amikacin free base (Santa Cruz Biotechnology, Inc., Dallas, TX), Pyridine-3,4-diamine (Amifampridine, 1Plus Chem, San Diego, CA). The following APIs were obtained from A Fine Chemical (China) and used without further purification: Naloxone hydrochloride dihydrate, eletriptan hydrobromide, rotigotine hydrochloride, tigecycline, and caspofungin acetate. Chemicals obtained from A FIne Chemical were subjected to 1H and 13C NMR, melting point analysis and HPLC.

### Consumables

The following consumables were used: Corning 0.20 micron syringe filters, Corning 0.45 micron syringe filters, Shaotong disposable hypodermic needles, 3 mL luer-lock disposable syringes (Air-Tite Products Co., Inc.), Fischer brand 5 mL borosilicate glass test tubes, Fischer brand 15 mL borosilicate glass test tubes, Chemglass Life Sciences 20 mL scintillation vials, Amcor parafilm, Fisher brand 9″ pasteur pipettes, Lichen Cottage brand 200 μL filtered pipette tips, Lichen Cottage brand 1000 μL filtered pipette tips, Sartorius 10 μL pipette tips, Agilent screw top 2 mL amber vials, Agilent PTFE/red silicone septa screw caps, Thermo Scientific 2 mL depyrogenated sterile empty vials, Thermo Scientific 5 mL depyrogenated sterile empty vials, Pyrex 0.8-1.1×90 mm capillary tubes, Kimtech kimwipes, Thermo Scientific microcentrifuge tubes, Globe Scientific 15 mL conical tubes, Globe Scientific 50 ml conical tubes.

### pH and Mass Measurements

pH readings were obtained by an Orion 3 star (Thermo Scientific, USA) PH meter equipped with either a Thermo pH electrode (9142BN) or an Orion 8103BNUWP Ross Ultra Semi-micro pH probe (Thermo Scientific, USA) containing 3M KCl ROSS Orion filling solution (Thermos Scientific, USA). An Ohaus ADVENTURER AX124 analytical balance (Ohaus, New Jersey, USA) was utilized for mass measurements. Samples were weighed on 3×3-inch low nitrogen weighing Fisher brand paper (Fisher brand, Pittsburgh, USA). In general, a mass of at least 5.0 mg was weighed for any sample to minimize error. Both the balance and pH meter were calibrated immediately prior to use. Balance calibration was confirmed with a 5.0 mg standard weight (Troemner, Thorofare, NJ) with 5.0±0.1 mg cutoff.

### Melting Point

Melting points were obtained on DigiMelt apparatus of Stanford Research Systems (Sunnyvale, CA, USA). Solids were added to a capillary tube and placed inside the apparatus. The apparatus was heated at a ramp rate of 2° C. per minute and samples were visually observed for melting. The melting point range was determined by recording the temperature at first indication of melting and the temperature when the sample completely melted.

### Nuclear Magnetic Resonance Spectroscopy

1H and 13C NMR spectra data were obtained on a Bruker Avance III with PA BBO 400S1 BBF—H-D-05 Z plus probe (Bruker Corporation, Billerica, MA, USA). Samples were prepared at a concentration of ˜20 mg/mL in DMSO-d6 (Sigma-Aldrich, St. Louis, MO), CDCl3 (Sigma-Aldrich, St. Louis, MO), or D20 (MagniSolv™, Sigma-Aldrich, St. Louis, MO). Chemical shifts were reported in parts per million (ppm) and spectra were standardized to either residual solvent signals or tetramethylsilane (TMS).

### Moisture Readings

Moisture readings were obtained on a Mettler Toledo C20 Coulometric Karl Fischer titrator (Mettler Toledo, Columbus, OH, USA) charged with HYDRANAL™-Coulomat AG reagent from Honeywell Fluka™. A sample of HYDRANAL™ (10 mg water/g solution) from Honeywell Fluka™, Captisol® (Cydex Pharmaceuticals, Lawrence, KS), and sodium tartrate (Aqualine™ Primary Standard, Fisher Scientific, Fair Lawn, NJ) were run each time as positive controls. Measurements were made on masses of more than 20 mg.

### Osmolality

Osmolality readings were obtained on an Advanced Instruments Advanced™ Micro Osmometer Model 3300 (Advanced Instruments, Inc. Norwood, MA, USA). Saline solutions (0.9% and 3%) were run as positive controls. Each sample was calculated as the mean from back-to-back triplicate measurements. When replicate batches were measured, data was reported as mean±SEM.

### High Performance Liquid Chromatography

HPLC analyses were performed on an Agilent 1260 Infinity system that included a 1260 quaternary pump VL, a 1260 ALS autosampler, a 1260 Thermostatted Column Compartment, and a 1200 DAD Multiple Wavelength Detector (Agilent Technologies, Santa Clara, CA, USA). The detection wavelength was set at 220 nm. Separation for compounds were achieved using a Zorbax Eclipse Plus-C18 analytical column (5 μm, 4.6×150 mm) from Agilent (Agilent Technologies, Santa Clara, CA, USA). Mobile phase A consisted of 10 mM aqueous ammonium formate buffer titrated to pH 4.5 and mobile phase B consisted of acetonitrile. The injection volume of samples was 10 μL, flow rate was 1.0 mL/min, and the column temperature was set at 25° C. All samples were injected in duplicate with a wash of the injector (50:50 A:B) between runs. Run time was 10 minutes with a mobile phase ratio (isocratic) of 50% A and 50% B. Samples were injected in duplicate with an injector wash (50:50 A:B) between runs. Chromatograms were analyzed using the Agilent Chem Station Software (Agilent Technologies, Santa Clara, CA, USA).

### High Resolution Mass Spectroscopy (HRMS)

HRMS data were obtained on a Thermo Orbitrap Exactive Mass Spectrometer with an Orbitrap mass analyzer. The instrument was calibrated using electrospray ionization with Pierce™ LTQ ESI Positive Ion Calibration Solution from ThermoFisher Scientific. Samples were introduced into the instrument and ionized via an Atmospheric Solids Analysis Probe (ASAP). Data was analyzed in the Thermo Xcalibur Qual Browser software and identity was confirmed if there was less than 5 ppm error.

### Characterization of Active Pharmaceutical Ingredients (APIs)

Table 1 lists the melting point ranges of various APIs.

TABLE 1
Melting Point Ranges of APIs

| | Literature Melting | Experimental Melting |
|---|---|---|
| APIs | Point Range (° C.) | Point Range (° C.) |
| Tigecycline | 163-173° C.^{[1]} | 182.1-183.9° C. |
| Naloxone HCl | >181° C. with | 257.5° C. with |
| Dihydrate | decomposition^{[2]} | decomposition |
| Caspofungin | 204-206° C.^{[3]} | 186.5° C. with |
| Acetate | | decomposition |
| Rotigotine HCl | 177-179° C.^{[4]} | 184.8-185.3° C. |
| | 186.5-187.5° C.^{[5]} | |
| Eletriptan HBr | 169-171° C.^{[6]} | 168.5-170.5° C. |

^{[1]}AK Scientific, Inc. https://aksci.com/
^{[2]}Toronto Research Chemicals https://www.trc-canada.com/
^{[3]}Santa Cruz Biotechnology. https://www.scbt.com/home
^{[4]}Bio-Connect Life Sciences. https://shop.bio-connect.nl/bioconnect.home.aspx
^{[5]}A Smart Chem-Search Engine. https://www.chemsrc.com/en/
^{[6]}Chemical Book. https://www.chemicalbook.com/ProductIndex_EN.aspx

Table 2 lists the pH and osmolality values of various API formulations. Formulations were prepared on a 2 mL scale in most cases (1 mL or 5 mL scale for some). pH was titrated using 1M or 2M NaOH, or 1M or 2M HCl in some cases.

TABLE 2
pH and Osmolality of API Formulations.

| | | | Captisol ®-Na⁺-API Freebase | | | |
|---|---|---|---|---|---|---|
| | | | Formulation | | | |
| | | | (70 mg/mL API Salt in FB | | | |
| | | | equivalent, 1.1 molar | | API Salt | |
| | CapAcid-API Freebase | | equivalent Captisol ®-Na⁺) | | (70 mg/mL API Salt in FB | |
| | Formulation | | or | | equivalent) | |
| | (70 mg/mL API FB, | | *(70 mg/mL API FB + 6M | | or | |
| | 1.1 molar equivalent | | HCl, 1.1 molar equivalent | | *(70 mg/mL API FB + molar | |
| | CapAcid) | | Captisol ®-Na⁺) | | equivalent of 6M HCl) | |
| | Osmolality | | Osmolality | | Osmolality | |
| APIs | (mOsm/kg) | pH | (mOsm/kg) | pH | (mOsm/kg) | pH |
| Tigecycline | 1: 99.3 | 1: 6.97 | 330.3* | 7.00* | 138.3* | 5.50* |
| | 2: 92.7 | 2: 6.99 | | | | |
| Naloxone | 139.7 | 6.01 | 531.7 | 5.98 | 279 | 4.39 |
| Amikacin | 1: 268 | 1: 6.99 | 504.3* | 6.96* | 287* | 1.33* |
| | 2: 252.3 | 2: 6.95 | | | | |
| 1-Amantadine | 411.6 | 6.97 | 1,446.7 | 6.99 | 832 | 5.67 |
| 2-Amantadine | 435.7 | 7.06 | 1492.7 | 7.01 | 826.7 | 6.94 |
| Rimantadine | 301.7 | 6.98 | 297.3*** | 7.14*** | 195.3*** | 5.28*** |
| Amifampridine | 532.7** | 6.98** | 1,023.3* | 7.01* | 740.3*\hat{ } | 8.77*\hat{ } |
| Caspofungin | 59** | 7.06** | 209.3 | 7.04 | 94 | 5.86 |
| Melevodopa | 605.7 | 6.97 | N/A | N/A | N/A | N/A |

*70 mg/mL API FB + 1M equivalent of 6M HCl.
\hat{ }an extra 45 μL of 6M HCl was required to dissolve.
**Titrated with aqueous HCl (1M equivalents for caspofungin, 2M equivalents for amifampridine) for the CapAcid and Captisol ® formulations, as the initial pH, prior to titration, was >7.00.
***20 mg/mL FB equivalents, as this was found to be the highest concentration that would reliably go into solution.

### General Procedure for the Preparation of API FB-CapAcid Formulations

The procedure was performed on a 2 mL scale. In a 5 mL borosilicate glass test tube which contained a Teflon-coated magnetic stir bar, Cap Acid (1.1 molar equivalents, H⁺ equivalents, moisture corrected, “active” refers to the mass without correction for moisture) was dissolved in HPLC grade water (1.6-1.7 mL), API FB (140 mg, 70 mg/mL) was added and the mixture was stirred until a homogenous transparent solution formed. Benzethonium chloride (20 μL of a 1% solution, 0.01% final concentration) was added via micropipette. The pH was adjusted to ˜7.00 with 2M sodium hydroxide, or either 1M or 2M HCl in some cases, and then the mixture was transferred to a 2 mL volumetric flask to be diluted with HPLC grade water. The final pH after dilution was recorded, and the solution was filtered through either a 0.45 micron or 0.20 micron syringe filter, and osmolality was immediately measured (in triplicate) along with 0.9% saline and 3% saline standards.

### General Procedure for the Preparation of API Salt Captisol®-Na⁺ Formulations.

The procedure was performed on a 2 mL scale

Method 1: In a 5 mL borosilicate glass test tube which contained a Teflon-coated magnetic stir bar, Captisol®-Na+ (1.1 molar equivalents, H+ equivalents, moisture corrected) was dissolved in HPLC grade water (1.6-1.7 mL), API salt (HCl in most cases) (70 mg/mL FB equivalents) was added and the mixture was stirred until a homogenous transparent solution formed. Benzethonium chloride (20 μL of a 1% solution, giving a 0.01% final concentration) was added via micropipette. The pH was adjusted to ˜7.00 with 1M or 2M sodium hydroxide, or either 1M or 2M HCl in some cases, and then the mixture was transferred to a 2 mL volumetric flask to be diluted with HPLC grade water. The final pH after dilution was recorded, the solution was filtered through a 0.20 micron syringe filter, and osmolality was immediately measured (in triplicate) along with 0.9% saline and 3% saline standards.

Method 2: In a 5 mL borosilicate glass test tube which contained a Teflon-coated magnetic stir bar, Captisol®-Na+ (1.1 molar equivalents, H+ equivalents, moisture corrected) was dissolved in HPLC grade water (1.6-1.7 mL), API FB (140 mg, 70 mg/mL) was added, as well as 1 molar equivalent of 6M HCl, and the mixture was stirred until a homogenous transparent solution formed. Benzethonium chloride (20 μL of a 1% solution, giving a 0.01% final concentration) was added via micropipette. The pH was adjusted to ˜7.00 with 1M or 2M sodium hydroxide, or either 1M or 2M HCl in some cases, and then the mixture transferred to a 2 mL volumetric flask to be diluted with HPLC grade water. The final pH after dilution was recorded, the solution was filtered through a 0.20 micron syringe filter, and osmolality immediately measured (in triplicate) along with 0.9% saline and 3% saline standards.

### General Procedure for the Preparation of API FB-HCl Formulations

The procedure was performed on a 2 mL scale,

Method 1: In a 5 mL borosilicate glass test tube with a Teflon-coated magnetic stir bar, API Salt (HCl in most cases) (70 mg/mL FB equivalents) was diluted to 2 mL with HPLC grade water in a 2 mL volumetric flask. The pH of the solution was immediately measured. The solution was then filtered through a 0.20 micron syringe filter, and osmolality was immediately measured (in triplicate) along with 0.9% saline and 3% saline standards.

Method 2: In a 5 mL borosilicate glass test tube with a Teflon-coated magnetic stir bar, 1 molar equivalent relative to the API (FB equivalents) of HCl (6M) was added to HPLC grade water (1.6-1.7 mL), API FB (140 mg, 70 mg/mL) was added and the mixture was stirred until a homogenous transparent solution formed. The solution was transferred to a 2 mL volumetric flask and diluted to the target volume with HPLC grade water. The pH of the solution was immediately measured. The solution was then filtered through a 0.20 micron syringe filter, and osmolality immediately measured (in triplicate) along with 0.9% saline and 3% saline standards.

Specific procedures different from the general procedure described above for certain cases were disclosed below.

### Tigecycline

The formulation of Tigecycline FB CapAcid was prepared as follows. The preparation procedure for Tigecycline FB CapAcid Formulation was modified from the general procedure. A solution of 70 mg/mL Tigecycline FB (69.9 mg, 0.12 mmol) and 1.1 equivalents of CapAcid, H⁺ equivalents, (43.7 mg moisture corrected, 40.8 mg active, 0.02 mmol, 0.13 mmol H⁺) was prepared by dissolving CapAcid in 700 μL of HPLC grade water, then adding the freebase and stirring until a homogenous solution formed. Benzethonium chloride (10 μL, 1 mg/mL, 0.01% final concentration) was added to the solution. NaOH (2M) was added until the pH was 7.00±0.01. The solution was diluted to 1 mL and the final pH was determined to be 6.99. The solution was bright orange, as is the Tigecycline FB powder. The osmolality was recorded the next day, and the solution had turned yellow-brown. The solution was filtered through a 0.45 micron syringe filter. The osmolality of this solution was 158.7 mOsmol/kg.

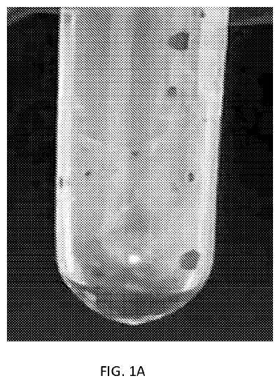

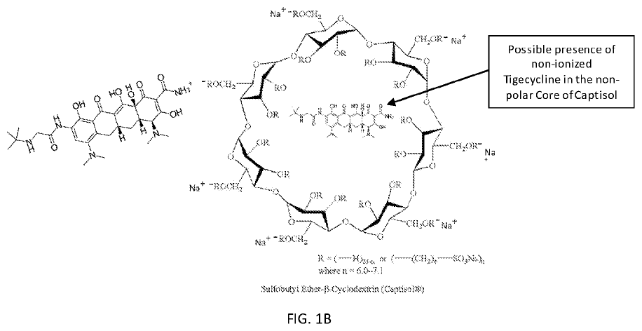

The formulation of a Tigecycline FB (70 mg/mL) with 1.1 molar equivalents of CapAcid (H⁺ equivalents) was shown in FIG. 1A and the formulation showed degradation after 24 hours.

The solution was prepared a second time on a 5 mL scale and the osmolality measured immediately. A solution of 70 mg/mL tigecycline FB (350.1 mg, 0.60 mmol) and 1.1 equivalents of CapAcid, H⁺ equivalents, (213.9 mg moisture corrected, 204.4 mg active, 0.10 mmol, 0.66 mmol H+) was prepared by dissolving CapAcid in 4.5 mL of HPLC grade water, then adding the freebase and stirring until a homogenous solution formed. Benzethonium chloride (50 μL, 1 mg/mL, 0.01% final concentration) was added to the solution. The pH of this solution was 5.19. NaOH (2M) was added until the pH was 7.00±0.01. The solution was diluted to 5 mL and the final pH was determined to be 6.97. The solution was filtered through a 0.45 micron syringe filter. The osmolality was 99.3 mOsmol/kg.

This solution was prepared a second time (N=2). A solution of 70 mg/mL tigecycline FB (350.4 mg, 0.60 mmol) and 1.1 equivalents of Cap Acid, H⁺ equivalents, (222.3 mg moisture corrected, 204.5 mg active, 0.10 mmol, 0.66 mmol H⁺) was prepared by dissolving Cap Acid in 4.5 mL of HPLC grade H₂O, then adding the freebase and stirring until a homogenous solution formed. Benzethonium chloride (50 μL, 1 mg/mL, 0.01% final concentration) was added to the solution. The pH of this solution was 5.37. NaOH (2M) was added until the pH was 7.00±0.01. The solution was diluted to 5 mL and the final pH was determined to be 6.99. The solution was filtered through a 0.45 micron syringe filter. The osmolality was 92.7 mOsmol/kg.

A formulation of Tigecycline HCl Captisol®-Na+ was prepared following the general procedure. A solution of 70 mg/mL tigecycline (140.1 mg, 0.24 mmol), 1 equivalent of 6M HCl (39.8 μL, 0.24 mmol), and 1.1 equivalents of Captisol®-Na⁺, Na⁺ equivalents, (91.3 mg moisture corrected, 87.5 mg active, 0.04 mmol, 0.26 mmol H⁺) was prepared by dissolving the Captisol®-Na⁺ in 1.7 mL of HPLC water, dissolving the freebase in this solution, and then adding the 6M HCl as well as 20 μL of a benzethonium chloride solution (1 mg/mL, 0.01% final concentration). The pH of this solution was 5.61. A solution of NaOH (1M) was added until the pH was 7.00±0.01. The solution was diluted to 2 mL and the final pH was determined to be 7.00. The solution was filtered through a 0.20 micron syringe filter. The osmolality was 330.3 mOsmol/kg.

A formulation of Tigecycline HCl was prepared following the general procedure. A solution of 70 mg/mL tigecycline (140.1 mg, 0.24 mmol) and 1 equivalent of 6M HCl (39.8 μL, 0.24 mmol) was prepared by adding the 6M HCl to 1.7 mL of HPLC water, adding the freebase and allowing it to dissolve, then diluting the solution to 2 mL in a volumetric flask. The pH was 5.50, the solution was then filtered through a 0.20 micron syringe filter, and the osmolality was 138.3 mOsmol/kg.

The structure of the resulting Tigecycline-Captisol® salt is shown in FIG. 1B.

### Naloxone

Naloxone freebase was prepared as follows. Ammonium chloride (13.4 g) was dissolved in deionized water (100 mL) and ammonium hydroxide (17 mL) was slowly added to this solution. Naloxone HCl Dihydrate (2.5 g, 6.87 mmol) was dissolved in deionized water (400 mL). The ammonium solution was added slowly to the naloxone solution until it was at a pH of approximately 8 by litmus paper. Crystals immediately formed once the ammonium solution was added. The solution was put on ice for 20 minutes to allow any suspended crystals to precipitate and fall to the bottom. The crystals were recovered by vacuum filtration, washed with HPLC grade water and left to dry overnight to give a white solid (2.01 g, 89.3% yield). The melting point range was determined to be 179.4-180.8° C. A reference melting point range is 177-184° C. (Santa Cruz Biotechnology. https://www.scbt.com/home). The same procedure was used with 5.00 g (13.74 mmol) naloxone HCl dihydrate in 400 ml distilled (DI) water. The recovered product was a white solid (4.16 g, 92.5% yield).

The formulation of Naloxone FB Cap Acid was prepared as follows. A solution of 70 mg/mL naloxone FB (139.9 mg, 0.43 mmol) and 1.1 equivalents of CapAcid, H⁺ equivalents, (153.2 mg moisture corrected, 146.2 mg active, 0.07 mmol, 0.47 mmol H⁺) was prepared by dissolving CapAcid in 1.6 mL of HPLC grade water, then adding the freebase and stirring until a homogenous solution formed. Benzethonium chloride solution (20 μL, 1 mg/mL, 0.01% final concentration) was added to the solution. The pH of the solution was measured at 1.58. A solution of NaOH (2M) was added slowly in portions until the pH was 6.00±0.01 (another analysis showed the freebase crashed out shortly past pH 6.00). The solution was then diluted to 2 mL in a volumetric flask and the final pH was determined to be 6.01. Next, the solution was filtered through a 0.45 micron syringe filter and the osmolality was measured. The osmolality was 139.7 mOsm/kg.

The formulation of Naloxone HCl Captisol®-Na+ was prepared as follows. A solution of 70 mg/mL Naloxone HCl (FB equivalent) (155.6 mg, 0.43 mmol) and 1.1 equivalents of Captisol®-Na⁺, Na⁺ equivalents, (163.6 mg moisture corrected, 156.6 mg active, 0.08 mmol, 0.49 mmol H⁺) was prepared by dissolving Captisol®-Na⁺ in 1.7 mL of HPLC-grade H₂O, then adding the salt in small portions and stirring via a teflon coated magnetic stir bar, until a homogenous solution formed. A solution of benzethonium chloride (20 μL, 1 mg/mL, 0.01% final concentration) was added to the solution. The pH of this solution was 3.70. A solution of NaOH (2M) was added until the pH was 6.00±0.01. The solution was diluted to 2 mL and the final pH was determined to be 5.98. The solution was filtered through a 0.20 micron syringe filter. The osmolality was 531.7 mOsmol/kg.

The formulation of Naloxone HCl was prepared as follows. A solution of 70 mg/mL (FB equivalents) of naloxone HCl (155.5 mg, 0.43 mmol) was prepared by adding dissolving it in a 2 mL volumetric flask with HPLC grade water. The pH of the solution was 4.39. The solution was filtered through a 0.20 micron syringe filter. The osmolality was 279 mOsmol/kg.

The formulation of Naloxone Cap Acid Stoichiometric Salt was prepared as follows. Naloxone FB (1.0 g, 3.06 mmol) and 1 equivalent of CapAcid, H⁺ equivalents, (1.03 g moisture corrected, 0.95 g active, 0.47 mmol, 3.06 mmol H⁺) were dissolved in HPLC-grade water. A solution of NaOH (2M) was added in small portions until the pH was 5.50. The solution was filtered through a 0.45 micron filter and lyophilized. The recovered product was a white solid (1.87 g, 92.0% yield) that was ground into a powder with a glass stir rod.

The formulation of Naloxone Cap Acid Stoichiometric Salt in 0.9% Saline was prepared as follows. Solutions of 25, 50, and 75 mg/mL Naloxone-Cap stoichiometric salt (FB equivalents) were prepared in clean, 5 mL borosilicate glass test tubes. The tubes were cleaned with water and soap, rinsed with ethanol, and dried with heat. The test tubes were allowed to cool and covered with parafilm until ready for use. Each solution was made up in 700 μL of 0.9% saline solution with benzethonium chloride solution (10 μL, 1 mg/mL, 0.01% final concentration) added. The initial pH, prior to titration, was recorded (in case any NaOH needed to be added to get the pH to be greater than 5.0) and then each solution was diluted to a final volume of 1 mL in a volumetric flask and the resulting final pH measured. The solutions were then filtered through a 0.20 micron syringe filter into the prepared clean test tubes and were immediately sealed with parafilm and wrapped in foil to protect from light. An aliquot of each sample was saved to measure osmolality.

TABLE 3
pH and Osmolality of Naloxone CapAcid Stoichiometric
Salt in 0.9% Saline at Different Concentrations

| Concentration (mg/mL), | | |
|---|---|---|
| FB Equivalents | Final pH | Osmolality (mOsmol/kg) |
| 25 | 5.41 | 338.3 |
| 50 | 5.23 | 370.0 |
| 75 | 5.13 | 403.7 |

*Performed on a 1 mL scale

The formulation of Naloxone CapAcid Stoichiometric Salt in water was prepared as follows. Solutions of 25, 50, and 75 mg/mL Naloxone-Cap stoichiometric salt (FB equivalents) were prepared in clean, 5 mL borosilicate glass test tubes. The tubes were cleaned with water and soap, rinsed with ethanol, and dried with heat. The test tubes were allowed to cool and covered with parafilm until ready for use. The naloxone stoichiometric salt was 49.2% Naloxone, which was used to adjust the mass of stoichiometric salt needed in order to achieve the correct concentration. Each solution was made up of 1.7 mL of HPLC water with benzethonium chloride (20 μL, 1 mg/mL, 0.01% final concentration) added. The initial pH, prior to titration, was recorded (in case any NaOH needed to be added to get the pH to be greater than 5) and then each solution was diluted to 2 mL and the final pH was recorded. The solutions were filtered through a 0.20 micron syringe filter and then injected into sterile injection vials, and a small aliquot was saved for measuring the osmolality. The vials were stored at room temperature in the dark.

TABLE 4
pH and Osmolality of Naloxone CapAcid Stoichiometric
Salt in HPLC Grade Water at Different Concentrations

| Concentration (mg/mL), | | |
|---|---|---|
| FB Equivalents | Final pH | Osmolality (mOsmol/kg) |
| 25 | 5.54 | 54.7 |
| 50 | 5.34 | 89.0 |
| 75 | 5.21 | 123 |

*Performed on a 2 mL scale

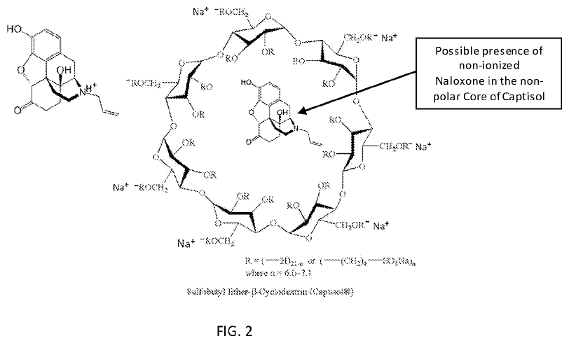

The structure of the resulting Naloxone-Captisol® salt is shown in FIG. 2.

### Amikacin

The formulation of Amikacin FB CapAcid was prepared according to two procedures below.

The first procedure is as follows. A solution of 70 mg/mL amikacin FB (140.0 mg, 0.24 mmol) and 1.1 equivalents of CapAcid, H⁺ equivalents, (88.9 mg moisture corrected, 81.7 mg active, 0.04 mmol, 0.26 mmol H⁺, H⁺ equivalents) was prepared by dissolving CapAcid in 1.7 mL of HPLC grade water, then adding the freebase and stirring until a homogenous solution formed. Benzethonium chloride (20 μL, 1 mg/mL, 0.01% final concentration) was added to the solution. The pH of this solution was 1.13. NaOH (2M) was added until the pH was 7.00±0.01. The solution was diluted to 2 mL in a volumetric flask and the final pH was determined to be 6.99. The solution was filtered through a 0.45 micron syringe filter. The osmolality was 268 mOsmol/kg.

The second procedure is as follows. A solution of 70 mg/mL amikacin FB (350.1 mg, 0.60 mmol) and 1.1 equivalents of CapAcid, H⁺ equivalents, (222.4 mg moisture corrected, 204. 3 mg active, 0.10 mmol, 0.66 mmol H⁺) was prepared by dissolving CapAcid in 4.5 mL of HPLC grade water, then adding the freebase and stirring until a homogenous solution formed. Benzethonium chloride (20 μL, 1 mg/mL, 0.01% final concentration) was added to the solution. The pH of this solution was 1.25. NaOH (2M) was added until the pH was 7.00±0.01. The solution was diluted to 5 mL and the final pH was determined to be 6.95. The solution was filtered through a 0.20 micron syringe filter. The osmolality was 252.2 mOsmol/kg.

The formulation of Amikacin HCl Captisol®-Na⁺ was prepared as follows. A solution of 70 mg/mL Amikacin (140.1 mg, 0.24 mmol), 1 equivalent of 6M HCl (39.8 μL, 0.24 mmol), and 1.1 equivalent Captisol®-Na⁺, Na⁺ equivalents, (91.4 mg moisture corrected, 87.5 mg active, 0.04 mmol, 0.26 mmol H⁺) was prepared by dissolving the Captisol®-Na⁺ in 1.7 mL of HPLC water, dissolving the freebase in this solution, and then adding 6M HCl as well as benzethonium chloride solution (20 μL, 1 mg/mL, 0.01% final concentration). The pH of this solution was 1.28. A solution of NaOH (2M) was added until the pH was 7.00±0.01. The solution was diluted to 2 mL and the final pH was determined to be 6.96. The solution was filtered through a 0.20 micron syringe filter. The osmolality was 504.3 mOsmol/kg.

The formulation of Amikacin HCl was prepared as follows. A solution of 70 mg/mL Amikacin (140.0 mg, 0.24 mmol) and 1 equivalent of 6M HCl (39.8 μL, 0.24 mmol) was prepared by adding the 6M HCl to 1.7 mL of HPLC water, adding the freebase and allowing it to dissolve, then diluting the solution to 2 mL in a volumetric flask. The pH was 1.33, the solution was then filtered through a 0.20 micron syringe filter, and the osmolality was 287 mOsmol/kg.

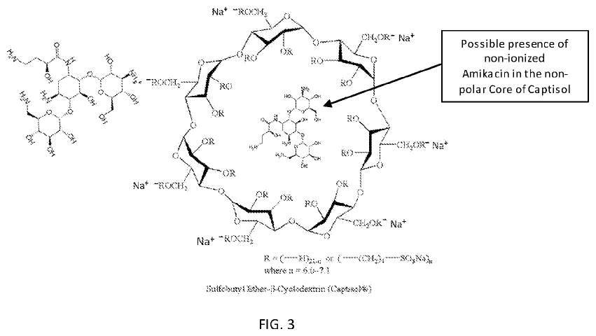

The structure of the resulting Amikacin-Captisol® salt is shown in FIG. 3.

### 1-Amantadine

1-Amantadine Freebase was prepared using the following methods.

### Method 1

1-Amantadine HCl (4.01 g, 21.3 mmol) was dissolved in approximately 100 mL of distilled (DI) water. A solution of 10% KOH was added (approx. 10-20 mL) until the pH was >10 by litmus paper. Upon the addition of the KOH solution, the solution turned milky white. It was extracted three times with 75 mL ethyl acetate, after which the solution became colorless and transparent. The organic extracts were washed with brine (50 mL), dried with a few scoops of anhydrous sodium sulfate, and solvent was removed under vacuum. The recovered white powder (293.3 mg, 7.12% yield) was collected via vacuum filtration, washed with HPLC water and allowed to dry in the hood overnight.

### Methods 2 and 3

1-Amantadine HCl (1.02 g, 5.4 mmol) was dissolved in ˜100 mL of DI water. A solution of 10% KOH was added (˜50 mL) to force precipitation. The solution became cloudy, but it did not form a precipitate that can be filtered. Solid KOH (approx. 10 g) was added and a precipitate formed. The solution was placed in an ice bath for 20 minutes. Approximately half of the solution and precipitate were filtered, and the solution slowly came out drop wise. The filtered precipitate was left in the hood to dry overnight, and the other half of the precipitate in solution was saved for the next day. After drying, the product could not be removed from the filter paper, and was not used. Instead, the other half of the solution was used for an extraction with ether. Diethyl ether (˜50 mL) was added to the solution and the precipitate dissolved. This solution was added to a separatory funnel. 1-Amantadine HCl (2.01 g, 10.7 mmol) was dissolved in ˜50 mL DI water and a solution of 10% KOH was added (˜20 mL) until the pH was >10 by litmus paper. This solution was added to the funnel with the previous solution. Another 50 mL of ether was added and the freebase extracted. The solution was extracted once more with ether (˜75 mL). The organic layer was washed with brine (˜50 mL), dried with a few scoops of sodium sulfate, and the solvent was removed under vacuum. The recovered product was a white powder (774.2 mg, 31.7% yield). The melting point range was 206.3-208.7° C. A reference melting point range is 206-208° C. (Loudon, G. Marc; Radhakrishna, A. S.; Almond, Merrick R.; Blodgett, James K.; Boutin, Raymond H. Conversion of aliphatic amides into amines with [I,I-bis(trifluoroacetoxy) iodo]benzene. 1. Scope of the reaction J. Org. Chem. 1984, 49 (22), 4272-4276.).

A formulation of 1-Amantadine FB Cap Acid was prepared as follows. A solution of 70 mg/mL 1-amantadine FB (139.9 mg, 0.92 mmol) and 1.1 equivalents of CapAcid, H⁺ equivalents, (346.6 mg moisture corrected, 3 16.4 mg active, 0.16 mmol, 1.02 mmol H⁺) was prepared by dissolving CapAcid in 1.7 mL of HPLC-grade H₂O, then adding the freebase in small portions and stirring via a teflon coated magnetic stir bar, until a homogenous solution formed. A solution of benzethonium chloride (20 μL, 1 mg/mL, 0.01% final concentration) was added to the solution. The pH of this solution was 1.43. A solution of NaOH (2M) was added until the pH was 7.00±0.01. The solution was diluted to 2 mL and the final pH was determined to be 6.97. The solution was filtered through a 0.20 micron syringe filter. The osmolality was 411.6 mOsmol/kg.

A formulation of 1-Amantadine HCl Captisol®-Na+ was prepared as follows. A solution of 70 mg/mL 1-amantadine HCl (FB equivalent) (173.7 mg, 0.93 mmol) and 1.1 equivalents of Captisol®-Na⁺, Na⁺ equivalents, (340.4 mg moisture corrected, 325.9 mg active, 0.15 mmol, 0.98 mmol H⁺) was prepared by dissolving Captisol®-Na⁺ in 1.7 mL of HPLC-grade H₂O, then adding the salt in small portions and stirring via a teflon coated magnetic stir bar, until a homogenous solution formed. A solution of benzethonium chloride (20 μL, 1 mg/mL, 0.01% final concentration) was added to the solution. The pH of this solution was 3.93. A solution of NaOH (2M) was added until the pH was 7.00±0.01. The solution was diluted to 2 mL and the final pH was determined to be 6.99. The solution was filtered through a 0.20 micron syringe filter. The osmolality was 1,446.7 mOsmol/kg.

A formulation of 1-Amantadine HCl was prepared as follows. A solution of 70 mg/mL 1-amantadine HCl, FB equivalents (173.7 mg, 0.93 mmol) was prepared by dissolving it in HPLC grade water in a 2 mL volumetric flask. The pH was 5.67 and the solution was filtered through a 0.20 micron syringe filter. The osmolality was 832 mOsm/kg.

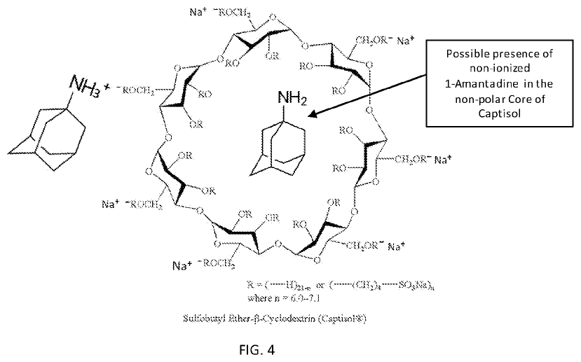

The structure of the resulting 1-Amantadine-Captisol® salt is shown in FIG. 4.

### 2-Amantadine

The preparation of 2-Amantadine Freebase was described as follows. 2-Amantadine HCl (4.00 g, 21.3 mmol) was dissolved in approximately 100 mL of DI water. A solution of 10% KOH was added (approx. 10-20 mL) until the pH was >10 by litmus paper. Upon the addition of the KOH solution, the solution turned milky white. It was extracted three times with 75 mL ethyl acetate, after which the solution became colorless and transparent. The organic extracts were washed with brine (50 mL), dried with a few scoops of anhydrous sodium sulfate, and solvent was removed under vacuum. The recovered white powder was collected via vacuum filtration, washed with HPLC-grade H2O and allowed to dry in the hood overnight (3.22 g, 72.4% yield). The melting point range was determined to be 212.3-216.7° C. A reference melting point range is 221-225° C. (Hussein, A. Q; Herzberger, S.; Jochims, J. C. Chem. Ber. 1979, 112, 1102-1109).

A formulation of 2-Amantadine FB CapAcid was prepared as follows. A solution of 70 mg/mL 2-amantadine FB (139.9 mg, 0.92 mmol) and 1.1 equivalents of CapAcid, H⁺ equivalents, (344.3 mg moisture corrected, 3 16.4 mg active, 0.16 mmol, 1.02 mmol H⁺) was prepared by dissolving CapAcid in 1.6 mL of HPLC-grade H₂O, then adding the freebase in small portions and stirring via a teflon coated magnetic stir bar, until a homogenous solution formed. A solution of benzethonium chloride (20 μL, 1 mg/mL, 0.01% final concentration) was added to the solution. The pH of this solution was 1.29. A solution of NaOH (2M) was added until the pH was 7.00±0.01. The solution was diluted to 2 mL and the final pH was determined to be 7.06. The solution was filtered through a 0.20 micron syringe filter. The osmolality was 435.7 mOsmol/kg.

A formulation of 2-Amantadine HCl Captisol®-Na+ was prepared as follows. A solution of 70 mg/mL 2-amantadine HCl (FB equivalent) (173.7 mg, 0.93 mmol) and 1.1 equivalents of Captisol®-Na⁺, Na⁺ equivalents, (340.5 mg moisture corrected, 325.9 mg active, 0.15 mmol, 0.98 mmol H⁺) was prepared by dissolving Captisol®-Na⁺ in 1.7 mL of HPLC-grade H₂O, then adding the salt in small portions and stirring via a teflon coated magnetic stir bar, until a homogenous solution formed. A solution of benzethonium chloride (20 μL, 1 mg/mL, 0.01% final concentration) was added to the solution. The initial pH was 5.63. A solution of NaOH (2M) was added until the pH was 7.00±0.01. The solution was diluted to 2 mL and the final pH was determined to be 7.01. The solution was filtered through a 0.20 micron syringe filter. The osmolality was 1,492.7 mOsmol/kg.

A formulation of 2-Amantadine HCl was prepared as follows. A solution of 70 mg/mL 2-amantadine HCl, FB equivalents (173.7 mg, 0.93 mmol) was prepared by dissolving it in HPLC grade water in a 2 mL volumetric flask. The pH was 6.94 and the solution was filtered through a 0.20 micron syringe filter. The osmolality was 826.7 mOsm/kg.

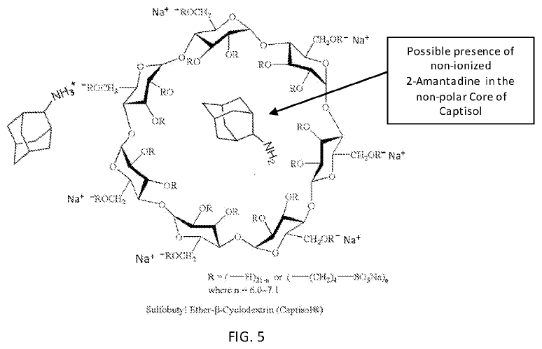

The structure of the resulting 2-Amantadine-Captisol® salt is shown in FIG. 5.

### Rimantadine

The preparation of Rimantadine Freebase was described as follows. Rimantadine HCl (701.2 mg, 3.24 mmol) was dissolved in approximately 175 mL of DI water. A solution of 10% KOH was added (approximately 10-20 mL) until the pH was >10 by litmus paper. Upon the addition of the KOH solution, the solution turned milky white. It was extracted three times with 75 mL ethyl acetate. The organic extracts were washed with brine (50 mL), dried with a few scoops of anhydrous sodium sulfate, and solvent removed under vacuum. The recovered product was an oil with a slight yellow tint (0.44 g, 75.6% yield).

A formulation of Rimantadine FB CapAcid was prepared as follows. A solution of 70 mg/mL rimantadine FB (70.1 mg, 0.39 mmol) and 1.1 equivalents of CapAcid, H⁺ equivalents, (146.2 mg moisture corrected, 133.5 mg active, 0.07 mmol, 0.43 mmol H⁺) was prepared by dissolving CapAcid in in 700 μL of HPLC-grade H2O, then adding the freebase in small portions and stirring via a teflon coated magnetic stir bar, until a homogenous solution formed. A solution of benzethonium chloride (10 μL, 1 mg/mL, 0.01% final concentration) was added to the solution. The pH of this solution was 1.93. A solution of NaOH (2M) was added until the pH was 6.85. The solution was diluted to 1 mL and the final pH was determined to be 6.98. The solution was filtered through a 0.20 micron syringe filter. The osmolality was 301.7 mOsmol/kg

A formulation of Rimantadine HCl Captisol®-Na⁺ was prepared as follows. A solution of 20 mg/mL rimantadine HCl (FB equivalent) (24.0 mg, 0.11 mmol) and 1.1 equivalents of Captisol®-Na⁺, Na⁺ equivalents, (42.6 mg moisture corrected, 40.8 mg active, 0.02 mmol, 0.12 mmol H⁺) was prepared by dissolving Captisol®-Na⁺ in in 1.7 mL of HPLC-grade H₂O, then adding the salt in small portions and stirring via a teflon coated magnetic stir bar, until a homogenous solution formed. A solution of benzethonium chloride (20 μL, 1 mg/mL, 0.01% final concentration) was added to the solution. The pH of this solution was 4.65. A solution of NaOH (2M) was added until the pH was 7.00±0.01. The solution was diluted to 1 mL and the final pH was determined to be 7.14. The solution was filtered through a 0.20 micron syringe filter. The osmolality was 297.3 mOsmol/kg.

A formulation of Rimantadine HCl was prepared as follows. A solution of 20 mg/mL rimantadine HCl, FB equivalents (48.3 mg, 0.22 mmol) was prepared by dissolving it in HPLC grade water in a 2 mL volumetric flask. The pH was 5.28 and the solution was filtered through a 0.20 micron syringe filter. The osmolality was 195.3 mOsm/kg.

### Concentration Test

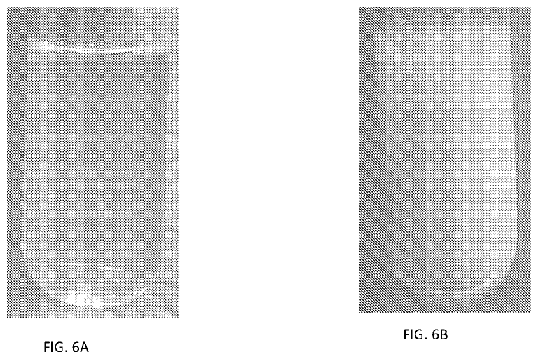

A solution of 50 mg/mL rimantadine HCl, FB equivalents, (120.3 mg, 0.56 mmol), was prepared by adding 1.7 mL of HPLC grade water and an addition of a solution of benzethonium chloride (20 μL, 1 mg/mL, 0.01% final concentration). After stirring for a few hours, the Rimantadine HCl did not dissolve. It was diluted until it did dissolve, which took a total of 4.6 mL of HPLC (including the initial 1.7 mL). The solution was left to stir overnight. After stirring overnight, it was completely dissolved, as shown in FIG. 6A. The solution was diluted to 5 mL, making the final concentration 20 mg/mL. The pH was 6.83 and the solution was filtered through a 0.20 micron syringe filter. The osmolality was 205 mOsm/kg.

A separate test was performed on another 50 mg/mL solution of rimantadine HCl, FB equivalents (60.2 mg, 0.28 mmol), dissolved in 800 μL of HPLC grade water and an addition of a solution of benzethonium chloride (10 μL, 1 mg/mL, 0.01% final concentration). This solution was left to stir overnight, however it did not dissolve, as shown in FIG. 6B.

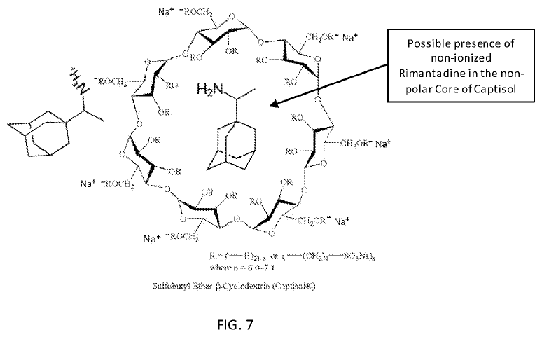

The structure of the resulting Rimantadine-Captisol® salt is shown in FIG. 7.

### Amifampridine

A formulation of Amifampridine CapAcid was prepared as follows. A solution of 70 mg/mL amifampridine FB (140.1 mg, 0.68 mmol) and 1.1 equivalents of CapAcid, H⁺ equivalents, (251.3 mg moisture corrected, 231.1 mg active, 0.11 mmol, 0.74 mmol H⁺) was prepared by dissolving CapAcid in 1.5 mL of HPLC grade water, then adding the freebase and stirring until a homogenous solution formed. Benzethonium chloride (20 μL, 1 mg/mL solution, 0.01% final concentration) was added to the solution. The pH of the solution was 9.19. HCl (2M) was added in small portions, until the pH was 7.00±0.01. The solution was diluted to 2 mL and the final pH was determined to be 6.98. The solution was filtered through a 0.20 micron syringe filter. The osmolality was 532.7 mOsmol/kg.

A formulation of Amifampridine HCl Captisol®-Na⁺ was prepared as follows. A solution of 70 mg/mL amifampridine (140.1 mg, 0.68 mmol), 1 equivalent of 6M HCl (112.8 μL, 0.68 mmol), and 1.1 equivalent Captisol®-Na⁺, Na⁺ equivalents, (258.4 mg moisture corrected, 247.4 mg active, 0.11 mmol, 0.74 mmol H+) was prepared by dissolving the Captisol®-Na⁺ in 1.7 mL of HPLC grade water, dissolving the freebase in this solution, and then adding the 6M HCl as well as benzethonium chloride (20 μL, 1 mg/mL solution, 0.01% final concentration). The pH of this solution was 9.27. A solution of HCl (2M) was added until the pH was 7.00±0.01. The solution was diluted to 2 mL and the final pH was determined to be 7.01. The solution was filtered through a 0.20 micron syringe filter. The osmolality was 1023.3 mOsmol/kg.

A formulation of Amifampridine HCl was prepared as follows. A solution of 70 mg/mL amifampridine (139.9 mg, 0.68 mmol) and 1 equivalent of 6M HCl (112.8 μL, 0.68 mmol) was prepared by adding the 6M HCl to 1.7 mL of HPLC grade water, adding the freebase and allowing it to dissolve, then diluting the solution to 2 mL in a volumetric flask. The pH was 8.77, the solution was then filtered through a 0.20 micron syringe filter, and the osmolality was 740.3 mOsmol/kg.

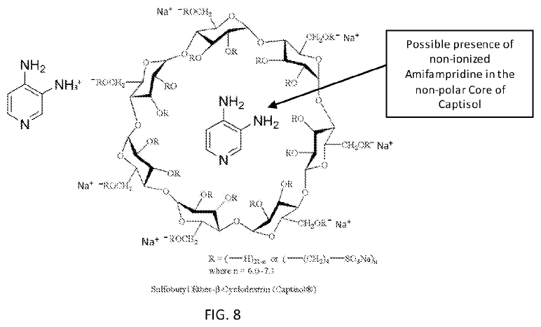

The structure of the resulting Amifampridine-Captisol® salt is shown in FIG. 8.

### Caspofungin

The isolation of Caspofungin Free Base was described as follows. Procedure 1: Caspofungin acetate (2.00 g, 1.65 mmol) was dissolved in approximately 50 mL of DI water. Ammonium hydroxide (5 drops, approximately 125 μL, approximatedly 2.2 mmol) was added to the solution until the pH was >10 by litmus paper. The solution was placed in an ice bath for 15 minutes to allow crystals to form. The crystals were filtered by vacuum and washed with a small amount of HPLC grade water, collected in two crops, and lyophilized (300 mg, 16.6% yield).

The first batch was scratched off filter paper and lyophilized. The result was a fluffy white solid, approximately 270 mg recovery. For the second batch, the filter paper was dissolved in around 10 mL HPLC grade water, filtered through a 0.45 micron filter, and lyophilized. The result was a coarse white solid, approximate 30 mg recovery.

Procedure 2: Caspofungin acetate (3.50 g, 2.88 mmol) was dissolved in 11 mL of HPLC grade water in a beaker. The beaker was placed in an ice bath and ammonium hydroxide (6 drops, approximately 150 μL, approximately 2.65 mmol) was added to the solution until it was basic by litmus paper. A large solid mass of product formed and the solution remained in the ice bath for 15 minutes. The solution was decanted off. The solid was rinsed twice with HPLC grade water (1 mL) and stirred to get rid of excess ammonium hydroxide. The solid was lyophilized for 3 days and a white powder (0.72 g, 22.9% yield) was recovered. The decomposition melting point was determined to be 181.5° C.

A formulation of Caspofungin FB CapAcid was prepared as follows. A solution of 70 mg/mL caspofungin FB (140.0 mg, 0.13 mmol) and 1.1 equivalents of Cap Acid, H⁺ equivalents, (47.6 mg moisture corrected, 43.8 mg active, 0.02 mmol, 0.14 mmol H⁺) was prepared by dissolving CapAcid in 1.6 mL of HPLC grade water, then adding the freebase and stirring until a homogenous solution formed. Benzethonium chloride (20 μL, 1 mg/mL solution, 0.01% final concentration) was added to the solution. The pH of this solution was 7.78. HCl (1M) was added until the pH was 7.00±0.01. The solution was diluted to 2 mL and the final pH was determined to be 7.06. The solution was filtered through a 0.20 micron syringe filter. The osmolality was 59 mOsmol/kg.

A formulation of Caspofungin Acetate Captisol®-Na⁺ was prepared as follows. A solution of 70 mg/mL caspofungin Acetate (FB equivalent) (155.5 mg, 0.13 mmol) and 1.1 equivalents of Captisol®-Na⁺, Na⁺ equivalents, (48.9 mg moisture corrected, 46.9 mg active, 0.02 mmol, 0.14 mmol H+) was prepared by dissolving Captisol®-Na+ in in 1.7 mL of HPLC-grade H₂O, then adding the salt in small portions and stirring via a teflon coated magnetic stir bar, until a homogenous solution formed. A solution of benzethonium chloride (20 μL, 1 mg/mL solution, 0.01% final concentration) was added to the solution. The pH of this solution was 6.13. A solution of NaOH (2M) was added until the pH was 7.00±0.01. The solution was diluted to 2 mL and the final pH was determined to be 7.04. The solution was filtered through a 0.20 micron syringe filter. The osmolality was 209.3 mOsmol/kg.

A formulation of Caspofungin Acetate was prepared as follows. A solution of 70 mg/mL (FB equivalents) of caspofungin Acetate (155.4 mg, 0.13 mmol) was prepared by dissolving it in a 2 mL volumetric flask with HPLC grade water. The pH of the solution was 5.86. The solution was filtered through a 0.20 micron syringe filter. The osmolality was 94 mOsmol/kg.

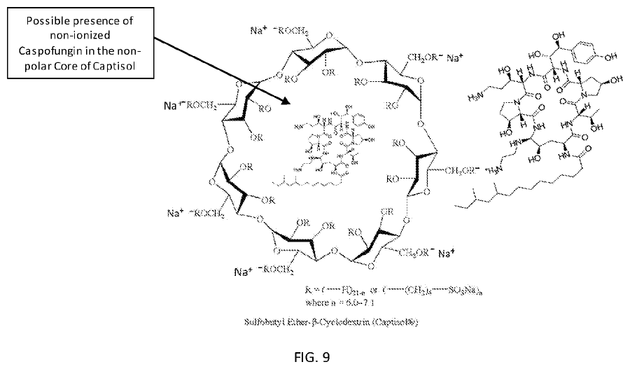

The structure of the resulting Caspofungin-Captisol® salt is shown in FIG. 9.

### Melevodopa

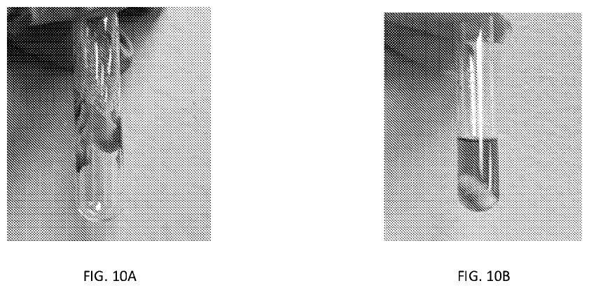

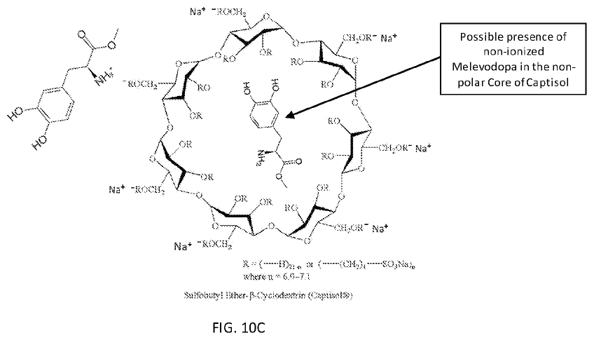

The extraction of melevodopa is described as follows. Melevodopa HCl (2.08 g, 8.40 mmol) was dissolved in 200 mL of DI water. Ammonium hydroxide was added to the solution until the pH was >10 by litmus paper. Upon addition of the ammonium hydroxide, the melevodopa solution turned transparent brown. The solution was extracted three times with ethyl acetate (75 mL) (each time, the aqueous layer became a darker brown), washed with brine (50 mL), and dried with sodium sulfate. It was then placed on the rotavap to evaporate the ethyl acetate. A couple of drops of concentrated HCl was added to the aqueous layer, and then the solution was extracted twice with ethyl acetate, washed with brine, and dried with sodium sulfate. The solution was added to the first crop, concentrated, and further dried under high-vacuum (<0.01 mbar). The product was a sticky orange oil (0.29 g, 7.1% ethyl acetate by NMR, 15.22% yield taking solvent into account), as show in FIG. 10A. HPLC showed ˜37.2% of an impurity. The material was used without further purification.

A formulation of Melevodopa FB Cap Acid was prepared as follows. A solution of 70 mg/mL melevodopa FB (74.8 mg with 7.1% solvent taken into account, 0.33 mmol) and 1.1 equivalents of CapAcid, H⁺ equivalents, (123.3 mg moisture corrected, 113.3 mg active, 0.06 mmol, 0.36 mmol H⁺) was prepared by dissolving CapAcid in 800 μL of HPLC grade water, then adding the freebase and stirring until a homogenous solution formed. Benzethonium chloride (20 μL, 1 mg/mL solution, 0.01% final concentration) was added to the solution. The pH of this solution was 1.22. NaOH (2M) was added until the pH was 7.00±0.01. The solution was diluted to 1 mL and the final pH was determined to be 6.97. The solution was filtered through a 0.45 micron syringe filter. The osmolality was 605.7 mOsmol/kg. The resulting solution was shown in FIG. 10B.

The structure of the resulting Melevodopa-Captisol® salt is shown in FIG. 10C.

### Eletriptan

The extraction of Eletriptan FB is described as follows. Eletriptan HBr (5.00 g, 10.80 mmol) was dissolved in DI water (1.2 L), and about 50% of it dissolved. The dissolved solution was decanted off and 10% NaOH solution was added until the pH of the solution was >10 by litmus paper. It was then extracted three times with ethyl acetate (75 mL), washed with brine (50 mL), and dried with anhydrous sodium sulfate. The solution was evaporated under vacuum. DI water was added to the remaining undissolved eletriptan HBr until most was dissolved. The solution was decanted from the small amount of remaining solids and treated the same as the initial extraction. The organic extractions were combined and the solvent was removed under vacuum, the resulting amber colored oil was kept under high-vac overnight (3.69 g, 3% ethyl acetate by NMR, 86.7% yield taking solvent into account).

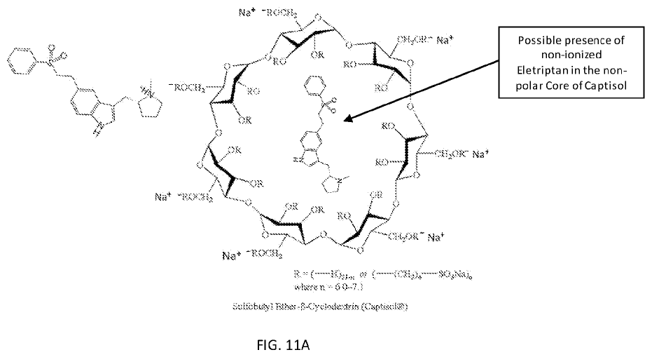

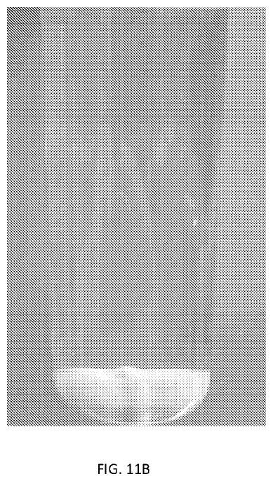

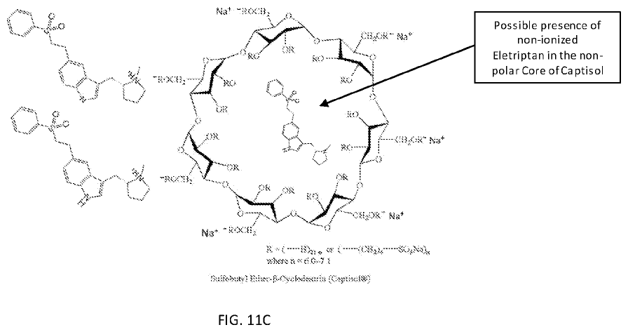

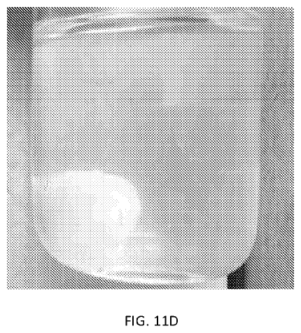

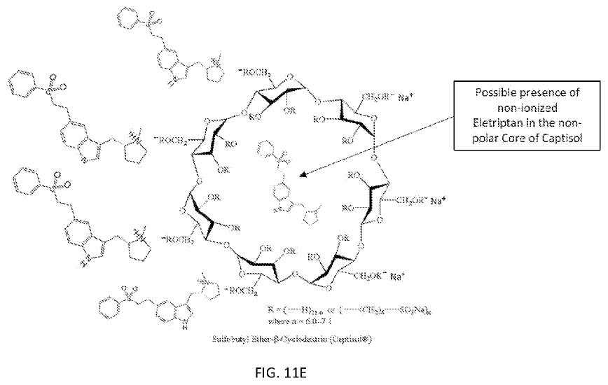

A formulation of Eletriptan-Cap Acid with a 1:1 Ratio of API to CapAcid was prepared as follows. CapAcid (574.6 mg moisture corrected, 1 molar equivalent relative to the cyclodextrin, 528.1 mg active, 0.26 mmol), was dissolved in HPLC grade water (3 mL). Eletriptan FB (100.2 mg, 0.26 mmol) was added to the solution and stirred until fully dissolved (˜30 mins). The solution was transparent with a slight orange tint. The pH of this solution was 0.48. NaOH (2M) was added until the pH was ˜6.00. This gave a concentration of 33.3 mg/mL of eletriptan FB equivalents. The final pH was 6.06 and the solution was filtered through a 0.20 micron syringe filter and lyophilized for 2 days. The product was a white solid that was ground into a powder (0.57 g, 83.5% yield). The moisture content (KF titration) was 1.12%. The structure of the resulting Eletriptan-Captisol® salt with a 1:1 Ratio of API to CapAcid is shown in FIG. 11A.

A formulation of Eletriptan-Cap Acid at 2:1 Ratio of API to CapAcid was prepared as follows. CapAcid (278.3 mg moisture corrected, 0.5 molar equivalent relative to the cyclodextrin, 264.1 mg active, 0.13 mmol), was dissolved in HPLC grade water (6 mL). Eletriptan FB (99.9 mg, 0.26 mmol) was added to the solution. After stirring for 1 hour, most of the freebase dissolved, and the volume was increased to 10 mL. After stirring for another hour, the freebase was almost entirely dissolved except for a small amount stuck to the stir bar and the sides of the test tube. The volume was increased to 12 mL and the solution stirred for 1 more hour. The solution was transferred to a scintillation vial and the pH of this solution was 1.42, and it was transparent with a slight orange tint. NaOH (2M) was added to titrate the solution to a pH of around 6. This gave an approximate concentration of 8.3 mg/mL eletriptan FB equivalent and the resulting solution is shown in FIG. 11B. The final pH was 6.08. The solution was filtered through a 0.20 micron syringe filter and the solution lyophilized. The recovered product was a white solid that was ground into a powder with a glass stir rod (0.32 g, 83.1% yield). The moisture content (KF titration) was 5.53%. The structure of the resulting Eletriptan-Captisol® salt with a 2:1 Ratio of API to CapAcid is shown in FIG. 11C.

A formulation of Eletriptan CapAcid with a ratio of 4:1 of API to CapAcid was prepared as follows. CapAcid (143.6 mg, 0.25 molar equiv. relative to the cyclodextrin, 132.0 mg active, 0.07 mmol) was dissolved in HPLC grade water (20 mL). Eletriptan FB (100.0 mg, 0.26 mmol) was added to the solution. Most of the freebase remained on the surface of the CapAcid solution and would not mix in, even after stirring for 1 hour. The volume was increased to 30 mL and the freebase fell to the bottom of the beaker; however it still did not dissolve. After stirring for 2 days, most of the FB dissolved and the solution was cloudy. The resulting solution was shown in FIG. 11D. The pH of this solution was 2.34. NaOH (2M) was added to titrate the solution to a target pH of 6.00. The final pH was 6.01. The solution was filtered through a 0.20 micron syringe filter and lyophilized. The recovered product was a white solid that was ground into a powder with a glass stir rod (0.22 g, 88.5% yield). The moisture content (KF titration) was 6.70%.

The structure of the resulting Eletriptan-Captisol® salt with a 4:1 Ratio of API to CapAcid is shown in FIG. 11E.

### Rotigotine

Rotigotine Freebase was prepared as follows. Rotigotine HCl (1.0 g, 2.84 mmol) was dissolved in 1.6 L of DI water, with some undissolved. The dissolved solution was decanted off and ammonium hydroxide was added until the solution was basic by litmus paper. The solution was extracted three times with ethyl acetate, washed with DI water to get rid of excess ammonium, washed with brine, and then dried with sodium sulfate. The solvent was evaporated under vacuum and then placed under high-vacuum overnight. The provided a sticky orange oil, and the percent solvent was determined to be 5.6% by NMR. It was placed back on the high-vac for two days and it turned into a light brown solid (0.78 g, 86.7% yield). The solid was free of solvent at this point, as determined via NMR. The melting point range was determined to be 77.1-79.3° C. A reference melting point was 78° C. (Chemical Book. https://www.chemicalbook.com/ProductIndex_EN.aspx).

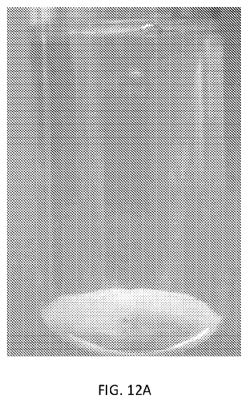

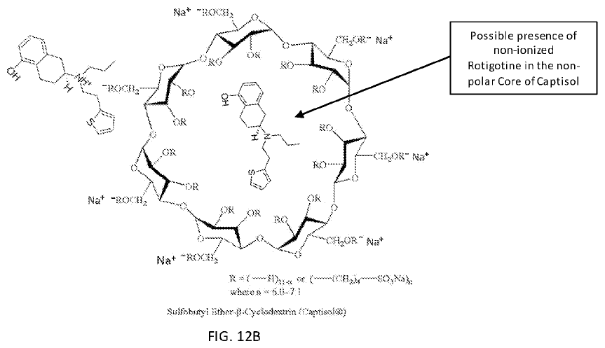

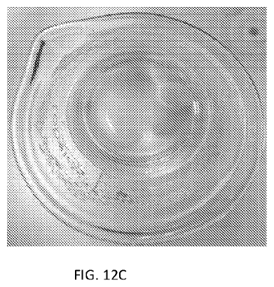

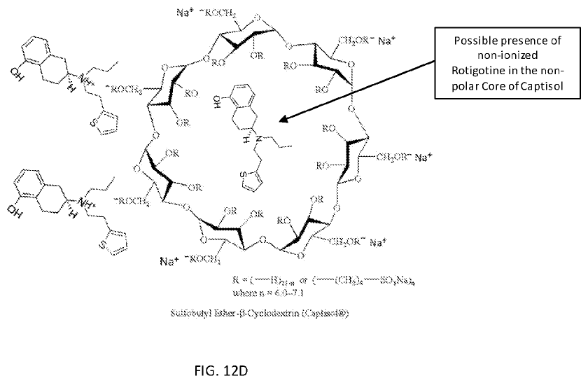

A formulation of Rotigotine-CapAcid with a ratio of 1:1 equivalent of API to CapAcid was prepared as follows. CapAcid (696.8 mg moisture corrected, 1 molar equivalent relative to the cyclodextrin, 640.3 mg active, 0.32 mmol) was dissolved in HPLC grade H₂O (3 mL). Rotigotine FB (100.1 mg, 0.32 mmol) was added to the solution. After stirring for 1 hour, most of the freebase dissolved, however the volume was increased to 6 mL. After stirring for another hour, the freebase was almost entirely dissolved except for a small amount stuck to the stir bar and the sides of the test tube. The volume was increased to 10 mL (concentration of 10 mg/mL rotigotine FB equivalents) and the solution stirred for 1 more hour. The resulting solution was shown in FIG. 12A. The solution was transferred to a scintillation vial and the pH of this solution was 0.93, and it was transparent with a slight orange tint. NaOH (2M) was added to titrate the solution to a pH of around 5.50. The final pH was 5.49. The solution was filtered through a 0.20 micron syringe filter and the solution lyophilized. The recovered product was a white solid that was ground into a powder with a glass stir rod (0.52 g, 66.7% yield). The moisture content (KF titration) was 4.97%.

The structure of the resulting Rotigotine-Captisol® salt with a 1:1 Ratio of API to CapAcid is shown in FIG. 12B.

A formulation of Rotigotine CapAcid with a 2:1 Ratio of API to CapAcid was prepared as follows. CapAcid (348.5 mg moisture corrected, 0.5 molar equivalent relative to the cyclodextrin, 320.2 mg active, 0.16 mmol) was dissolved in HPLC grade water (20 mL). Rotigotine FB (100.1 mg, 0.32 mmol) was added to the solution. A large ball of solid remained on the bottom of the beaker while smaller chunks remained suspended. The solution stirred for 1 hour and the appearance did not change. The volume was increased to 30 mL (concentration of 3 mg/mL rotigotine FB equivalents) and some of the freebase dissolved but there was still a large amount undissolved. After stirring for 2 days, most of the FB dissolved. The resulting solution was shown in FIG. 12C. The pH of this solution was 1.66. NaOH (2M) was added to titrate the solution to a pH of around 5.50. The final pH was 5.51. The solution was filtered through a 0.20 micron syringe filter and then lyophilized. The recovered product was a white solid that was ground into a powder with a glass stir rod (0.44 g, 98.0% yield). The moisture content (KF titration) was 6.34%.

The structure of the resulting Rotigotine-Captisol® salt with a 2:1 Ratio of API to CapAcid is shown in FIG. 12D.

### Copanlisib

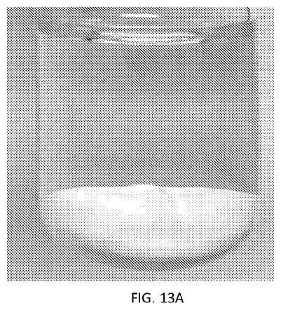

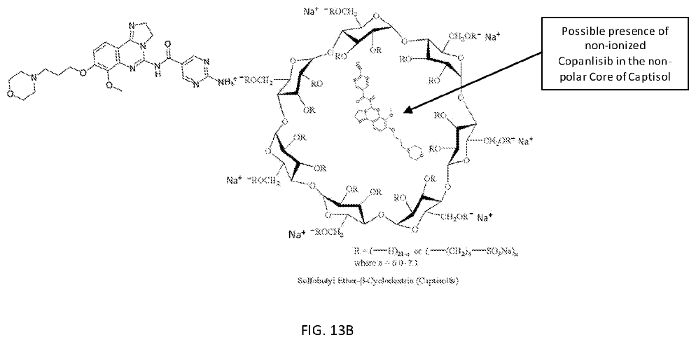

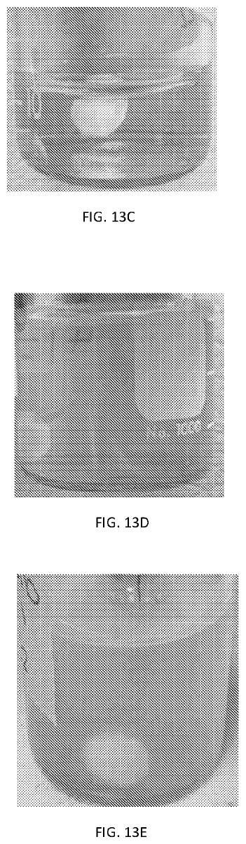

A formulation of Copanlisib-CapAcid with a 1:1 ratio of API to the cyclodextrin was prepared as follows. CapAcid (460.5 mg moisture corrected, 1 molar equivalent relative to the cyclodextrin, 420.4 mg active, 0.21 mmol) was dissolved in HPLC grade water (3 mL). Copanlisib FB (100.1 mg, 0.21 mmol) was added to the solution. After stirring for 1 hour, all of the freebase dissolved, and the homogenous solution had a yellow tint with a concentration of 33.3 mg/mL copanlisib FB equivalents, as shown in FIG. 13A. The pH of this solution was 0.76. NaOH (2M) was added to titrate the solution to a pH of around 6. The final pH was 6.13. The solution was filtered through a 0.20 micron syringe filter and lyophilized. The recovered product was a yellow tinted solid that was ground into a fine powder with a glass stir rod (0.48 g, 92.2% yield).

The structure of the resulting Copanlisib-Captisol® salt with a 1:1 Ratio of API to Cap Acid is shown in FIG. 13B.

The following procedure was used in an attempt to produce a formulation of Copanlisib CapAcid at a ratio of 2:1 relative to the Cyclodextrin. CapAcid (230.3 mg moisture corrected, 0.5 molar equivalent relative to the cyclodextrin, 210.2 mg active, 0.11 mmol) was dissolved in HPLC grade water (10 mL). Copanlisib FB (100.0 mg, 0.21 mmol) was added to the solution. After stirring for 1 hour, some of the freebase dissolved, however there was still a large undissolved quantity, as shown in FIG. 13C. The volume was increased to 20 mL. After stirring for an additional 3 hours, the solution became cloudy with some undissolved oil at the bottom of the beaker. The volume was increased to 30 mL (a target concentration of 3 mg/mL copanlisib FB equivalents) and the solution stirred overnight. After stirring overnight, the solution was clear with a small amount of undissolved oil at the bottom of the beaker and stuck to the stir bar, as shown in FIG. 13D. The pH of this solution was 2.10. While titrating the pH with 2M NaOH (20 μL), there was precipitation and the solution became cloudy (pH was 2.20 at this stage), as shown in FIG. 13E.

### Nafamostat

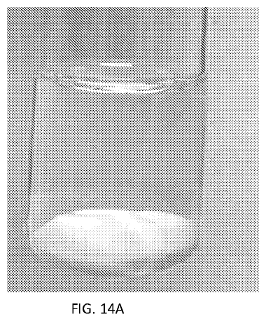

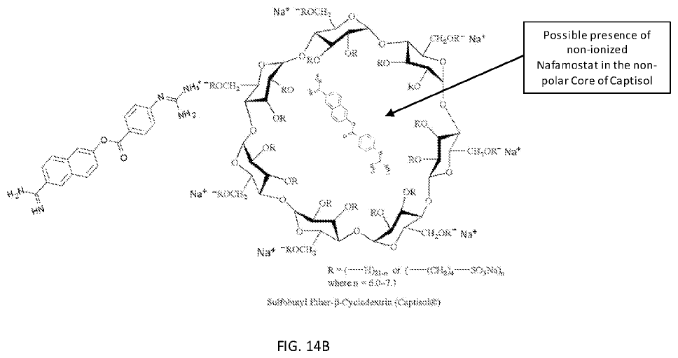

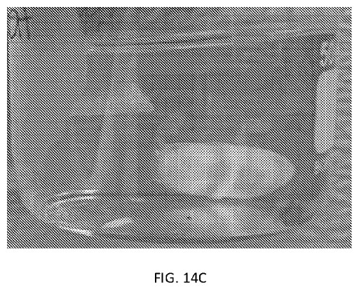

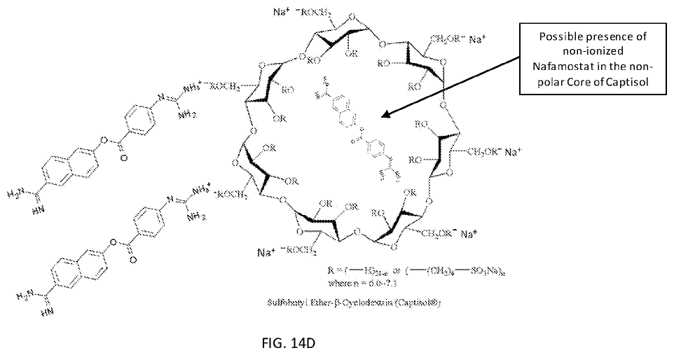

A formulation of Nafamostat Cap Acid at 1:1 Ratio of API to Cap Acid was prepared as follows. CapAcid (632.8 mg moisture corrected, 1 molar equivalent relative to the cyclodextrin, 581.5 mg active, 0.29 mmol) was dissolved in HPLC grade water (3 mL). Nafamostat FB (99.9 mg, 0.29 mmol) was added to the solution. After stirring for 1 hour, all of the freebase dissolved giving a concentration of 33.3 mg/mL nafamostat FB equivalents, as shown in FIG. 14A. The pH of this solution was 0.49. NaOH (2M) was added to titrate the solution to a pH of around 7. The final pH was 6.99. The solution was filtered through a 0.20 micron syringe filter and lyophilized. The recovered product was a white solid that was ground into a powder with a glass stir rod (0.63 g, 89.2% yield). The moisture content (KF titration) was 3.53%.

The structure of the resulting Nafamostat-Captisol® salt with a 1:1 Ratio of API to Cap Acid is shown in FIG. 14B.

A formulation of Nafamostat-Cap Acid at 2:1 ratio of API to CapAcid was prepared as follows. CapAcid (318.6 mg moisture corrected, 0.5 molar equivalent relative to the cyclodextrin, 290.8 mg active, 0.14 mmol) was dissolved in HPLC grade water (10 mL). Nafamostat FB (100.1 mg, 0.29 mmol) was added to the solution. After stirring for 1 hour, the solution was cloudy and there were some undissolved chunks of freebase. The volume was increased to 20 mL. After stirring for 3 more hours, more freebase had dissolved, but the solution was still cloudy. The volume was increased to 30 mL, and the solution was left to stir overnight. After stirring overnight, the solution was clear and all the freebase had dissolved, giving a concentration of 3 mg/mL nafamostat FB equivalents, as shown in FIG. 14C. The pH of this solution was 1.55. NaOH (2M) was added to titrate the solution to a pH of around 7. The final pH was 6.96. The solution was filtered through a 0.20 micron syringe filter and then lyophilized. The recovered product was a white solid that was ground to a powder with a glass stirring rod (0.39 g, 96.7% yield). The moisture content (KF titration) was 3.14%.

The structure of the resulting Nafamostat-Captisol® salt with a 2:1 Ratio of API to CapAcid is shown in FIG. 14D.

A Formulation of Nafamostat-Captisol®-Na+ Mixture was prepared as follows. Captisol® Sodium (325.3 mg, 1 molar equivalent relative to the cyclodextrin, 311.3 mg active, 0.14 mmol) was dissolved in HPLC grade water (3 mL). Nafamostat FB (50.0 mg, 0.14 mmol) was added to the solution to give a target concentration of ˜16.7 mg/mL. After stirring for 30 minutes, the freebase had dissolved, as shown FIG. 14E. It was allowed to stir for 48 hours like the other API FB Captisol® Na+ solutions. After 48 hours, the solution was filtered through a 0.20 micron syringe filter and the osmolality was 354.3 mOsm/Kg. The structure of the resulting Nafamostat-Captisol® salt with a 2:1 Ratio of API to CapAcid is the same as that shown in FIG. 14D.

### Remdesivir

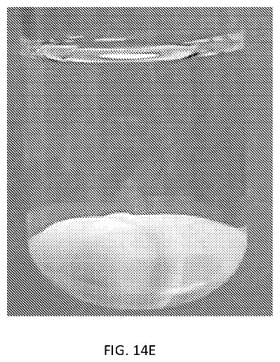

The following procedure was used in an attempt to produce a formulation of Remdesivir-CapAcid at a 1:1 ratio of API to CapAcid. CapAcid (367.3 mg moisture corrected, 1 molar equiv. relative to the cyclodextrin, 335.2 mg active, 0.17 mmol) was dissolved in HPLC grade water (3 mL). Remdesivir FB (100.0 mg, 0.17 mmol) was added to the solution. After stirring for 1 hour, all of the freebase dissolved to give a concentration of 33.3 mg/mL remdesivir FB equivalents, as shown in FIG. 15A. The pH of this solution was 0.72. NaOH (2M) was added to titrate the solution to a pH of around 7. The freebase began to crash out at a pH of 1.83, as shown in FIG. 15B, so the pH was not brought higher.

### Midazolam

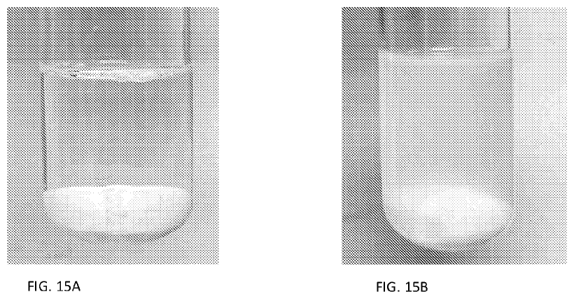

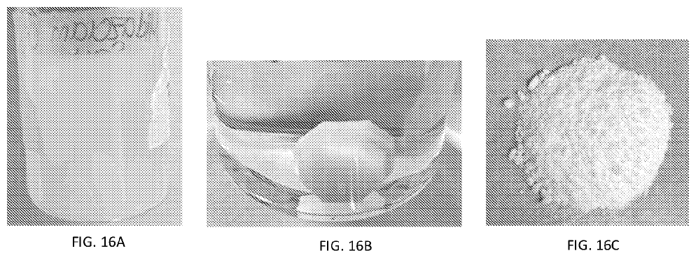

A formulation of Midazolam CapAcid at 1:1 Ratio of API to CapAcid was prepared as follows. CapAcid (312.8 mg moisture corrected, 1 molar equivalent relative to H⁺, 287.0 mg active, 0.14 mmol, 0.92 mmol H⁺) was dissolved in HPLC grade water (10 mL). Midazolam FB (300.8 mg, 0.92 mmol) was added to the solution and stirred (magnetic stirring via a teflon coated stir bar). After stirring for 30 minutes, the solution was cloudy suggesting incomplete dissolution, as shown in FIG. 16A. CapAcid (386.9 mg added, 677.9 mg total moisture corrected in solution, 621.9 total active in solution, 0.31 mmol, 2.00 mmol H+) was then added to provide a 3:1 ratio of midazolam to CapAcid (relative to the Captisol cyclodextrin). Addition of CapAcid produced a transparent colorless solution upon stirring, see FIG. 16B.

An aqueous solution of NaOH (2 M) was added in small portions via a micropipette. The highest achievable pH without precipitation of midazolam freebase solids was 3.15. This required the addition of 340 μL of 2M NaOH (0.68 mmol). After this point, another 20 μL of 2M NaOH was added (360 μL total, 0.72 mmol total), and solids began to crash out and would not redissolve with stirring. The pH was measured to be 2.92 at this point. A solution of 0.5 M HCl (10 μL, 0.005 mmol) was added in 1 μL portions via a micropipette until the solid redissolved. The final pH of the solution was 2.86. The solution was then placed in the freezer for several days and then was lyophilized for 48 hours. The recovered product was a white solid (916.4 mg, 92.5% recovery) that was ground into a lightly colored white powder (shown in FIG. 16C) with a glass stir rod. The product was stored in a 20 mL borosilicate glass scintillation vial, sealed with parafilm and covered in aluminum foil to protect from light, and stored in a −20° C. freezer.

### SUMMARY

This resulted above showed that different stoichiometric API:CapAcid salt ratios showed different solubilities and that lower stochiometric ratios of API relative to CapAcid may have improved solubilities over higher stochiometric ratios. Also, the results suggested that solubilities were not the result of simple complexation of the API by the cyclodextrin; given that ratios of greater than 1:1 were still soluble. Rather it suggested that SBEBCD has solubilizing power as an anion.

Going forward, the results indicate that these different stoichiometry API-Cap Acid salts will show different solubilities and that these will be improved relative to the API freebase complexed to Captisol® Na⁺ salt. Furthermore, even APIs which are unable to complex to the beta-cyclodextrin pore would be able to form API-CapAcid salts as long as they contain a basic nitrogen that can form a cation. Furthermore, the solubility of API-CapAcid salts will in general be improved over existing API anion salts in common use (e.g. HCl, HBr, mesylate, tosylate, fumarate, acetate, etc). This has implications for parenteral formulations as well as formulations prepared for oral administration. Higher solubility of these forms as well as faster rates of dissolution will influence important pharmacokinetic properties including Cₘₐₓ, Tₘₐₓ as well bioavailability. Amorphous forms often show improved PK properties over crystalline forms. The API:Cap Acid salts are believed to be amorphous—it would be unlikely they could form crystalline materials given the inherent heterogeneity of the SBEBCD molecule (i.e., it is a mixture of various isomers and differing degrees of cyclodextrin side chain modification). Furthermore, visual inspection indicated the solids appeared to be non-crystalline and amorphous.

To evaluate the improved solubility profile of the API-CapAcid salts, solubility analysis may be conducted in water (as well as simulated physiological fluids) and HPLC, LC/MS, NMR or UV-VIS spectroscopy may be used to quantify the API and calculate the solubility. The concentrations of API:Captisol® solutions at different molar ratios would then be compared with those obtained from different molar ratios of API FB:CapAcid, API:cyclodextrin complexes, as well as commercial API salt forms. The rate of dissolution of the API-CapAcid salts (at different stochiometric ratios) can be similarly calculate using comparable analytical methods. In vivo pharmacokinetic experiments can then be performed in various species on top candidates (determined from in vitro measurements) to calculate PK parameters including Cₘₐₓ, Tₘₐₓ, AUC (and bioavailability calculated). These will be compared to standard commercial API salts.

Of note, when the API:CapAcid salts described herein were made, any excess H⁺ from incomplete stoichiometric ratios were always neutralized. So, for example, if 3 moles of API FB are added relative to the H⁺ on the CapAcid (i.e., there are 6.5 H⁺ per Captisol®), an additional 3.5 molar equivalents of H+ will remain on each (average substituted) cyclodextrin molecule of Captisol®. Without neutralization of this excess H⁺, the resulting amorphous solids (which are often hydrates) and especially the solutions prepared from them would be highly acidic (pH between 1-3). This could cause tissue irritation especially when given parenterally, contribute to rapid decomposition and instability of the API as well as the cyclodextrin ring (via hydrolysis), and it could reduce the solubility in water of the API:Cap salts relative to the Na⁺ neutralized form. These salt forms were highly acidic as when they were prepared in solution (which is always highly acidic) and pH was measured before titration with NaOH. To test the adverse effect of excess H⁺ on these salts, both forms (H⁺ vs. Na⁺ neutralized form) of specific APIs can be prepared and their stability and solubility can be measured.

Table 5 lists the solubility and pKa values of the various APIs described above.

TABLE 5
Solubility in Water and pKa's of APIs

| | Successful in | | | | |
|---|---|---|---|---|---|
| | a 70 mg/mL | | | | |
| | API-CapAcid | Reported | | Reported | |
| | Formulation | Freebase | | Salt | |
| API | (Y/N) | Solubility | | Solubility | pKa |
| Tigecycline | Y | 0.45 | mg/mL⁹ | | 8.97¹⁰ |
| | | | | | (predicted) |
| Naloxone | Y | 1.415 | mg/mL¹¹ | 50 mg/mL¹³ | 7.9¹² |
| | | | | (HCl Dihydrate) | |
| Amikacin | Y | 50 | mg/mL^{6, 11} | 50 mg/mL¹³ | 8.1¹⁴ |
| | | | | (Disulfate) | |
| 1-Amantadine | Y | 6.29 | mg/mL¹¹ | 50 mg/mL¹³ | 10.1⁶ |
| | | | | (HCl) | 10.58¹¹ |
| Rimantadine | Y | 0.00915 mg/mL¹⁰ | | ~20 mg/mL | 10.14¹⁰ |
| | | (predicted) | | (HCl)^{#} | (predicted) |
| Amifampridine | Y | 7 | mg/mL¹⁵ | 159 mg/mL¹⁰ | 9.25¹⁰ |
| | | | | (predicted) | (predicted) |
| | | | | (Dihydrochloride) | |
| Caspofungin | Y | 0.28 | mg/mL¹¹ | 100 mg/mL¹⁵ | 9.76¹⁰ |
| | | | | (Acetate) | (predicted) |
| Eletriptan | N | 0.00118 mg/mL^{10, 11} | | <1 mg/mL¹⁶ | 8.37¹⁰ |
| | | (predicted) | | (HBr) | (predicted) |
| Rotigotine | N | 0.00904 mg/mL¹⁰ | | 3.51 mg/mL¹⁷ | 10.03 |
| | | (predicted) | | (HCl) | (phenol) |
| | | | | | 10.97 |
| | | | | | (amine)¹¹ |
| Copanlisib | N | Insoluble¹⁹ | | 0.023 mg/mL¹⁸ | 6.88¹⁰ |
| | | | | (Dihydrochloride) | (predicted) |
| Nafamostat | N | 0.0341 mg/mL¹⁰ | | >10 mg/mL¹³ | 11.32¹⁰ |
| | | (predicted) | | 25 mg/mL²⁰ | (predicted) |
| | | | | (Mesylate) | |
| Remdesivir | N | Insoluble¹⁵ | | — | 0.65¹⁰ |
| | | | | | (predicted) |
| Flumazenil | N | 1.2 | mg/mL²¹ | — | 2.87²¹ |

^{#}Observed experimentally.
¹AK Scientific, Inc. https://aksci.com/
²Toronto Research Chemicals https://www.trc-canada.com/
³Santa Cruz Biotechnology. https://www.scbt.com/home
⁴Bio-Connect Life Sciences. https://shop.bio-connect.nl/bioconnect.home.aspx
⁵A Smart Chem-Search Engine. https://www.chemsrc.com/en/
⁶Chemical Book. https://www.chemicalbook.com/ProductIndex_EN.aspx
⁷Loudon, G. Marc; Radhakrishna, A. S.; Almond, Merrick R.; Blodgett, James K.; Boutin, Raymond H. Conversion of aliphatic amides into amines with [I,I-bis(trifluoroacetoxy)iodo]benzene. 1. Scope of the reaction J. Org. Chem. 1984, 49 (22), 4272-4276.
⁸Hussein, A. Q; Herzberger, S.; Jochims, J. C. Chem. Ber. 1979, 112, 1102-1109.
⁹Toku-E. https://www.toku-e.com/content/product-documents/Product%20Data%20Sheet%20-%20T022.pdf#:~:text=Tigecycline%20is%20soluble%20in%20water,%3E3%20mg%2FmL).
¹⁰Drug Bank. https://go.drugbank.com/
¹¹PubChem. https://pubchem.ncbi.nlm.nih.gov/
¹²Clarke's Analysis of Drugs and Poisons; Moffat, A. C., Osselton, M. D., Widdop, B., 3rd Ed; Pharmaceutical Press, London, UK, 2004. ISB 0 85369 473 7.
¹³Sigma Aldrich. https://www.sigmaaldrich.com/US/en?gclid=Cj0KCQjwmuiTBhDoARIsAPiv6L_VtwSGiFemrhnERNz0yJapnVgihIfAwnvmcUUa-IAR6LZ00T0rMXAaAo4QEALw_wcB
¹⁴Drug Information System. http://www.druginfosys.com/drug.aspx?drugcode=45&type=1
¹⁵Selleckchem. https://www.selleckchem.com/
¹⁶My Bio Source. https://www.mybiosource.com/
¹⁷Hello Bio. https://hellobio.com/
¹⁸Freundlieb, J. Formulations of Copanlisib. European Patent Application, EP 3 498 266 A1, 19 Jun. 2019.
¹⁹https://www.apexbt.com/bay-80-6946-copanlisib.html
²⁰https://www.enzolifesciences.com/fileadmin/reports/Datasheet-BML-PI143.pdf (Accessed May 14, 2022)
²¹https://go.drugbank.com/drugs/DB01205 (Accessed May 14, 2022)

Compounds that had a solubility at or below 0.03 mg/mL as freebase (or below 4 mg/mL as the salt form) were generally not soluble as 70 mg/mL solutions of API-CapAcid formulations, in contrast those with a higher solubility were. While it is not surprising that highly soluble API salts, would be soluble as the CapAcid salt, it is surprising and unexpected that more moderately soluble APIs (e.g., DXM and rimantidine) showed dramatic improvement in solubility as stoichiometric (6.5:1) CapAcid salts present in the 70 mg/mL formulations (in fact these formulations contain a slight excess of Captisol® for buffering capacity (serving as a reserve for added base that would allow complexation of any freebase formed, and a reserve of freebase form to serve as a reserve for neutralizing added acid). It should be noted that the solubility of the API salt may be a better predictor of successful 70 mg/mL formulations, than the freebase solubility, given rimantadine freebase appeared to be an outlier-however the only freebase value located for rimantidine was a predicted value. Thus, it appeared that it is possible to predict the feasibility of the API-Cap salt formulations using data on the solubility of the API.

A challenge was that reliable solubility data for certain APIs was difficult to locate (especially for the freebase forms). As such in a number of instances only predicted values could be located and in the case of rimantidine HCl this value was inconsistent with our empirical results. Likewise, the drugbank predicted value for tigecycline was 0.01455 mg/mL, whereas a literature value was 0.45 mg/mL. The solubility of a range of poor to moderately to highly soluble APIs as freebases as well as common commercial API salt forms will be determined. Their solubility will be compared to their ability to form soluble API-CapAcid salts at different stoichiometric ratios (from 6.5:1 to 1:1 API:CapAcid) using HPLC, LC/MS, NMR, and/or UV-VIS.

There does not seem to be a clear trend with pKa with the exception of compounds with a low pKa like remdesivir (and flumazenil) which were unsuccessful, suggesting inadequate salt formation. A pKa difference between acid and base is generally required for effective salt formation. Based on the results describe ed herein, it appears the pKa cut-off for formation of the API-CapAcid salt may be a pKa of a basic nitrogen below 3.

### Example 2: Complexing Agent Formulations of APIs in Ethanol

In order to make sure that the issue of the poorly soluble API-Cap formulations prepared at the 70 mg/mL concentration was not due to a failure of the API-Cap salt to form in aqueous conditions, these salts were also prepared in ethanol. This would ensure better solubility of the API FB ensuring a chance to react with the solubilized CapAcid in solution. In general, the ethanol solutions exhibited more complete dissolution than those done under aqueous conditions, however some cases, there was insoluble material even with the ethanol preparation (it is likely the API-Cap salts were forming and precipitating out rather than the FB not dissolving). The resulting solid API-CapAcid salts were then obtained as solids by evaporation of the ethanol, followed by lyophilization. When the resulting solids dissolved in water to a target concentration of 70 mg/mL (FB equivalents), these exhibited (by visual inspection) the same poor solubility as those solutions prepared directly in water. This suggested that the issues observed were due to poor solubility of the API-CapAcid salts that form. Thus, some amines due to poor solubility remained poorly soluble as the Cap Acid salt. Table 5 above shows solubility data on the FB or commercial API salts, on APIs for formulations that worked versus those that did not. APIs with poor solubility as the FB or API salt form, were observed to be the APIs most likely to fail. Whereas those with moderate-to-good solubility were successful. In many cases the API-Cap Acid salts showed substantially improved solubility over known API salts (e.g., DXM, rimantidine). Rimantidine-Cap formulation was prepared at 70 mg/mL without issue despite the fact that rimantidine HCl was only found to be soluble at 20 mg/mL (Table 2). Furthermore the 70 mg/mL rimantidine-Cap formulation was isosmotic and PH neutral. API-CapAcid salts likely show improved solubility over standard API-acid salts used in the art (e.g., HCl, HBr, fumarate, acetate, mesylate, etc). Furthermore, given the multiple ionic side chains in SBEBCD, the solubility can be tuned and optimized by using different API:SBEBCD ratios (e.g., 6.5:1, 5:1, 4:1, 3:1, 2:1, 1:1).

### General Procedure

In a scintillation vial, a solution of API FB (100 mg) and CapAcid (1 molar equivalent, moisture corrected) was prepared by dissolving Cap Acid in 200 proof ethanol (5 mL) and then adding the freebase API. If the freebase did not dissolve, another addition of ethanol (5 mL) was added. Whether or not the freebase dissolved after the second addition of ethanol, the initial pH, prior to titration, was recorded. The vial was then wrapped in foil and left uncovered in the hood to allow the ethanol to evaporate. After the ethanol fully evaporated, a small amount of HPLC grade water (3-5 mL) was added to each to see if the product would dissolve or become a suspension in the water. The solution was then lyophilized to remove the water. The product was ground with a stir rod and 70±0.1 mg was added to 1 mL of water to see if it would dissolve.

### Eletriptan

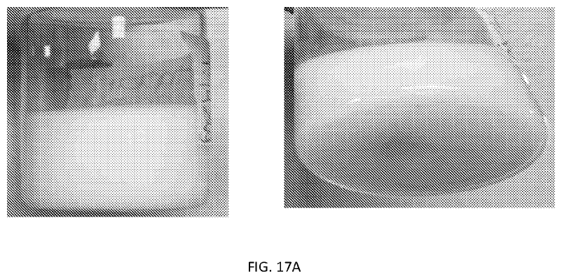

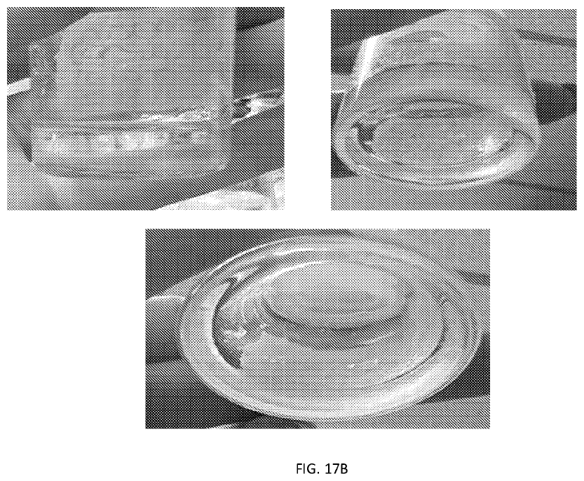

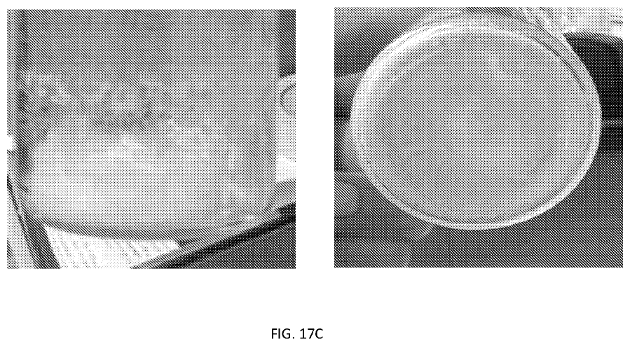

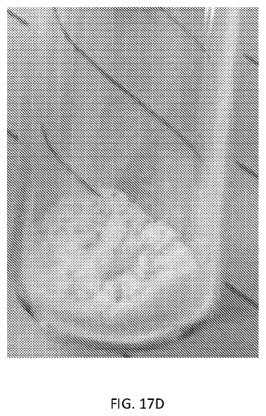

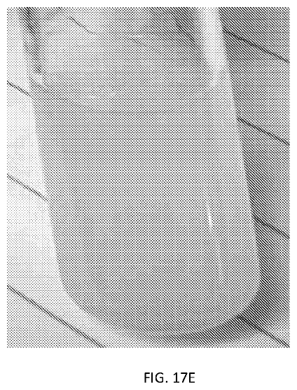

In a scintillation vial, CapAcid (1 molar equivalent, 88.4 mg moisture corrected, 81.2 mg active, 0.04 mmol, 0.26 mmol H⁺) was dissolved in 200 proof ethanol (5 mL). Eletriptan FB (100.0 mg, 0.26 mmol) was added to the solution. The solution became milky white with some brown oil at the bottom of the vial. Another addition of ethanol (5 mL) was added and the appearance did not change, even with stirring, as shown in FIG. 17A. The initial pH, prior to titration, was determined to be 8.02. The vial was wrapped in foil and left uncovered in the hood to allow the ethanol to evaporate, this took several days. Once all the ethanol was evaporated, an orange oil was left and a small amount of water was added, as shown in FIG. 17B. The oil did not mix with the water. The product was lyophilized, as shown in FIG. 17C. The recovery was 0.19 g. The product was fluffy and white with some brown on the bottom, which was ground with a glass stir rod, as shown in FIG. 17D. 70±0.1 mg of these solids were added to a test tube, followed by HPLC grade water (1 mL) and the solution vortexed, as shown in FIG. 17E. The solution became cloudy but a large amount of oil remained at the bottom.

### Carvedilol

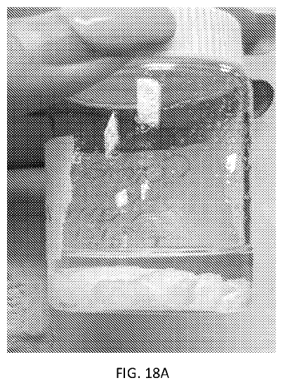

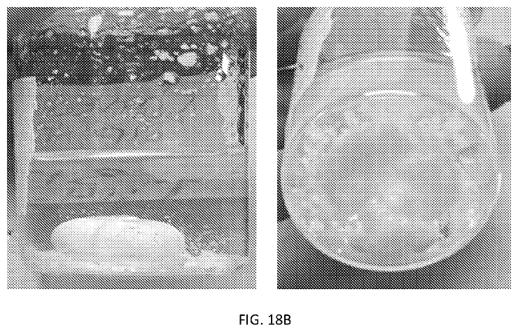

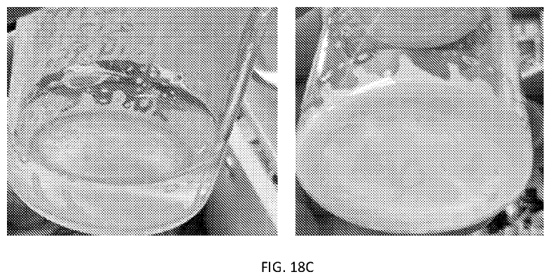

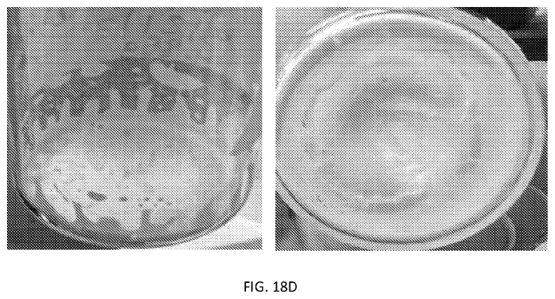

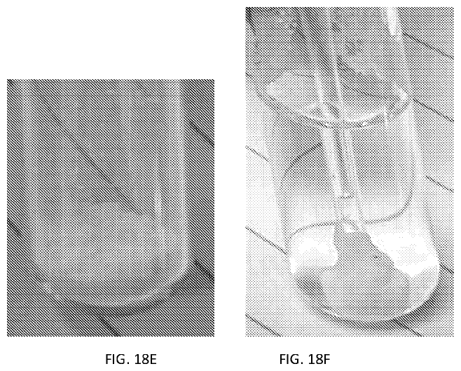

Method 1: In a scintillation vial, CapAcid (1 molar equivalent, 83.1 mg moisture corrected, 76.5 mg active, 0.04 mmol, 0.25 mmol H+) was dissolved in 200 proof ethanol (5 mL). Carvedilol FB (100.1 mg, 0.25 mmol) was added to the solution. A fluffy white solid fell to the bottom of the vial, as shown in FIG. 18A. Another addition of ethanol (5 mL) was added and more dissolved, but there was still a large amount of solid left, as shown in FIG. 18B. The pH of this solution was 0.82. The vial was wrapped in foil and left uncovered in the hood to allow the ethanol to evaporate, this took several days. Once all the ethanol was evaporated, a white solid was left and a small amount of water was added, as shown in FIG. 18C. The solid did not mix with the water. The product was lyophilized to give 0.19 g of a white solid, as shown in FIG. 18D. The solid was ground to a powder with a glass stir rod and 70±0.1 mg was added to a test tube, as shown in FIG. 18E. HPLC grade water (1 mL) was added to the test tube and vortexed. An insoluble solution resulted, as shown in FIG. 18F.

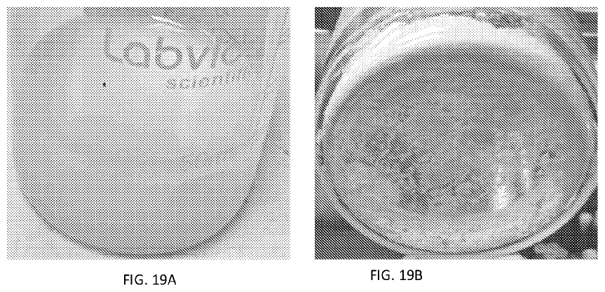

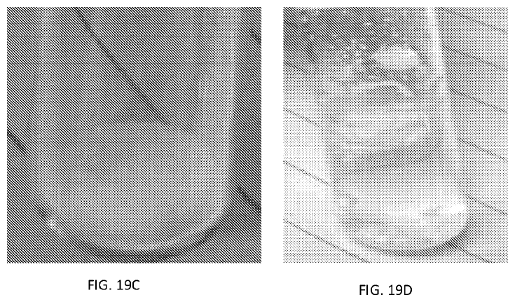

Method 2: In a beaker, Carvedilol FB (100.0 mg, 0.25 mmol) was dissolved in 200 proof ethanol (22 mL), and all of it dissolved and the solution was clear. CapAcid (1 molar equivalent, 83.1 mg moisture corrected, 76.5 mg active, 0.04 mmol, 0.25 mmol H⁺) was added to the solution. The solution became milky white, as shown in FIG. 19A. The pH of this solution was 7.56. The beaker was wrapped in foil and left uncovered in the hood to allow the ethanol to evaporate, this took one day. Once all the ethanol was evaporated, a white solid was left and a small amount of water was added. The solid did not dissolve in the water. The material was then lyophilized to give 0.21 g of a white solid, as shown in FIG. 19B. These solids were ground to a powder with a glass stir rod, as shown in FIG. 19C. In order to test the solubility, 70.0±0.1 mg was added to a test tube. HPLC grade water (1 mL) was added to the test tube and vortexed. An insoluble solution resulted, as shown in FIG. 19D.

### Copanlisib

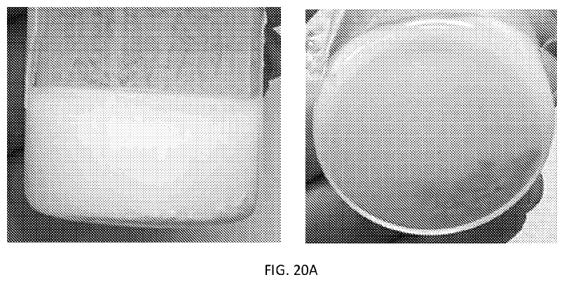

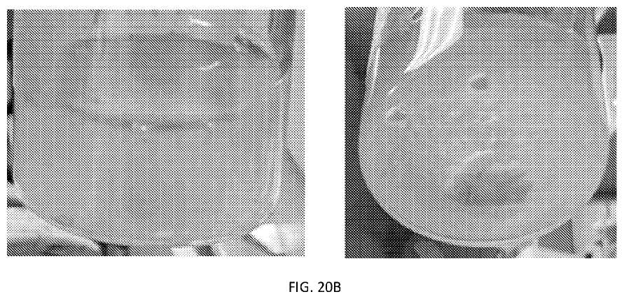

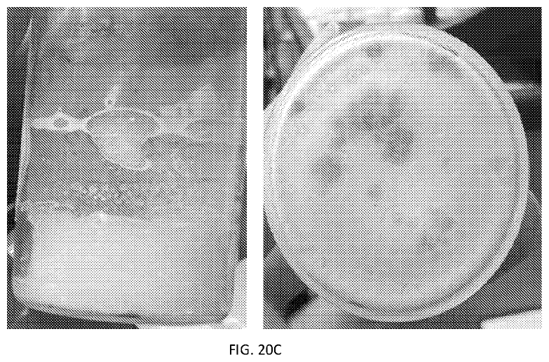

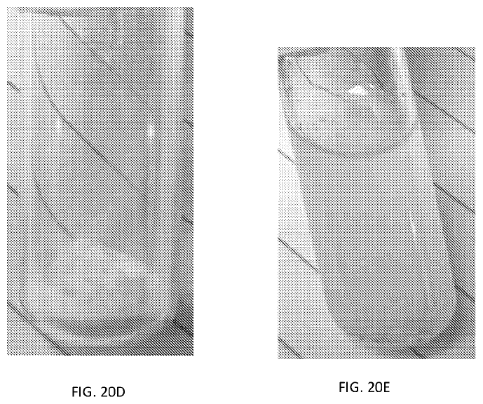

In a scintillation vial, CapAcid (1 molar equivalent, 70.4 mg moisture corrected, 64.5 mg active, 0.03 mmol, 0.21 mmol H⁺) was dissolved in 200 proof ethanol (5 mL). Copanlisib FB (100.1 mg, 0.21 mmol) was added to the solution. The solution became milky white and cloudy with a large amount of tan solid (undissolved FB) fell to the bottom of the vial, as shown in FIG. 20A. Another addition of ethanol (5 mL) was added and the appearance did not change. The pH of this solution was 0.66. The vial was wrapped in foil and left uncovered in the hood to allow the ethanol to evaporate, this took several days. Once all the ethanol was evaporated, a tan solid was left and a small amount of water was added. The solution became cloudy but most of the solid did not mix with the water, as shown in FIG. 20B. The product was lyophilized to give 0.16 g of a fluffy white solid with brown spots at the bottom of the vial, as shown in FIG. 20C. The solids were ground with a glass stir rod and 70±0.1 mg was added to a test tube, as shown in FIG. 20D. HPLC grade water (1 mL) was added to the test tube and vortexed. The solution became cloudy with a brown precipitate at the bottom, as shown in FIG. 20E.

### Rotigotine

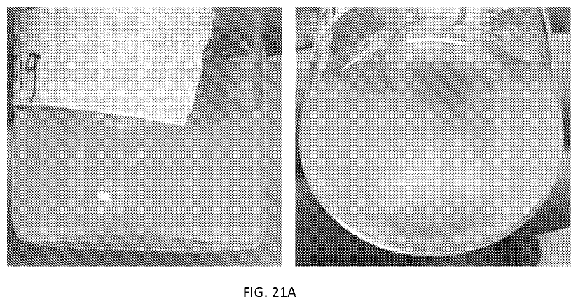

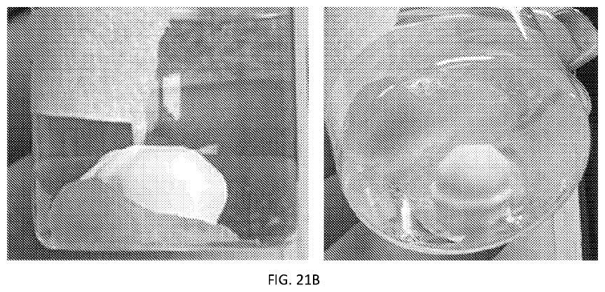

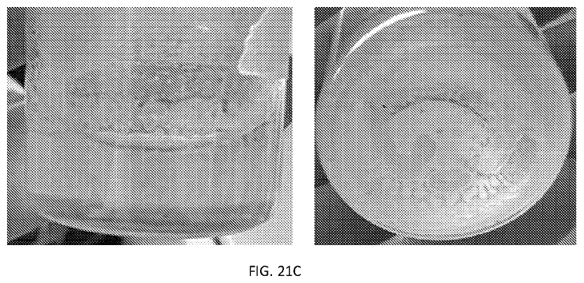

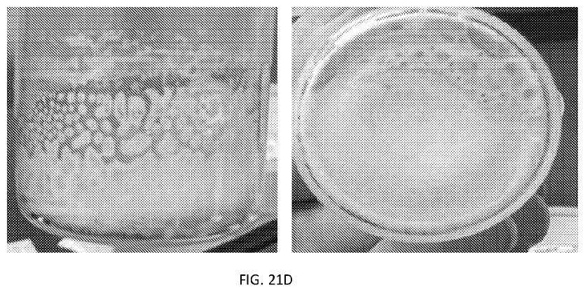

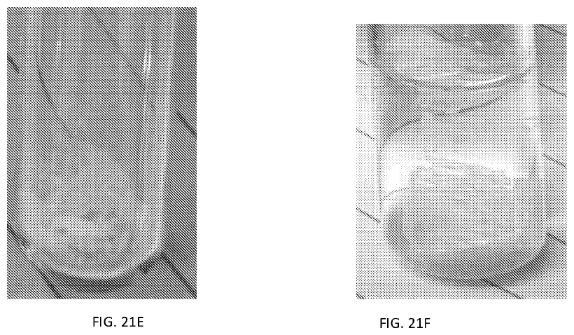

In a scintillation vial, CapAcid (1 molar equivalent, 107.3 mg moisture corrected, 98.5 mg active, 0.05 mmol, 0.32 mmol H⁺) was dissolved in 200 proof ethanol (5 mL). Rotigotine FB (99.9 mg, 0.32 mmol) was added to the solution. The solution became cloudy with a large ball of sticky tan solid at the bottom of the vial, as shown in FIG. 21A. Another addition of ethanol (5 mL) was added and the solution became clear but still had the large sticky brown ball of solid, as shown in FIG. 21B. The pH of this solution was 5.63. The vial was wrapped in foil and left uncovered in the hood to allow the ethanol to evaporate, this took several days. Once all the ethanol was evaporated, a brown oil was left and a small amount of water was added. The solution became cloudy but most of the oil did not mix with the water, as shown in FIG. 21C. The product was lyophilized to give 0.18 g of a white solid with brown spots at the bottom of the vial, as shown in FIG. 21D. The solid was ground with a glass stir rod and 70±0.1 mg was added to a test tube, as shown in FIG. 21E. HPLC grade water (1 mL) was added to the test tube and vortexed. The brown precipitate did not dissolve in water, as shown in FIG. 21F.

### Remdesivir

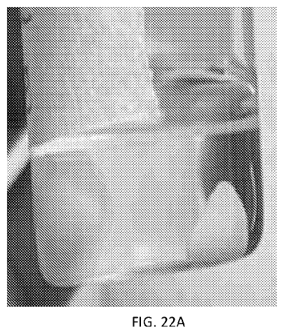

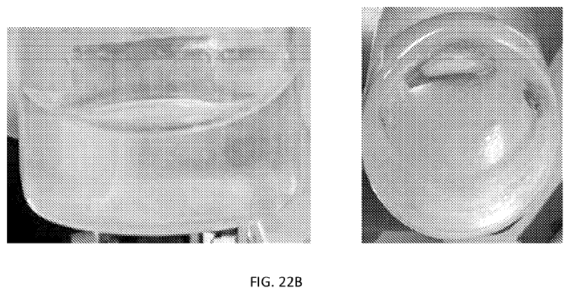

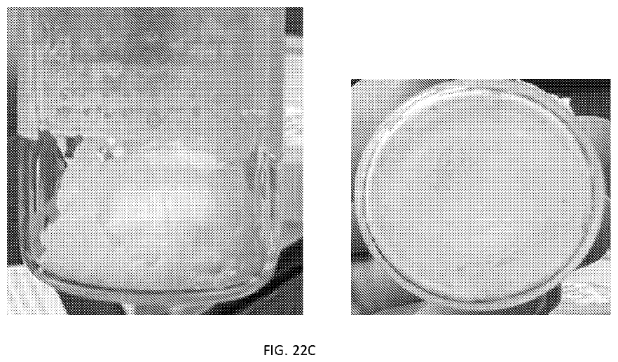

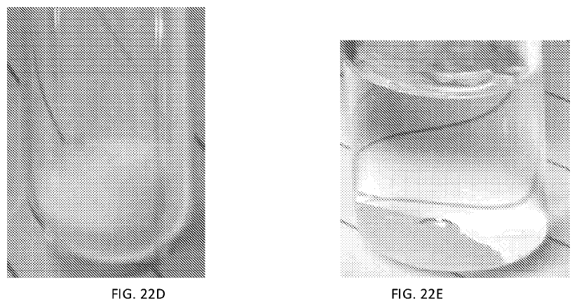

In a scintillation vial, CapAcid (1 molar equivalent, 56.0 mg moisture corrected, 51.6 mg active, 0.03 mmol, 0.17 mmol H⁺) was dissolved in 200 proof ethanol (5 mL). Remdesivir FB (99.9 mg, 0.17 mmol) was added to the solution. The solution remained clear with a large ball of white solid at the bottom of the vial, as shown in FIG. 22A. Another addition of ethanol (5 mL) was added and the appearance did not change. The pH of this solution was 1.18. The vial was wrapped in foil and left uncovered in the hood to allow the ethanol to evaporate, this took several days. Once all the ethanol was evaporated, a white solid was left and a small amount of water was added. The solution became slightly cloudy with some clear precipitate at the bottom, as shown in FIG. 22B. The product was lyophilized to give. 0.16 g of a fluffy white solid, as shown in FIG. 22C. The solid was ground with a glass stir rod and 70±0.1 mg was added to a test tube, as shown in FIG. 22D. HPLC grade water (1 mL) was added to the test tube and vortexed. The white precipitate did not dissolve in the water, as shown in FIG. 22E.

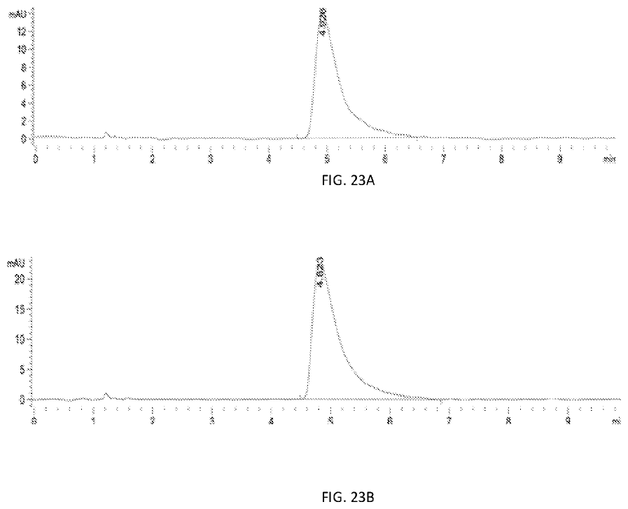

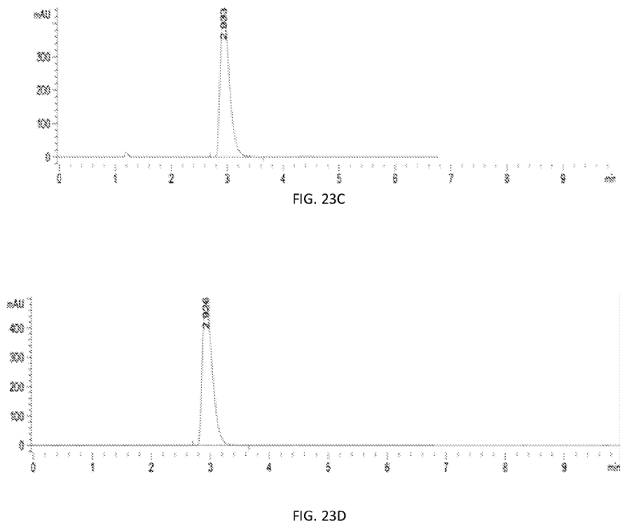

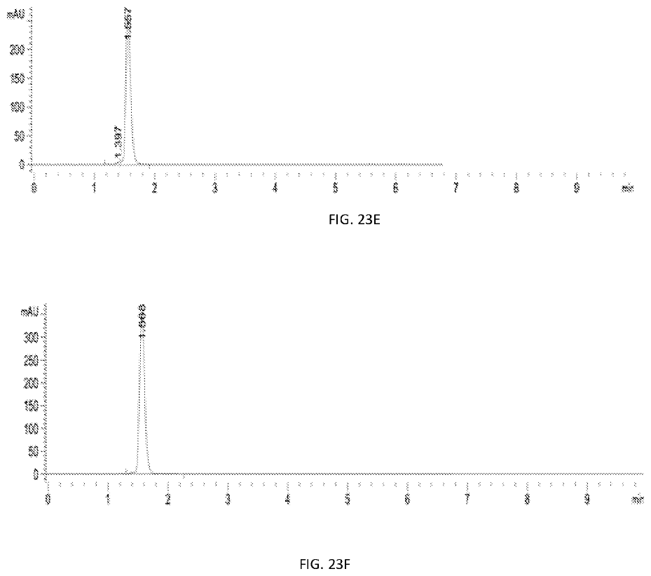

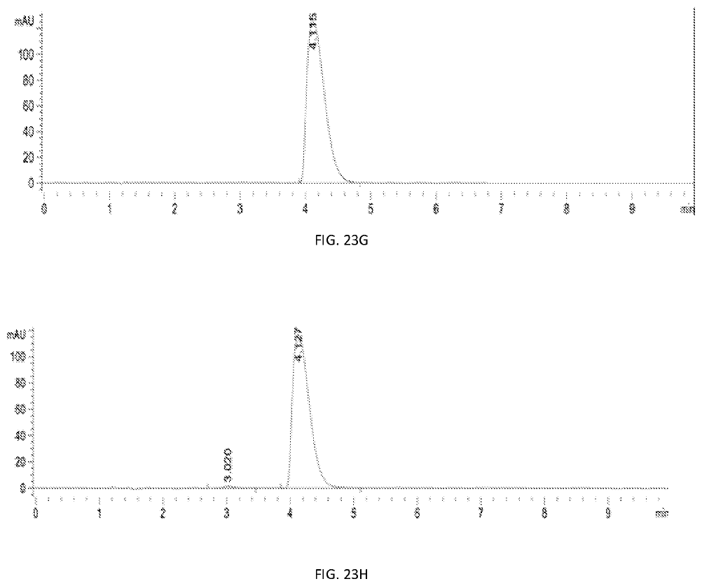

The HPLC analyses of various APIs used herein were shown: Caspofungin Acetate, (FIG. 23A), Caspofungin FB (FIG. 23B), Eletriptan HBr (FIG. 23C), Eletriptan FB (FIG. 23D), Naloxone HCl Dihydrate (FIG. 23E), Naloxone FB (FIG. 23F), Rotigotine HCl (FIG. 23G), Rotigotine FB (FIG. 23H).

### Example 2. Exemplary Complexing Agent Formulations of Compounds

This example illustrates various complexing agent formulations of compounds.

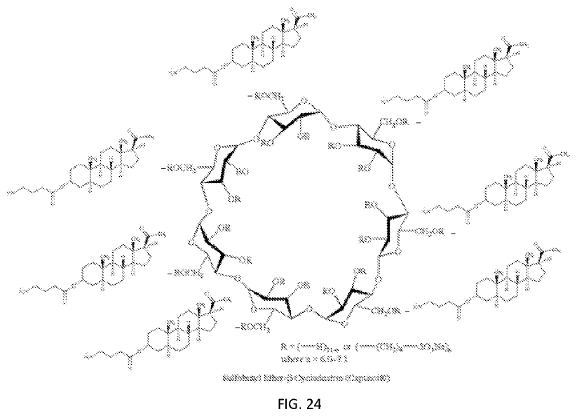

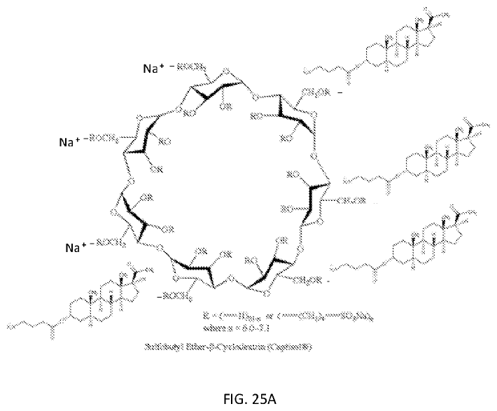

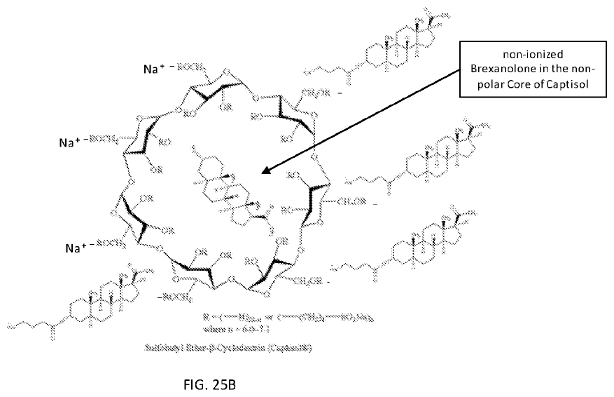

FIG. 24 illustrates a Brexanolone Prodrug Captisol Salt at 6-7:1 molar ratio of API to CapAcid, in which the brexanolone is conjugated to the γ-aminobutyric acid (GABA) having a protonated nitrogen atom.

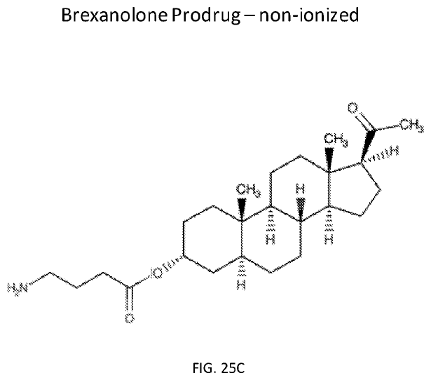

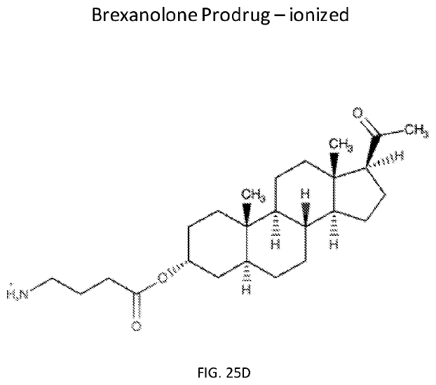

FIGS. 25A and 25B illustrate a Brexanolone Prodrug Captisol Salt at 4:1 molar ratio of API to CapAcid without (FIG. 25A) or with (FIG. 25B) an ionized brexanolone complexed to the non-polar pore of CapAcid, in which the brexanolone is conjugated to the γ-aminobutyric acid (GABA) having a protonated nitrogen atom.

FIG. 25C illustrates non-ionized Brexanolone Prodrug in which the brexanolone is conjugated to the γ-aminobutyric acid (GABA) with a deprotonated nitrogen atom. FIG. 25D illustrates ionized Brexanolone Prodrug in which the brexanolone is conjugated to the γ-aminobutyric acid (GABA) with a protonated nitrogen atom.

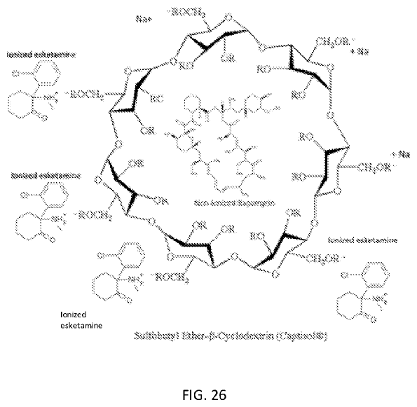

FIG. 26 illustrates an Esketamine-Captisol Salt at 4:1 molar ratio of Esketamine to CapAcid with non-ionized rapamycin complexed to the non-polar pore and cationic balance provided by sodium.

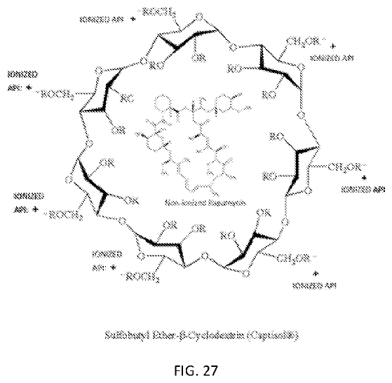

FIG. 27 illustrates a generalized ionized API-Captisol Salt at 6-7:1 molar ratio of API to CapAcid with non-ionized rapamycin complexed to the non-polar pore and rapamycin is used to represent generic a non-ionized API.

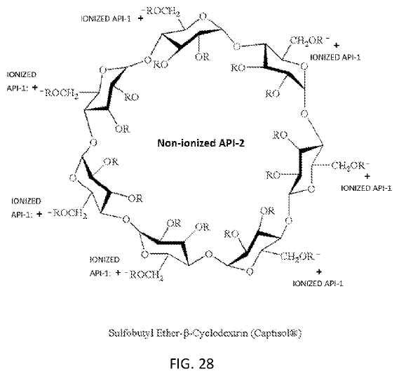

FIG. 28 illustrates an ionized API-1-Captisol Salt at 6-7:1 molar ratio of API-1 to CapAcid with non-ionized API-2 complexed to the non-polar pore.

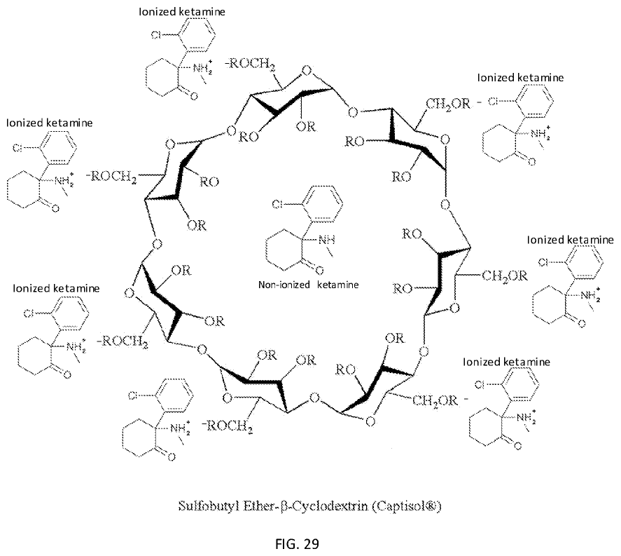

FIG. 29 illustrates a Ketamine-Captisol Salt at 6-7:1 molar ratio of ketamine to CapAcid with non-ionized ketamine complexed to the non-polar pore.

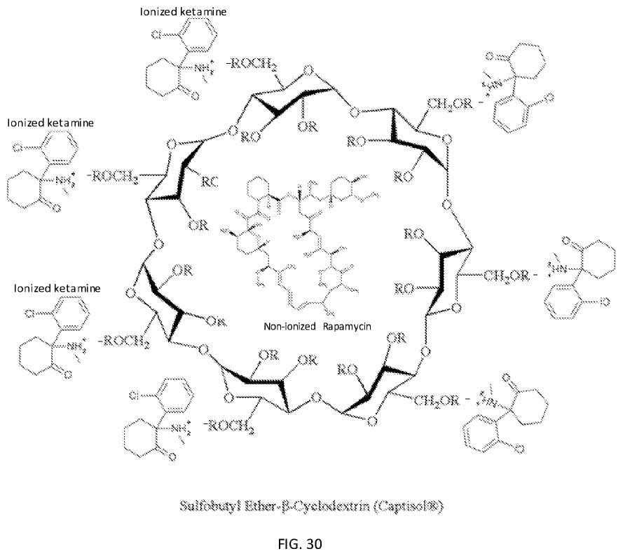

FIG. 30 illustrates a Ketamine-Captisol Salt at 6-7:1 molar ratio of ketamine to CapAcid with non-ionized rapamycin complexed to the non-polar pore.

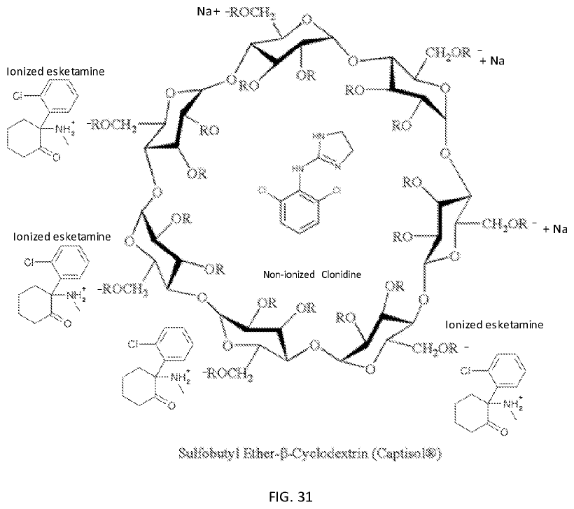

FIG. 31 shows an example of Esketamine-Captisol Salt at 4:1 ratio of API to CapAcid with non-ionized clonidine in complexing pore and cationic balance provided by sodium.

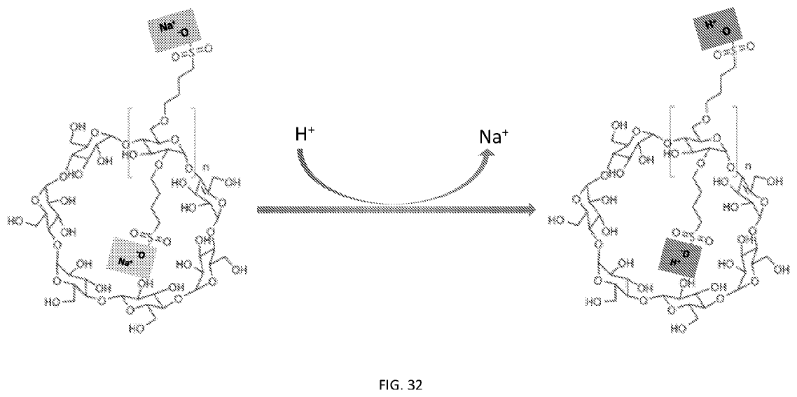

FIG. 32 illustrates a resin ion exchange process to replace the sodium ions occupying the acidic sites of complexing agent with protons. In some embodiments, the resin ion exchange process creates a Captisol-Ammonium salt. In some embodiments, the resin ion exchange process is used to raise and buffer the pH of the formulation to overcome the 5.6 pKa of stock Captisol, which is non-ideal as regards to tuning to subcutaneous tissue tolerance and comfort for extended release. In some embodiments, the resin ion exchange process creates a conjugate acid out of a cyclodextrin which is used to ionize ketamine thereby removing excess sodium and lower formulation osmolality to improve desiccation related subcutaneous tissue tolerance.

While preferred embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed in practicing the invention. It is intended that the following claims define the scope of the invention and that methods and structures within the scope of these claims and their equivalents be covered thereby.

Although the disclosure has been described with reference to the above examples, it will be understood that modifications and variations are encompassed within the spirit and scope of the disclosure. Accordingly, the disclosure is limited only by the following claims.

What is claimed is:

1. A pharmaceutical composition, comprising:
(i) a pharmaceutical compound, or an enantiomer, a mixture of enantiomers, or an isotopic variant thereof, wherein the pharmaceutical compound comprises a protonated nitrogen atom; and
(ii) a complexing agent, wherein the complexing agent is an acid-substituted cyclodextrin comprising a plurality of acidic functional groups, wherein the plurality of acidic functional groups comprise an acidic group which acts as a counterion for the protonated nitrogen atom of the pharmaceutical compound, wherein the pharmaceutical composition has a molar ratio of the complexing agent to the pharmaceutical compound that is from about 1:1 to about 1:4.

2. The pharmaceutical composition of claim 1, wherein the complexing agent comprises a substituted cyclodextrin.

3. The pharmaceutical composition of claim 2, wherein the complexing agent comprises a cyclodextrin substituted with at least one acidic functional group.

4. The pharmaceutical composition of claim 3, wherein the cyclodextrin is substituted with 3 to 8 acidic functional groups.

5. The pharmaceutical composition of claim 4, wherein the cyclodextrin is sulfobutylether-β-cyclodextrin

6. The pharmaceutical composition of claim 1, wherein the pharmaceutical composition has lower osmolality than a composition comprising a salt of the pharmaceutical compound and a salt of the complexing agent.

7. The pharmaceutical composition of claim 1, wherein the pharmaceutical is formulated for subcutaneous, intramuscular, sublingual, oral, rectal, transvaginal, or intranasal administration.

8. The pharmaceutical composition of claim 1, wherein the pharmaceutical composition has an osmolality of no more than about 850 mOsm/kg.

9. The pharmaceutical composition of claim 8, wherein the pharmaceutical composition has a pH of about 4 to about 7.

10. The pharmaceutical composition of claim 1, wherein the complexing agent is present in an amount of about 10 mg/mL to about 600 mg/mL.

11. The pharmaceutical composition of claim 1, wherein the complexing agent acts as the counterion to between 1 to 4 molecules of the pharmaceutical compound.

12. The pharmaceutical composition of claim 1, wherein the complexing agent further comprises a non-polar pore.

13. The pharmaceutical composition of claim 12, wherein the pharmaceutical composition further comprises an additional molar equivalent of the pharmaceutical compound, wherein the additional molar equivalent of the pharmaceutical compound is unionized and complexed to the non-polar pore.

14. The pharmaceutical composition of claim 1, wherein the ratio of complexing agent to the pharmaceutical compound is about 1:1.

15. The pharmaceutical composition of claim 1, wherein the ratio of complexing agent to the pharmaceutical compound is about 1:2.

16. The pharmaceutical composition of claim 1, wherein the ratio of complexing agent to the pharmaceutical compound is about 1:3.

17. The pharmaceutical composition of claim 1, wherein the ratio of complexing agent to the pharmaceutical compound is about 1:4.

18. The pharmaceutical composition of claim 1, wherein the pharmaceutical compound has a solubility of less than about 50 mg/ml as salt in an aqueous medium.

19. The pharmaceutical composition of claim 1, wherein the pharmaceutical compound has a solubility of less than about 10 mg/ml as salt in an aqueous medium.

20. The pharmaceutical composition of claim 1, wherein the pharmaceutical compound has a solubility of less than about 5 mg/ml as salt in an aqueous medium.

21. The pharmaceutical composition of claim 1, wherein the pharmaceutical compound has a solubility of less than about 0.5 mg/ml as salt in an aqueous medium.

22. The pharmaceutical composition of claim 1, wherein the pharmaceutical compound has a solubility of less than about 0.1 mg/ml as salt in an aqueous medium.

23. The pharmaceutical composition of claim 1, wherein a precipitate forms when an amount of the pharmaceutical compound is mixed with the pharmaceutical composition in an aqueous medium and becomes ionized after being mixed with the pharmaceutical composition.

24. The pharmaceutical composition of claim 23, wherein the amount of the pharmaceutical compound being mixed with the pharmaceutical composition is about 1 molar equivalent compared to the complexing agent of the pharmaceutical composition.

25. The pharmaceutical composition of claim 23, wherein the amount of the pharmaceutical compound being mixed with the pharmaceutical composition is about 2 molar equivalents compared to the complexing agent of the pharmaceutical composition.

26. The pharmaceutical composition of claim 23, wherein the amount of the pharmaceutical compound being mixed with the pharmaceutical composition is about 3 molar equivalents compared to the complexing agent of the pharmaceutical composition.

27. The pharmaceutical composition of claim 1, wherein the pharmaceutical compound comprises rotigotine, eletriptan, copanlisib, nafamostat (nafamostat mesylate), melevodopa, tigecycline, naloxone, amikacin, 1-amantadine, 2-amantadine, rimantadine, amifampridine, rapamycin, clonidine, or caspofungin.

28. A pharmaceutically acceptable salt of a compound pharmaceutical comprising:
(i) a pharmaceutical compound, or an enantiomer, a mixture of enantiomers, or an isotopic variant thereof; or a pharmaceutically acceptable salt, solvate or hydrate thereof, wherein the pharmaceutical compound comprises a protonated nitrogen atom; and
(ii) a conjugate base of a complexing agent comprising a plurality of acidic functional groups, wherein at least one acidic functional group of the plurality of acidic functional groups acts as a counterion of the pharmaceutical compound, wherein the molar ratio of the conjugate base of the complexing agent to the pharmaceutical compound is from about 1:1 to about 1:4.

29. The pharmaceutically acceptable salt of claim 28, wherein the salt is in a crystalline form, an amorphous form, a lyophilized powder, dissolved or suspended in an aqueous medium, or dissolved or suspended in an organic solvent.

30. The pharmaceutically acceptable salt of claim 28, wherein the complexing agent comprises a substituted cyclodextrin.

31. The pharmaceutically acceptable salt of claim 28, wherein the pharmaceutical compound comprises rotigotine, eletriptan, copanlisib, nafamostat (nafamostat mesylate), melevodopa, tigecycline, naloxone, amikacin, 1-amantadine, 2-amantadine, rimantadine, amifampridine, rapamycin, clonidine, rapamycin, clonidine, or caspofungin.

32. The pharmaceutically acceptable salt of claim 28, wherein the conjugate base of the complexing agent further comprises a non-polar pore.

33. The pharmaceutically acceptable salt of claim 32, wherein the pharmaceutically acceptable salt further comprises an additional molar equivalent of the pharmaceutical compound compared to the conjugate base of the complexing agent, wherein the additional molar equivalent of the pharmaceutical compound is unionized and complexed to the non-polar pore.

34. A pharmaceutically acceptable salt of a pharmaceutical compound having the formula:
[A]ₐ[B]
wherein:
A is a pharmaceutical compound comprising at least one basic nitrogen atom;
B is a complexing agent comprising a plurality of acidic functional groups; and
a is a number from 1-4, wherein the number is selected such that a portion, but not all, of the acidic functional groups of B act as a counterion to the basic nitrogen atom of A.

35. The pharmaceutically acceptable salt of claim 34, wherein the at least one basic nitrogen atom is comprised in a heterocycle.

36. The pharmaceutically acceptable salt of claim 33, wherein the pharmaceutical compound comprises only a single basic nitrogen atom.

37. The pharmaceutically acceptable salt of any one of claims 34-36, wherein a is equal to 1, 2, 3, or 4.

38. The pharmaceutically acceptable salt of any one of claims 34-37, wherein the pharmaceutical compound comprises two or more basic nitrogen atoms.

39. The pharmaceutically acceptable salt of any one of claims 34-38, wherein the complexing agent is a cyclodextrin.

40. The pharmaceutically acceptable salt of any one of claims 34-39, wherein the complexing agent is a compound of Formula (I): 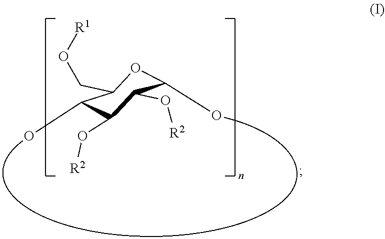
wherein:
each R¹ is independently H or optionally substituted alkyl; wherein at least one R¹ is substituted with an acidic functional group;
each R² is independently H or optionally substituted alkyl; and
n is 6, 7, or 8;
or a stereoisomer, a mixture of stereoisomers, or an isotopic variant thereof; or a pharmaceutically acceptable salt, solvate or hydrate thereof.

41. The pharmaceutically acceptable salt of any one of claims 34-40, wherein the complexing agent is SBEBCD.

42. The pharmaceutically acceptable salt of any one of claims 34-41, wherein the pharmaceutical compound is rotigotine, eletriptan, copanlisib, nafamostat (nafamostat mesylate), melevodopa, tigecycline, naloxone, amikacin, 1-amantadine, 2-amantadine, rimantadine, amifampridine, rapamycin, clonidine, or caspofungin.

43. The pharmaceutically acceptable salt of claim 34, wherein the complexing agent further comprises a non-polar pore.

44. The pharmaceutically acceptable salt of claim 43, wherein the pharmaceutically acceptable salt further comprises an additional molar equivalent of the pharmaceutical compound compared to the complexing agent, wherein the additional molar equivalent of the pharmaceutical compound is unionized and complexed to the non-polar pore.

45. A pharmaceutical composition, comprising:
(i) a pharmaceutical compound, or an enantiomer, a mixture of enantiomers, or an isotopic variant thereof, wherein the pharmaceutical compound comprises a protonated nitrogen atom;
(ii) a complexing agent, wherein the complexing agent comprises a plurality of acidic functional groups, wherein the plurality of acidic functional groups comprise a conjugate base of an acid which acts as a counterion for the protonated nitrogen atom of the pharmaceutical compound, wherein the pharmaceutical composition has a molar ratio of the complexing agent to the pharmaceutical compound that is from about 1:1 to about 1:4; and
(iii) an additional molar equivalent of the pharmaceutical compound, wherein the additional molar equivalent of the pharmaceutical compound is unionized.

46. The pharmaceutical composition of claim 45, wherein the pharmaceutical compound comprises rotigotine, eletriptan, copanlisib, nafamostat (nafamostat mesylate), melevodopa, tigecycline, naloxone, amikacin, 1-amantadine, 2-amantadine, rimantadine, amifampridine, rapamycin, clonidine, or caspofungin.

47. The pharmaceutical composition of claim 45, wherein the complexing agent comprises a substituted cyclodextrin.

48. The pharmaceutical composition of claim 47, wherein the complexing agent comprises a cyclodextrin substituted with at least one acidic functional group.

49. The pharmaceutical composition of claim 48, wherein the cyclodextrin is substituted with 3 to 8 acidic functional groups.

50. The pharmaceutical composition of claim 47, wherein the cyclodextrin is sulfobutylether-β-cyclodextrin

51. The pharmaceutical composition of claim 45, wherein the pharmaceutical composition has lower osmolality than a composition comprising a salt of the pharmaceutical compound and a salt of the complexing agent.

52. The pharmaceutical composition of claim 45, wherein the pharmaceutical is formulated for subcutaneous, intramuscular, sublingual, oral, rectal, transvaginal, or intranasal administration.

53. The pharmaceutical composition of claim 45, wherein the pharmaceutical composition has an osmolality of no more than about 850 mOsm/kg.

54. The pharmaceutical composition of claim 53, wherein the pharmaceutical composition has a pH of about 4 to about 7.

55. The pharmaceutical composition of claim 45, wherein the complexing agent is present in an amount of about 10 mg/mL to about 600 mg/mL.

56. The pharmaceutical composition of claim 45, wherein the complexing agent acts as the counterion to between 1 to 4 molecules of the pharmaceutical compound.

57. The pharmaceutical composition of claim 45, wherein the complexing agent further comprises a non-polar pore.

58. The pharmaceutical composition of claim 57, wherein the additional molar equivalent of the unionized pharmaceutical compound is complexed to the non-polar pore.

59. The pharmaceutical composition of claim 45, wherein the molar ratio of complexing agent to the pharmaceutical compound is about 1:1.

60. The pharmaceutical composition of claim 45, wherein the molar ratio of complexing agent to the pharmaceutical compound is about 1:2.

61. The pharmaceutical composition of claim 45, wherein the molar ratio of complexing agent to the pharmaceutical compound is about 1:3.

62. The pharmaceutical composition of claim 45, wherein the molar ratio of complexing agent to the pharmaceutical compound is about 1:4.

63. The pharmaceutical composition of claim 45, wherein the pharmaceutical compound has a solubility of less than about 50 mg/ml as salt in an aqueous medium.

64. The pharmaceutical composition of claim 45, wherein the pharmaceutical compound has a solubility of less than about 10 mg/ml as salt in an aqueous medium.

65. The pharmaceutical composition of claim 45, wherein the pharmaceutical compound has a solubility of less than about 5 mg/ml as salt in an aqueous medium.

66. The pharmaceutical composition of claim 45, wherein the pharmaceutical compound has a solubility of less than about 0.5 mg/ml as salt in an aqueous medium.

67. The pharmaceutical composition of claim 45, wherein the pharmaceutical compound has a solubility of less than about 0.1 mg/ml as salt in an aqueous medium.

68. The pharmaceutical composition of claim 45, wherein a precipitate forms when an amount of the pharmaceutical compound is mixed with the pharmaceutical composition in an aqueous medium and becomes ionized after being mixed with the pharmaceutical composition.

69. The pharmaceutical composition of claim 68, wherein the amount of the pharmaceutical compound being mixed with the pharmaceutical composition is about 1 molar equivalent compared to the complexing agent of the pharmaceutical composition.

70. The pharmaceutical composition of claim 68, wherein the amount of the pharmaceutical compound being mixed with the pharmaceutical composition is about 2 molar equivalents compared to the complexing agent of the pharmaceutical composition.

71. The pharmaceutical composition of claim 68, wherein the amount of the pharmaceutical compound being mixed with the pharmaceutical composition is about 3 molar equivalents compared to the complexing agent of the pharmaceutical composition.

72. A method of preparing a pharmaceutical composition, comprising combining in a suitable liquid medium:
a) a free base form of a pharmaceutical compound, or an enantiomer, a mixture of enantiomers, or an isotopic variant thereof, wherein the pharmaceutical compound comprises at least one basic nitrogen atom; and
b) a free acid form of a complexing agent comprising at least one acidic functional group, wherein the molar ratio of the complexing agent to the pharmaceutical compound is from about 1:1 to about 1:4.

73. The method of claim 72, further comprising the step of adding an additional molar equivalent of the free base form of the pharmaceutical compound to the suitable liquid medium.

74. The method of claim 73, wherein the adding the additional molar equivalent of the free base form of the pharmaceutical compound occurs after removing the liquid medium from the pharmaceutical composition.

75. The method of claim 73, wherein the additional molar equivalent of the free base form of the pharmaceutical compound is unionized after being added.

76. The method of claim 72, wherein a precipitate forms after the additional molar equivalent of the free base form of the pharmaceutical compound is added and becomes ionized.

77. The method of claim 76, wherein the additional molar equivalent of the free base form of the pharmaceutical compound is about 1 molar equivalent compared to the complexing agent.

78. The method of claim 76, wherein the additional molar equivalent of the free base form of the pharmaceutical compound is about 2 molar equivalents compared to the complexing agent.

79. The method of claim 76, wherein the additional molar equivalent of the free base form of the pharmaceutical compound is about 3 molar equivalents compared to the complexing agent.

80. The method of claim 72, wherein the pharmaceutical compound has a solubility of less than about 50 mg/ml as salt in an aqueous medium.

81. The method of claim 72, wherein the pharmaceutical compound has a solubility of less than about 10 mg/ml as salt in an aqueous medium.

82. The method of claim 72, wherein the pharmaceutical compound has a solubility of less than about 5 mg/ml as salt in an aqueous medium.

83. The method of claim 72, wherein the pharmaceutical compound has a solubility of less than about 0.5 mg/ml as salt in an aqueous medium.

84. The method of claim 72, wherein the pharmaceutical compound has a solubility of less than about 0.1 mg/ml as salt in an aqueous medium.

85. The method of claim 72, wherein the complexing agent is sulfobutylether-β-cyclodextrin.

86. The method of claim 72, further comprising subjecting the pharmaceutical composition to an ion exchange process to generate a conjugate acid form of the complexing agent.

87. The method of claim 86, wherein the ion exchange process comprises a resin ion exchange process.

88. A pharmaceutical composition, comprising:
(i) a pharmaceutical compound, or an enantiomer, a mixture of enantiomers, or an isotopic variant thereof, wherein the pharmaceutical compound comprises a prodrug comprising an unionized substance conjugated to a chemical entity, wherein the chemical entity comprises a protonated nitrogen atom; and
(ii) a complexing agent, wherein the complexing agent is an acid-substituted cyclodextrin comprising a plurality of acidic functional groups, wherein the plurality of acidic functional groups comprise an acidic group which acts as a counterion for the protonated nitrogen atom of the pharmaceutical compound.

89. The pharmaceutical composition of claim 88, wherein the complexing agent comprises a substituted cyclodextrin.

90. The pharmaceutical composition of claim 89, wherein the complexing agent comprises a cyclodextrin substituted with at least one acidic functional group.

91. The pharmaceutical composition of claim 90, wherein the cyclodextrin is substituted with 3 to 8 acidic functional groups.

92. The pharmaceutical composition of claim 91, wherein the cyclodextrin is sulfobutylether-β-cyclodextrin

93. The pharmaceutical composition of claim 88, wherein the pharmaceutical composition has lower osmolality than (i) a composition comprising a salt of the pharmaceutical compound; or (ii) a composition comprising the pharmaceutical compound in freebase form that is complexing to a non-polar pore of the complexing agent, wherein the pharmaceutical compound has the same concentration in the pharmaceutical composition, the composition comprising the salt of the pharmaceutical compound, and the composition comprising the pharmaceutical compound in freebase form.

94. The pharmaceutical composition of claim 88, wherein the pharmaceutical composition is formulated for subcutaneous, intramuscular, sublingual, oral, rectal, transvaginal, or intranasal administration.

95. The pharmaceutical composition of claim 88, wherein the pharmaceutical composition has a molar ratio of complexing agent to the pharmaceutical compound that is from about 1:4 to about 1:10.

96. The pharmaceutical composition of claim 88, wherein the pharmaceutical composition has an osmolality of no more than about 850 mOsm/kg.

97. The pharmaceutical composition of claim 96, wherein the pharmaceutical composition has a pH of about 4 to about 7.

98. The pharmaceutical composition of claim 88, wherein the complexing agent is present in an amount of about 10 mg/mL to about 600 mg/mL.

99. The pharmaceutical composition of claim 88, wherein the pharmaceutical composition further comprises about 0.1 to about 20 molar equivalents of the unionized substance compared to the complexing agent.

100. The pharmaceutical composition of claim 88, wherein the unionized substance comprises brexanolone.

101. The pharmaceutical composition of claim 88, wherein the chemical entity comprises γ-aminobutyric acid (GABA).

102. The pharmaceutical composition of claim 99, wherein the complexing agent further comprises a non-polar pore.

103. The pharmaceutical composition of claim 102, wherein the about 0.1 to about 20 molar equivalents of the unionized substance is complexed to the non-polar pore.

104. The pharmaceutical composition of claim 88, wherein the unionized substance is cleaved off from the GABA and released from the pharmaceutical composition after the pharmaceutical composition is administered to an individual.

105. A pharmaceutical composition, comprising:
(i) a first pharmaceutical compound, or an enantiomer, a mixture of enantiomers, or an isotopic variant thereof, wherein the pharmaceutical compound comprises a protonated nitrogen atom;
(ii) a complexing agent, wherein the complexing agent comprises a plurality of acidic functional groups, wherein the plurality of acidic functional groups comprise a conjugate base of an acid which acts as a counterion for the protonated nitrogen atom of the first pharmaceutical compound; and
(iii) a second pharmaceutical compound, wherein the second pharmaceutical compound is unionized.

106. The pharmaceutical composition of claim 105, wherein the complexing agent is sulfobutylether-β-cyclodextrin.

107. The pharmaceutical composition of claim 105, wherein the first pharmaceutical compound comprises ketamine.

108. The pharmaceutical composition of claim 105, wherein the second pharmaceutical compound comprises rapamycin.

109. The pharmaceutical composition of claim 105, wherein the first pharmaceutical compound comprises ketamine and the second pharmaceutical compound comprises rapamycin.

110. The pharmaceutical composition of claim 105, wherein the first pharmaceutical compound comprises no ketamine and the second pharmaceutical compound comprises rapamycin.

111. The pharmaceutical composition of claim 105, wherein the complexing agent comprises a substituted cyclodextrin.

112. The pharmaceutical composition of claim 111, wherein the complexing agent comprises a cyclodextrin substituted with at least one acidic functional group.

113. The pharmaceutical composition of claim 112, wherein the cyclodextrin is substituted with 3 to 8 acidic functional groups.

114. The pharmaceutical composition of claim 113, wherein the cyclodextrin is sulfobutylether-β-cyclodextrin

115. The pharmaceutical composition of claim 105, wherein the pharmaceutical composition has lower osmolality than a composition comprising a salt of the first pharmaceutical compound, a salt of the complexing agent and a salt of the second pharmaceutical compound.

116. The pharmaceutical composition of claim 105, wherein the pharmaceutical composition is formulated for subcutaneous, intramuscular, sublingual, oral, rectal, transvaginal, or intranasal administration.

117. The pharmaceutical composition of claim 105, wherein the pharmaceutical composition has a molar ratio of the complexing agent to the first pharmaceutical compound that is from about 1:4 to about 1:10.

118. The pharmaceutical composition of claim 105, wherein the pharmaceutical composition has a molar ratio of the complexing agent to the second pharmaceutical compound that is about 1:1.

119. The pharmaceutical composition of claim 105, wherein the pharmaceutical composition has an osmolality of no more than about 850 mOsm/kg.

120. The pharmaceutical composition of claim 119, wherein the pharmaceutical composition has a pH of about 4 to about 7.

121. The pharmaceutical composition of claim 105, wherein the complexing agent is present in an amount of about 10 mg/mL to about 600 mg/mL.

122. The pharmaceutical composition of claim 105, further comprising an amount of the first pharmaceutical compound in an unionized form.

123. The pharmaceutical composition of any one of claims 105-122, wherein the complexing agent comprises a non-polar pore.

124. The pharmaceutical composition of claim 123, wherein the second pharmaceutical compound is complexed to the non-polar pore.

125. The pharmaceutical composition of claim 123, wherein the first pharmaceutical compound in the unionized form is complexed to the non-polar pore.

126. The pharmaceutical composition of claim 105, wherein the second pharmaceutical compound comprises clonidine.

127. The pharmaceutical composition of claim 105, wherein the first pharmaceutical compound comprises ketamine and the second pharmaceutical compound comprises clonidine.

128. The pharmaceutical composition of claim 105, wherein the first pharmaceutical compound comprises no ketamine and the second pharmaceutical compound comprises clonidine.
